(19) United States
(12) Patent Application Publication
(10) Pub. No.: US 2021/0009560 A1
(43) Pub. Date: Jan. 14, 2021

(54) CRYSTALLINE FORMS OF MODULATORS OF CFTR

(71) Applicant: Vertex Pharmaceuticals Incorporated, Boston, MA (US)

(72) Inventors: Elaine Chungmin LEE, Winchester, MA (US); Mettachit NAVAMAL, Belmont, MA (US); Kwame Wiredu NTI-ADDAE, Tewksbury, MA (US); Yi SHI, Natick, MA (US); Muna SHRESTHA, Belmont, MA (US); Beili ZHANG, San Diego, CA (US); Carl ZWICKER, Wakefield, MA (US)

(21) Appl. No.: 17/043,217

(22) PCT Filed: Mar. 29, 2019
(86) PCT No.: PCT/US2019/024890

§ 371 Date: Sep. 29, 2020

Related U.S. Application Data
(60) Provisional application No. 62/650,782, filed on Mar. 30, 2018.

(51) Int. Cl. C07D 401/14; C07D 401/04; A61P 11/00

(52) U.S. Cl. CPC C07D 401/14; C07B 2200/13; A61P 11/00; C07D 401/04

(57) ABSTRACT

Crystalline Forms of Compound (I); crystalline Forms of Compound (II) and crystalline forms of pharmaceutically acceptable salts of any of the foregoing are disclosed. Pharmaceutical compositions comprising the same, methods of treating cystic fibrosis using the same, and methods for making the same are also disclosed.

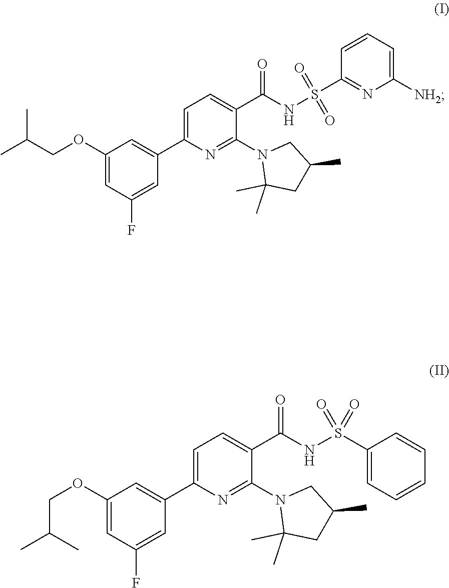 [SMILES: CC(C)COc1cc(F)cc(-c2ccc(C(=O)NS(=O)(=O)c3cccc(N)n3)c(N3C[C@@H](C)CC3(C)C)n2)c1 ; CC(C)COc1cc(F)cc(-c2ccc(C(=O)NS(=O)(=O)c3ccccc3)c(N3C[C@@H](C)CC3(C)C)n2)c1]

This application claims priority to U.S. provisional application 62/650,782, filed Mar. 30, 2018, the disclosure of which is incorporated herein by reference in its entirety.

Disclosed herein are crystalline forms of Compound (I), crystalline forms of Compound (II), crystalline forms of pharmaceutically acceptable salts of any of the foregoing, and crystalline forms of deuterated analogs of any of the foregoing, which are modulators of Cystic Fibrosis Transmembrane Conductance Regulator (CFTR), compositions comprising the same, methods of using the same, and processes for making the same.

Cystic fibrosis (CF) is a recessive genetic disease that affects approximately 70,000 children and adults worldwide. Despite progress in the treatment of CF, there is no cure.

In patients with CF, mutations in CFTR endogenously expressed in respiratory epithelia lead to reduced apical anion secretion causing an imbalance in ion and fluid transport. The resulting decrease in anion transport contributes to enhanced mucus accumulation in the lung and accompanying microbial infections that ultimately cause death in CF patients. In addition to respiratory disease, CF patients typically suffer from gastrointestinal problems and pancreatic insufficiency that, if left untreated, result in death. In addition, the majority of males with cystic fibrosis are infertile, and fertility is reduced among females with cystic fibrosis.

Sequence analysis of the CFTR gene has revealed a variety of disease causing mutations (Cutting, G. R. et al. (1990) Nature 346:366-369; Dean, M. et al. (1990) Cell 61:863:870; and Kerem, B-S. et al. (1989) Science 245:1073-1080; Kerem, B-S et al. (1990) Proc. Natl. Acad. Sci. USA 87:8447-8451). To date, greater than 2000 mutations in the CF gene have been identified; currently, the CFTR2 database contains information on only 322 of these identified mutations, with sufficient evidence to define 281 mutations as disease causing. The most prevalent disease-causing mutation is a deletion of phenylalanine at position 508 of the CFTR amino acid sequence, and is commonly referred to as the F508del mutation. This mutation occurs in approximately 70% of the cases of cystic fibrosis and is associated with severe disease.

The deletion of residue 508 in CFTR prevents the nascent protein from folding correctly. This results in the inability of the mutant protein to exit the endoplasmic reticulum (ER) and traffic to the plasma membrane. As a result, the number of CFTR channels for anion transport present in the membrane is far less than observed in cells expressing wild-type CFTR, i.e., CFTR having no mutations. In addition to impaired trafficking, the mutation results in defective channel gating. Together, the reduced number of channels in the membrane and the defective gating lead to reduced anion and fluid transport across epithelia. (Quinton, P. M. (1990), FASEB J. 4: 2709-2727). The channels that are defective because of the F508del mutation are still functional, albeit less functional than wild-type CFTR channels. (Dalemans et al. (1991), Nature Lond. 354: 526-528; Pasyk and Foskett (1995), J. Cell. Biochem. 270: 12347-50). In addition to F508del, other disease causing mutations in CFTR that result in defective trafficking, synthesis, and/or channel gating could be up- or down-regulated to alter anion secretion and modify disease progression and/or severity.

CFTR is a cAMP/ATP-mediated anion channel that is expressed in a variety of cell types, including absorptive and secretory epithelia cells, where it regulates anion flux across the membrane, as well as the activity of other ion channels and proteins. In epithelial cells, normal functioning of CFTR is critical for the maintenance of electrolyte transport throughout the body, including respiratory and digestive tissue. CFTR is composed of approximately 1480 amino acids that encode a protein which is made up of a tandem repeat of transmembrane domains, each containing six transmembrane helices and a nucleotide binding domain. The two transmembrane domains are linked by a large, polar, regulatory (R)-domain with multiple phosphorylation sites that regulate channel activity and cellular trafficking.

Chloride transport takes place by the coordinated activity of ENaC and CFTR present on the apical membrane and the Na⁺-K⁺-ATPase pump and Cl− channels expressed on the basolateral surface of the cell. Secondary active transport of chloride from the luminal side leads to the accumulation of intracellular chloride, which can then passively leave the cell via Cl⁻ channels, resulting in a vectorial transport. Arrangement of Na⁺/2Cl⁻/K⁺ co-transporter, Na⁺-K⁺-ATPase pump and the basolateral membrane K⁺ channels on the basolateral surface and CFTR on the luminal side coordinate the secretion of chloride via CFTR on the luminal side. Because water is probably never actively transported itself, its flow across epithelia depends on tiny transepithelial osmotic gradients generated by the bulk flow of sodium and chloride.

Compound (I) and pharmaceutically acceptable salts thereof are potent CFTR modulators. Compound (I) is (S)—N-((6-aminopyridin-2-yl)sulfonyl)-6-(3-fluoro-5-isobutoxyphenyl)-2-(2,2,4-trimethylpyrrolidin-1-yl)nicotinamide and has the following structure:

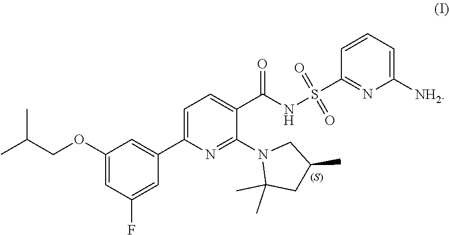

Compound (II) and pharmaceutically acceptable salts thereof are potent CFTR modulators. Compound (II) is (S)-6-(3-fluoro-5-isobutoxyphenyl)-N-(phenylsulfonyl)-2-(2,2,4-trimethylpyrrolidin-1-yl)nicotinamide and has the following structure:

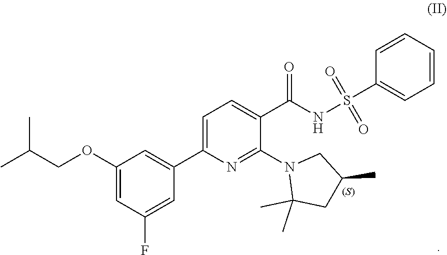

Crystalline forms are of interest in the pharmaceutical industry, where the control of the crystalline form(s) of the active ingredient may be desirable or even required. Reproducible processes for producing a compound with a particular crystalline form in high purity may be desirable for compounds intended to be used in pharmaceuticals, as different crystalline forms may possess different properties. For example, different crystalline forms may possess different chemical, physical, and/or pharmaceutical properties. In some embodiments, one or more crystalline forms disclosed herein may exhibit a higher level of purity, chemical stability, and/or physical stability compared to the forms produced in WO 2016/057572. Certain crystalline forms (e.g., crystalline Form A of Compound I and Form A2 of Compound II) may exhibit lower hygroscopicity than the forms produced in WO 2016/057572. Thus, the crystalline forms of this disclosure may provide advantages over the forms produced in WO 2016/057572 during drug substance manufacturing, storage, and handling.

Accordingly, there is a need for novel crystalline forms of compounds useful for treatment of CFTR mediated diseases.

Disclosed herein are novel crystalline forms of Compound (I), crystalline forms of Compound (II), crystalline forms of pharmaceutically acceptable salts of any of the foregoing, and crystalline forms of deuterated analogs of any of the foregoing, compositions comprising the same, and methods of using and making the same.

### BRIEF DESCRIPTION OF THE DRAWINGS

FIG. 1 shows an X-ray powder diffractogram of crystalline Form A of Compound (I).

FIG. 2 shows a differential scanning calorimetry (DSC) plot of crystalline Form A of Compound (I).

FIG. 3 shows a thermogravimetric analysis (TGA) plot of crystalline Form A of Compound (I).

FIG. 4 shows a ball and stick plot of crystalline Form A of Compound (I).

FIG. 5 shows an X-ray powder diffractogram of crystalline Form B of Compound (I).

FIG. 6 shows a DSC plot of crystalline Form B of Compound (I).

FIG. 7 shows a TGA plot of crystalline Form B of Compound (I).

FIG. 8 shows an X-ray powder diffractogram of crystalline Form H of Compound (I).

FIG. 9 shows a DSC plot of crystalline Form H of Compound (I).

FIG. 10 shows a TGA plot of crystalline Form H of Compound (I).

FIG. 11 shows a ball and stick plot of crystalline Form H of Compound (I).

FIG. 12 shows an X-ray powder diffractogram of crystalline Form S of Compound (I).

FIG. 13 shows a DSC plot of crystalline Form S of Compound (I).

FIG. 14 shows a TGA plot of crystalline Form S of Compound (I).

FIG. 15 shows an X-ray powder diffractogram of crystalline Form MS of Compound (I).

FIG. 16 shows a DSC plot of crystalline Form MS of Compound (I).

FIG. 17 shows a TGA plot of crystalline Form MS of Compound (I).

FIG. 18 shows an X-ray powder diffractogram of crystalline Form A2 of Compound (II).

FIG. 19 shows a DSC plot of crystalline Form A2 of Compound (II).

FIG. 20 shows a TGA plot of crystalline Form A2 of Compound (II).

FIG. 21 shows a ball and stick plot of crystalline Form A2 of Compound (II).

FIG. 22 shows an X-ray powder diffractogram of crystalline Form IP of Compound (II).

FIG. 23 shows a DSC plot of crystalline Form IP of Compound (II).

FIG. 24 shows a TGA plot of crystalline Form IP of Compound (II).

FIG. 25 shows a ball and stick plot of crystalline Form IP of Compound (II).

FIG. 26 shows an X-ray powder diffractogram of crystalline Form NPR of Compound (II).

FIG. 27 shows an X-ray powder diffractogram of crystalline Form 2B of Compound (II).

FIG. 28 shows a DSC plot of crystalline Form 2B of Compound (II).

FIG. 29 shows a TGA plot of crystalline Form 2B of Compound (II).

FIG. 30 shows an X-ray powder diffractogram of crystalline Form MP of Compound (II).

FIG. 31 shows a DSC plot of crystalline Form MP of Compound (II).

FIG. 32 shows a TGA plot of crystalline Form MP of Compound (II).

FIG. 33 shows an X-ray powder diffractogram of crystalline Form NP of Compound (II).

FIG. 34 shows a DSC plot of crystalline Form NP of Compound (II).

FIG. 35 shows a TGA plot of crystalline Form NP of Compound (II).

FIG. 36 shows an X-ray powder diffractogram of crystalline Form EE of Compound (II).

FIG. 37 shows a DSC plot of crystalline Form EE of Compound (II).

FIG. 38 shows a TGA plot of crystalline Form EE of Compound (II).

FIG. 39 shows an X-ray powder diffractogram of crystalline Form E of Compound (II).

FIG. 40 shows an X-ray powder diffractogram of crystalline Form T of Compound (II).

FIG. 41 shows an X-ray powder diffractogram of crystalline Form AC of Compound (II).

FIG. 42 shows a DSC plot of crystalline Form AC of Compound (II).

FIG. 43 shows a TGA plot of crystalline Form AC of Compound (II).

FIG. 44 is a representative list of CFTR genetic mutations.

FIG. 45 shows an X-ray powder diffractogram of crystalline Forms C of Compound (I).

FIG. 46 shows a DSC trace for crystalline Forms C of Compound (I).

FIG. 47 shows a TGA plot for crystalline Forms C of Compound (I).

FIG. 48 shows an X-ray powder diffractogram of crystalline Forms FC of Compound (I).

FIG. 49 shows a DSC trace for crystalline Forms FC of Compound (I).

FIG. 50 shows a TGA plot for crystalline Forms FC of Compound (I).

FIG. 51 shows an X-ray powder diffractogram of amorphous Compound (I).

FIG. 52 shows a DSC trace for amorphous Compound (I).

FIG. 53 shows a TGA plot for amorphous Compound (I).

FIG. 54 shows an X-ray powder diffractogram of amorphous Compound (II).

FIG. 55 shows a DSC trace for amorphous Compound (II).

FIG. 56 shows a TGA plot for amorphous Compound (II).

### DEFINITIONS

As used herein, “Compound (I)” refers to a compound having a chemical name (S)—N-((6-aminopyridin-2-yl)sulfonyl)-6-(3-fluoro-5-isobutoxyphenyl)-2-(2,2,4-trimethylpyrrolidin-1-yl)nicotinamide, which has the following structure:

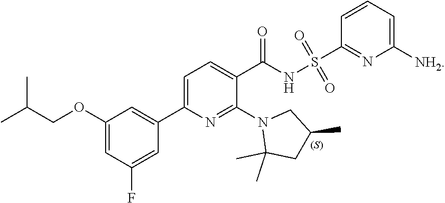

As used herein, “Compound (II)” refers to a compound having a chemical name (S)-6-(3-fluoro-5-isobutoxyphenyl)-N-(phenylsulfonyl)-2-(2,2,4-trimethylpyrrolidin-1-yl)nicotinamide, which has the following structure:

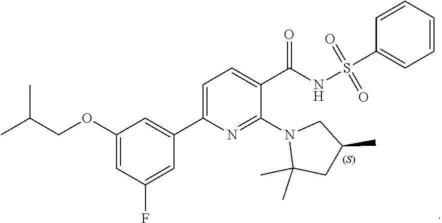

As used herein, the term “pharmaceutically acceptable salt” refers to a salt form of a compound of this disclosure wherein the salt is nontoxic. Pharmaceutically acceptable salts of Compound (I) and pharmaceutically acceptable salts of Compound (II) of this disclosure include those derived from suitable inorganic and organic acids and bases. Pharmaceutically acceptable salts are well known in the art. For example, S. M. Berge, et al. describe pharmaceutically acceptable salts in detail in J. Pharmaceutical Sciences, 1977, 66, 1-19.

Suitable pharmaceutically acceptable salts are, for example, those disclosed in S. M. Berge, et al. J. Pharmaceutical Sciences, 1977, 66, 1-19. For example, Table 1 of that article provides the following pharmaceutically acceptable salts:

| | | |
|---|---|---|
| Acetate | Iodide | Benzathine |
| Benzenesulfonate | Isethionate | Chloroprocaine |
| Benzoate | Lactate | Choline |
| Bicarbonate | Lactobionate | Diethanolamine |
| Bitartrate | Malate | Ethylenediamine |
| Bromide | Maleate | Meglumine |
| Calcium edetate | Mandelate | Procaine |
| Camsylate | Mesylate | Aluminum |
| Carbonate | Methylbromide | Calcium |
| Chloride | Methylnitrate | Lithium |
| Citrate | Methylsulfate | Magnesium |
| Dihydrochloride | Mucate | Potassium |
| Edetate | Napsylate | Sodium |
| Edisylate | Nitrate | Zinc |
| Estolate | Pamoate (Embonate) | |
| Esylate | Pantothenate | |
| Fumarate | Phosphate/diphosphate | |
| Gluceptate | Polygalacturonate | |
| Gluconate | Salicylate | |
| Glutamate | Stearate | |
| Glycollylarsanilate | Subacetate | |
| Hexylresorcinate | Succinate | |
| Hydrabamine | Sulfate | |
| Hydrobromide | Tannate | |
| Hydrochloride | Tartrate | |
| Hydroxynaphthoate | Teociate | |
| | Triethiodide | |

Non-limiting examples of pharmaceutically acceptable salts derived from appropriate acids include: salts formed with inorganic acids, such as hydrochloric acid, hydrobromic acid, phosphoric acid, sulfuric acid, or perchloric acid; salts formed with organic acids, such as acetic acid, oxalic acid, maleic acid, tartaric acid, citric acid, succinic acid or malonic acid; and salts formed by using other methods used in the art, such as ion exchange. Non-limiting examples of pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, and valerate salts. Pharmaceutically acceptable salts derived from appropriate bases include alkali metal, alkaline earth metal, ammonium, and N⁺(C₁₋₄ alkyl)₄ salts. This disclosure also envisions the quaternization of any basic nitrogen-containing groups of the compounds disclosed herein. Suitable non-limiting examples of alkali and alkaline earth metal salts include sodium, lithium, potassium, calcium, and magnesium. Further non-limiting examples of pharmaceutically acceptable salts include ammonium, quaternary ammonium, and amine cations formed using counterions such as halide, hydroxide, carboxylate, sulfate, phosphate, nitrate, lower alkyl sulfonate and aryl sulfonate. Other suitable, non-limiting examples of pharmaceutically acceptable salts include besylate and glucosamine salts.

As used herein, the term “ambient conditions” means room temperature, open air condition and uncontrolled humidity condition.

As used herein, the terms “crystal form,” “crystalline form,” and “Form” interchangeably refer to a crystal structure (or polymorph) having a particular molecular packing arrangement in the crystal lattice. Crystalline forms can be identified and distinguished from each other by one or more characterization techniques including, for example, X-ray powder diffraction (XRPD), single crystal X-ray diffraction, differential scanning calorimetry (DSC), dynamic vapor sorption (DVS), and/or thermogravimetric analysis (TGA). Accordingly, as used herein, the terms “crystalline Form [X] of Compound ([Y])” and “crystalline Form [C] of a [pharmaceutically acceptable] salt of Compound ([Y])” refer to unique crystalline forms that can be identified and distinguished from each other by one or more characterization techniques including, for example, X-ray powder diffraction (XRPD), single crystal X-ray diffraction, differential scanning calorimetry (DSC), dynamic vapor sorption (DVS), and/or thermogravimetric analysis (TGA). In some embodiments, the novel crystalline forms are characterized by an X-ray powder diffractogram having one or more signals at one or more specified two-theta values (°2θ).

As used herein, the terms “solvate” and “pseudo-polymorph” interchangeably refer to a crystal form comprising one or more molecules of a compound of the present disclosure and, incorporated into the crystal lattice, one or more molecules of a solvent or solvents in stoichiometric or nonstoichiometric amounts. When the solvent is water, the solvate is referred to as a “hydrate”.

As used herein, the term “XRPD” refers to the analytical characterization method of X-ray powder diffraction. XRPD patterns can be recorded at ambient conditions in transmission or reflection geometry using a diffractometer.

As used herein, the terms “X-ray powder diffractogram,” “X-ray powder diffraction pattern,” “XRPD pattern” interchangeably refer to an experimentally obtained pattern plotting signal positions (on the abscissa) versus signal intensities (on the ordinate). For an amorphous material, an X-ray powder diffractogram may include one or more broad signals; and for a crystalline material, an X-ray powder diffractogram may include one or more signals, each identified by its angular value as measured in degrees 2θ (°2θ), depicted on the abscissa of an X-ray powder diffractogram, which may be expressed as “a signal at . . . degrees two-theta,” “a signal at [a] two-theta value(s) of . . . ” and/or “a signal at at least . . . two-theta value(s) chosen from . . . .”

A “signal” or “peak” as used herein refers to a point in the XRPD pattern where the intensity as measured in counts is at a local. One of ordinary skill in the art would recognize that one or more signals (or peaks) in an XRPD pattern may overlap and may, for example, not be apparent to the naked eye. Indeed, one of ordinary skill in the art would recognize that some art-recognized methods are capable of and suitable for determining whether a signal exists in a pattern, such as Rietveld refinement.

As used herein, “a signal at . . . degrees two-theta,” “a signal at [a] two-theta value[ ] of . . . ” and/or “a signal at at least . . . two-theta value(s) chosen from . . . .” refer to X-ray reflection positions as measured and observed in X-ray powder diffraction experiments (°2θ).

The repeatability of the angular values is in the range of ±0.2° 20, i.e., the angular value can be at the recited angular value +0.2 degrees two-theta, the angular value −0.2 degrees two-theta, or any value between those two end points (angular value +0.2 degrees two-theta and angular value −0.2 degrees two-theta).

The terms “signal intensities” and “peak intensities” interchangeably refer to relative signal intensities within a given X-ray powder diffractogram. Factors that can affect the relative signal or peak intensities include sample thickness and preferred orientation (e.g., the crystalline particles are not distributed randomly).

The term “X-ray powder diffractogram having a signal at . . . two-theta values” as used herein refers to an XRPD pattern that contains X-ray reflection positions as measured and observed in X-ray powder diffraction experiments (°2θ).

As used herein, the term “amorphous” refers to a solid material having no long range order in the position of its molecules. Amorphous solids are generally supercooled liquids in which the molecules are arranged in a random manner so that there is no well-defined arrangement, e.g., molecular packing, and no long range order. For example, an amorphous material is a solid material having no sharp characteristic signal(s) in its X-ray power diffractogram (i.e., is not crystalline as determined by XRPD). Instead, one or more broad peaks (e.g., halos) appear in its diffractogram. Broad peaks are characteristic of an amorphous solid. See, e.g., US 2004/0006237 for a comparison of diffractograms of an amorphous material and crystalline material.

As used herein, an X-ray powder diffractogram is “substantially similar to that in [a particular] Figure” when at least 90%, such as at least 95%, at least 98%, or at least 99%, of the signals in the two diffractograms overlap. In determining “substantial similarity,” one of ordinary skill in the art will understand that there may be variation in the intensities and/or signal positions in XRPD diffractograms even for the same crystalline form. Thus, those of ordinary skill in the art will understand that the signal maximum values in XRPD diffractograms (in degrees two-theta (°2θ) referred to herein) generally mean that value reported ±0.2 degrees 2θ of the reported value, an art-recognized variance.

As used herein, a crystalline form is “substantially pure” when it accounts for an amount by weight equal to or greater than 90% of the sum of all solid form(s) in a sample as determined by a method in accordance with the art, such as quantitative XRPD. In some embodiments, the solid form is “substantially pure” when it accounts for an amount by weight equal to or greater than 95% of the sum of all solid form(s) in a sample. In some embodiments, the solid form is “substantially pure” when it accounts for an amount by weight equal to or greater than 99% of the sum of all solid form(s) in a sample.

As used herein, the term “DSC” refers to the analytical method of Differential Scanning calorimetry.

As used herein, the term “onset of decomposition” refers to the intersection point of the baseline before transition and the interflection tangent.

As used herein, the term “glass transition temperature” or “Tg” refers to the temperature above which a glassy amorphous solid becomes rubbery.

As used herein, the term “TGA” refers to the analytical method of Thermo Gravimetric (or thermogravimetric) Analysis.

As used herein, the term “solvent” refers to any liquid in which the product is at least partially soluble (solubility of product >1 g/l).

As used herein, the term “anti-solvent” refers to any liquid in which the product is insoluble or at maximum sparingly soluble (solubility of product <0.01 mol/l).

As used herein, the term “anti-solvent crystallization” refers to a process wherein supersaturation is achieved and, as a result thereof, crystallization is induced by addition of an antisolvent to the product solution.

As used herein, “CFTR” means cystic fibrosis transmembrane conductance regulator.

As used herein, “mutations” can refer to mutations in the CFTR gene or the CFTR protein. A “CFTR gene mutation” refers to a mutation in the CFTR gene, and a “CFTR protein mutation” refers to a mutation in the CFTR protein. A genetic defect or mutation, or a change in the nucleotides in a gene in general results in a mutation in the CFTR protein translated from that gene, or a frame shift(s).

The term “F508del” refers to a mutant CFTR protein which is lacking the amino acid phenylalanine at position 508.

As used herein, a patient who is “homozygous” for a particular gene mutation has the same mutation on each allele.

As used herein, a patient who is “heterozygous” for a particular gene mutation has this mutation on one allele, and a different mutation on the other allele.

As used herein, the term “modulator” refers to a compound that increases the activity of a biological compound such as a protein. For example, a CFTR modulator is a compound that increases the activity of CFTR. The increase in activity resulting from a CFTR modulator includes but is not limited to compounds that correct, potentiate, stabilize and/or amplify CFTR.

As used herein, the term “CFTR corrector” refers to a compound that facilitates the processing and trafficking of CFTR to increase the amount of CFTR at the cell surface. Compound (I), Compound (II), Compound (III) and their pharmaceutically acceptable salts thereof disclosed herein are CFTR correctors.

As used herein, the term “CFTR potentiator” refers to a compound that increases the channel activity of CFTR protein located at the cell surface, resulting in enhanced ion transport. Compound (V) disclosed herein is a CFTR potentiator.

As used herein, the term “active pharmaceutical ingredient” (“API”) refers to a biologically active compound.

The terms “patient” and “subject” are used interchangeably and refer to an animal including humans.

The terms “effective dose” and “effective amount” are used interchangeably herein and refer to that amount of a compound that produces the desired effect for which it is administered (e.g., improvement in CF or a symptom of CF, or lessening the severity of CF or a symptom of CF). The exact amount of an effective dose will depend on the purpose of the treatment, and will be ascertainable by one skilled in the art (see, e.g., Lloyd (1999) The Art, Science and Technology of Pharmaceutical Compounding).

As used herein, the terms “treatment,” “treating,” and the like generally mean the improvement of CF or its symptoms or lessening the severity of CF or its symptoms in a subject. “Treatment,” as used herein, includes, but is not limited to, the following: increased growth of the subject, increased weight gain, reduction of mucus in the lungs, improved pancreatic and/or liver function, reduction of chest infections, and/or reductions in coughing or shortness of breath. Improvements in or lessening the severity of any of these symptoms can be readily assessed according to standard methods and techniques known in the art.

The terms “about” and “approximately”, when used in connection with doses, amounts, or weight percent of ingredients of a composition or a dosage form, include the value of a specified dose, amount, or weight percent or a range of the dose, amount, or weight percent that is recognized by one of ordinary skill in the art to provide a pharmacological effect equivalent to that obtained from the specified dose, amount, or weight percent.

As used herein, the term “room temperature” or “ambient temperature” means 15° C. to 30° C.

As stated above, disclosed herein are crystalline forms of Compound (I):

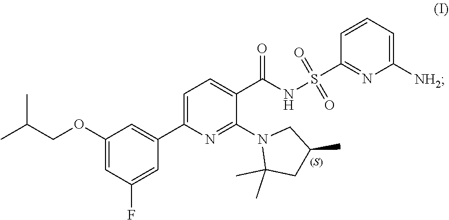

crystalline forms of Compound (II):

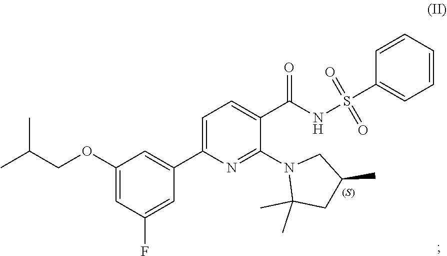

and
crystalline forms of pharmaceutically acceptable salts any of the foregoing, either as an isomeric mixture or enantioenriched (e.g., >90% ee, >95% ee, or >98% ee) isomers.

### Crystalline Forms of Compound (I)

### Crystalline Form A of Compound (I)

In some embodiments, the present disclosure provides crystalline Form A of Compound (I):

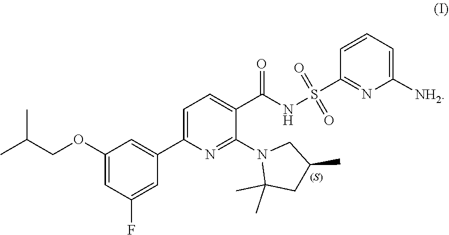

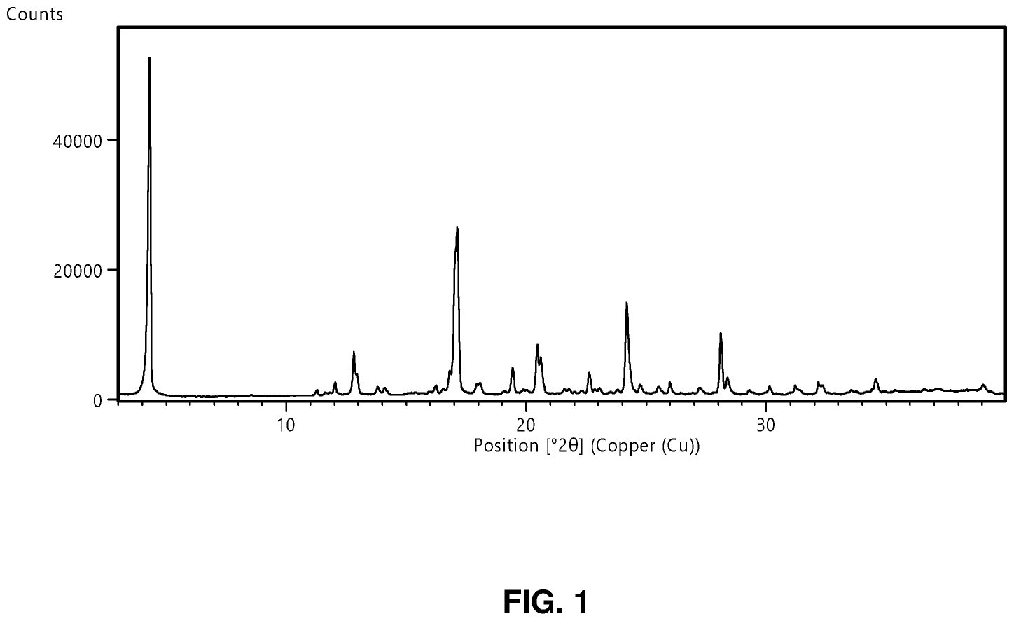

FIG. 1 shows an X-ray powder diffractogram of crystalline Form A of Compound (I) at ambient conditions.

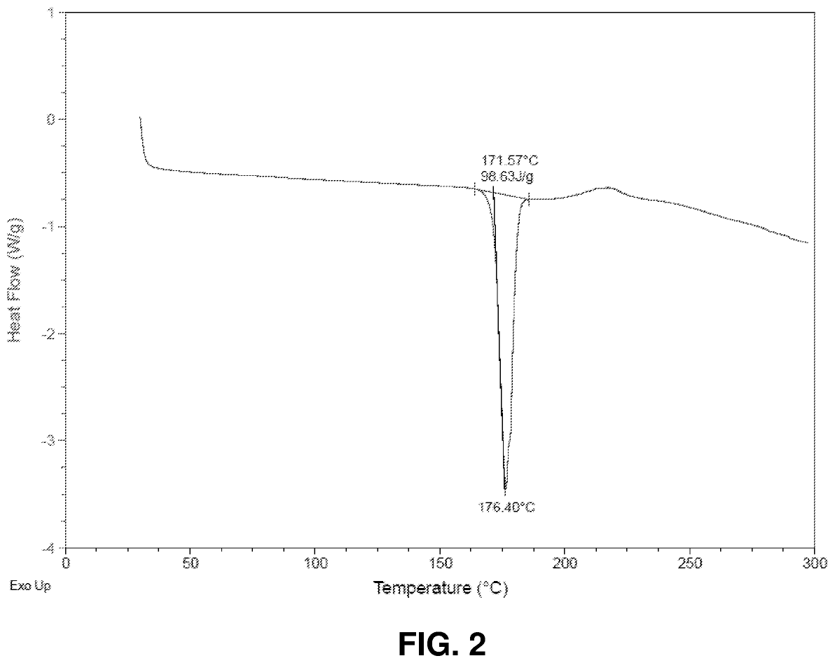

FIG. 2 shows a DSC trace of the crystalline Form A of Compound (I). In some embodiments, crystalline Form A of Compound (I) is characterized by a DSC having an onset of melting temperature of 171.6° C. and/or a peak temperature of 176° C.

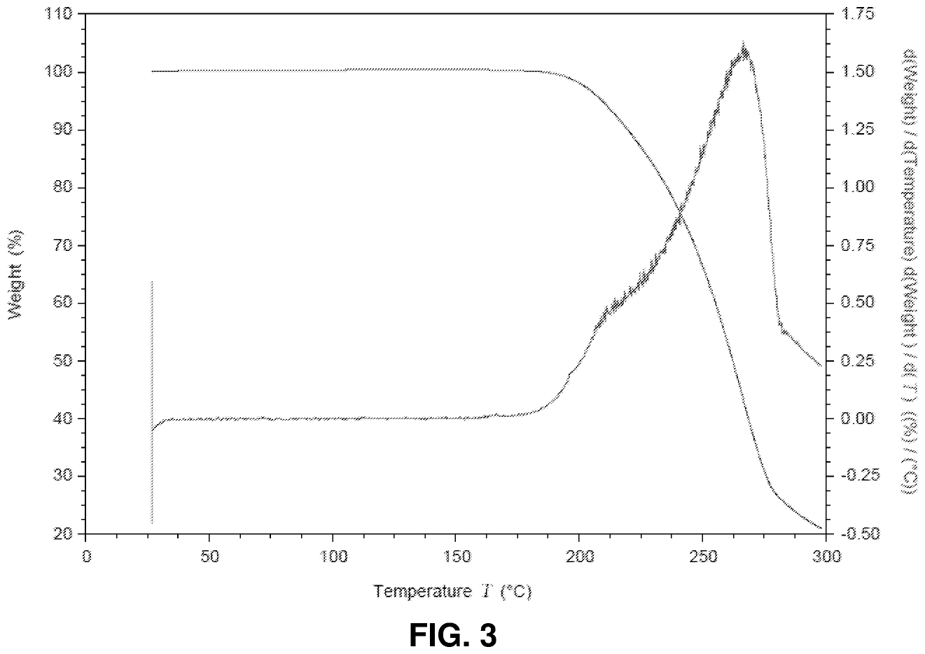

FIG. 3 shows the results of a TGA of crystalline Form A of Compound (I). In some embodiments, crystalline Form A of Compound (I) is characterized by a TGA having an onset of decomposition temperature of about 200° C.

In some embodiments, crystalline Form A of Compound (I) is in substantially pure form. In some embodiments, crystalline Form A of Compound (I) is characterized by an X-ray powder diffractogram generated by an X-ray powder diffraction analysis with an incident beam of Cu Kα radiation.

In some embodiments, crystalline Form A of Compound (I) is characterized by an X-ray powder diffractogram having a signal at 4.3±0.2 degrees two-theta. In some embodiments, crystalline Form A of Compound (I) is characterized by an X-ray powder diffractogram having a signal at 12.8±0.2 degrees two-theta. In some embodiments, crystalline Form A of Compound (I) is characterized by an X-ray powder diffractogram having a signal at 17.1±0.2 degrees two-theta. In some embodiments, crystalline Form A of Compound (I) is characterized by an X-ray powder diffractogram having a signal at 20.5±0.2 degrees two-theta. In some embodiments, crystalline Form A of Compound (I) is characterized by an X-ray powder diffractogram having a signal at 24.2±0.2 degrees two-theta. In some embodiments, crystalline Form A of Compound (I) is characterized by an X-ray powder diffractogram having a signal at 28.1±0.2 degrees two-theta.

In some embodiments, crystalline Form A of Compound (I) is characterized by an X-ray powder diffractogram having a signal at two-theta values of 4.3±0.2, 12.8±0.2, 17.1±0.2, 20.5±0.2, 24.2±0.2, and 28.1±0.2. In some embodiments, crystalline Form A of Compound (I) is characterized by an X-ray powder diffractogram having a signal at at least five two-theta values chosen from 4.3±0.2, 12.8±0.2, 17.1±0.2, 20.5±0.2, 24.2±0.2, and 28.1±0.2. In some embodiments, crystalline Form A of Compound (I) is characterized by an X-ray powder diffractogram having a signal at at least four two-theta values chosen from 4.3±0.2, 12.8±0.2, 17.1±0.2, 20.5±0.2, 24.2±0.2, and 28.1±0.2. In some embodiments, crystalline Form A of Compound (I) is characterized by an X-ray powder diffractogram having a signal at at least three two-theta values chosen from 4.3±0.2, 12.8±0.2, 17.1±0.2, 20.5±0.2, 24.2±0.2, and 28.1±0.2. In some embodiments, crystalline Form A of Compound (I) is characterized by an X-ray powder diffractogram having a signal at at least two two-theta values chosen from 4.3±0.2, 12.8±0.2, 17.1±0.2, 20.5±0.2, 24.2±0.2, and 28.1±0.2. In some embodiments, crystalline Form A of Compound (I) is characterized by an X-ray powder diffractogram having a signal at at least one two-theta value chosen from 4.3±0.2, 12.8±0.2, 17.1±0.2, 20.5±0.2, 24.2±0.2, and 28.1±0.2.

In some embodiments, crystalline Form A of Compound (I) is characterized by an X-ray powder diffractogram having a signal at two-theta values of 4.3±0.2, 17.1±0.2, 20.5±0.2, 24.2±0.2, and 28.1±0.2. In some embodiments, crystalline Form A of Compound (I) is characterized by an X-ray powder diffractogram having a signal at at least four two-theta values chosen from 4.3±0.2, 17.1±0.2, 20.5±0.2, 24.2±0.2, and 28.1±0.2. In some embodiments, crystalline Form A of Compound (I) is characterized by an X-ray powder diffractogram having a signal at at least three two-theta values chosen from 4.3±0.2, 17.1±0.2, 20.5±0.2, 24.2±0.2, and 28.1±0.2. In some embodiments, crystalline Form A of Compound (I) is characterized by an X-ray powder diffractogram having a signal at at least two two-theta values chosen from 4.3±0.2, 17.1±0.2, 20.5±0.2, 24.2±0.2, and 28.1±0.2. In some embodiments, crystalline Form A of Compound (I) is characterized by an X-ray powder diffractogram having a signal at at least one two-theta value chosen from 4.3±0.2, 17.1±0.2, 20.5±0.2, 24.2±0.2, and 28.1±0.2.

In some embodiments, crystalline Form A of Compound (I) is characterized by an X-ray powder diffractogram having a signal at two-theta values of 4.3±0.2, 17.1±0.2, and 24.2±0.2. In some embodiments, crystalline Form A of Compound (I) is characterized by an X-ray powder diffractogram having a signal at at least two two-theta values chosen from 4.3±0.2, 17.1±0.2, and 24.2±0.2. In some embodiments, crystalline Form A of Compound (I) is characterized by an X-ray powder diffractogram having a signal at at least one two-theta value chosen from 4.3±0.2, 17.1±0.2, and 24.2±0.2.

In some embodiments, crystalline Form A of Compound (I) is characterized by an X-ray powder diffractogram having a signal at two-theta values of 4.3±0.2, 12.8±0.2, 17.1±0.2, and 20.5±0.2. In some embodiments, crystalline Form A of Compound (I) is characterized by an X-ray powder diffractogram having a signal at at least three two-theta values chosen from 4.3±0.2, 12.8±0.2, 17.1±0.2, and 20.5±0.2. In some embodiments, crystalline Form A of Compound (I) is characterized by an X-ray powder diffractogram having a signal at two-theta values of 4.3±0.2, 17.1±0.2, and 20.5±0.2. In some embodiments, crystalline Form A of Compound (I) is characterized by an X-ray powder diffractogram having a signal at two-theta values of 4.3±0.2, 12.8±0.2, and 20.5±0.2.

In some embodiments, crystalline Form A of Compound (I) is characterized by an X-ray powder diffractogram substantially similar to that in FIG. 1.

In some embodiments, crystalline Form A of Compound (I) is characterized by a triclinic crystal system. In some embodiments, crystalline Form A of Compound (I) is characterized as belonging to a P1 space group. In some embodiments, crystalline Form A of Compound (I) is characterized by having a unit cell characterized by three edges of 8.6335±0.0012 Å, 15.4405±0.0019 Å, and 21.977±0.003 Å.

In some embodiments, crystalline Form A of Compound (I) is characterized by having a unit cell with the following characteristics measured at 100° K and 0.71073 Å:

| | |
|---|---|
| Crystal System: | Triclinic |
| Space Group: | P1 |
| a (Å): | 8.6335(12) |
| b (Å): | 15.4405(19) |
| c (Å): | 21.977(3) |
| α (°): | 109.618(4) |
| β (°): | 94.608(4) |
| γ (°): | 91.419(4) |
| V (Å3): | 2746.4(6) |
| Z/Z′: | ¼ |

In some embodiments, crystalline Form A of Compound (I) is characterized by having a unit cell characterized by three inequivalent angles. In some embodiments, crystalline Form A of Compound (I) is characterized by having a unit cell characterized with an angle, α, of 109.618±0.004°. In some embodiments, crystalline Form A of Compound (I) is characterized by having a unit cell characterized with an angle, β, of 94.608±0.004°. In some embodiments, crystalline Form A of Compound (I) is characterized by having a unit cell characterized with an angle, γ, of 91.419±0.004°.

In some embodiments, crystalline Form A of Compound (I) is characterized by having a unit cell with volume of 2746.4±0.6 Å³. In some embodiments, crystalline Form A of Compound (I) is characterized by having a unit cell with volume of 2746 Å³.

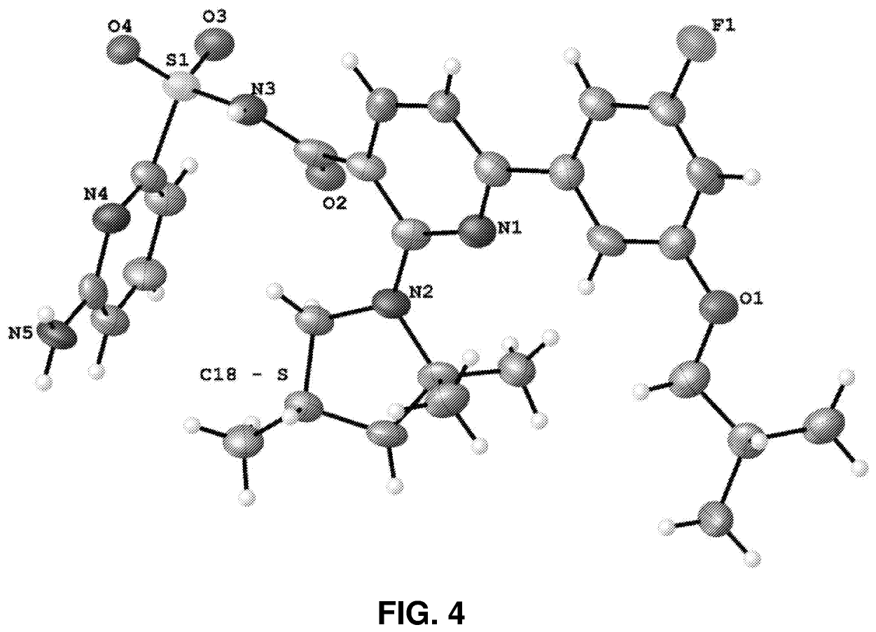

In some embodiments, crystalline Form A of Compound (I) is characterized by a single crystal structure substantially similar to that in FIG. 4.

In some embodiments, the present disclosure provides crystalline Form A of Compound (I) prepared by a process comprising crystallizing Compound (I) from a mixture of ethanol, water, and Compound (I). In some embodiments, the crystallization is performed at room temperature. In some embodiments, the crystallization is performed at a temperature above room temperature. In some embodiments, crystallization is induced upon the addition of a seed crystal.

In some embodiments, the present disclosure provides methods of preparing crystalline Form A of Compound (I) comprising crystallizing Compound (I) from a mixture of ethanol, water, and Compound (I). In some embodiments, the crystallization is performed at room temperature. In some embodiments, the crystallization is performed at a temperature above room temperature. In some embodiments, crystallization is induced upon the addition of a seed crystal.

### Crystalline Form B of Compound (I)

In some embodiments, the present disclosure provides crystalline Form B of Compound (I):

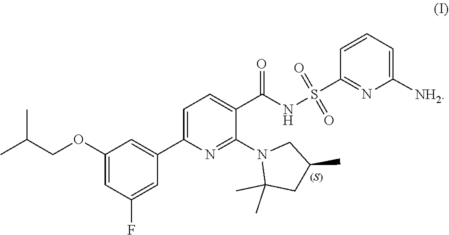

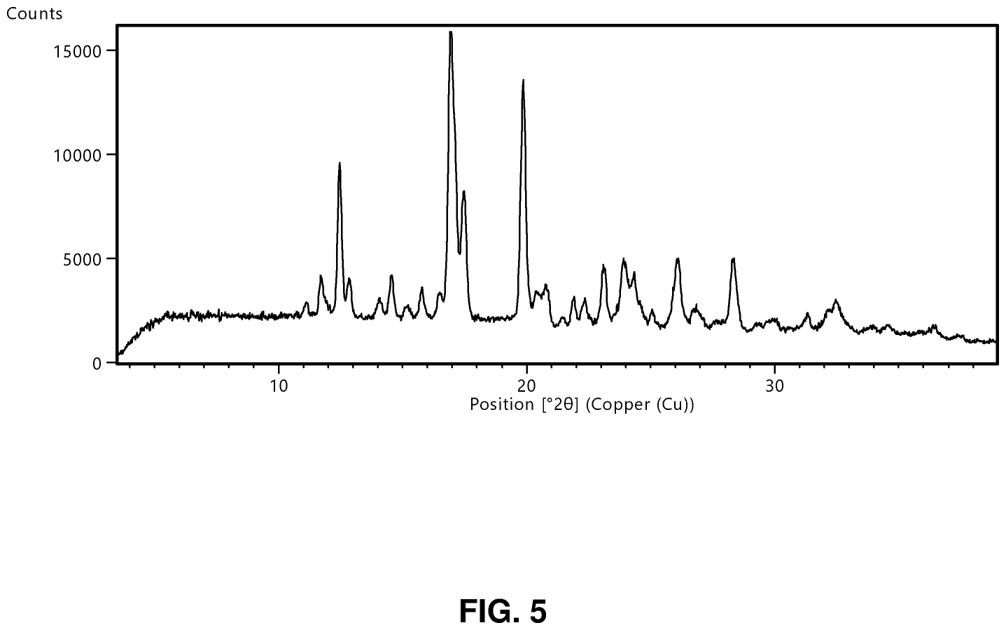

FIG. 5 shows an X-ray powder diffractogram of crystalline Form B of Compound (I) at ambient conditions.

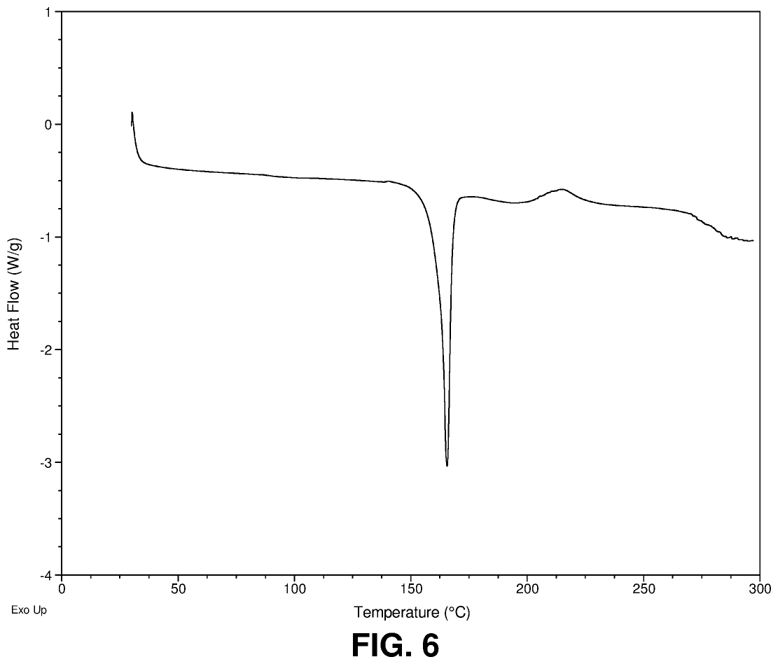

FIG. 6 shows a DSC trace of the crystalline Form B of Compound (I). In some embodiments, crystalline Form B of Compound (I) is characterized by a DSC having an onset of melting temperature of 162.7° C. and/or a peak temperature of 165.6° C.

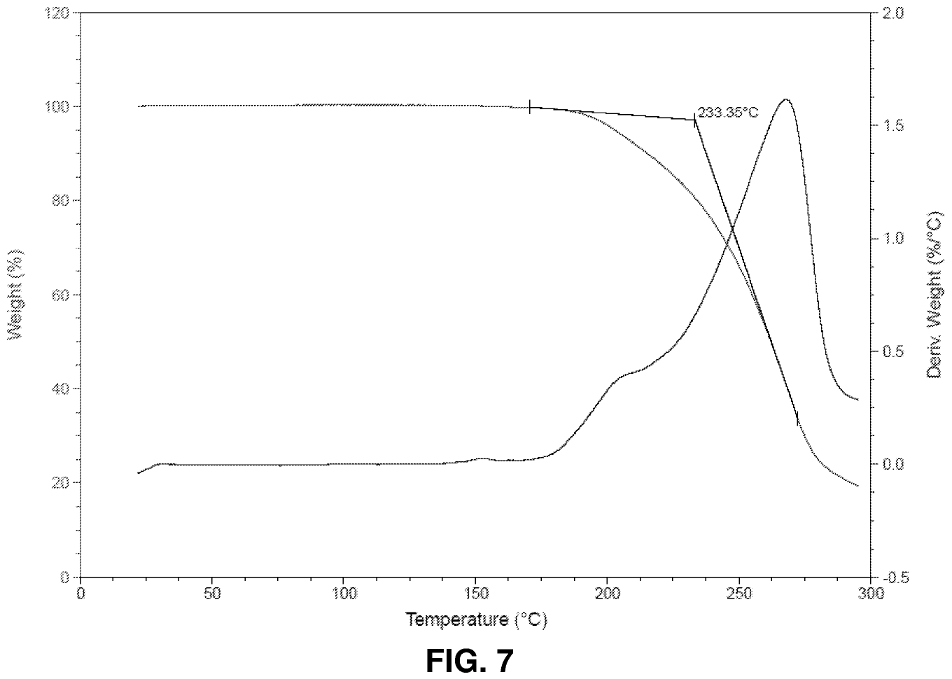

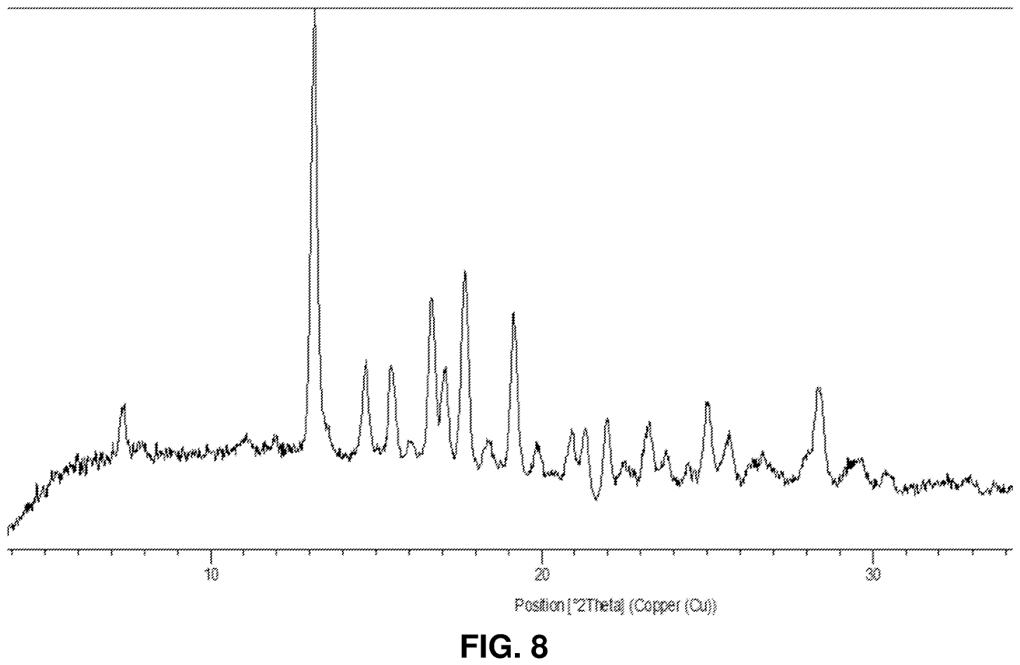

FIG. 7 shows TGA results of crystalline Form B of Compound (I). In some embodiments, crystalline Form B of Compound (I) is characterized by a TGA having an onset of decomposition temperature of about 200° C.

In some embodiments, crystalline Form B of Compound (I) is in substantially pure form. In some embodiments, crystalline Form B of Compound (I) is characterized by an X-ray powder diffractogram generated by an X-ray powder diffraction analysis with an incident beam of Cu Kα radiation.

In some embodiments, crystalline Form B of Compound (I) is characterized by an X-ray powder diffractogram having a signal at 12.4±0.2 degrees two-theta. In some embodiments, crystalline Form B of Compound (I) is characterized by an X-ray powder diffractogram having a signal at 14.5±0.2 degrees two-theta. In some embodiments, crystalline Form B of Compound (I) is characterized by an X-ray powder diffractogram having a signal at 16.9±0.2 degrees two-theta. In some embodiments, crystalline Form B of Compound (I) is characterized by an X-ray powder diffractogram having a signal at 17.5±0.2 degrees two-theta. In some embodiments, crystalline Form B of Compound (I) is characterized by an X-ray powder diffractogram having a signal at 19.8±0.2 degrees two-theta. In some embodiments, crystalline Form B of Compound (I) is characterized by an X-ray powder diffractogram having a signal at 23.1±0.2 degrees two-theta. In some embodiments, crystalline Form B of Compound (I) is characterized by an X-ray powder diffractogram having a signal at 23.9±0.2 degrees two-theta. In some embodiments, crystalline Form B of Compound (I) is characterized by an X-ray powder diffractogram having a signal at 26.1±0.2 degrees two-theta. In some embodiments, crystalline Form B of Compound (I) is characterized by an X-ray powder diffractogram having a signal at 28.3±0.2 degrees two-theta.

In some embodiments, crystalline Form B of Compound (I) is characterized by an X-ray powder diffractogram having a signal at two-theta values of 12.4±0.2, 14.5±0.2, 16.9±0.2, 17.5±0.2, 19.8±0.2, 23.1±0.2, 23.9±0.2, 26.1±0.2, and 28.3±0.2. In some embodiments, crystalline Form B of Compound (I) is characterized by an X-ray powder diffractogram having a signal at at least eight two-theta values chosen from 12.4±0.2, 14.5±0.2, 16.9±0.2, 17.5±0.2, 19.8±0.2, 23.1±0.2, 23.9±0.2, 26.1±0.2, and 28.3±0.2. In some embodiments, crystalline Form B of Compound (I) is characterized by an X-ray powder diffractogram having a signal at at least seven two-theta values chosen from 12.4±0.2, 14.5±0.2, 16.9±0.2, 17.5±0.2, 19.8±0.2, 23.1±0.2, 23.9±0.2, 26.1±0.2, and 28.3±0.2. In some embodiments, crystalline Form B of Compound (I) is characterized by an X-ray powder diffractogram having a signal at at least six two-theta values chosen from 12.4±0.2, 14.5±0.2, 16.9±0.2, 17.5±0.2, 19.8±0.2, 23.1±0.2, 23.9±0.2, 26.1±0.2, and 28.3±0.2. In some embodiments, crystalline Form B of Compound (I) is characterized by an X-ray powder diffractogram having a signal at at least five two-theta values chosen from 12.4±0.2, 14.5±0.2, 16.9±0.2, 17.5±0.2, 19.8±0.2, 23.1±0.2, 23.9±0.2, 26.1±0.2, and 28.3±0.2. In some embodiments, crystalline Form B of Compound (I) is characterized by an X-ray powder diffractogram having a signal at at least four two-theta values chosen from 12.4±0.2, 14.5±0.2, 16.9±0.2, 17.5±0.2, 19.8±0.2, 23.1±0.2, 23.9±0.2, 26.1±0.2, and 28.3±0.2. In some embodiments, crystalline Form B of Compound (I) is characterized by an X-ray powder diffractogram having a signal at at least three two-theta values chosen from 12.4±0.2, 14.5±0.2, 16.9±0.2, 17.5±0.2, 19.8±0.2, 23.1±0.2, 23.9±0.2, 26.1±0.2, and 28.3±0.2. In some embodiments, crystalline Form B of Compound (I) is characterized by an X-ray powder diffractogram having a signal at at least two two-theta values chosen from 12.4±0.2, 14.5±0.2, 16.9±0.2, 17.5±0.2, 19.8±0.2, 23.1±0.2, 23.9±0.2, 26.1±0.2, and 28.3±0.2. In some embodiments, crystalline Form B of Compound (I) is characterized by an X-ray powder diffractogram having a signal at at least one two-theta value chosen from 12.4±0.2, 14.5±0.2, 16.9±0.2, 17.5±0.2, 19.8±0.2, 23.1±0.2, 23.9±0.2, 26.1±0.2, and 28.3±0.2.

In some embodiments, crystalline Form B of Compound (I) is characterized by an X-ray powder diffractogram having a signal at two-theta values of 12.4±0.2, 16.9±0.2, 17.5±0.2, 19.8±0.2, 23.1±0.2, and 26.1±0.2. In some embodiments, crystalline Form B of Compound (I) is characterized by an X-ray powder diffractogram having a signal at at least five two-theta values chosen from 12.4±0.2, 16.9±0.2, 17.5±0.2, 19.8±0.2, 23.1±0.2, and 26.1±0.2. In some embodiments, crystalline Form B of Compound (I) is characterized by an X-ray powder diffractogram having a signal at at least four two-theta values chosen from 12.4±0.2, 16.9±0.2, 17.5±0.2, 19.8±0.2, 23.1±0.2, and 26.1±0.2. In some embodiments, crystalline Form B of Compound (I) is characterized by an X-ray powder diffractogram having a signal at at least three two-theta values chosen from 12.4±0.2, 16.9±0.2, 17.5±0.2, 19.8±0.2, 23.1±0.2, and 26.1±0.2. In some embodiments, crystalline Form B of Compound (I) is characterized by an X-ray powder diffractogram having a signal at at least two two-theta values chosen from 12.4±0.2, 16.9±0.2, 17.5±0.2, 19.8±0.2, 23.1±0.2, and 26.1±0.2. In some embodiments, crystalline Form B of Compound (I) is characterized by an X-ray powder diffractogram having a signal at at least one two-theta value chosen from 12.4±0.2, 16.9±0.2, 17.5±0.2, 19.8±0.2, 23.1±0.2, and 26.1±0.2.

In some embodiments, crystalline Form B of Compound (I) is characterized by an X-ray powder diffractogram having a signal at two-theta values of 12.4±0.2, 19.8±0.2, and 23.1±0.2. In some embodiments, crystalline Form B of Compound (I) is characterized by an X-ray powder diffractogram having a signal at at least two two-theta values chosen from 12.4±0.2, 19.8±0.2, and 23.1±0.2. In some embodiments, crystalline Form B of Compound (I) is characterized by an X-ray powder diffractogram having a signal at at least one two-theta value chosen from 12.4±0.2, 19.8±0.2, and 23.1±0.2.

In some embodiments, crystalline Form B of Compound (I) is characterized by an X-ray powder diffractogram substantially similar to that in FIG. 5.

In some embodiments, the present disclosure provides crystalline Form B of Compound (I) prepared by a process comprising crystallizing Compound (I) from a mixture of isopropylacetate and Compound (I). In some embodiments, the present disclosure provides crystalline Form B of Compound (I) prepared by a process comprising crystallizing Compound (I) from a mixture of isopropylacetate, n-heptane, and Compound (I).

In some embodiments, the present disclosure provides methods of preparing Form B of Compound (I) comprising crystallizing Compound (I) from a mixture of isopropylacetate and Compound (I). In some embodiments, the present disclosure provides methods of preparing Form B of Compound (I) comprising crystallizing Compound (I) from a mixture of isopropylacetate, n-heptane, and Compound (I).

### Crystalline Form H of Compound (I)

In some embodiments, the present disclosure provides crystalline Form H of Compound (I):

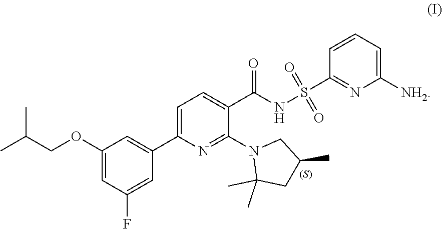

In some embodiments, crystalline Form H of Compound (I) is a hydrate of Compound (I).

FIG. 8 shows an X-ray powder diffractogram of crystalline Form H of Compound (I) at ambient conditions.

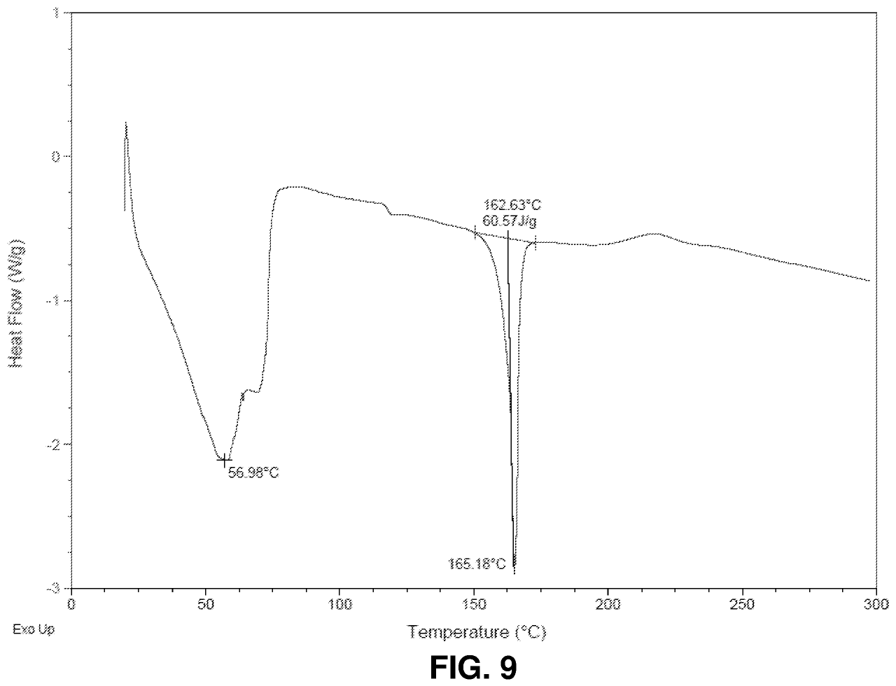

FIG. 9 shows a DSC trace of the crystalline Form H of Compound (I). In some embodiments, crystalline Form H of Compound (I) is characterized by a DSC having a peak temperature of 57.0° C. and/or a peak temperature of 165.2° C.

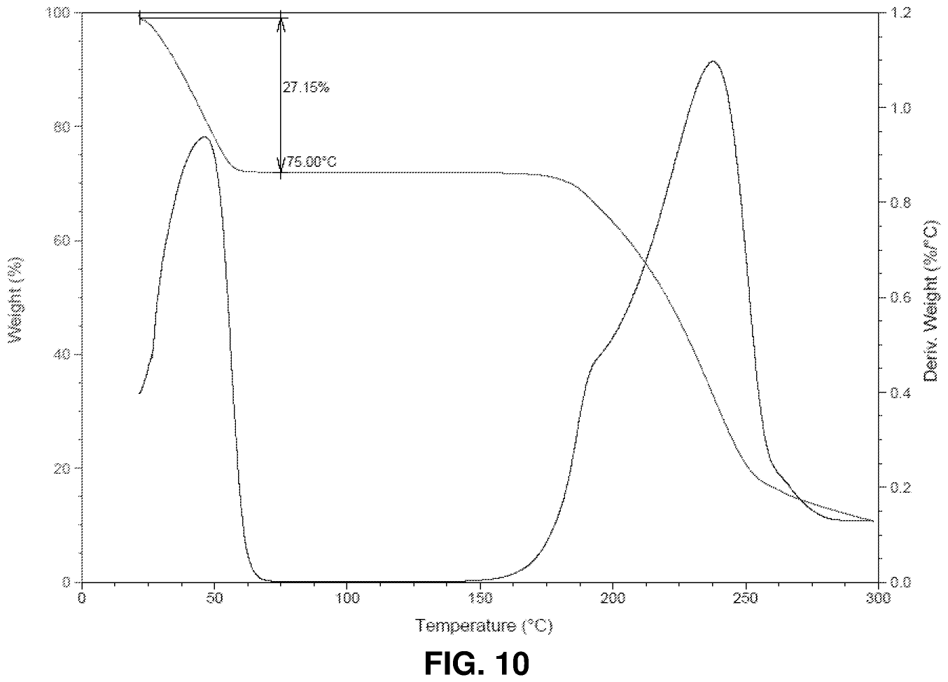

FIG. 10 shows TGA results of crystalline Form H of Compound (I).

In some embodiments, crystalline Form H of Compound (I) is in substantially pure form.

In some embodiments, crystalline Form H of Compound (I) is characterized by an X-ray powder diffractogram generated by an X-ray powder diffraction analysis with an incident beam of Cu Kα radiation.

In some embodiments, crystalline Form H of Compound (I) is characterized by an X-ray powder diffractogram having a signal at 13.2±0.2 degrees two-theta. In some embodiments, crystalline Form H of Compound (I) is characterized by an X-ray powder diffractogram having a signal at 14.8±0.2 degrees two-theta. In some embodiments, crystalline Form H of Compound (I) is characterized by an X-ray powder diffractogram having a signal at 15.5±0.2 degrees two-theta. In some embodiments, crystalline Form H of Compound (I) is characterized by an X-ray powder diffractogram having a signal at 16.8±0.2 degrees two-theta. In some embodiments, crystalline Form H of Compound (I) is characterized by an X-ray powder diffractogram having a signal at 17.2±0.2 degrees two-theta. In some embodiments, crystalline Form H of Compound (I) is characterized by an X-ray powder diffractogram having a signal at 17.8±0.2 degrees two-theta. In some embodiments, crystalline Form H of Compound (I) is characterized by an X-ray powder diffractogram having a signal at 19.2±0.2 degrees two-theta. In some embodiments, crystalline Form H of Compound (I) is characterized by an X-ray powder diffractogram having a signal at 22.1±0.2 degrees two-theta. In some embodiments, crystalline Form H of Compound (I) is characterized by an X-ray powder diffractogram having a signal at 25.1±0.2 degrees two-theta. In some embodiments, crystalline Form H of Compound (I) is characterized by an X-ray powder diffractogram having a signal at 28.5±0.2 degrees two-theta.

In some embodiments, crystalline Form H of Compound (I) is characterized by an X-ray powder diffractogram having a signal at two-theta values of 13.2±0.2, 14.8±0.2, 15.5±0.2, 16.8±0.2, 17.2±0.2, 17.8±0.2, 19.2±0.2, 22.1±0.2, 25.1±0.2, and 28.5±0.2. In some embodiments, crystalline Form H of Compound (I) is characterized by an X-ray powder diffractogram having a signal at at least nine two-theta values chosen from 13.2±0.2, 14.8±0.2, 15.5±0.2, 16.8±0.2, 17.2±0.2, 17.8±0.2, 19.2±0.2, 22.1±0.2, 25.1±0.2, and 28.5±0.2. In some embodiments, crystalline Form H of Compound (I) is characterized by an X-ray powder diffractogram having a signal at at least eight two-theta values chosen from 13.2±0.2, 14.8±0.2, 15.5±0.2, 16.8±0.2, 17.2±0.2, 17.8±0.2, 19.2±0.2, 22.1±0.2, 25.1±0.2, and 28.5±0.2. In some embodiments, crystalline Form H of Compound (I) is characterized by an X-ray powder diffractogram having a signal at at least seven two-theta values chosen from 13.2±0.2, 14.8±0.2, 15.5±0.2, 16.8±0.2, 17.2±0.2, 17.8±0.2, 19.2±0.2, 22.1±0.2, 25.1±0.2, and 28.5±0.2. In some embodiments, crystalline Form H of Compound (I) is characterized by an X-ray powder diffractogram having a signal at at least six two-theta values chosen from 13.2±0.2, 14.8±0.2, 15.5±0.2, 16.8±0.2, 17.2±0.2, 17.8±0.2, 19.2±0.2, 22.1±0.2, 25.1±0.2, and 28.5±0.2. In some embodiments, crystalline Form H of Compound (I) is characterized by an X-ray powder diffractogram having a signal at at least five two-theta values chosen from 13.2±0.2, 14.8±0.2, 15.5±0.2, 16.8±0.2, 17.2±0.2, 17.8±0.2, 19.2±0.2, 22.1±0.2, 25.1±0.2, and 28.5±0.2. In some embodiments, crystalline Form H of Compound (I) is characterized by an X-ray powder diffractogram having a signal at at least four two-theta values chosen from 13.2±0.2, 14.8±0.2, 15.5±0.2, 16.8±0.2, 17.2±0.2, 17.8±0.2, 19.2±0.2, 22.1±0.2, 25.1±0.2, and 28.5±0.2. In some embodiments, crystalline Form H of Compound (I) is characterized by an X-ray powder diffractogram having a signal at at least three two-theta values chosen from 13.2±0.2, 14.8±0.2, 15.5±0.2, 16.8±0.2, 17.2±0.2, 17.8±0.2, 19.2±0.2, 22.1±0.2, 25.1±0.2, and 28.5±0.2. In some embodiments, crystalline Form H of Compound (I) is characterized by an X-ray powder diffractogram having a signal at at least two two-theta values chosen from 13.2±0.2, 14.8±0.2, 15.5±0.2, 16.8±0.2, 17.2±0.2, 17.8±0.2, 19.2±0.2, 22.1±0.2, 25.1±0.2, and 28.5±0.2. In some embodiments, crystalline Form H of Compound (I) is characterized by an X-ray powder diffractogram having a signal at at least one two-theta value chosen from 13.2±0.2, 14.8±0.2, 15.5±0.2, 16.8±0.2, 17.2±0.2, 17.8±0.2, 19.2±0.2, 22.1±0.2, 25.1±0.2, and 28.5±0.2.

In some embodiments, crystalline Form H of Compound (I) is characterized by an X-ray powder diffractogram having a signal at two-theta values of 13.2±0.2, 14.8±0.2, 16.8±0.2, 17.8±0.2, 19.2±0.2, and 28.5±0.2. In some embodiments, crystalline Form H of Compound (I) is characterized by an X-ray powder diffractogram having a signal at at least five two-theta values chosen from 13.2±0.2, 14.8±0.2, 16.8±0.2, 17.8±0.2, 19.2±0.2, and 28.5±0.2. In some embodiments, crystalline Form H of Compound (I) is characterized by an X-ray powder diffractogram having a signal at at least four two-theta values chosen from 13.2±0.2, 14.8±0.2, 16.8±0.2, 17.8±0.2, 19.2±0.2, and 28.5±0.2. In some embodiments, crystalline Form H of Compound (I) is characterized by an X-ray powder diffractogram having a signal at at least three two-theta values chosen from 13.2±0.2, 14.8±0.2, 16.8±0.2, 17.8±0.2, 19.2±0.2, and 28.5±0.2. In some embodiments, crystalline Form H of Compound (I) is characterized by an X-ray powder diffractogram having a signal at at least two two-theta values chosen from 13.2±0.2, 14.8±0.2, 16.8±0.2, 17.8±0.2, 19.2±0.2, and 28.5±0.2. In some embodiments, crystalline Form H of Compound (I) is characterized by an X-ray powder diffractogram having a signal at at least one two-theta value chosen from 13.2±0.2, 14.8±0.2, 16.8±0.2, 17.8±0.2, 19.2±0.2, and 28.5±0.2.

In some embodiments, crystalline Form H of Compound (I) is characterized by an X-ray powder diffractogram having a signal at two-theta values of 13.2±0.2, 17.8±0.2, and 19.2±0.2. In some embodiments, crystalline Form H of Compound (I) is characterized by an X-ray powder diffractogram having a signal at at least two two-theta value chosen from 13.2±0.2, 17.8±0.2, and 19.2±0.2. In some embodiments, crystalline Form H of Compound (I) is characterized by an X-ray powder diffractogram having a signal at at least one two-theta value chosen from 13.2±0.2, 17.8±0.2, and 19.2±0.2.

In some embodiments, crystalline Form H of Compound (I) is characterized by an X-ray powder diffractogram substantially similar to that in FIG. 8.

In some embodiments, crystalline Form H of Compound (I) is characterized by a monoclinic crystal system. In some embodiments, crystalline Form H of Compound (I) is characterized as belonging to a C 1 2 1 space group. In some embodiments, crystalline Form H of Compound (I) is characterized by having a unit cell characterized by three edges of 48.396±0.006 Å, 9.0743±0.0010 Å, and 13.3215±0.0014 Å.

In some embodiments, crystalline Form H of Compound (I) is characterized by having a unit cell with the following characteristics measured at 100° K and 0.71073 Å:

| Crystal | Monoclinic |
|---|---|
| System: | |
| Space | C 1 2 1 |
| Group: | |
| a (Å): | 48.396(6) |
| b (Å): | 9.0743(10) |
| c (Å): | 13.3215(14) |
| α (°): | 90 |
| β (°): | 94.034(3) |
| γ (°): | 90 |
| V (Å3): | 5835.7(11) |
| Z/Z′: | 4/2 |

In some embodiments, crystalline Form H of Compound (I) is characterized by having a unit cell characterized by two equivalent angles. In some embodiments, crystalline Form H of Compound (I) is characterized by having a unit cell characterized with an angle, α, of 90°. In some embodiments, crystalline Form H of Compound (I) is characterized by having a unit cell characterized with an angle, β, of 94.034±0.003°. In some embodiments, crystalline Form H of Compound (I) is characterized by having a unit cell characterized with an angle, γ, of 90°.

In some embodiments, crystalline Form H of Compound (I) is characterized by having a unit cell with volume of 5835.7±0.11 Å³. In some embodiments, crystalline Form H of Compound (I) is characterized by having a unit cell with volume of 5836 Å³.

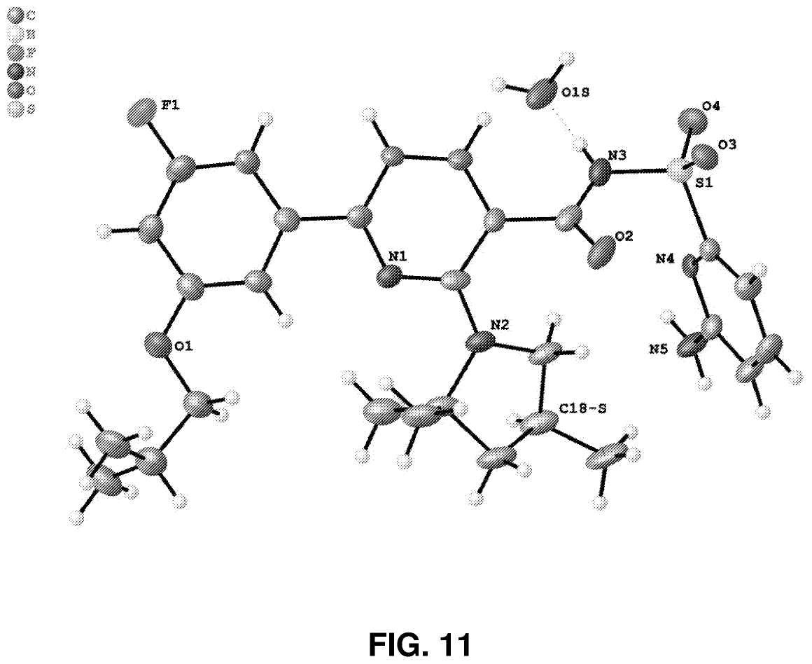

In some embodiments, crystalline Form H of Compound (I) is characterized by a single crystal structure substantially similar to that in FIG. 11.

In some embodiments, the present disclosure provides crystalline Form H of Compound (I) prepared by a process comprising crystallizing Compound (I) from a mixture of methanol, water, and Compound (I). In some embodiments, the crystallizing occurs at room temperature.

In some embodiments, the present disclosure provides methods of preparing Crystalline Form H of Compound (I) comprising crystallizing Compound (I) from a mixture of methanol, water, and Compound (I). In some embodiments, the crystallizing occurs at room temperature.

### Crystalline Form S of Compound (I)

In some embodiments, the present disclosure provides crystalline Form S of Compound (I):

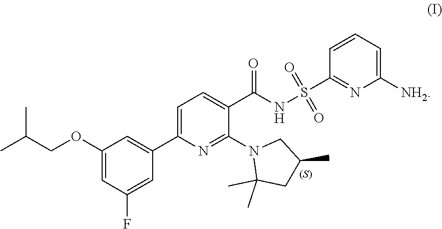

In some embodiments, crystalline Form S is a dioxane/heptane solvate of Compound (I).

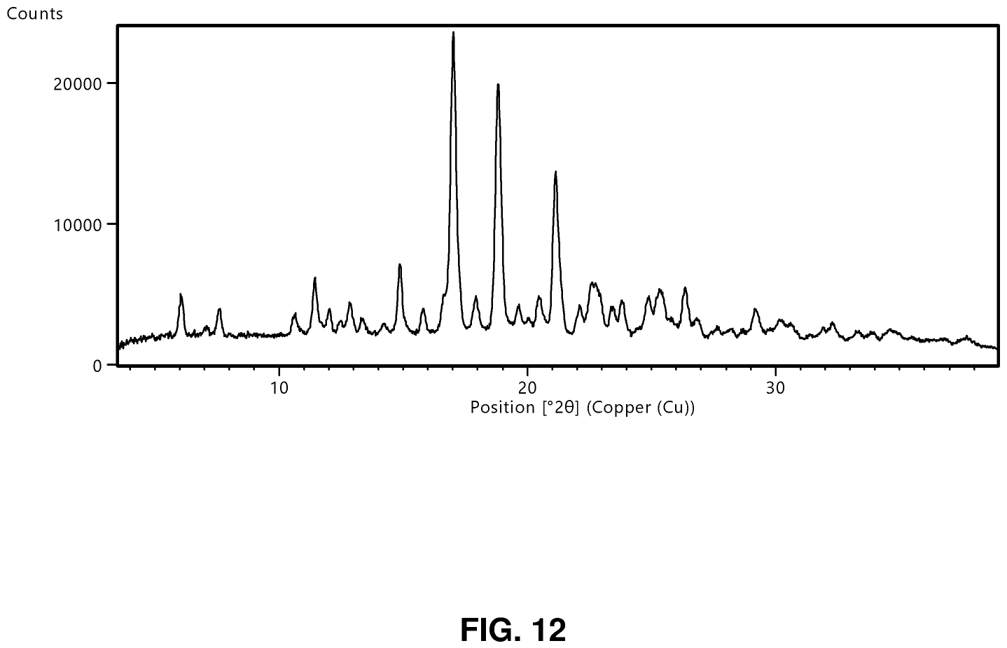

FIG. 12 shows an X-ray powder diffractogram of crystalline Form S of Compound (I) at ambient conditions.

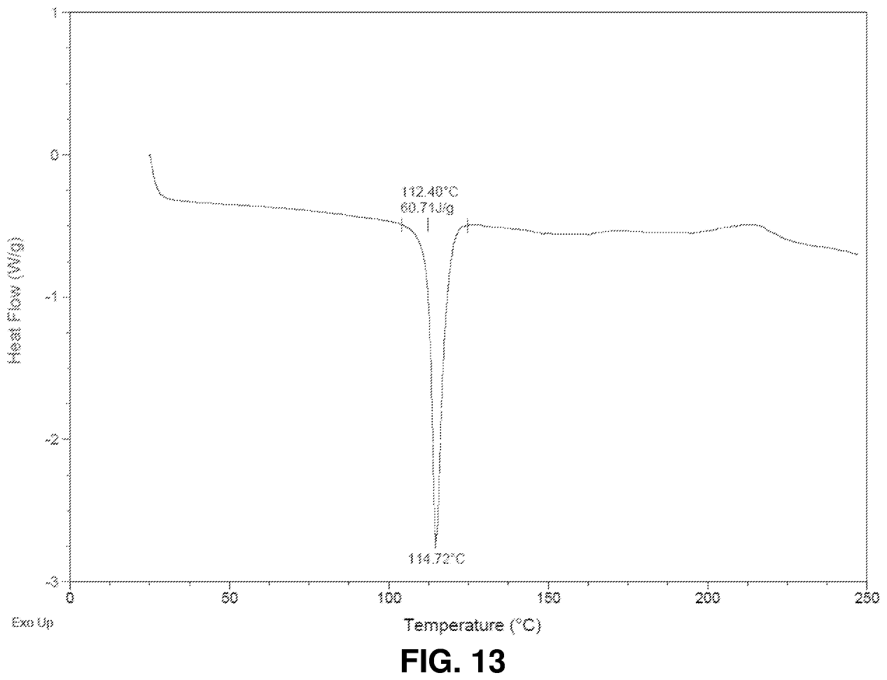

FIG. 13 shows a DSC trace of the crystalline Form S of Compound (I). In some embodiments, crystalline Form S of Compound (I) is characterized by a DSC having a peak temperature of 114.7° C.

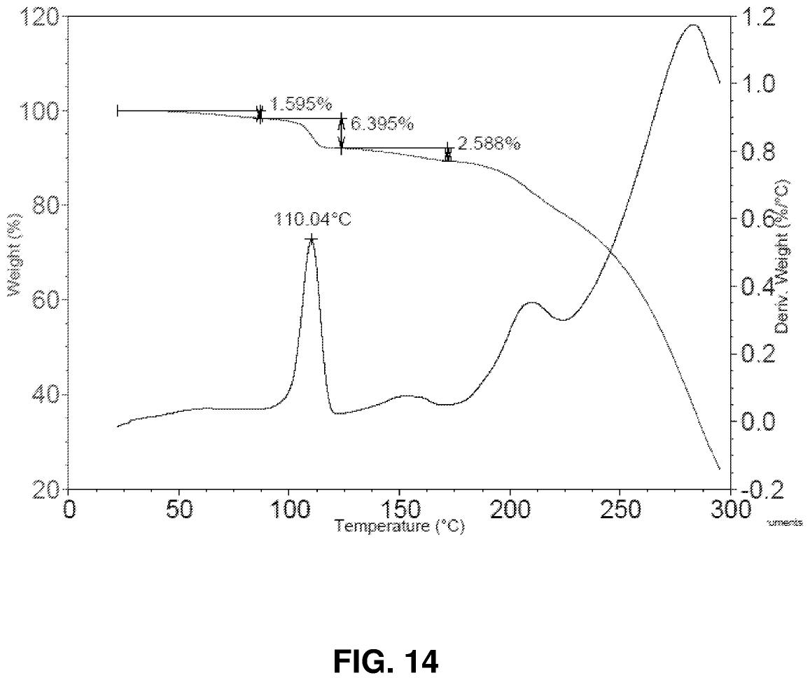

FIG. 14 shows TGA results of crystalline Form S of Compound (I).

In some embodiments, crystalline Form S of Compound (I) is in substantially pure form. In some embodiments, crystalline Form S of Compound (I) is characterized by an X-ray powder diffractogram generated by an X-ray powder diffraction analysis with an incident beam of Cu Kα radiation.

In some embodiments, crystalline Form S of Compound (I) is characterized by an X-ray powder diffractogram having a signal at 11.5±0.2 degrees two-theta. In some embodiments, crystalline Form S of Compound (I) is characterized by an X-ray powder diffractogram having a signal at 14.9±0.2 degrees two-theta. In some embodiments, crystalline Form S of Compound (I) is characterized by an X-ray powder diffractogram having a signal at 16.6±0.2 degrees two-theta. In some embodiments, crystalline Form S of Compound (I) is characterized by an X-ray powder diffractogram having a signal at 17.0±0.2 degrees two-theta. In some embodiments, crystalline Form S of Compound (I) is characterized by an X-ray powder diffractogram having a signal at 18.8±0.2 degrees two-theta. In some embodiments, crystalline Form S of Compound (I) is characterized by an X-ray powder diffractogram having a signal at 20.5±0.2 degrees two-theta. In some embodiments, crystalline Form S of Compound (I) is characterized by an X-ray powder diffractogram having a signal at 21.2±0.2 degrees two-theta. In some embodiments, crystalline Form S of Compound (I) is characterized by an X-ray powder diffractogram having a signal at 22.6±0.2 degrees two-theta. In some embodiments, crystalline Form S of Compound (I) is characterized by an X-ray powder diffractogram having a signal at 26.4±0.2 degrees two-theta. In some embodiments, crystalline Form S of Compound (I) is characterized by an X-ray powder diffractogram having a signal at 25.3±0.2 degrees two-theta.

In some embodiments, crystalline Form S of Compound (I) is characterized by an X-ray powder diffractogram having a signal at two-theta values of 11.5±0.2, 14.9±0.2, 16.6±0.2, 17.0±0.2, 18.8±0.2, 20.5±0.2, 21.2±0.2, 22.6±0.2, 26.4±0.2, and 25.3±0.2. In some embodiments, crystalline Form S of Compound (I) is characterized by an X-ray powder diffractogram having a signal at at least nine two-theta values chosen from 11.5±0.2, 14.9±0.2, 16.6±0.2, 17.0±0.2, 18.8±0.2, 20.5±0.2, 21.2±0.2, 22.6±0.2, 26.4±0.2, and 25.3±0.2. In some embodiments, crystalline Form S of Compound (I) is characterized by an X-ray powder diffractogram having a signal at at least eight two-theta values chosen from 11.5±0.2, 14.9±0.2, 16.6±0.2, 17.0±0.2, 18.8±0.2, 20.5±0.2, 21.2±0.2, 22.6±0.2, 26.4±0.2, and 25.3±0.2. In some embodiments, crystalline Form S of Compound (I) is characterized by an X-ray powder diffractogram having a signal at at least seven two-theta values chosen from 11.5±0.2, 14.9±0.2, 16.6±0.2, 17.0±0.2, 18.8±0.2, 20.5±0.2, 21.2±0.2, 22.6±0.2, 26.4±0.2, and 25.3±0.2. In some embodiments, crystalline Form S of Compound (I) is characterized by an X-ray powder diffractogram having a signal at at least six two-theta values chosen from 11.5±0.2, 14.9±0.2, 16.6±0.2, 17.0±0.2, 18.8±0.2, 20.5±0.2, 21.2±0.2, 22.6±0.2, 26.4±0.2, and 25.3±0.2. In some embodiments, crystalline Form S of Compound (I) is characterized by an X-ray powder diffractogram having a signal at at least five two-theta values chosen from 11.5±0.2, 14.9±0.2, 16.6±0.2, 17.0±0.2, 18.8±0.2, 20.5±0.2, 21.2±0.2, 22.6±0.2, 26.4±0.2, and 25.3±0.2. In some embodiments, crystalline Form S of Compound (I) is characterized by an X-ray powder diffractogram having a signal at at least four two-theta values chosen from 11.5±0.2, 14.9±0.2, 16.6±0.2, 17.0±0.2, 18.8±0.2, 20.5±0.2, 21.2±0.2, 22.6±0.2, 26.4±0.2, and 25.3±0.2. In some embodiments, crystalline Form S of Compound (I) is characterized by an X-ray powder diffractogram having a signal at at least three two-theta values chosen from 11.5±0.2, 14.9±0.2, 16.6±0.2, 17.0±0.2, 18.8±0.2, 20.5±0.2, 21.2±0.2, 22.6±0.2, 26.4±0.2, and 25.3±0.2. In some embodiments, crystalline Form S of Compound (I) is characterized by an X-ray powder diffractogram having a signal at at least two two-theta values chosen from 11.5±0.2, 14.9±0.2, 16.6±0.2, 17.0±0.2, 18.8±0.2, 20.5±0.2, 21.2±0.2, 22.6±0.2, 26.4±0.2, and 25.3±0.2. In some embodiments, crystalline Form S of Compound (I) is characterized by an X-ray powder diffractogram having a signal at at least one two-theta value chosen from 11.5±0.2, 14.9±0.2, 16.6±0.2, 17.0±0.2, 18.8±0.2, 20.5±0.2, 21.2±0.2, 22.6±0.2, 26.4±0.2, and 25.3±0.2.

In some embodiments, crystalline Form S of Compound (I) is characterized by an X-ray powder diffractogram having a signal at two-theta values of 11.5±0.2, 14.9±0.2, 17.0±0.2, 18.8±0.2, 21.2±0.2, and 22.6±0.2. In some embodiments, crystalline Form S of Compound (I) is characterized by an X-ray powder diffractogram having a signal at at least five two-theta values chosen from 11.5±0.2, 14.9±0.2, 17.0±0.2, 18.8±0.2, 21.2±0.2, and 22.6±0.2. In some embodiments, crystalline Form S of Compound (I) is characterized by an X-ray powder diffractogram having a signal at at least four two-theta values chosen from 11.5±0.2, 14.9±0.2, 17.0±0.2, 18.8±0.2, 21.2±0.2, and 22.6±0.2. In some embodiments, crystalline Form S of Compound (I) is characterized by an X-ray powder diffractogram having a signal at at least three two-theta values chosen from 11.5±0.2, 14.9±0.2, 17.0±0.2, 18.8±0.2, 21.2±0.2, and 22.6±0.2. In some embodiments, crystalline Form S of Compound (I) is characterized by an X-ray powder diffractogram having a signal at at least two two-theta values chosen from 11.5±0.2, 14.9±0.2, 17.0±0.2, 18.8±0.2, 21.2±0.2, and 22.6±0.2. In some embodiments, crystalline Form S of Compound (I) is characterized by an X-ray powder diffractogram having a signal at at least one two-theta value chosen from 11.5±0.2, 14.9±0.2, 17.0±0.2, 18.8±0.2, 21.2±0.2, and 22.6±0.2.

In some embodiments, crystalline Form S of Compound (I) is characterized by an X-ray powder diffractogram having a signal at two-theta values of 14.9±0.2, 18.8±0.2, and 21.2±0.2. In some embodiments, crystalline Form S of Compound (I) is characterized by an X-ray powder diffractogram having a signal at at least two two-theta values chosen from 17.0±0.2, 18.8±0.2, and 21.2±0.2. In some embodiments, crystalline Form S of Compound (I) is characterized by an X-ray powder diffractogram having a signal at at least one two-theta value chosen from 17.0±0.2, 18.8±0.2, and 21.2±0.2.

In some embodiments, crystalline Form S of Compound (I) is characterized by an X-ray powder diffractogram substantially similar to that in FIG. 12.

In some embodiments, the present disclosure provides crystalline Form S of Compound (I) prepared by a process comprising crystallizing Compound (I) from a mixture of 1,4-dioxane, heptane, and Compound (I). In some embodiments, the crystallizing occurs at room temperature.

In some embodiments, the present disclosure provides methods of preparing crystalline Form S of Compound (I) comprising crystallizing Compound (I) from a mixture of 1,4-dioxane, heptane, and Compound (I). In some embodiments, the crystallizing occurs at room temperature.

### Crystalline Form MS of Compound (I)

In some embodiments, the present disclosure provides crystalline Form MS of Compound (I):

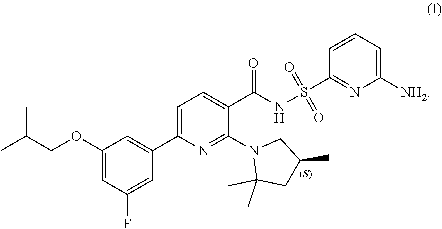

In some embodiments, crystalline Form MS is a methanol solvate of Compound (I).

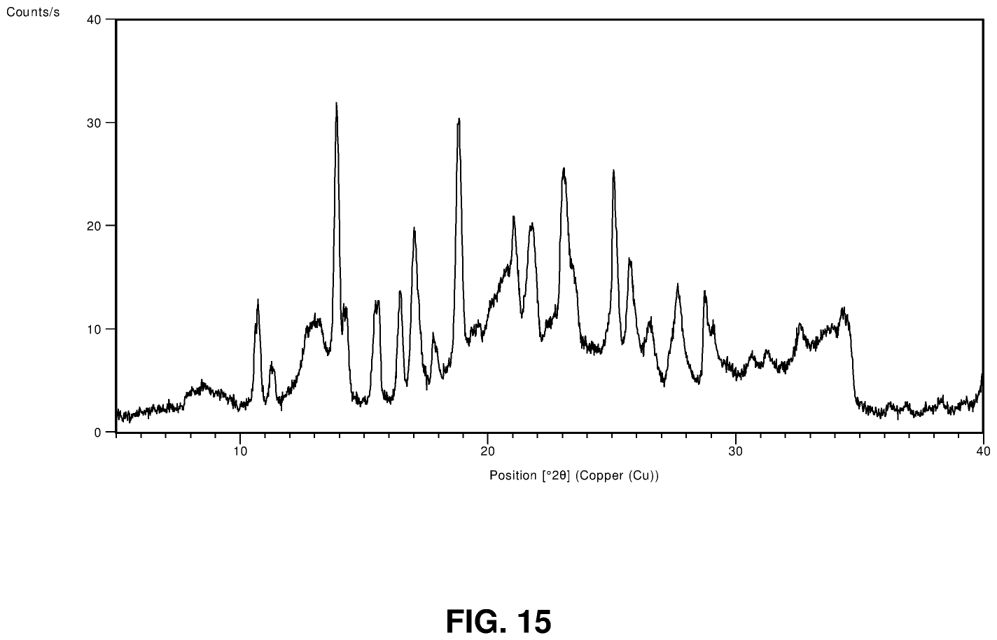

FIG. 15 shows an X-ray powder diffractogram of crystalline Form MS of Compound (I) at ambient conditions.

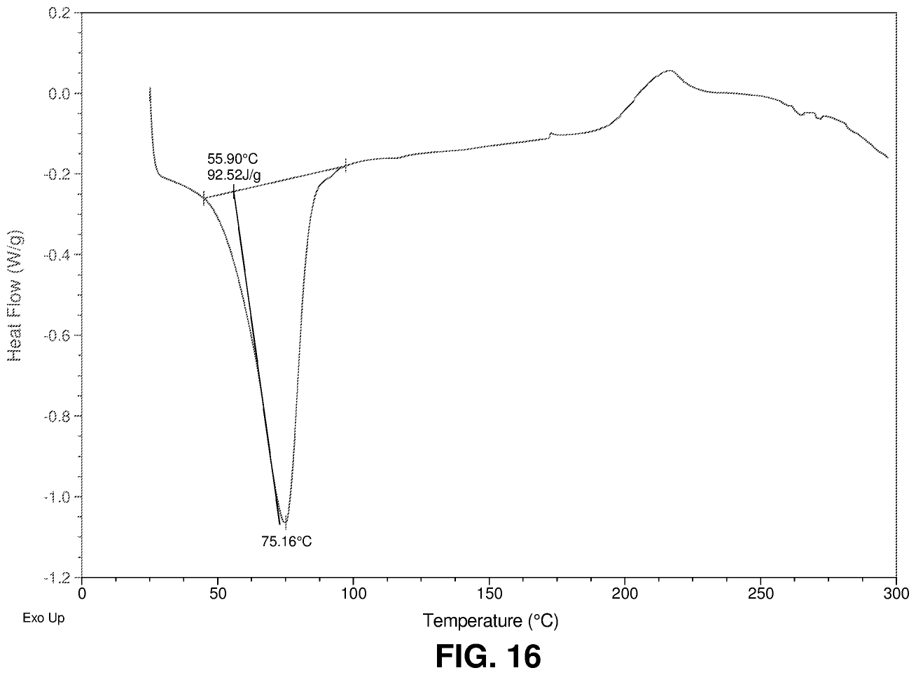

FIG. 16 shows a DSC trace of the crystalline Form MS of Compound (I). In some embodiments, crystalline Form MS of Compound (I) is characterized by a DSC having an onset of decomposition temperature of 55.9° C. and/or a peak temperature of 75.2° C.

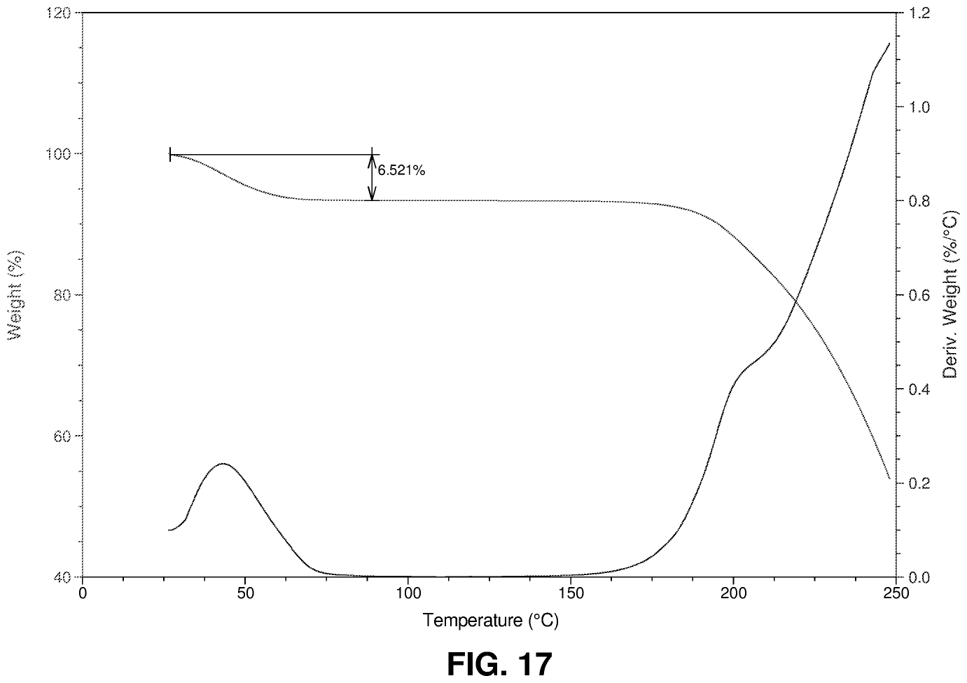

FIG. 17 shows TGA results of crystalline Form MS of Compound (I). In some embodiments, crystalline Form MS of Compound (I) is characterized by a TGA having an onset of decomposition temperature of about 175° C.

In some embodiments, crystalline Form MS of Compound (I) is in substantially pure form.

In some embodiments, crystalline Form MS of Compound (I) is characterized by an X-ray powder diffractogram generated by an X-ray powder diffraction analysis with an incident beam of Cu Kα radiation.

In some embodiments, crystalline Form MS of Compound (I) is characterized by an X-ray powder diffractogram having a signal at 13.9±0.2 degrees two-theta. In some embodiments, crystalline Form MS of Compound (I) is characterized by an X-ray powder diffractogram having a signal at 15.6±0.2 degrees two-theta. In some embodiments, crystalline Form MS of Compound (I) is characterized by an X-ray powder diffractogram having a signal at 16.5±0.2 degrees two-theta. In some embodiments, crystalline Form MS of Compound (I) is characterized by an X-ray powder diffractogram having a signal at 17.0±0.2 degrees two-theta. In some embodiments, crystalline Form MS of Compound (I) is characterized by an X-ray powder diffractogram having a signal at 18.8±0.2 degrees two-theta. In some embodiments, crystalline Form MS of Compound (I) is characterized by an X-ray powder diffractogram having a signal at 21.0±0.2 degrees two-theta. In some embodiments, crystalline Form MS of Compound (I) is characterized by an X-ray powder diffractogram having a signal at 21.8±0.2 degrees two-theta. In some embodiments, crystalline Form MS of Compound (I) is characterized by an X-ray powder diffractogram having a signal at 23.1±0.2 degrees two-theta. In some embodiments, crystalline Form MS of Compound (I) is characterized by an X-ray powder diffractogram having a signal at 25.1±0.2 degrees two-theta. In some embodiments, crystalline Form MS of Compound (I) is characterized by an X-ray powder diffractogram having a signal at 25.7±0.2 degrees two-theta. In some embodiments, crystalline Form MS of Compound (I) is characterized by an X-ray powder diffractogram having a signal at 27.7±0.2 degrees two-theta. In some embodiments, crystalline Form MS of Compound (I) is characterized by an X-ray powder diffractogram having a signal at 28.7±0.2 degrees two-theta.

In some embodiments, crystalline Form MS of Compound (I) is characterized by an X-ray powder diffractogram having a signal at two-theta values of 13.9±0.2, 15.6±0.2, 16.5±0.2, 17.0±0.2, 18.8±0.2, 21.0±0.2, 21.8±0.2, 23.1±0.2, 25.1±0.2, 25.7±0.2, 27.7±0.2, and 28.7±0.2. In some embodiments, crystalline Form MS of Compound (I) is characterized by an X-ray powder diffractogram having a signal at at least eleven two-theta values chosen from 13.9±0.2, 15.6±0.2, 16.5±0.2, 17.0±0.2, 18.8±0.2, 21.0±0.2, 21.8±0.2, 23.1±0.2, 25.1±0.2, 25.7±0.2, 27.7±0.2, and 28.7±0.2. In some embodiments, crystalline Form MS of Compound (I) is characterized by an X-ray powder diffractogram having a signal at at least ten two-theta values chosen from 13.9±0.2, 15.6±0.2, 16.5±0.2, 17.0±0.2, 18.8±0.2, 21.0±0.2, 21.8±0.2, 23.1±0.2, 25.1±0.2, 25.7±0.2, 27.7±0.2, and 28.7±0.2. In some embodiments, crystalline Form MS of Compound (I) is characterized by an X-ray powder diffractogram having a signal at at least nine two-theta values chosen from 13.9±0.2, 15.6±0.2, 16.5±0.2, 17.0±0.2, 18.8±0.2, 21.0±0.2, 21.8±0.2, 23.1±0.2, 25.1±0.2, 25.7±0.2, 27.7±0.2, and 28.7±0.2. In some embodiments, crystalline Form MS of Compound (I) is characterized by an X-ray powder diffractogram having a signal at at least eight two-theta values chosen from 13.9±0.2, 15.6±0.2, 16.5±0.2, 17.0±0.2, 18.8±0.2, 21.0±0.2, 21.8±0.2, 23.1±0.2, 25.1±0.2, 25.7±0.2, 27.7±0.2, and 28.7±0.2. In some embodiments, crystalline Form MS of Compound (I) is characterized by an X-ray powder diffractogram having a signal at at least seven two-theta values chosen from 13.9±0.2, 15.6±0.2, 16.5±0.2, 17.0±0.2, 18.8±0.2, 21.0±0.2, 21.8±0.2, 23.1±0.2, 25.1±0.2, 25.7±0.2, 27.7±0.2, and 28.7±0.2. In some embodiments, crystalline Form MS of Compound (I) is characterized by an X-ray powder diffractogram having a signal at at least six two-theta values chosen from 13.9±0.2, 15.6±0.2, 16.5±0.2, 17.0±0.2, 18.8±0.2, 21.0±0.2, 21.8±0.2, 23.1±0.2, 25.1±0.2, 25.7±0.2, 27.7±0.2, and 28.7±0.2. In some embodiments, crystalline Form MS of Compound (I) is characterized by an X-ray powder diffractogram having a signal at at least five two-theta values chosen from 13.9±0.2, 15.6±0.2, 16.5±0.2, 17.0±0.2, 18.8±0.2, 21.0±0.2, 21.8±0.2, 23.1±0.2, 25.1±0.2, 25.7±0.2, 27.7±0.2, and 28.7±0.2. In some embodiments, crystalline Form MS of Compound (I) is characterized by an X-ray powder diffractogram having a signal at at least four two-theta values chosen from 13.9±0.2, 15.6±0.2, 16.5±0.2, 17.0±0.2, 18.8±0.2, 21.0±0.2, 21.8±0.2, 23.1±0.2, 25.1±0.2, 25.7±0.2, 27.7±0.2, and 28.7±0.2. In some embodiments, crystalline Form MS of Compound (I) is characterized by an X-ray powder diffractogram having a signal at at least three two-theta values chosen from 13.9±0.2, 15.6±0.2, 16.5±0.2, 17.0±0.2, 18.8±0.2, 21.0±0.2, 21.8±0.2, 23.1±0.2, 25.1±0.2, 25.7±0.2, 27.7±0.2, and 28.7±0.2. In some embodiments, crystalline Form MS of Compound (I) is characterized by an X-ray powder diffractogram having a signal at at least two two-theta values chosen from 13.9±0.2, 15.6±0.2, 16.5±0.2, 17.0±0.2, 18.8±0.2, 21.0±0.2, 21.8±0.2, 23.1±0.2, 25.1±0.2, 25.7±0.2, 27.7±0.2, and 28.7±0.2. In some embodiments, crystalline Form MS of Compound (I) is characterized by an X-ray powder diffractogram having a signal at at least one two-theta value chosen from 13.9±0.2, 15.6±0.2, 16.5±0.2, 17.0±0.2, 18.8±0.2, 21.0±0.2, 21.8±0.2, 23.1±0.2, 25.1±0.2, 25.7±0.2, 27.7±0.2, and 28.7±0.2.

In some embodiments, crystalline Form MS of Compound (I) is characterized by an X-ray powder diffractogram having a signal at two-theta values of 13.9±0.2, 17.0±0.2, 18.8±0.2, 21.0±0.2, 21.8±0.2, 23.1±0.2, 25.1±0.2, and 25.7±0.2. In some embodiments, crystalline Form MS of Compound (I) is characterized by an X-ray powder diffractogram having a signal at at least seven two-theta values chosen from 13.9±0.2, 17.0±0.2, 18.8±0.2, 21.0±0.2, 21.8±0.2, 23.1±0.2, 25.1±0.2, and 25.7±0.2. In some embodiments, crystalline Form MS of Compound (I) is characterized by an X-ray powder diffractogram having a signal at at least six two-theta values chosen from 13.9±0.2, 17.0±0.2, 18.8±0.2, 21.0±0.2, 21.8±0.2, 23.1±0.2, 25.1±0.2, and 25.7±0.2. In some embodiments, crystalline Form MS of Compound (I) is characterized by an X-ray powder diffractogram having a signal at at least five two-theta values chosen from 13.9±0.2, 17.0±0.2, 18.8±0.2, 21.0±0.2, 21.8±0.2, 23.1±0.2, 25.1±0.2, and 25.7±0.2. In some embodiments, crystalline Form MS of Compound (I) is characterized by an X-ray powder diffractogram having a signal at at least four two-theta values chosen from 13.9±0.2, 17.0±0.2, 18.8±0.2, 21.0±0.2, 21.8±0.2, 23.1±0.2, 25.1±0.2, and 25.7±0.2. In some embodiments, crystalline Form MS of Compound (I) is characterized by an X-ray powder diffractogram having a signal at at least three two-theta values chosen from 13.9±0.2, 17.0±0.2, 18.8±0.2, 21.0±0.2, 21.8±0.2, 23.1±0.2, 25.1±0.2, and 25.7±0.2. In some embodiments, crystalline Form MS of Compound (I) is characterized by an X-ray powder diffractogram having a signal at at least two two-theta values chosen from 13.9±0.2, 17.0±0.2, 18.8±0.2, 21.0±0.2, 21.8±0.2, 23.1±0.2, 25.1±0.2, and 25.7±0.2. In some embodiments, crystalline Form MS of Compound (I) is characterized by an X-ray powder diffractogram having a signal at at least one two-theta value chosen from 13.9±0.2, 17.0±0.2, 18.8±0.2, 21.0±0.2, 21.8±0.2, 23.1±0.2, 25.1±0.2, and 25.7±0.2.

In some embodiments, crystalline Form MS of Compound (I) is characterized by an X-ray powder diffractogram having a signal at two-theta values of 13.9±0.2, 18.8±0.2, 23.1±0.2, and 25.1±0.2. In some embodiments, crystalline Form MS of Compound (I) is characterized by an X-ray powder diffractogram having a signal at at least three two-theta values chosen from 13.9±0.2, 18.8±0.2, 23.1±0.2, and 25.1±0.2. In some embodiments, crystalline Form MS of Compound (I) is characterized by an X-ray powder diffractogram having a signal at at least two two-theta values chosen from 13.9±0.2, 18.8±0.2, 23.1±0.2, and 25.1±0.2. In some embodiments, crystalline Form MS of Compound (I) is characterized by an X-ray powder diffractogram having a signal at at least one two-theta value chosen from 13.9±0.2, 18.8±0.2, 23.1±0.2, and 25.1±0.2.

In some embodiments, crystalline Form MS of Compound (I) is characterized by an X-ray powder diffractogram having a signal at two-theta values of 13.9±0.2, 25.1±0.2, and 25.7±0.2. In some embodiments, crystalline Form MS of Compound (I) is characterized by an X-ray powder diffractogram having a signal at at least two two-theta values chosen from 13.9±0.2, 25.1±0.2, and 25.7±0.2. In some embodiments, crystalline Form MS of Compound (I) is characterized by an X-ray powder diffractogram having a signal at at least one two-theta value chosen from 13.9±0.2, 25.1±0.2, and 25.7±0.2.

In some embodiments, crystalline Form MS of Compound (I) is characterized by an X-ray powder diffractogram substantially similar to that in FIG. 15.

In some embodiments, the present disclosure provides crystalline Form MS of Compound (I) prepared by a process comprising isolating Compound (I) from a mixture of dicloromethane, methanol, and Compound (I). In some embodiments, the mixture is a 9 to 1 (w/w) mixture of dichloromethane to methanol.

In some embodiments, the present disclosure provides methods of preparing crystalline Form MS of Compound (I) comprising isolating Compound (I) from a mixture of dicloromethane, methanol, and Compound (I). In some embodiments, the mixture is a 9 to 1 (w/w) mixture of dichloromethane to methanol.

### Crystalline Forms of Compound (II)

### Crystalline Form A2 of Compound (II)

In some embodiments, the present disclosure provides crystalline Form A2 of Compound (II):

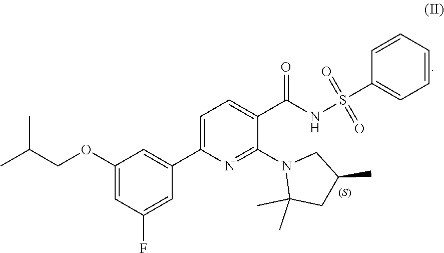

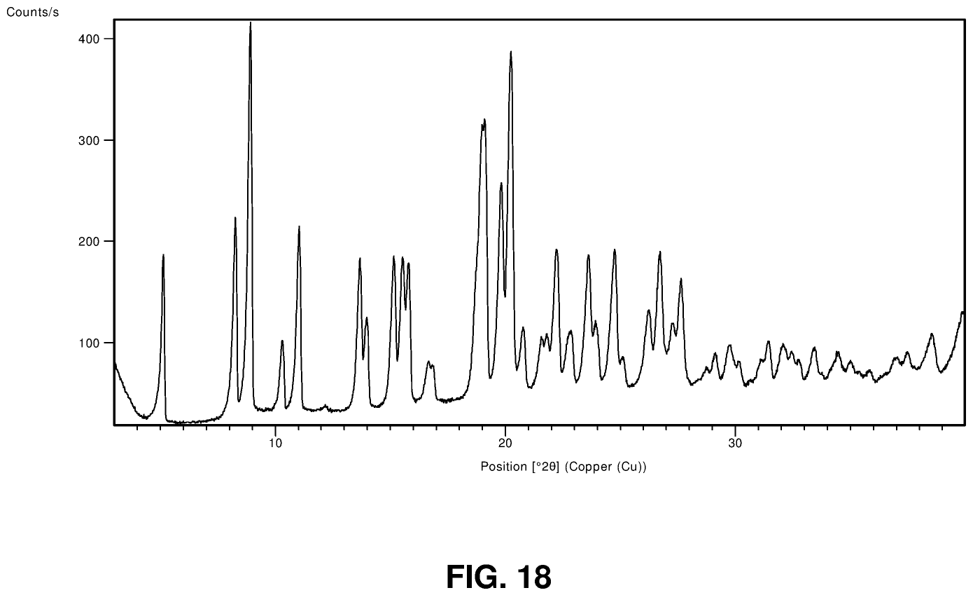

FIG. 18 shows an X-ray powder diffractogram of crystalline Form A2 of Compound (II) at ambient conditions.

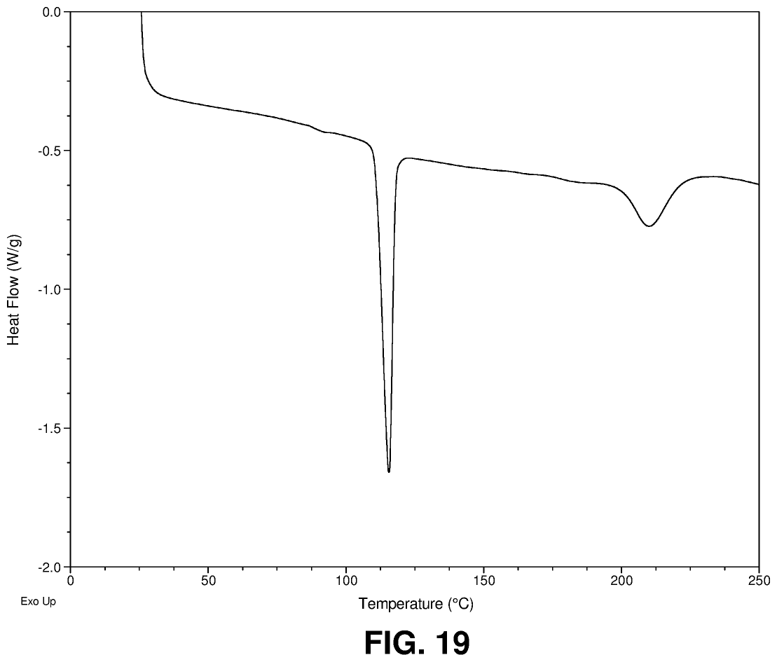

FIG. 19 shows a DSC trace of the crystalline Form A2 of Compound (II). In some embodiments, crystalline Form A2 of Compound (II) is characterized by a DSC having an onset of melting temperature of 111° C. and/or a peak temperature of 116° C.

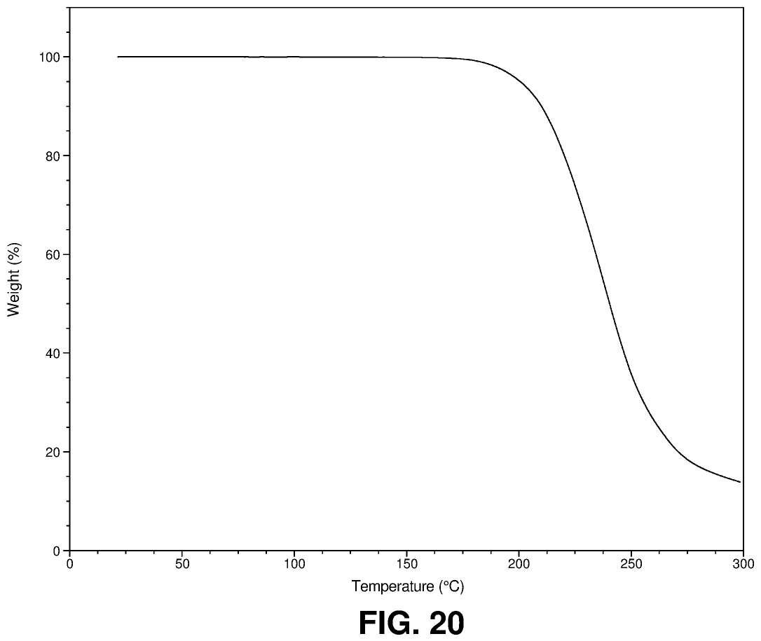

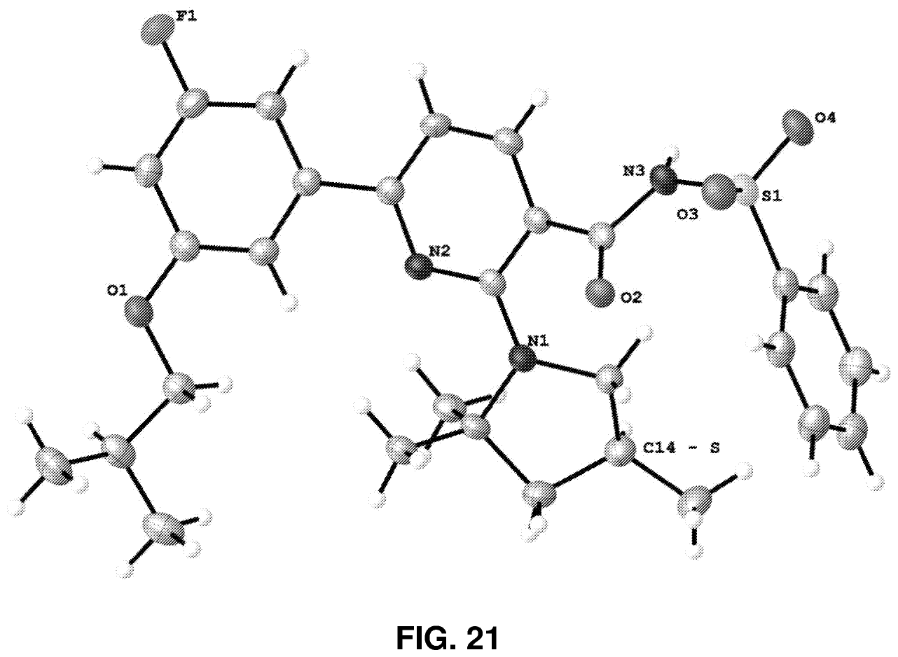

FIG. 20 shows the results of a TGA of crystalline Form A2 of Compound (II). In some embodiments, crystalline Form A2 of Compound (II) is characterized by a TGA having an onset of decomposition temperature of about 200° C.

In some embodiments, crystalline Form A2 of Compound (II) is in substantially pure form. In some embodiments, crystalline Form A2 of Compound (II) is characterized by an X-ray powder diffractogram generated by an X-ray powder diffraction analysis with an incident beam of Cu Kα radiation.

In some embodiments, crystalline Form A2 of Compound (II) is characterized by an X-ray powder diffractogram having a signal at 8.4±0.2 degrees two-theta. In some embodiments, crystalline Form A2 of Compound (II) is characterized by an X-ray powder diffractogram having a signal at 9.0±0.2 degrees two-theta. In some embodiments, crystalline Form A2 of Compound (II) is characterized by an X-ray powder diffractogram having a signal at 11.2±0.2 degrees two-theta. In some embodiments, crystalline Form A2 of Compound (II) is characterized by an X-ray powder diffractogram having a signal at 13.8±0.2 degrees two-theta. In some embodiments, crystalline Form A2 of Compound (II) is characterized by an X-ray powder diffractogram having a signal at 15.3±0.2 degrees two-theta. In some embodiments, crystalline Form A2 of Compound (II) is characterized by an X-ray powder diffractogram having a signal at 15.7±0.2 degrees two-theta. In some embodiments, crystalline Form A2 of Compound (II) is characterized by an X-ray powder diffractogram having a signal at 19.3±0.2 degrees two-theta. In some embodiments, crystalline Form A2 of Compound (II) is characterized by an X-ray powder diffractogram having a signal at 20.0±0.2 degrees two-theta. In some embodiments, crystalline Form A2 of Compound (II) is characterized by an X-ray powder diffractogram having a signal at 20.4±0.2 degrees two-theta. In some embodiments, crystalline Form A2 of Compound (II) is characterized by an X-ray powder diffractogram having a signal at 23.7±0.2 degrees two-theta. In some embodiments, crystalline Form A2 of Compound (II) is characterized by an X-ray powder diffractogram having a signal at 24.9±0.2 degrees two-theta. In some embodiments, crystalline Form A2 of Compound (II) is characterized by an X-ray powder diffractogram having a signal at 27.8±0.2 degrees two-theta.

In some embodiments, crystalline Form A2 of Compound (II) is characterized by an X-ray powder diffractogram having a signal at two-theta values of 8.4±0.2, 9.0±0.2, 11.2±0.2, 13.8±0.2, 15.3±0.2, 15.7±0.2, 19.3±0.2, 20.0±0.2, 20.4±0.2, 23.7±0.2, 24.9±0.2, and 27.8±0.2. In some embodiments, crystalline Form A2 of Compound (II) is characterized by an X-ray powder diffractogram having a signal at at least eleven two-theta values chosen from 8.4±0.2, 9.0±0.2, 11.2±0.2, 13.8±0.2, 15.3±0.2, 15.7±0.2, 19.3±0.2, 20.0±0.2, 20.4±0.2, 23.7±0.2, 24.9±0.2, and 27.8±0.2. In some embodiments, crystalline Form A2 of Compound (II) is characterized by an X-ray powder diffractogram having a signal at at least ten two-theta values chosen from 8.4±0.2, 9.0±0.2, 11.2±0.2, 13.8±0.2, 15.3±0.2, 15.7±0.2, 19.3±0.2, 20.0±0.2, 20.4±0.2, 23.7±0.2, 24.9±0.2, and 27.8±0.2. In some embodiments, crystalline Form A2 of Compound (II) is characterized by an X-ray powder diffractogram having a signal at at least nine two-theta values chosen from 8.4±0.2, 9.0±0.2, 11.2±0.2, 13.8±0.2, 15.3±0.2, 15.7±0.2, 19.3±0.2, 20.0±0.2, 20.4±0.2, 23.7±0.2, 24.9±0.2, and 27.8±0.2. In some embodiments, crystalline Form A2 of Compound (II) is characterized by an X-ray powder diffractogram having a signal at at least eight two-theta values chosen from 8.4±0.2, 9.0±0.2, 11.2±0.2, 13.8±0.2, 15.3±0.2, 15.7±0.2, 19.3±0.2, 20.0±0.2, 20.4±0.2, 23.7±0.2, 24.9±0.2, and 27.8±0.2. In some embodiments, crystalline Form A2 of Compound (II) is characterized by an X-ray powder diffractogram having a signal at at least seven two-theta values chosen from 8.4±0.2, 9.0±0.2, 11.2±0.2, 13.8±0.2, 15.3±0.2, 15.7±0.2, 19.3±0.2, 20.0±0.2, 20.4±0.2, 23.7±0.2, 24.9±0.2, and 27.8±0.2. In some embodiments, crystalline Form A2 of Compound (II) is characterized by an X-ray powder diffractogram having a signal at at least six two-theta values chosen from 8.4±0.2, 9.0±0.2, 11.2±0.2, 13.8±0.2, 15.3±0.2, 15.7±0.2, 19.3±0.2, 20.0±0.2, 20.4±0.2, 23.7±0.2, 24.9±0.2, and 27.8±0.2. In some embodiments, crystalline Form A2 of Compound (II) is characterized by an X-ray powder diffractogram having a signal at at least five two-theta values chosen from 8.4±0.2, 9.0±0.2, 11.2±0.2, 13.8±0.2, 15.3±0.2, 15.7±0.2, 19.3±0.2, 20.0±0.2, 20.4±0.2, 23.7±0.2, 24.9±0.2, and 27.8±0.2. In some embodiments, crystalline Form A2 of Compound (II) is characterized by an X-ray powder diffractogram having a signal at at least four two-theta values chosen from 8.4±0.2, 9.0±0.2, 11.2±0.2, 13.8±0.2, 15.3±0.2, 15.7±0.2, 19.3±0.2, 20.0±0.2, 20.4±0.2, 23.7±0.2, 24.9±0.2, and 27.8±0.2. In some embodiments, crystalline Form A2 of Compound (II) is characterized by an X-ray powder diffractogram having a signal at at least three two-theta values chosen from 8.4±0.2, 9.0±0.2, 11.2±0.2, 13.8±0.2, 15.3±0.2, 15.7±0.2, 19.3±0.2, 20.0±0.2, 20.4±0.2, 23.7±0.2, 24.9±0.2, and 27.8±0.2. In some embodiments, crystalline Form A2 of Compound (II) is characterized by an X-ray powder diffractogram having a signal at at least two two-theta values chosen from 8.4±0.2, 9.0±0.2, 11.2±0.2, 13.8±0.2, 15.3±0.2, 15.7±0.2, 19.3±0.2, 20.0±0.2, 20.4±0.2, 23.7±0.2, 24.9±0.2, and 27.8±0.2. In some embodiments, crystalline Form A2 of Compound (II) is characterized by an X-ray powder diffractogram having a signal at at least one two-theta value chosen from 8.4±0.2, 9.0±0.2, 11.2±0.2, 13.8±0.2, 15.3±0.2, 15.7±0.2, 19.3±0.2, 20.0±0.2, 20.4±0.2, 23.7±0.2, 24.9±0.2, and 27.8±0.2.

In some embodiments, crystalline Form A2 of Compound (II) is characterized by an X-ray powder diffractogram having a signal at two-theta values of 9.0±0.2, 11.2±0.2, 13.8±0.2, 15.3±0.2, 15.7±0.2, 19.3±0.2, 20.0±0.2, 20.4±0.2, 23.7±0.2, and 24.9±0.2. In some embodiments, crystalline Form A2 of Compound (II) is characterized by an X-ray powder diffractogram having a signal at at least nine two-theta values chosen from 9.0±0.2, 11.2±0.2, 13.8±0.2, 15.3±0.2, 15.7±0.2, 19.3±0.2, 20.0±0.2, 20.4±0.2, 23.7±0.2, and 24.9±0.2. In some embodiments, crystalline Form A2 of Compound (II) is characterized by an X-ray powder diffractogram having a signal at at least eight two-theta values chosen from 9.0±0.2, 11.2±0.2, 13.8±0.2, 15.3±0.2, 15.7±0.2, 19.3±0.2, 20.0±0.2, 20.4±0.2, 23.7±0.2, and 24.9±0.2. In some embodiments, crystalline Form A2 of Compound (II) is characterized by an X-ray powder diffractogram having a signal at at least seven two-theta values chosen from 9.0±0.2, 11.2±0.2, 13.8±0.2, 15.3±0.2, 15.7±0.2, 19.3±0.2, 20.0±0.2, 20.4±0.2, 23.7±0.2, and 24.9±0.2. In some embodiments, crystalline Form A2 of Compound (II) is characterized by an X-ray powder diffractogram having a signal at at least six two-theta values chosen from 9.0±0.2, 11.2±0.2, 13.8±0.2, 15.3±0.2, 15.7±0.2, 19.3±0.2, 20.0±0.2, 20.4±0.2, 23.7±0.2, and 24.9±0.2. In some embodiments, crystalline Form A2 of Compound (II) is characterized by an X-ray powder diffractogram having a signal at at least five two-theta values chosen from 9.0±0.2, 11.2±0.2, 13.8±0.2, 15.3±0.2, 15.7±0.2, 19.3±0.2, 20.0±0.2, 20.4±0.2, 23.7±0.2, and 24.9±0.2. In some embodiments, crystalline Form A2 of Compound (II) is characterized by an X-ray powder diffractogram having a signal at at least four two-theta values chosen from 9.0±0.2, 11.2±0.2, 13.8±0.2, 15.3±0.2, 15.7±0.2, 19.3±0.2, 20.0±0.2, 20.4±0.2, 23.7±0.2, and 24.9±0.2. In some embodiments, crystalline Form A2 of Compound (II) is characterized by an X-ray powder diffractogram having a signal at at least three two-theta values chosen from 9.0±0.2, 11.2±0.2, 13.8±0.2, 15.3±0.2, 15.7±0.2, 19.3±0.2, 20.0±0.2, 20.4±0.2, 23.7±0.2, and 24.9±0.2. In some embodiments, crystalline Form A2 of Compound (II) is characterized by an X-ray powder diffractogram having a signal at at least two two-theta values chosen from 9.0±0.2, 11.2±0.2, 13.8±0.2, 15.3±0.2, 15.7±0.2, 19.3±0.2, 20.0±0.2, 20.4±0.2, 23.7±0.2, and 24.9±0.2. In some embodiments, crystalline Form A2 of Compound (II) is characterized by an X-ray powder diffractogram having a signal at at least one two-theta value chosen from 9.0±0.2, 11.2±0.2, 13.8±0.2, 15.3±0.2, 15.7±0.2, 19.3±0.2, 20.0±0.2, 20.4±0.2, 23.7±0.2, and 24.9±0.2.

In some embodiments, crystalline Form A2 of Compound (II) is characterized by an X-ray powder diffractogram having a signal at two-theta values of 9.0±0.2, 11.2±0.2, 13.8±0.2, 15.3±0.2, 15.7±0.2, 19.3±0.2, and 20.0±0.2. In some embodiments, crystalline Form A2 of Compound (II) is characterized by an X-ray powder diffractogram having a signal at at least six two-theta values chosen from 9.0±0.2, 11.2±0.2, 13.8±0.2, 15.3±0.2, 15.7±0.2, 19.3±0.2, and 20.0±0.2. In some embodiments, crystalline Form A2 of Compound (II) is characterized by an X-ray powder diffractogram having a signal at at least five two-theta values chosen from 9.0±0.2, 11.2±0.2, 13.8±0.2, 15.3±0.2, 15.7±0.2, 19.3±0.2, and 20.0±0.2. In some embodiments, crystalline Form A2 of Compound (II) is characterized by an X-ray powder diffractogram having a signal at at least four two-theta values chosen from 9.0±0.2, 11.2±0.2, 13.8±0.2, 15.3±0.2, 15.7±0.2, 19.3±0.2, and 20.0±0.2. In some embodiments, crystalline Form A2 of Compound (II) is characterized by an X-ray powder diffractogram having a signal at at least three two-theta values chosen from 9.0±0.2, 11.2±0.2, 13.8±0.2, 15.3±0.2, 15.7±0.2, 19.3±0.2, and 20.0±0.2. In some embodiments, crystalline Form A2 of Compound (II) is characterized by an X-ray powder diffractogram having a signal at at least two two-theta values chosen from 9.0±0.2, 11.2±0.2, 13.8±0.2, 15.3±0.2, 15.7±0.2, 19.3±0.2, and 20.0±0.2. In some embodiments, crystalline Form A2 of Compound (II) is characterized by an X-ray powder diffractogram having a signal at at least one two-theta value chosen from 9.0±0.2, 11.2±0.2, 13.8±0.2, 15.3±0.2, 15.7±0.2, 19.3±0.2, and 20.0±0.2.

In some embodiments, crystalline Form A2 of Compound (II) is characterized by an X-ray powder diffractogram having a signal at two-theta values of 9.0±0.2, 11.2±0.2, 13.8±0.2, 15.3±0.2, 15.7±0.2, and 19.3±0.2. In some embodiments, crystalline Form A2 of Compound (II) is characterized by an X-ray powder diffractogram having a signal at at least five two-theta values chosen from 9.0±0.2, 11.2±0.2, 13.8±0.2, 15.3±0.2, 15.7±0.2, and 19.3±0.2. In some embodiments, crystalline Form A2 of Compound (II) is characterized by an X-ray powder diffractogram having a signal at at least four two-theta values chosen from 9.0±0.2, 11.2±0.2, 13.8±0.2, 15.3±0.2, 15.7±0.2, and 19.3±0.2. In some embodiments, crystalline Form A2 of Compound (II) is characterized by an X-ray powder diffractogram having a signal at at least three two-theta values chosen from 9.0±0.2, 11.2±0.2, 13.8±0.2, 15.3±0.2, 15.7±0.2, and 19.3±0.2. In some embodiments, crystalline Form A2 of Compound (II) is characterized by an X-ray powder diffractogram having a signal at at least two two-theta values chosen from 9.0±0.2, 11.2±0.2, 13.8±0.2, 15.3±0.2, 15.7±0.2, and 19.3±0.2. In some embodiments, crystalline Form A2 of Compound (II) is characterized by an X-ray powder diffractogram having a signal at at least one two-theta value chosen from 9.0±0.2, 11.2±0.2, 13.8±0.2, 15.3±0.2, 15.7±0.2, and 19.3±0.2.

In some embodiments, crystalline Form A2 of Compound (II) is characterized by an X-ray powder diffractogram having a signal at two-theta values of 8.4±0.2, 9.0±0.2, 11.2±0.2, 13.8±0.2, 15.3±0.2, 15.7±0.2, and 19.3±0.2. In some embodiments, crystalline Form A2 of Compound (II) is characterized by an X-ray powder diffractogram having a signal at at least six two-theta values chosen from 8.4±0.2, 9.0±0.2, 11.2±0.2, 13.8±0.2, 15.3±0.2, 15.7±0.2, and 19.3±0.2. In some embodiments, crystalline Form A2 of Compound (II) is characterized by an X-ray powder diffractogram having a signal at at least five two-theta values chosen from 8.4±0.2, 9.0±0.2, 11.2±0.2, 13.8±0.2, 15.3±0.2, 15.7±0.2, and 19.3±0.2. In some embodiments, crystalline Form A2 of Compound (II) is characterized by an X-ray powder diffractogram having a signal at at least four two-theta values chosen from 8.4±0.2, 9.0±0.2, 11.2±0.2, 13.8±0.2, 15.3±0.2, 15.7±0.2, and 19.3±0.2. In some embodiments, crystalline Form A2 of Compound (II) is characterized by an X-ray powder diffractogram having a signal at at least three two-theta values chosen from 8.4±0.2, 9.0±0.2, 11.2±0.2, 13.8±0.2, 15.3±0.2, 15.7±0.2, and 19.3±0.2. In some embodiments, crystalline Form A2 of Compound (II) is characterized by an X-ray powder diffractogram having a signal at at least two two-theta values chosen from 8.4±0.2, 9.0±0.2, 11.2±0.2, 13.8±0.2, 15.3±0.2, 15.7±0.2, and 19.3±0.2. In some embodiments, crystalline Form A2 of Compound (II) is characterized by an X-ray powder diffractogram having a signal at at least one two-theta value chosen from 8.4±0.2, 9.0±0.2, 11.2±0.2, 13.8±0.2, 15.3±0.2, 15.7±0.2, and 19.3±0.2.

In some embodiments, crystalline Form A2 of Compound (II) is characterized by an X-ray powder diffractogram having a signal at two-theta values of 9.0±0.2, 11.2±0.2, 13.8±0.2, 15.7±0.2, and 20.0±0.2. In some embodiments, crystalline Form A2 of Compound (II) is characterized by an X-ray powder diffractogram having a signal at at least four two-theta values chosen from 9.0±0.2, 11.2±0.2, 13.8±0.2, 15.7±0.2, and 20.0±0.2. In some embodiments, crystalline Form A2 of Compound (II) is characterized by an X-ray powder diffractogram having a signal at at least three two-theta values chosen from 9.0±0.2, 11.2±0.2, 13.8±0.2, 15.7±0.2, and 20.0±0.2. In some embodiments, crystalline Form A2 of Compound (II) is characterized by an X-ray powder diffractogram having a signal at at least two two-theta values chosen from 9.0±0.2, 11.2±0.2, 13.8±0.2, 15.7±0.2, and 20.0±0.2. In some embodiments, crystalline Form A2 of Compound (II) is characterized by an X-ray powder diffractogram having a signal at at least one two-theta value chosen from 9.0±0.2, 11.2±0.2, 13.8±0.2, 15.7±0.2, and 20.0±0.2.

In some embodiments, crystalline Form A2 of Compound (II) is characterized by an X-ray powder diffractogram substantially similar to that in FIG. 18.

In some embodiments, crystalline Form A2 of Compound (II) is characterized by a hexagonal crystal system. In some embodiments, crystalline Form A2 of Compound (II) is characterized as belonging to a hexagonal space group P61 (No. 169). In some embodiments, crystalline Form A2 of Compound (II) is characterized by having a unit cell characterized by three edges of 19.4681±0.0007 Å, 19.4681±0.0007 Å, and 13.3151±0.0005 Å.

In some embodiments, crystalline Form A2 of Compound (II) is characterized by having a unit cell with the following characteristics measured at 100 K and 0.71073 A:

| Crystal | Hexagonal |
|---|---|
| System: | |
| Space | P6₁ |
| Group: | |
| a (Å): | 19.4681(7) |
| b (Å): | 19.4681(7) |
| c (Å): | 13.3151(5) |
| α (°): | 90 |
| β (°): | 90 |
| γ (°): | 120 |
| V (Å3): | 4370.4(4) |
| Z/Z′: | 6/1 |

In some embodiments, crystalline Form A2 of Compound (II) is characterized by having a unit cell characterized with an angle, α, of 90°. In some embodiments, crystalline Form A2 of Compound (II) is characterized by having a unit cell characterized with an angle, β, of 90°. In some embodiments, crystalline Form A2 of Compound (II) is characterized by having a unit cell characterized with an angle, γ, of 120°.

In some embodiments, crystalline Form A2 of Compound (II) is characterized by having a unit cell with volume of 4370.4±0.4 Å³. In some embodiments, crystalline Form A2 of Compound (II) is characterized by having a unit cell with volume of 4370 Å³.

In some embodiments, crystalline Form A2 of Compound (II) is characterized by a single crystal structure substantially similar to that in FIG. 21.

In some embodiments, crystalline Form A2 of Compound (II) is characterized by an infrared absorption spectrum having one or more signals at wavenumbers (cm⁻¹) chosen from 1044, 1134, 1245, 1331, 1443, 1548, 1577, 1610, 1650, 2865, 2957, and 3068.

In some embodiments, the present disclosure provides crystalline Form A2 of Compound (II) prepared by a process comprising crystallizing Compound (II) from a mixture of ethanol water, and Compound (II). In some embodiments, the crystallizing is carried out above room temperature. In some embodiments, the crystallizing is carried out at room temperature.

In some embodiments, the present disclosure provides methods of preparing crystalline Form A2 of Compound (II) comprising crystallizing Compound (II) from a mixture of ethanol, water, and Compound (II). In some embodiments, the crystallizing is carried out above room temperature. In some embodiments, the crystallizing is carried out at room temperature.

### Crystalline Form IP of Compound (II)

In some embodiments, the present disclosure provides crystalline Form IP of Compound (II):

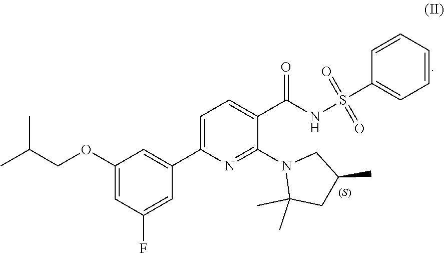

In some embodiments, crystalline Form IP is an iso-propanol solvate of Compound (II).

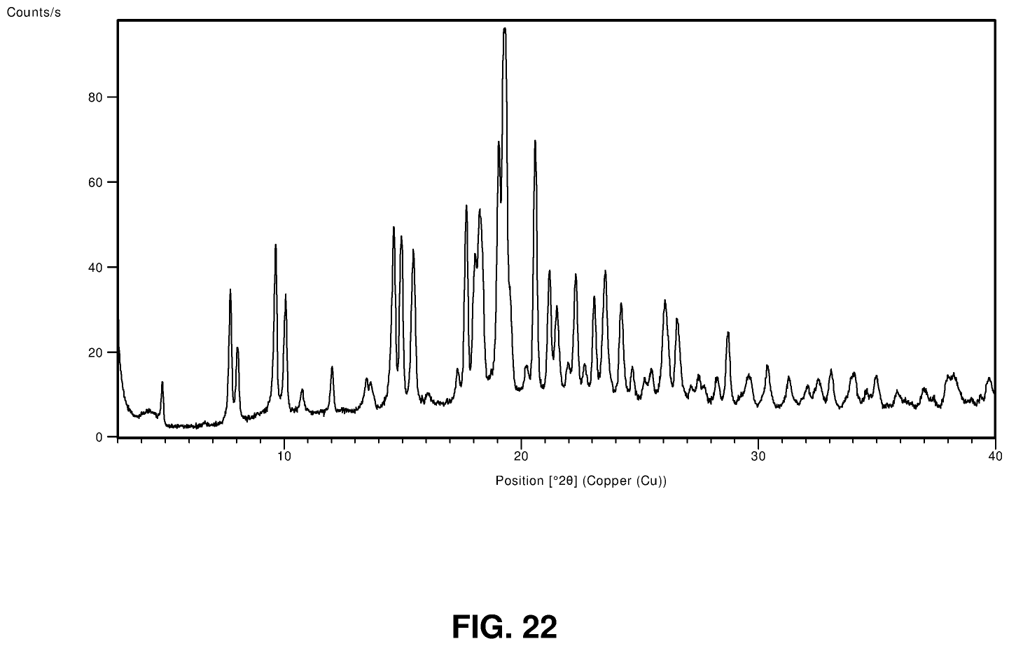

FIG. 22 shows an X-ray powder diffractogram of crystalline Form IP of Compound (II) at ambient conditions.

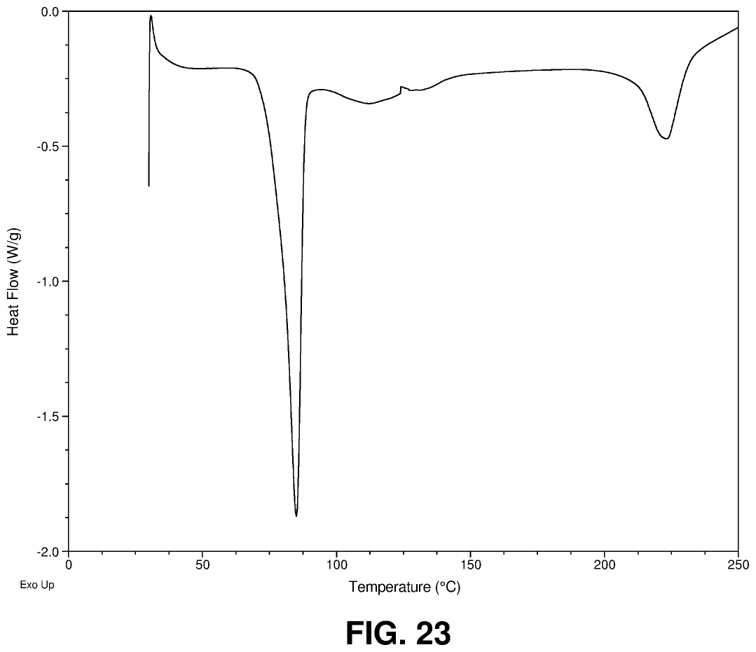

FIG. 23 shows a DSC trace of the crystalline Form IP of Compound (II). In some embodiments, crystalline Form IP of Compound (II) is characterized by a DSC having an onset of desolvation temperature of 78.4° C. and/or a peak temperature of 85° C.

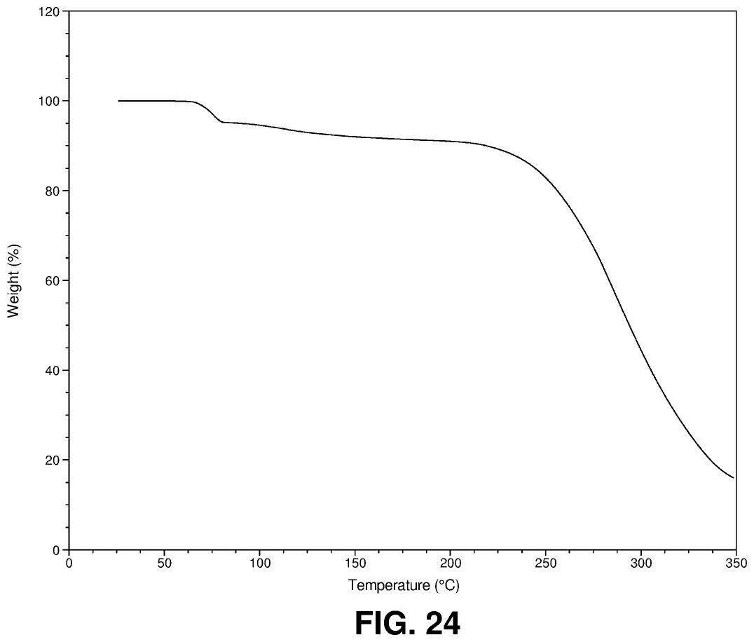

FIG. 24 shows the results of a TGA of crystalline Form IP of Compound (II). In some embodiments, crystalline Form IP of Compound (II) is characterized by a TGA having an onset of decomposition temperature of about 200° C.

In some embodiments, crystalline Form IP of Compound (II) is in substantially pure form. In some embodiments, crystalline Form IP of Compound (II) is characterized by an X-ray powder diffractogram generated by an X-ray powder diffraction analysis with an incident beam of Cu Kα radiation.

In some embodiments, crystalline Form IP of Compound (II) is characterized by an X-ray powder diffractogram having a signal at 9.7±0.2 degrees two-theta. In some embodiments, crystalline Form IP of Compound (II) is characterized by an X-ray powder diffractogram having a signal at 10.1±0.2 degrees two-theta. In some embodiments, crystalline Form IP of Compound (II) is characterized by an X-ray powder diffractogram having a signal at 12.0±0.2 degrees two-theta. In some embodiments, crystalline Form IP of Compound (II) is characterized by an X-ray powder diffractogram having a signal at 14.6±0.2 degrees two-theta. In some embodiments, crystalline Form IP of Compound (II) is characterized by an X-ray powder diffractogram having a signal at 15.0±0.2 degrees two-theta. In some embodiments, crystalline Form IP of Compound (II) is characterized by an X-ray powder diffractogram having a signal at 17.7±0.2 degrees two-theta. In some embodiments, crystalline Form IP of Compound (II) is characterized by an X-ray powder diffractogram having a signal at 18.2±0.2 degrees two-theta. In some embodiments, crystalline Form IP of Compound (II) is characterized by an X-ray powder diffractogram having a signal at 19.0±0.2 degrees two-theta. In some embodiments, crystalline Form IP of Compound (II) is characterized by an X-ray powder diffractogram having a signal at 19.4±0.2 degrees two-theta. In some embodiments, crystalline Form IP of Compound (II) is characterized by an X-ray powder diffractogram having a signal at 20.6±0.2 degrees two-theta.

In some embodiments, crystalline Form IP of Compound (II) is characterized by an X-ray powder diffractogram having a signal at two-theta values of 9.7±0.2, 10.1±0.2, 12.0±0.2, 14.6±0.2, 15.0±0.2, 17.7±0.2, 18.2±0.2, 19.0±0.2, 19.4±0.2, and 20.6±0.2. In some embodiments, crystalline Form IP of Compound (II) is characterized by an X-ray powder diffractogram having a signal at at least nine two-theta values chosen from 9.7±0.2, 10.1±0.2, 12.0±0.2, 14.6±0.2, 15.0±0.2, 17.7±0.2, 18.2±0.2, 19.0±0.2, 19.4±0.2, and 20.6±0.2. In some embodiments, crystalline Form IP of Compound (II) is characterized by an X-ray powder diffractogram having a signal at at least eight two-theta values chosen from 9.7±0.2, 10.1±0.2, 12.0±0.2, 14.6±0.2, 15.0±0.2, 17.7±0.2, 18.2±0.2, 19.0±0.2, 19.4±0.2, and 20.6±0.2. In some embodiments, crystalline Form IP of Compound (II) is characterized by an X-ray powder diffractogram having a signal at at least seven two-theta values chosen from 9.7±0.2, 10.1±0.2, 12.0±0.2, 14.6±0.2, 15.0±0.2, 17.7±0.2, 18.2±0.2, 19.0±0.2, 19.4±0.2, and 20.6±0.2. In some embodiments, crystalline Form IP of Compound (II) is characterized by an X-ray powder diffractogram having a signal at at least six two-theta values chosen from 9.7±0.2, 10.1±0.2, 12.0±0.2, 14.6±0.2, 15.0±0.2, 17.7±0.2, 18.2±0.2, 19.0±0.2, 19.4±0.2, and 20.6±0.2. In some embodiments, crystalline Form IP of Compound (II) is characterized by an X-ray powder diffractogram having a signal at at least five two-theta values chosen from 9.7±0.2, 10.1±0.2, 12.0±0.2, 14.6±0.2, 15.0±0.2, 17.7±0.2, 18.2±0.2, 19.0±0.2, 19.4±0.2, and 20.6±0.2. In some embodiments, crystalline Form IP of Compound (II) is characterized by an X-ray powder diffractogram having a signal at at least four two-theta values chosen from 9.7±0.2, 10.1±0.2, 12.0±0.2, 14.6±0.2, 15.0±0.2, 17.7±0.2, 18.2±0.2, 19.0±0.2, 19.4±0.2, and 20.6±0.2. In some embodiments, crystalline Form IP of Compound (II) is characterized by an X-ray powder diffractogram having a signal at at least three two-theta values chosen from 9.7±0.2, 10.1±0.2, 12.0±0.2, 14.6±0.2, 15.0±0.2, 17.7±0.2, 18.2±0.2, 19.0±0.2, 19.4±0.2, and 20.6±0.2. In some embodiments, crystalline Form IP of Compound (II) is characterized by an X-ray powder diffractogram having a signal at at least two two-theta values chosen from 9.7±0.2, 10.1±0.2, 12.0±0.2, 14.6±0.2, 15.0±0.2, 17.7±0.2, 18.2±0.2, 19.0±0.2, 19.4±0.2, and 20.6±0.2. In some embodiments, crystalline Form IP of Compound (II) is characterized by an X-ray powder diffractogram having a signal at at least one two-theta value chosen from 9.7±0.2, 10.1±0.2, 12.0±0.2, 14.6±0.2, 15.0±0.2, 17.7±0.2, 18.2±0.2, 19.0±0.2, 19.4±0.2, and 20.6±0.2.

In some embodiments, crystalline Form IP of Compound (II) is characterized by an X-ray powder diffractogram having a signal at two-theta values of 10.1±0.2, 12.0±0.2, 14.6±0.2, 15.0±0.2, 19.0±0.2, 19.4±0.2, and 20.6±0.2. In some embodiments, crystalline Form IP of Compound (II) is characterized by an X-ray powder diffractogram having a signal at at least six two-theta values chosen from 10.1±0.2, 12.0±0.2, 14.6±0.2, 15.0±0.2, 19.0±0.2, 19.4±0.2, and 20.6±0.2. In some embodiments, crystalline Form IP of Compound (II) is characterized by an X-ray powder diffractogram having a signal at at least five two-theta values chosen from 10.1±0.2, 12.0±0.2, 14.6±0.2, 15.0±0.2, 19.0±0.2, 19.4±0.2, and 20.6±0.2. In some embodiments, crystalline Form IP of Compound (II) is characterized by an X-ray powder diffractogram having a signal at at least four two-theta values chosen from 10.1±0.2, 12.0±0.2, 14.6±0.2, 15.0±0.2, 19.0±0.2, 19.4±0.2, and 20.6±0.2. In some embodiments, crystalline Form IP of Compound (II) is characterized by an X-ray powder diffractogram having a signal at at least three two-theta values chosen from 10.1±0.2, 12.0±0.2, 14.6±0.2, 15.0±0.2, 19.0±0.2, 19.4±0.2, and 20.6±0.2. In some embodiments, crystalline Form IP of Compound (II) is characterized by an X-ray powder diffractogram having a signal at at least two two-theta values chosen from 10.1±0.2, 12.0±0.2, 14.6±0.2, 15.0±0.2, 19.0±0.2, 19.4±0.2, and 20.6±0.2. In some embodiments, crystalline Form IP of Compound (II) is characterized by an X-ray powder diffractogram having a signal at at least one two-theta value chosen from 10.1±0.2, 12.0±0.2, 14.6±0.2, 15.0±0.2, 19.0±0.2, 19.4±0.2, and 20.6±0.2.

In some embodiments, crystalline Form IP of Compound (II) is characterized by an X-ray powder diffractogram having a signal at two-theta values of 10.1±0.2, 12.0±0.2, 14.6±0.2, and 15.0±0.2. In some embodiments, crystalline Form IP of Compound (II) is characterized by an X-ray powder diffractogram having a signal at at least three two-theta values chosen from 10.1±0.2, 12.0±0.2, 14.6±0.2, and 15.0±0.2. In some embodiments, crystalline Form IP of Compound (II) is characterized by an X-ray powder diffractogram having a signal at at least two two-theta values chosen from 10.1±0.2, 12.0±0.2, 14.6±0.2, and 15.0±0.2. In some embodiments, crystalline Form IP of Compound (II) is characterized by an X-ray powder diffractogram having a signal at at least one two-theta value chosen from 10.1±0.2, 12.0±0.2, 14.6±0.2, and 15.0±0.2.

In some embodiments, crystalline Form IP of Compound (II) is characterized by an X-ray powder diffractogram substantially similar to that in FIG. 22.

In some embodiments, crystalline Form IP of Compound (II) is characterized by a monoclinic crystal system. In some embodiments, crystalline Form IP of Compound (II) is characterized as belonging to a monoclinic space group P 1 21 1. In some embodiments, crystalline Form IP of Compound (II) is characterized by having a unit cell characterized by three edges of 11.7883±0.0016 Å, 8.0019±0.0013 Å, and 18.931±0.003 Å.

In some embodiments, crystalline Form IP of Compound (II) is characterized by having a unit cell with the following characteristics measured at 296 K and 0.71073 Å:

| Crystal | Monoclinic |
|---|---|
| System: | |
| Space | P 1 21 1 |
| Group: | |
| a (Å): | 11.7883(16) |
| b (Å): | 8.0019(13) |
| c (Å): | 18.931(3) |
| α (°): | 90 |
| β (°): | 104.170(7) |
| γ (°): | 90 |
| V (Å3): | 1737.4(5) |
| Z/Z′: | 2/1 |

In some embodiments, crystalline Form IP of Compound (II) is characterized by having a unit cell characterized with an angle, α, of 90°. In some embodiments, crystalline Form IP of Compound (II) is characterized by having a unit cell characterized with an angle, β, of 104.170±0.007°. In some embodiments, crystalline Form IP of Compound (II) is characterized by having a unit cell characterized with an angle, γ, of 90°.

In some embodiments, crystalline Form IP of Compound (II) is characterized by having a unit cell with volume of 1731.4±0.5 Å³. In some embodiments, crystalline Form IP of Compound (II) is characterized by having a unit cell with volume of 1731 Å³.

In some embodiments, the present disclosure provides crystalline Form IP of Compound (II) prepared by a process comprising crystallizing Compound (II) from a mixture of isopropanol and Compound (II). In some embodiments, the crystallizing is carried out above room temperature. In some embodiments, the crystallizing is carried out at room temperature. In some embodiments, the crystallizing is induced by introduction of a seed crystal.

In some embodiments, the present disclosure provides methods of preparing crystalline Form IP of Compound (II) comprising crystallizing Compound (II) from a mixture of isopropanol and Compound (II). In some embodiments, the crystallizing is carried out above room temperature. In some embodiments, the crystallizing is carried out at room temperature. In some embodiments, the crystallizing is induced by introduction of a seed crystal.

### Crystalline Form NPR of Compound (II)

In some embodiments, the present disclosure provides crystalline Form NPR of Compound (II):

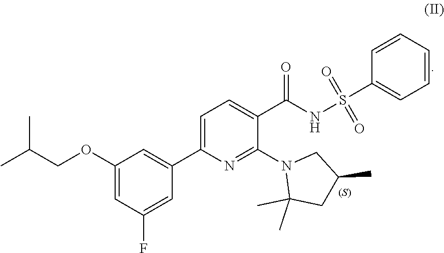

In some embodiments, crystalline Form NPR is an n-propanol solvate of Compound (II).

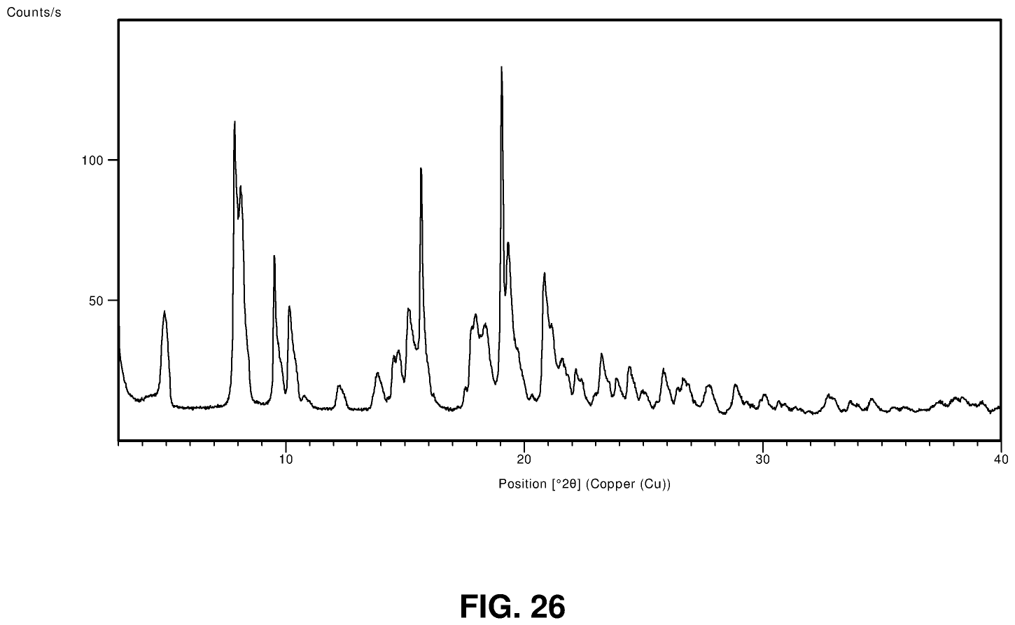

FIG. 26 shows an X-ray powder diffractogram of crystalline Form NPR of Compound (II).

In some embodiments, crystalline Form NPR of Compound (II) is in substantially pure form. In some embodiments, crystalline Form NPR of Compound (II) is characterized by an X-ray powder diffractogram generated by an X-ray powder diffraction analysis with an incident beam of Cu Kα radiation.

In some embodiments, crystalline Form NPR of Compound (II) is characterized by an X-ray powder diffractogram having a signal at 4.8±0.2 degrees two-theta. In some embodiments, crystalline Form NPR of Compound (II) is characterized by an X-ray powder diffractogram having a signal at 7.7±0.2 degrees two-theta. In some embodiments, crystalline Form NPR of Compound (II) is characterized by an X-ray powder diffractogram having a signal at 8.0±0.2 degrees two-theta. In some embodiments, crystalline Form NPR of Compound (II) is characterized by an X-ray powder diffractogram having a signal at 10.1±0.2 degrees two-theta. In some embodiments, crystalline Form NPR of Compound (II) is characterized by an X-ray powder diffractogram having a signal at 12.1±0.2 degrees two-theta. In some embodiments, crystalline Form NPR of Compound (II) is characterized by an X-ray powder diffractogram having a signal at 14.7±0.2 degrees two-theta. In some embodiments, crystalline Form NPR of Compound (II) is characterized by an X-ray powder diffractogram having a signal at 15.0±0.2 degrees two-theta. In some embodiments, crystalline Form NPR of Compound (II) is characterized by an X-ray powder diffractogram having a signal at 15.5±0.2 degrees two-theta. In some embodiments, crystalline Form NPR of Compound (II) is characterized by an X-ray powder diffractogram having a signal at 17.7±0.2 degrees two-theta. In some embodiments, crystalline Form NPR of Compound (II) is characterized by an X-ray powder diffractogram having a signal at 19.1±0.2 degrees two-theta. In some embodiments, crystalline Form NPR of Compound (II) is characterized by an X-ray powder diffractogram having a signal at 20.6±0.2 degrees two-theta.

In some embodiments, crystalline Form NPR of Compound (II) is characterized by an X-ray powder diffractogram having a signal at two-theta values of 4.8±0.2, 7.7±0.2, 8.0±0.2, 10.1±0.2, 12.1±0.2, 14.7±0.2, 15.0±0.2, 15.5±0.2, 17.7±0.2, 19.1±0.2, and 20.6±0.2. In some embodiments, crystalline Form NPR of Compound (II) is characterized by an X-ray powder diffractogram having a signal at at least ten two-theta values chosen from 4.8±0.2, 7.7±0.2, 8.0±0.2, 10.1±0.2, 12.1±0.2, 14.7±0.2, 15.0±0.2, 15.5±0.2, 17.7±0.2, 19.1±0.2, and 20.6±0.2. In some embodiments, crystalline Form NPR of Compound (II) is characterized by an X-ray powder diffractogram having a signal at at least nine two-theta values chosen from 4.8±0.2, 7.7±0.2, 8.0±0.2, 10.1±0.2, 12.1±0.2, 14.7±0.2, 15.0±0.2, 15.5±0.2, 17.7±0.2, 19.1±0.2, and 20.6±0.2. In some embodiments, crystalline Form NPR of Compound (II) is characterized by an X-ray powder diffractogram having a signal at at least eight two-theta values chosen from 4.8±0.2, 7.7±0.2, 8.0±0.2, 10.1±0.2, 12.1±0.2, 14.7±0.2, 15.0±0.2, 15.5±0.2, 17.7±0.2, 19.1±0.2, and 20.6±0.2. In some embodiments, crystalline Form NPR of Compound (II) is characterized by an X-ray powder diffractogram having a signal at at least seven two-theta values chosen from 4.8±0.2, 7.7±0.2, 8.0±0.2, 10.1±0.2, 12.1±0.2, 14.7±0.2, 15.0±0.2, 15.5±0.2, 17.7±0.2, 19.1±0.2, and 20.6±0.2. In some embodiments, crystalline Form NPR of Compound (II) is characterized by an X-ray powder diffractogram having a signal at at least six two-theta values chosen from 4.8±0.2, 7.7±0.2, 8.0±0.2, 10.1±0.2, 12.1±0.2, 14.7±0.2, 15.0±0.2, 15.5±0.2, 17.7±0.2, 19.1±0.2, and 20.6±0.2. In some embodiments, crystalline Form NPR of Compound (II) is characterized by an X-ray powder diffractogram having a signal at at least five two-theta values chosen from 4.8±0.2, 7.7±0.2, 8.0±0.2, 10.1±0.2, 12.1±0.2, 14.7±0.2, 15.0±0.2, 15.5±0.2, 17.7±0.2, 19.1±0.2, and 20.6±0.2. In some embodiments, crystalline Form NPR of Compound (II) is characterized by an X-ray powder diffractogram having a signal at at least four two-theta values chosen from 4.8±0.2, 7.7±0.2, 8.0±0.2, 10.1±0.2, 12.1±0.2, 14.7±0.2, 15.0±0.2, 15.5±0.2, 17.7±0.2, 19.1±0.2, and 20.6±0.2. In some embodiments, crystalline Form NPR of Compound (II) is characterized by an X-ray powder diffractogram having a signal at at least three two-theta values chosen from 4.8±0.2, 7.7±0.2, 8.0±0.2, 10.1±0.2, 12.1±0.2, 14.7±0.2, 15.0±0.2, 15.5±0.2, 17.7±0.2, 19.1±0.2, and 20.6±0.2. In some embodiments, crystalline Form NPR of Compound (II) is characterized by an X-ray powder diffractogram having a signal at at least two two-theta values chosen from 4.8±0.2, 7.7±0.2, 8.0±0.2, 10.1±0.2, 12.1±0.2, 14.7±0.2, 15.0±0.2, 15.5±0.2, 17.7±0.2, 19.1±0.2, and 20.6±0.2. In some embodiments, crystalline Form NPR of Compound (II) is characterized by an X-ray powder diffractogram having a signal at at least one two-theta value chosen from 4.8±0.2, 7.7±0.2, 8.0±0.2, 10.1±0.2, 12.1±0.2, 14.7±0.2, 15.0±0.2, 15.5±0.2, 17.7±0.2, 19.1±0.2, and 20.6±0.2.

In some embodiments, crystalline Form NPR of Compound (II) is characterized by an X-ray powder diffractogram having a signal at two-theta values of 4.8±0.2, 7.7±0.2, 8.0±0.2, 10.1±0.2, 12.1±0.2, 15.0±0.2, 15.5±0.2, 19.1±0.2, and 20.6±0.2. In some embodiments, crystalline Form NPR of Compound (II) is characterized by an X-ray powder diffractogram having a signal at at least eight two-theta values chosen from 4.8±0.2, 7.7±0.2, 8.0±0.2, 10.1±0.2, 12.1±0.2, 15.0±0.2, 15.5±0.2, 19.1±0.2, and 20.6±0.2. In some embodiments, crystalline Form NPR of Compound (II) is characterized by an X-ray powder diffractogram having a signal at at least seven two-theta values chosen from 4.8±0.2, 7.7±0.2, 8.0±0.2, 10.1±0.2, 12.1±0.2, 15.0±0.2, 15.5±0.2, 19.1±0.2, and 20.6±0.2. In some embodiments, crystalline Form NPR of Compound (II) is characterized by an X-ray powder diffractogram having a signal at at least six two-theta values chosen from 4.8±0.2, 7.7±0.2, 8.0±0.2, 10.1±0.2, 12.1±0.2, 15.0±0.2, 15.5±0.2, 19.1±0.2, and 20.6±0.2. In some embodiments, crystalline Form NPR of Compound (II) is characterized by an X-ray powder diffractogram having a signal at at least five two-theta values chosen from 4.8±0.2, 7.7±0.2, 8.0±0.2, 10.1±0.2, 12.1±0.2, 15.0±0.2, 15.5±0.2, 19.1±0.2, and 20.6±0.2. In some embodiments, crystalline Form NPR of Compound (II) is characterized by an X-ray powder diffractogram having a signal at at least four two-theta values chosen from 4.8±0.2, 7.7±0.2, 8.0±0.2, 10.1±0.2, 12.1±0.2, 15.0±0.2, 15.5±0.2, 19.1±0.2, and 20.6±0.2. In some embodiments, crystalline Form NPR of Compound (II) is characterized by an X-ray powder diffractogram having a signal at at least three two-theta values chosen from 4.8±0.2, 7.7±0.2, 8.0±0.2, 10.1±0.2, 12.1±0.2, 15.0±0.2, 15.5±0.2, 19.1±0.2, and 20.6±0.2. In some embodiments, crystalline Form NPR of Compound (II) is characterized by an X-ray powder diffractogram having a signal at at least two two-theta values chosen from 4.8±0.2, 7.7±0.2, 8.0±0.2, 10.1±0.2, 12.1±0.2, 15.0±0.2, 15.5±0.2, 19.1±0.2, and 20.6±0.2. In some embodiments, crystalline Form NPR of Compound (II) is characterized by an X-ray powder diffractogram having a signal at at least one two-theta value chosen from 4.8±0.2, 7.7±0.2, 8.0±0.2, 10.1±0.2, 12.1±0.2, 15.0±0.2, 15.5±0.2, 19.1±0.2, and 20.6±0.2.

In some embodiments, crystalline Form NPR of Compound (II) is characterized by an X-ray powder diffractogram having a signal at two-theta values of 4.8±0.2, 7.7±0.2, 8.0±0.2, 10.1±0.2, 12.1±0.2, 15.0±0.2, 19.1±0.2, and 20.6±0.2. In some embodiments, crystalline Form NPR of Compound (II) is characterized by an X-ray powder diffractogram having a signal at at least seven two-theta values chosen from 4.8±0.2, 7.7±0.2, 8.0±0.2, 10.1±0.2, 12.1±0.2, 15.0±0.2, 19.1±0.2, and 20.6±0.2. In some embodiments, crystalline Form NPR of Compound (II) is characterized by an X-ray powder diffractogram having a signal at at least six two-theta values chosen from 4.8±0.2, 7.7±0.2, 8.0±0.2, 10.1±0.2, 12.1±0.2, 15.0±0.2, 19.1±0.2, and 20.6±0.2. In some embodiments, crystalline Form NPR of Compound (II) is characterized by an X-ray powder diffractogram having a signal at at least five two-theta values chosen from 4.8±0.2, 7.7±0.2, 8.0±0.2, 10.1±0.2, 12.1±0.2, 15.0±0.2, 19.1±0.2, and 20.6±0.2. In some embodiments, crystalline Form NPR of Compound (II) is characterized by an X-ray powder diffractogram having a signal at at least four two-theta values chosen from 4.8±0.2, 7.7±0.2, 8.0±0.2, 10.1±0.2, 12.1±0.2, 15.0±0.2, 19.1±0.2, and 20.6±0.2. In some embodiments, crystalline Form NPR of Compound (II) is characterized by an X-ray powder diffractogram having a signal at at least three two-theta values chosen from 4.8±0.2, 7.7±0.2, 8.0±0.2, 10.1±0.2, 12.1±0.2, 15.0±0.2, 19.1±0.2, and 20.6±0.2. In some embodiments, crystalline Form NPR of Compound (II) is characterized by an X-ray powder diffractogram having a signal at at least two two-theta values chosen from 4.8±0.2, 7.7±0.2, 8.0±0.2, 10.1±0.2, 12.1±0.2, 15.0±0.2, 19.1±0.2, and 20.6±0.2. In some embodiments, crystalline Form NPR of Compound (II) is characterized by an X-ray powder diffractogram having a signal at at least one two-theta value chosen from 4.8±0.2, 7.7±0.2, 8.0±0.2, 10.1±0.2, 12.1±0.2, 15.0±0.2, 19.1±0.2, and 20.6±0.2.

In some embodiments, crystalline Form NPR of Compound (II) is characterized by an X-ray powder diffractogram having a signal at two-theta values of 10.1±0.2, 12.1±0.2, and 15.0±0.2. In some embodiments, crystalline Form NPR of Compound (II) is characterized by an X-ray powder diffractogram having a signal at at least two two-theta values chosen from 10.1±0.2, 12.1±0.2, and 15.0±0.2. In some embodiments, crystalline Form NPR of Compound (II) is characterized by an X-ray powder diffractogram having a signal at at least one two-theta value chosen from 10.1±10.1±0.2, 12.1±0.2, and 15.0±0.2.

In some embodiments, crystalline Form NPR of Compound (II) is characterized by an X-ray powder diffractogram substantially similar to that in FIG. 26.

In some embodiments, the present disclosure provides crystalline Form NPR of Compound (II) prepared by a process comprising crystallizing Compound (II) from a mixture of n-propanol and Compound (II). In some embodiments, the crystallizing is carried out at room temperature.

In some embodiments, the present disclosure provides methods of preparing crystalline Form NPR of Compound (II) comprising crystallizing Compound (II) from a mixture of n-propanol and Compound (II). In some embodiments, the crystallizing is carried out at room temperature.

### Crystalline Form 2B of Compound (II)

In some embodiments, the present disclosure provides crystalline Form 2B of Compound (II):

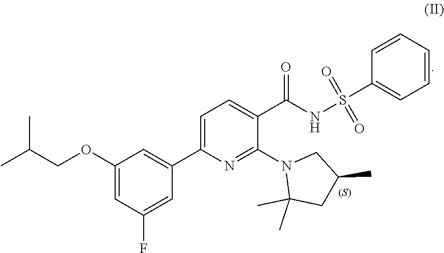

In some embodiments, crystalline Form 2B is a 2-butanol solvate of Compound (II).

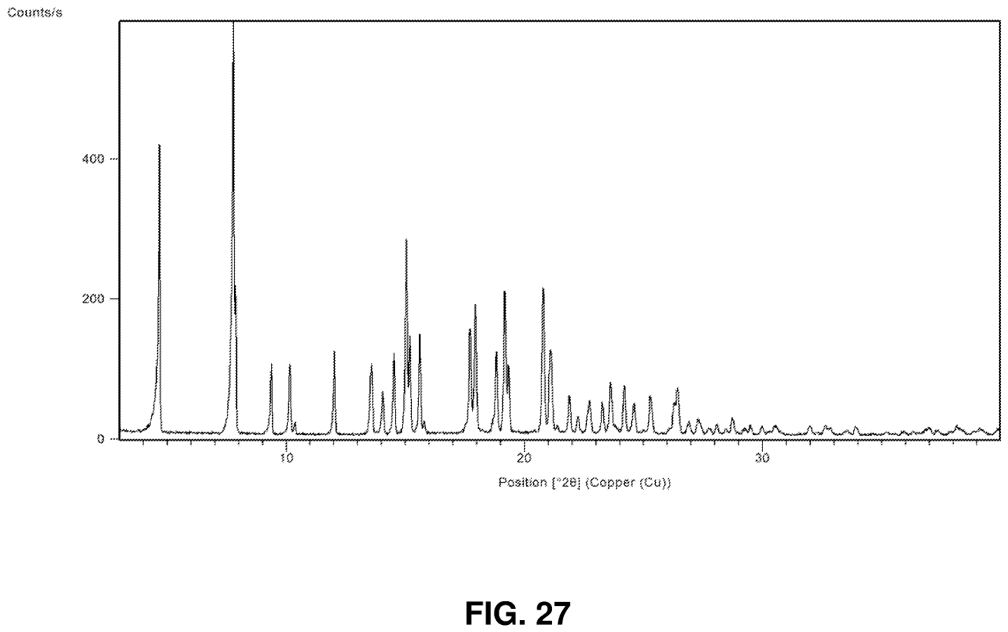

FIG. 27 shows an X-ray powder diffractogram of crystalline Form 2B of Compound (II) at ambient conditions.

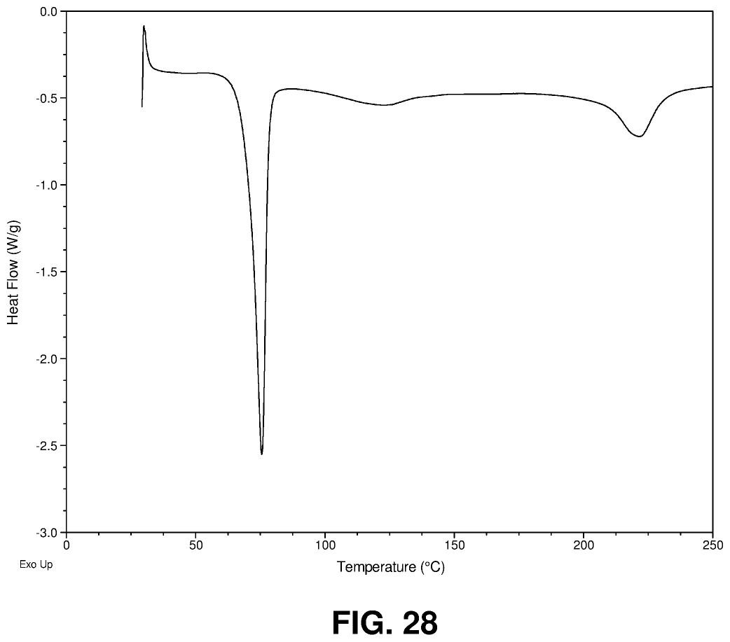

FIG. 28 shows a DSC trace of the crystalline Form 2B of Compound (II). In some embodiments, crystalline Form 2B of Compound (II) is characterized by a DSC having an onset of desolvation temperature of 97.5° C. and/or a peak temperature of 122.4° C.

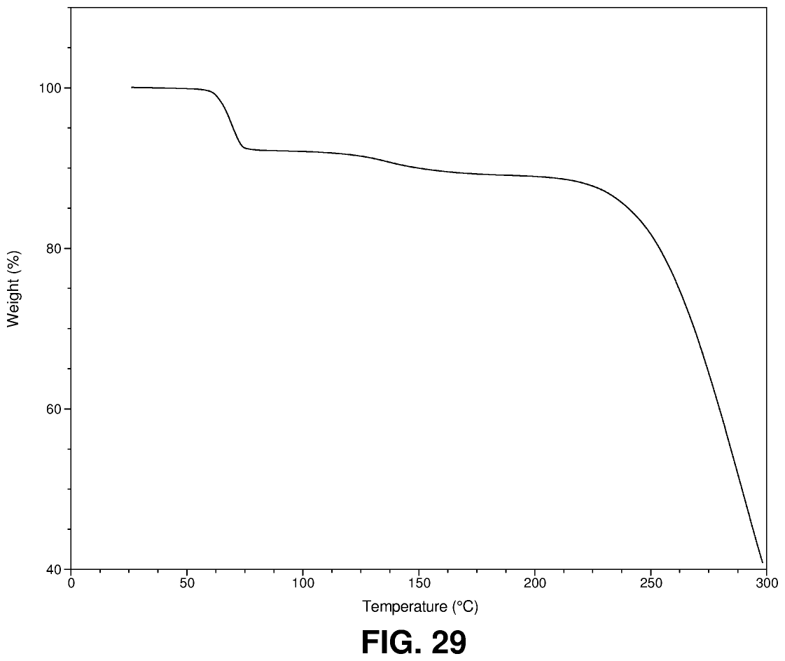

FIG. 29 shows the results of a TGA of crystalline Form 2B of Compound (II). In some embodiments, crystalline Form 2B of Compound (II) is characterized by a TGA having an onset of decomposition temperature of about 200° C.

In some embodiments, crystalline Form 2B of Compound (II) is in substantially pure form. In some embodiments, crystalline Form 2B of Compound (II) is characterized by an X-ray powder diffractogram generated by an X-ray powder diffraction analysis with an incident beam of Cu Kα radiation.

In some embodiments, crystalline Form 2B of Compound (II) is characterized by an X-ray powder diffractogram having a signal at 4.7±0.2 degrees two-theta. In some embodiments, crystalline Form 2B of Compound (II) is characterized by an X-ray powder diffractogram having a signal at 7.8±0.2 degrees two-theta. In some embodiments, crystalline Form 2B of Compound (II) is characterized by an X-ray powder diffractogram having a signal at 10.2±0.2 degrees two-theta. In some embodiments, crystalline Form 2B of Compound (II) is characterized by an X-ray powder diffractogram having a signal at 12.0±0.2 degrees two-theta. In some embodiments, crystalline Form 2B of Compound (II) is characterized by an X-ray powder diffractogram having a signal at 13.6±0.2 degrees two-theta. In some embodiments, crystalline Form 2B of Compound (II) is characterized by an X-ray powder diffractogram having a signal at 14.1±0.2 degrees two-theta. In some embodiments, crystalline Form 2B of Compound (II) is characterized by an X-ray powder diffractogram having a signal at 14.5±0.2 degrees two-theta. In some embodiments, crystalline Form 2B of Compound (II) is characterized by an X-ray powder diffractogram having a signal at 15.0±0.2 degrees two-theta. In some embodiments, crystalline Form 2B of Compound (II) is characterized by an X-ray powder diffractogram having a signal at 15.6±0.2 degrees two-theta. In some embodiments, crystalline Form 2B of Compound (II) is characterized by an X-ray powder diffractogram having a signal at 17.9±0.2 degrees two-theta. In some embodiments, crystalline Form 2B of Compound (II) is characterized by an X-ray powder diffractogram having a signal at 18.8±0.2 degrees two-theta. In some embodiments, crystalline Form 2B of Compound (II) is characterized by an X-ray powder diffractogram having a signal at 19.2±0.2 degrees two-theta. In some embodiments, crystalline Form 2B of Compound (II) is characterized by an X-ray powder diffractogram having a signal at 20.8±0.2 degrees two-theta.

In some embodiments, crystalline Form 2B of Compound (II) is characterized by an X-ray powder diffractogram having a signal at two-theta values of 4.7±0.2, 7.8±0.2, 10.2±0.2, 12.0±0.2, 13.6±0.2, 14.1±0.2, 14.5±0.2, 15.0±0.2, 15.6±0.2, 17.9±0.2, 18.8±0.2, 19.2±0.2, and 20.8±0.2. In some embodiments, crystalline Form 2B of Compound (II) is characterized by an X-ray powder diffractogram having a signal at at least twelve two-theta values chosen from 4.7±0.2, 7.8±0.2, 10.2±0.2, 12.0±0.2, 13.6±0.2, 14.1±0.2, 14.5±0.2, 15.0±0.2, 15.6±0.2, 17.9±0.2, 18.8±0.2, 19.2±0.2, and 20.8±0.2. In some embodiments, crystalline Form 2B of Compound (II) is characterized by an X-ray powder diffractogram having a signal at at least eleven two-theta values chosen from 4.7±0.2, 7.8±0.2, 10.2±0.2, 12.0±0.2, 13.6±0.2, 14.1±0.2, 14.5±0.2, 15.0±0.2, 15.6±0.2, 17.9±0.2, 18.8±0.2, 19.2±0.2, and 20.8±0.2. In some embodiments, crystalline Form 2B of Compound (II) is characterized by an X-ray powder diffractogram having a signal at at least ten two-theta values chosen from 4.7±0.2, 7.8±0.2, 10.2±0.2, 12.0±0.2, 13.6±0.2, 14.1±0.2, 14.5±0.2, 15.0±0.2, 15.6±0.2, 17.9±0.2, 18.8±0.2, 19.2±0.2, and 20.8±0.2. In some embodiments, crystalline Form 2B of Compound (II) is characterized by an X-ray powder diffractogram having a signal at at least nine two-theta values chosen from 4.7±0.2, 7.8±0.2, 10.2±0.2, 12.0±0.2, 13.6±0.2, 14.1±0.2, 14.5±0.2, 15.0±0.2, 15.6±0.2, 17.9±0.2, 18.8±0.2, 19.2±0.2, and 20.8±0.2. In some embodiments, crystalline Form 2B of Compound (II) is characterized by an X-ray powder diffractogram having a signal at at least eight two-theta values chosen from 4.7±0.2, 7.8±0.2, 10.2±0.2, 12.0±0.2, 13.6±0.2, 14.1±0.2, 14.5±0.2, 15.0±0.2, 15.6±0.2, 17.9±0.2, 18.8±0.2, 19.2±0.2, and 20.8±0.2. In some embodiments, crystalline Form 2B of Compound (II) is characterized by an X-ray powder diffractogram having a signal at at least seven two-theta values chosen from 4.7±0.2, 7.8±0.2, 10.2±0.2, 12.0±0.2, 13.6±0.2, 14.1±0.2, 14.5±0.2, 15.0±0.2, 15.6±0.2, 17.9±0.2, 18.8±0.2, 19.2±0.2, and 20.8±0.2. In some embodiments, crystalline Form 2B of Compound (II) is characterized by an X-ray powder diffractogram having a signal at at least six two-theta values chosen from 4.7±0.2, 7.8±0.2, 10.2±0.2, 12.0±0.2, 13.6±0.2, 14.1±0.2, 14.5±0.2, 15.0±0.2, 15.6±0.2, 17.9±0.2, 18.8±0.2, 19.2±0.2, and 20.8±0.2. In some embodiments, crystalline Form 2B of Compound (II) is characterized by an X-ray powder diffractogram having a signal at at least five two-theta values chosen from 4.7±0.2, 7.8±0.2, 10.2±0.2, 12.0±0.2, 13.6±0.2, 14.1±0.2, 14.5±0.2, 15.0±0.2, 15.6±0.2, 17.9±0.2, 18.8±0.2, 19.2±0.2, and 20.8±0.2. In some embodiments, crystalline Form 2B of Compound (II) is characterized by an X-ray powder diffractogram having a signal at at least four two-theta values chosen from 4.7±0.2, 7.8±0.2, 10.2±0.2, 12.0±0.2, 13.6±0.2, 14.1±0.2, 14.5±0.2, 15.0±0.2, 15.6±0.2, 17.9±0.2, 18.8±0.2, 19.2±0.2, and 20.8±0.2. In some embodiments, crystalline Form 2B of Compound (II) is characterized by an X-ray powder diffractogram having a signal at at least three two-theta values chosen from 4.7±0.2, 7.8±0.2, 10.2±0.2, 12.0±0.2, 13.6±0.2, 14.1±0.2, 14.5±0.2, 15.0±0.2, 15.6±0.2, 17.9±0.2, 18.8±0.2, 19.2±0.2, and 20.8±0.2. In some embodiments, crystalline Form 2B of Compound (II) is characterized by an X-ray powder diffractogram having a signal at at least two two-theta values chosen from 4.7±0.2, 7.8±0.2, 10.2±0.2, 12.0±0.2, 13.6±0.2, 14.1±0.2, 14.5±0.2, 15.0±0.2, 15.6±0.2, 17.9±0.2, 18.8±0.2, 19.2±0.2, and 20.8±0.2. In some embodiments, crystalline Form 2B of Compound (II) is characterized by an X-ray powder diffractogram having a signal at at least one two-theta value chosen from 4.7±0.2, 7.8±0.2, 10.2±0.2, 12.0±0.2, 13.6±0.2, 14.1±0.2, 14.5±0.2, 15.0±0.2, 15.6±0.2, 17.9±0.2, 18.8±0.2, 19.2±0.2, and 20.8±0.2.

In some embodiments, crystalline Form 2B of Compound (II) is characterized by an X-ray powder diffractogram having a signal at two-theta values of 4.7±0.2, 7.8±0.2, 10.2±0.2, 12.0±0.2, 14.1±0.2, 15.0±0.2, 15.6±0.2, 17.9±0.2, 19.2±0.2, and 20.8±0.2. In some embodiments, crystalline Form 2B of Compound (II) is characterized by an X-ray powder diffractogram having a signal at at least nine two-theta values chosen from 4.7±0.2, 7.8±0.2, 10.2±0.2, 12.0±0.2, 14.1±0.2, 15.0±0.2, 15.6±0.2, 17.9±0.2, 19.2±0.2, and 20.8±0.2. In some embodiments, crystalline Form 2B of Compound (II) is characterized by an X-ray powder diffractogram having a signal at at least eight two-theta values chosen from 4.7±0.2, 7.8±0.2, 10.2±0.2, 12.0±0.2, 14.1±0.2, 15.0±0.2, 15.6±0.2, 17.9±0.2, 19.2±0.2, and 20.8±0.2. In some embodiments, crystalline Form 2B of Compound (II) is characterized by an X-ray powder diffractogram having a signal at at least seven two-theta values chosen from 4.7±0.2, 7.8±0.2, 10.2±0.2, 12.0±0.2, 14.1±0.2, 15.0±0.2, 15.6±0.2, 17.9±0.2, 19.2±0.2, and 20.8±0.2. In some embodiments, crystalline Form 2B of Compound (II) is characterized by an X-ray powder diffractogram having a signal at at least six two-theta values chosen from 4.7±0.2, 7.8±0.2, 10.2±0.2, 12.0±0.2, 14.1±0.2, 15.0±0.2, 15.6±0.2, 17.9±0.2, 19.2±0.2, and 20.8±0.2. In some embodiments, crystalline Form 2B of Compound (II) is characterized by an X-ray powder diffractogram having a signal at at least five two-theta values chosen from 4.7±0.2, 7.8±0.2, 10.2±0.2, 12.0±0.2, 14.1±0.2, 15.0±0.2, 15.6±0.2, 17.9±0.2, 19.2±0.2, and 20.8±0.2. In some embodiments, crystalline Form 2B of Compound (II) is characterized by an X-ray powder diffractogram having a signal at at least four two-theta values chosen from 4.7±0.2, 7.8±0.2, 10.2±0.2, 12.0±0.2, 14.1±0.2, 15.0±0.2, 15.6±0.2, 17.9±0.2, 19.2±0.2, and 20.8±0.2. In some embodiments, crystalline Form 2B of Compound (II) is characterized by an X-ray powder diffractogram having a signal at at least three two-theta values chosen from 4.7±0.2, 7.8±0.2, 10.2±0.2, 12.0±0.2, 14.1±0.2, 15.0±0.2, 15.6±0.2, 17.9±0.2, 19.2±0.2, and 20.8±0.2. In some embodiments, crystalline Form 2B of Compound (II) is characterized by an X-ray powder diffractogram having a signal at at least two two-theta values chosen from 4.7±0.2, 7.8±0.2, 10.2±0.2, 12.0±0.2, 14.1±0.2, 15.0±0.2, 15.6±0.2, 17.9±0.2, 19.2±0.2, and 20.8±0.2. In some embodiments, crystalline Form 2B of Compound (II) is characterized by an X-ray powder diffractogram having a signal at at least one two-theta value chosen from 4.7±0.2, 7.8±0.2, 10.2±0.2, 12.0±0.2, 14.1±0.2, 15.0±0.2, 15.6±0.2, 17.9±0.2, 19.2±0.2, and 20.8±0.2.

In some embodiments, crystalline Form 2B of Compound (II) is characterized by an X-ray powder diffractogram having a signal at two-theta values of 4.7±0.2, 7.8±0.2, 10.2±0.2, 12.0±0.2, 14.1±0.2, 15.0±0.2, 19.2±0.2, and 20.8±0.2. In some embodiments, crystalline Form 2B of Compound (II) is characterized by an X-ray powder diffractogram having a signal at at least seven two-theta values chosen from 4.7±0.2, 7.8±0.2, 10.2±0.2, 12.0±0.2, 14.1±0.2, 15.0±0.2, 19.2±0.2, and 20.8±0.2. In some embodiments, crystalline Form 2B of Compound (II) is characterized by an X-ray powder diffractogram having a signal at at least six two-theta values chosen from 4.7±0.2, 7.8±0.2, 10.2±0.2, 12.0±0.2, 14.1±0.2, 15.0±0.2, 19.2±0.2, and 20.8±0.2. In some embodiments, crystalline Form 2B of Compound (II) is characterized by an X-ray powder diffractogram having a signal at at least five two-theta values chosen from 4.7±0.2, 7.8±0.2, 10.2±0.2, 12.0±0.2, 14.1±0.2, 15.0±0.2, 19.2±0.2, and 20.8±0.2. In some embodiments, crystalline Form 2B of Compound (II) is characterized by an X-ray powder diffractogram having a signal at at least four two-theta values chosen from 4.7±0.2, 7.8±0.2, 10.2±0.2, 12.0±0.2, 14.1±0.2, 15.0±0.2, 19.2±0.2, and 20.8±0.2. In some embodiments, crystalline Form 2B of Compound (II) is characterized by an X-ray powder diffractogram having a signal at at least three two-theta values chosen from 4.7±0.2, 7.8±0.2, 10.2±0.2, 12.0±0.2, 14.1±0.2, 15.0±0.2, 19.2±0.2, and 20.8±0.2. In some embodiments, crystalline Form 2B of Compound (II) is characterized by an X-ray powder diffractogram having a signal at at least two two-theta values chosen from 4.7±0.2, 7.8±0.2, 10.2±0.2, 12.0±0.2, 14.1±0.2, 15.0±0.2, 19.2±0.2, and 20.8±0.2. In some embodiments, crystalline Form 2B of Compound (II) is characterized by an X-ray powder diffractogram having a signal at at least one two-theta value chosen from 4.7±0.2, 7.8±0.2, 10.2±0.2, 12.0±0.2, 14.1±0.2, 15.0±0.2, 19.2±0.2, and 20.8±0.2.

In some embodiments, crystalline Form 2B of Compound (II) is characterized by an X-ray powder diffractogram having a signal at two-theta values of 10.2±0.2, 12.0±0.2, 14.1±0.2, and 15.0±0.2. In some embodiments, crystalline Form 2B of Compound (II) is characterized by an X-ray powder diffractogram having a signal at at least three two-theta values chosen from 10.2±0.2, 12.0±0.2, 14.1±0.2, and 15.0±0.2. In some embodiments, crystalline Form 2B of Compound (II) is characterized by an X-ray powder diffractogram having a signal at at least two two-theta values chosen from 10.2±0.2, 12.0±0.2, 14.1±0.2, and 15.0±0.2. In some embodiments, crystalline Form 2B of Compound (II) is characterized by an X-ray powder diffractogram having a signal at at least one two-theta value chosen from 10.2±0.2, 12.0±0.2, 14.1±0.2, and 15.0±0.2.

In some embodiments, crystalline Form IP of Compound (II) is characterized by an X-ray powder diffractogram substantially similar to that in FIG. 27.

In some embodiments, the present disclosure provides crystalline Form 2B of Compound (II) prepared by a process comprising crystallizing Compound (II) from a mixture of 2-butanol and Compound (II). In some embodiments, the crystallizing is carried out for a period of time of more than one week. In some embodiments, the crystallizing is carried out at room temperature.

In some embodiments, the present disclosure provides methods of preparing crystalline Form 2B of Compound (II) comprising crystallizing Compound (II) from a mixture of 2-butanol and Compound (II). In some embodiments, the crystallizing is carried out for a period of time of more than one week. In some embodiments, the crystallizing is carried out at room temperature.

### Crystalline Form MP of Compound (II)

In some embodiments, the present disclosure provides crystalline Form MP of Compound (II):

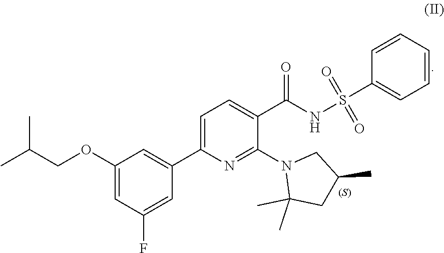

In some embodiments, crystalline Form MP is a 2-methyl-1-propanol solvate of Compound (II).

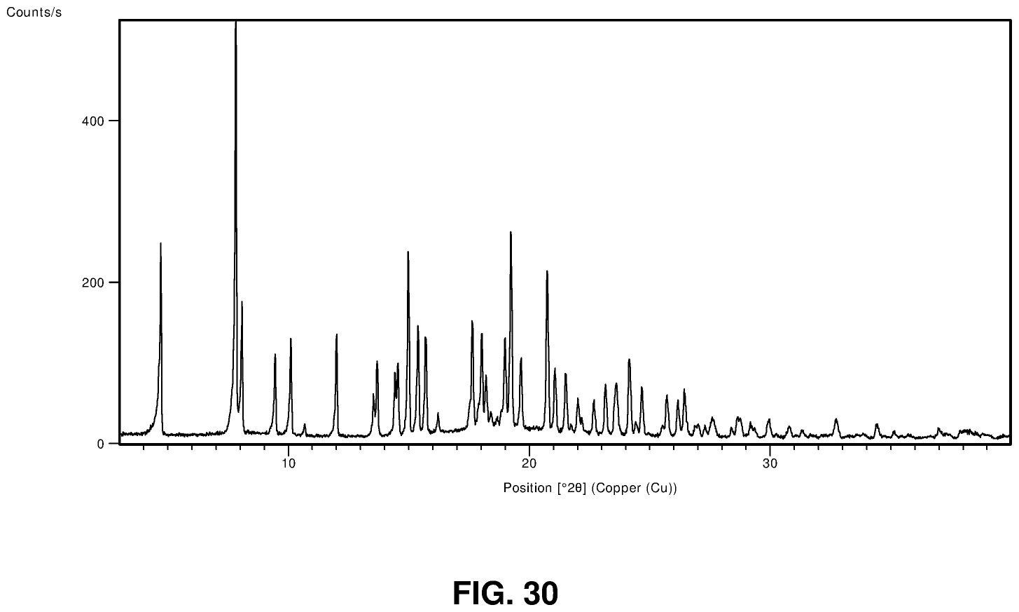

FIG. 30 shows an X-ray powder diffractogram of crystalline Form MP of Compound (II) at ambient conditions.

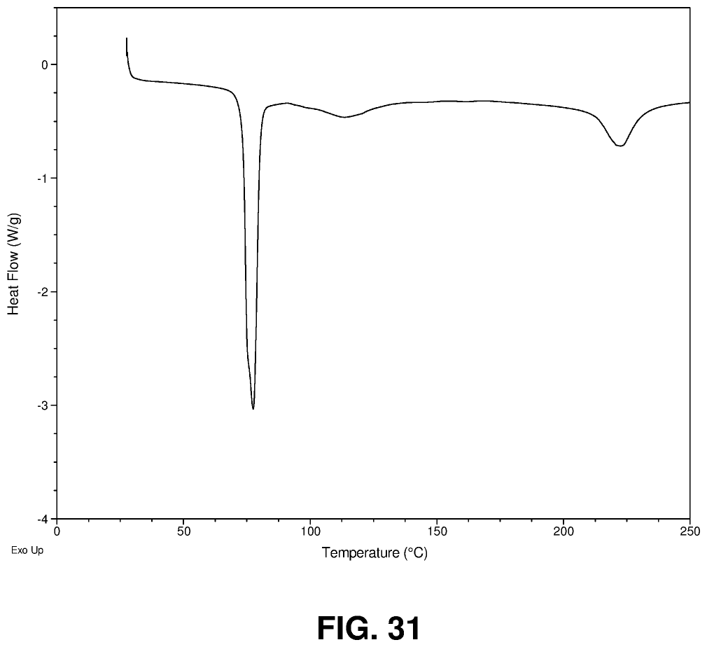

FIG. 31 shows a DSC trace of the crystalline Form MP of Compound (II). In some embodiments, crystalline Form MP of Compound (II) is characterized by a DSC having an onset of desolvation temperature of 73.5° C. and/or a peak temperature of 77.5° C. In some embodiments, crystalline Form MP of Compound (II) is characterized by a DSC having an onset of desolvation temperature of 96.8° C. and/or a peak temperature of 113.4° C.

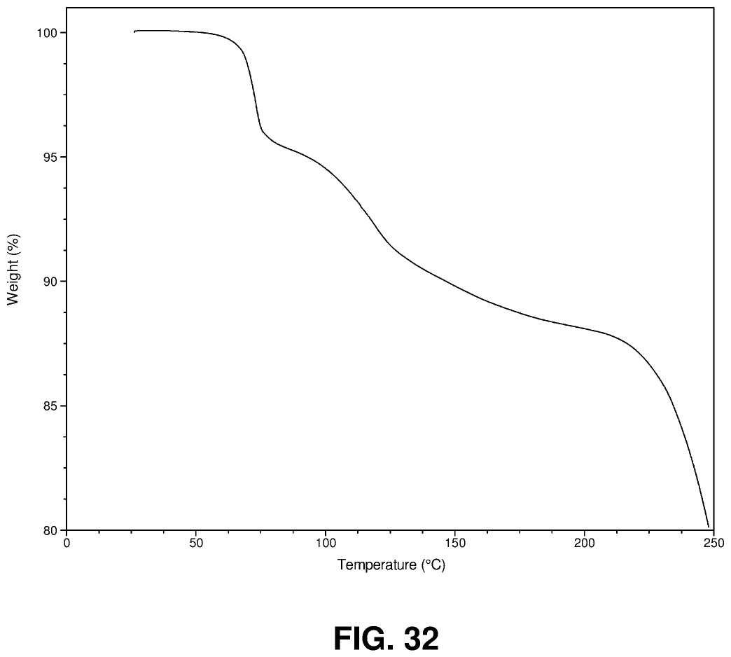

FIG. 32 shows the results of a TGA of crystalline Form MP of Compound (II). In some embodiments, crystalline Form MP of Compound (II) is characterized by a TGA having an onset of decomposition temperature of about 200° C.

In some embodiments, crystalline Form MP of Compound (II) is in substantially pure form. In some embodiments, crystalline Form MP of Compound (II) is characterized by an X-ray powder diffractogram generated by an X-ray powder diffraction analysis with an incident beam of Cu Kα radiation.

In some embodiments, crystalline Form MP of Compound (II) is characterized by an X-ray powder diffractogram having a signal at 4.7±0.2 degrees two-theta. In some embodiments, crystalline Form MP of Compound (II) is characterized by an X-ray powder diffractogram having a signal at 7.8±0.2 degrees two-theta. In some embodiments, crystalline Form MP of Compound (II) is characterized by an X-ray powder diffractogram having a signal at 8.1±0.2 degrees two-theta. In some embodiments, crystalline Form MP of Compound (II) is characterized by an X-ray powder diffractogram having a signal at 10.1±0.2 degrees two-theta. In some embodiments, crystalline Form MP of Compound (II) is characterized by an X-ray powder diffractogram having a signal at 12.0±0.2 degrees two-theta. In some embodiments, crystalline Form MP of Compound (II) is characterized by an X-ray powder diffractogram having a signal at 15.0±0.2 degrees two-theta. In some embodiments, crystalline Form MP of Compound (II) is characterized by an X-ray powder diffractogram having a signal at 15.4±0.2 degrees two-theta. In some embodiments, crystalline Form MP of Compound (II) is characterized by an X-ray powder diffractogram having a signal at 15.7±0.2 degrees two-theta. In some embodiments, crystalline Form MP of Compound (II) is characterized by an X-ray powder diffractogram having a signal at 17.6±0.2 degrees two-theta. In some embodiments, crystalline Form MP of Compound (II) is characterized by an X-ray powder diffractogram having a signal at 18.0±0.2 degrees two-theta. In some embodiments, crystalline Form MP of Compound (II) is characterized by an X-ray powder diffractogram having a signal at 19.2±0.2 degrees two-theta. In some embodiments, crystalline Form MP of Compound (II) is characterized by an X-ray powder diffractogram having a signal at 20.7±0.2 degrees two-theta.

In some embodiments, crystalline Form MP of Compound (II) is characterized by an X-ray powder diffractogram having a signal at two-theta values of 4.7±0.2, 7.8±0.2, 8.1±0.2, 10.1±0.2, 12.0±0.2, 15.0±0.2, 15.4±0.2, 15.7±0.2, 17.6±0.2, 18.0±0.2, 19.2±0.2, and 20.7±0.2. In some embodiments, crystalline Form MP of Compound (II) is characterized by an X-ray powder diffractogram having a signal at at least eleven two-theta values chosen from 4.7±0.2, 7.8±0.2, 8.1±0.2, 10.1±0.2, 12.0±0.2, 15.0±0.2, 15.4±0.2, 15.7±0.2, 17.6±0.2, 18.0±0.2, 19.2±0.2, and 20.7±0.2. In some embodiments, crystalline Form MP of Compound (II) is characterized by an X-ray powder diffractogram having a signal at at least ten two-theta values chosen from 4.7±0.2, 7.8±0.2, 8.1±0.2, 10.1±0.2, 12.0±0.2, 15.0±0.2, 15.4±0.2, 15.7±0.2, 17.6±0.2, 18.0±0.2, 19.2±0.2, and 20.7±0.2. In some embodiments, crystalline Form MP of Compound (II) is characterized by an X-ray powder diffractogram having a signal at at least nine two-theta values chosen from 4.7±0.2, 7.8±0.2, 8.1±0.2, 10.1±0.2, 12.0±0.2, 15.0±0.2, 15.4±0.2, 15.7±0.2, 17.6±0.2, 18.0±0.2, 19.2±0.2, and 20.7±0.2. In some embodiments, crystalline Form MP of Compound (II) is characterized by an X-ray powder diffractogram having a signal at at least eight two-theta values chosen from 4.7±0.2, 7.8±0.2, 8.1±0.2, 10.1±0.2, 12.0±0.2, 15.0±0.2, 15.4±0.2, 15.7±0.2, 17.6±0.2, 18.0±0.2, 19.2±0.2, and 20.7±0.2. In some embodiments, crystalline Form MP of Compound (II) is characterized by an X-ray powder diffractogram having a signal at at least seven two-theta values chosen from 4.7±0.2, 7.8±0.2, 8.1±0.2, 10.1±0.2, 12.0±0.2, 15.0±0.2, 15.4±0.2, 15.7±0.2, 17.6±0.2, 18.0±0.2, 19.2±0.2, and 20.7±0.2. In some embodiments, crystalline Form MP of Compound (II) is characterized by an X-ray powder diffractogram having a signal at at least six two-theta values chosen from 4.7±0.2, 7.8±0.2, 8.1±0.2, 10.1±0.2, 12.0±0.2, 15.0±0.2, 15.4±0.2, 15.7±0.2, 17.6±0.2, 18.0±0.2, 19.2±0.2, and 20.7±0.2. In some embodiments, crystalline Form MP of Compound (II) is characterized by an X-ray powder diffractogram having a signal at at least five two-theta values chosen from 4.7±0.2, 7.8±0.2, 8.1±0.2, 10.1±0.2, 12.0±0.2, 15.0±0.2, 15.4±0.2, 15.7±0.2, 17.6±0.2, 18.0±0.2, 19.2±0.2, and 20.7±0.2. In some embodiments, crystalline Form MP of Compound (II) is characterized by an X-ray powder diffractogram having a signal at at least four two-theta values chosen from 4.7±0.2, 7.8±0.2, 8.1±0.2, 10.1±0.2, 12.0±0.2, 15.0±0.2, 15.4±0.2, 15.7±0.2, 17.6±0.2, 18.0±0.2, 19.2±0.2, and 20.7±0.2. In some embodiments, crystalline Form MP of Compound (II) is characterized by an X-ray powder diffractogram having a signal at at least three two-theta values chosen from 4.7±0.2, 7.8±0.2, 8.1±0.2, 10.1±0.2, 12.0±0.2, 15.0±0.2, 15.4±0.2, 15.7±0.2, 17.6±0.2, 18.0±0.2, 19.2±0.2, and 20.7±0.2. In some embodiments, crystalline Form MP of Compound (II) is characterized by an X-ray powder diffractogram having a signal at at least two two-theta values chosen from 4.7±0.2, 7.8±0.2, 8.1±0.2, 10.1±0.2, 12.0±0.2, 15.0±0.2, 15.4±0.2, 15.7±0.2, 17.6±0.2, 18.0±0.2, 19.2±0.2, and 20.7±0.2. In some embodiments, crystalline Form MP of Compound (II) is characterized by an X-ray powder diffractogram having a signal at at least one two-theta value chosen from 4.7±0.2, 7.8±0.2, 8.1±0.2, 10.1±0.2, 12.0±0.2, 15.0±0.2, 15.4±0.2, 15.7±0.2, 17.6±0.2, 18.0±0.2, 19.2±0.2, and 20.7±0.2.

In some embodiments, crystalline Form MP of Compound (II) is characterized by an X-ray powder diffractogram having a signal at two-theta values of 4.7±0.2, 7.8±0.2, 8.1±0.2, 10.1±0.2, 12.0±0.2, 15.0±0.2, 19.2±0.2, and 20.7±0.2. In some embodiments, crystalline Form MP of Compound (II) is characterized by an X-ray powder diffractogram having a signal at at least seven two-theta values chosen from 4.7±0.2, 7.8±0.2, 8.1±0.2, 10.1±0.2, 12.0±0.2, 15.0±0.2, 19.2±0.2, and 20.7±0.2. In some embodiments, crystalline Form MP of Compound (II) is characterized by an X-ray powder diffractogram having a signal at at least six two-theta values chosen from 4.7±0.2, 7.8±0.2, 8.1±0.2, 10.1±0.2, 12.0±0.2, 15.0±0.2, 19.2±0.2, and 20.7±0.2. In some embodiments, crystalline Form MP of Compound (II) is characterized by an X-ray powder diffractogram having a signal at at least five two-theta values chosen from 4.7±0.2, 7.8±0.2, 8.1±0.2, 10.1±0.2, 12.0±0.2, 15.0±0.2, 19.2±0.2, and 20.7±0.2. In some embodiments, crystalline Form MP of Compound (II) is characterized by an X-ray powder diffractogram having a signal at at least four two-theta values chosen from 4.7±0.2, 7.8±0.2, 8.1±0.2, 10.1±0.2, 12.0±0.2, 15.0±0.2, 19.2±0.2, and 20.7±0.2. In some embodiments, crystalline Form MP of Compound (II) is characterized by an X-ray powder diffractogram having a signal at at least three two-theta values chosen from 4.7±0.2, 7.8±0.2, 8.1±0.2, 10.1±0.2, 12.0±0.2, 15.0±0.2, 19.2±0.2, and 20.7±0.2. In some embodiments, crystalline Form MP of Compound (II) is characterized by an X-ray powder diffractogram having a signal at at least two two-theta values chosen from 4.7±0.2, 7.8±0.2, 8.1±0.2, 10.1±0.2, 12.0±0.2, 15.0±0.2, 19.2±0.2, and 20.7±0.2. In some embodiments, crystalline Form MP of Compound (II) is characterized by an X-ray powder diffractogram having a signal at at least one two-theta value chosen from 4.7±0.2, 7.8±0.2, 8.1±0.2, 10.1±0.2, 12.0±0.2, 15.0±0.2, 19.2±0.2, and 20.7±0.2.

In some embodiments, crystalline Form MP of Compound (II) is characterized by an X-ray powder diffractogram having a signal at two-theta values of 4.7±0.2, 7.8±0.2, 10.1±0.2, 12.0±0.2, 15.0±0.2, 19.2±0.2, and 20.7±0.2. In some embodiments, crystalline Form MP of Compound (II) is characterized by an X-ray powder diffractogram having a signal at at least six two-theta values chosen from 4.7±0.2, 7.8±0.2, 10.1±0.2, 12.0±0.2, 15.0±0.2, 19.2±0.2, and 20.7±0.2. In some embodiments, crystalline Form MP of Compound (II) is characterized by an X-ray powder diffractogram having a signal at at least five two-theta values chosen from 4.7±0.2, 7.8±0.2, 10.1±0.2, 12.0±0.2, 15.0±0.2, 19.2±0.2, and 20.7±0.2. In some embodiments, crystalline Form MP of Compound (II) is characterized by an X-ray powder diffractogram having a signal at at least four two-theta values chosen from 4.7±0.2, 7.8±0.2, 10.1±0.2, 12.0±0.2, 15.0±0.2, 19.2±0.2, and 20.7±0.2. In some embodiments, crystalline Form MP of Compound (II) is characterized by an X-ray powder diffractogram having a signal at at least three two-theta values chosen from 4.7±0.2, 7.8±0.2, 10.1±0.2, 12.0±0.2, 15.0±0.2, 19.2±0.2, and 20.7±0.2. In some embodiments, crystalline Form MP of Compound (II) is characterized by an X-ray powder diffractogram having a signal at at least two two-theta values chosen from 4.7±0.2, 7.8±0.2, 10.1±0.2, 12.0±0.2, 15.0±0.2, 19.2±0.2, and 20.7±0.2. In some embodiments, crystalline Form MP of Compound (II) is characterized by an X-ray powder diffractogram having a signal at at least one two-theta value chosen from 4.7±0.2, 7.8±0.2, 10.1±0.2, 12.0±0.2, 15.0±0.2, 19.2±0.2, and 20.7±0.2.

In some embodiments, crystalline Form MP of Compound (II) is characterized by an X-ray powder diffractogram having a signal at two-theta values of 10.1±0.2, 12.0±0.2, and 15.0±0.2. In some embodiments, crystalline Form MP of Compound (II) is characterized by an X-ray powder diffractogram having a signal at at least two two-theta values chosen from 10.1±0.2, 12.0±0.2, and 15.0±0.2. In some embodiments, crystalline Form MP of Compound (II) is characterized by an X-ray powder diffractogram having a signal at at least one two-theta value chosen from 10.1±0.2, 12.0±0.2, and 15.0±0.2.

In some embodiments, crystalline Form MP of Compound (II) is characterized by an X-ray powder diffractogram substantially similar to that in FIG. 30.

In some embodiments, the present disclosure provides crystalline Form MP of Compound (II) prepared by a process comprising crystallizing Compound (II) from a mixture of 2-methyl-1-propanol and Compound (II). In some embodiments, the crystallizing is carried out for a period of time of more than \ one week. In some embodiments, the crystallizing is carried out at room temperature.

In some embodiments, the present disclosure provides methods of preparing crystalline Form MP of Compound (II) comprising crystallizing Compound (II) from a mixture of 2-methyl-1-propanol and Compound (II). In some embodiments, the crystallizing is carried out for a period of time of more than one week. In some embodiments, the crystallizing is carried out at room temperature.

### Crystalline Form NP of Compound (II)

In some embodiments, the present disclosure provides crystalline Form NP of Compound (II):

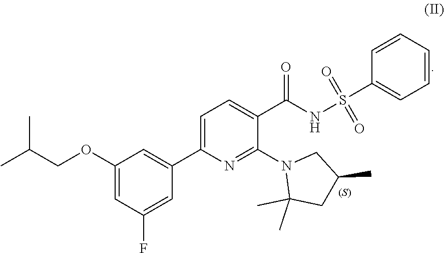

In some embodiments, crystalline Form NP is an n-pentanol solvate of Compound (II).

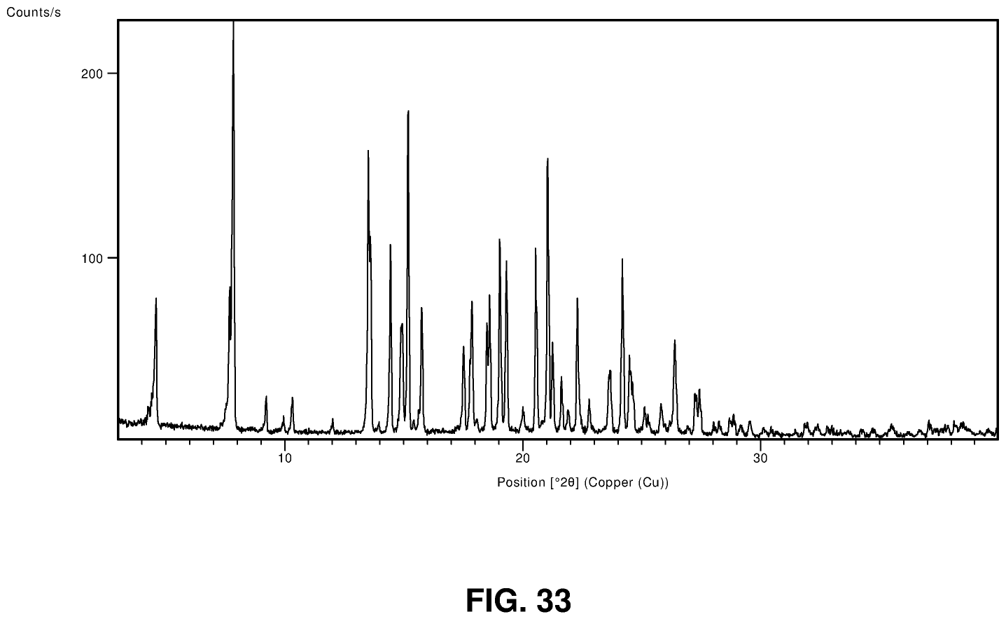

FIG. 33 shows an X-ray powder diffractogram of crystalline Form NP of Compound (II) at ambient conditions.

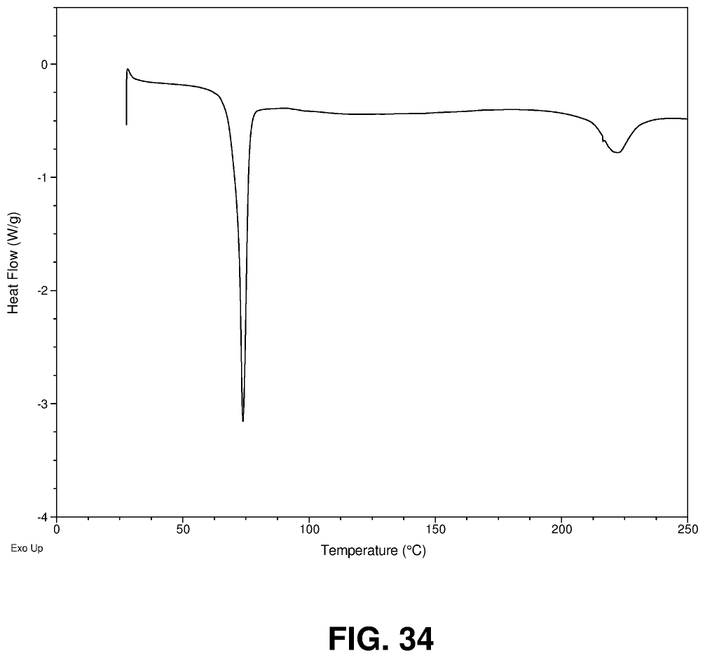

FIG. 34 shows a DSC trace of the crystalline Form NP of Compound (II). In some embodiments, crystalline Form NP of Compound (II) is characterized by a DSC having an onset of desolvation temperature of 71.5° C. and/or a peak temperature of 73.8° C.

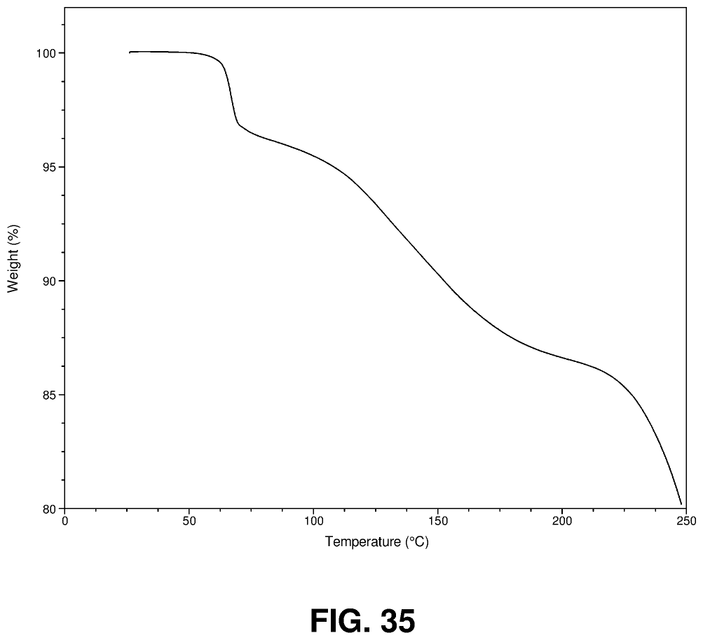

FIG. 35 shows the results of a TGA of crystalline Form NP of Compound (II). In some embodiments, crystalline Form NP of Compound (II) is characterized by a TGA having an onset of decomposition temperature of about 200° C.

In some embodiments, crystalline Form NP of Compound (II) is in substantially pure form. In some embodiments, crystalline Form NP of Compound (II) is characterized by an X-ray powder diffractogram generated by an X-ray powder diffraction analysis with an incident beam of Cu Kα radiation.

In some embodiments, crystalline Form NP of Compound (II) is characterized by an X-ray powder diffractogram having a signal at 7.8±0.2 degrees two-theta. In some embodiments, crystalline Form NP of Compound (II) is characterized by an X-ray powder diffractogram having a signal at 13.5±0.2 degrees two-theta. In some embodiments, crystalline Form NP of Compound (II) is characterized by an X-ray powder diffractogram having a signal at 14.4±0.2 degrees two-theta. In some embodiments, crystalline Form NP of Compound (II) is characterized by an X-ray powder diffractogram having a signal at 15.2±0.2 degrees two-theta. In some embodiments, crystalline Form NP of Compound (II) is characterized by an X-ray powder diffractogram having a signal at 18.6±0.2 degrees two-theta. In some embodiments, crystalline Form NP of Compound (II) is characterized by an X-ray powder diffractogram having a signal at 19.0±0.2 degrees two-theta. In some embodiments, crystalline Form NP of Compound (II) is characterized by an X-ray powder diffractogram having a signal at 19.3±0.2 degrees two-theta. In some embodiments, crystalline Form NP of Compound (II) is characterized by an X-ray powder diffractogram having a signal at 21.0±0.2 degrees two-theta. In some embodiments, crystalline Form NP of Compound (II) is characterized by an X-ray powder diffractogram having a signal at 26.4±0.2 degrees two-theta.

In some embodiments, crystalline Form NP of Compound (II) is characterized by an X-ray powder diffractogram having a signal at two-theta values of 7.8±0.2, 13.5±0.2, 14.4±0.2, 15.2±0.2, 18.6±0.2, 19.0±0.2, 19.3±0.2, 21.0±0.2, 26.4±0.2. In some embodiments, crystalline Form NP of Compound (II) is characterized by an X-ray powder diffractogram having a signal at at least eight two-theta values chosen from 7.8±0.2, 13.5±0.2, 14.4±0.2, 15.2±0.2, 18.6±0.2, 19.0±0.2, 19.3±0.2, 21.0±0.2, 26.4±0.2. In some embodiments, crystalline Form NP of Compound (II) is characterized by an X-ray powder diffractogram having a signal at at least seven two-theta values chosen from 7.8±0.2, 13.5±0.2, 14.4±0.2, 15.2±0.2, 18.6±0.2, 19.0±0.2, 19.3±0.2, 21.0±0.2, 26.4±0.2. In some embodiments, crystalline Form NP of Compound (II) is characterized by an X-ray powder diffractogram having a signal at at least six two-theta values chosen from 7.8±0.2, 13.5±0.2, 14.4±0.2, 15.2±0.2, 18.6±0.2, 19.0±0.2, 19.3±0.2, 21.0±0.2, 26.4±0.2. In some embodiments, crystalline Form NP of Compound (II) is characterized by an X-ray powder diffractogram having a signal at at least five two-theta values chosen from 7.8±0.2, 13.5±0.2, 14.4±0.2, 15.2±0.2, 18.6±0.2, 19.0±0.2, 19.3±0.2, 21.0±0.2, 26.4±0.2. In some embodiments, crystalline Form NP of Compound (II) is characterized by an X-ray powder diffractogram having a signal at at least four two-theta values chosen from 7.8±0.2, 13.5±0.2, 14.4±0.2, 15.2±0.2, 18.6±0.2, 19.0±0.2, 19.3±0.2, 21.0±0.2, 26.4±0.2. In some embodiments, crystalline Form NP of Compound (II) is characterized by an X-ray powder diffractogram having a signal at at least three two-theta values chosen from 7.8±0.2, 13.5±0.2, 14.4±0.2, 15.2±0.2, 18.6±0.2, 19.0±0.2, 19.3±0.2, 21.0±0.2, 26.4±0.2. In some embodiments, crystalline Form NP of Compound (II) is characterized by an X-ray powder diffractogram having a signal at at least two two-theta values chosen from 7.8±0.2, 13.5±0.2, 14.4±0.2, 15.2±0.2, 18.6±0.2, 19.0±0.2, 19.3±0.2, 21.0±0.2, 26.4±0.2. In some embodiments, crystalline Form NP of Compound (II) is characterized by an X-ray powder diffractogram having a signal at at least one two-theta value chosen from 7.8±0.2, 13.5±0.2, 14.4±0.2, 15.2±0.2, 18.6±0.2, 19.0±0.2, 19.3±0.2, 21.0±0.2, 26.4±0.2.

In some embodiments, crystalline Form NP of Compound (II) is characterized by an X-ray powder diffractogram having a signal at two-theta values of 4.7±0.2, 7.8±0.2, 8.1±0.2, 10.1±0.2, 12.0±0.2, 15.0±0.2, 19.2±0.2, and 20.7±0.2. In some embodiments, crystalline Form NP of Compound (II) is characterized by an X-ray powder diffractogram having a signal at at least seven two-theta values chosen from 4.7±0.2, 7.8±0.2, 8.1±0.2, 10.1±0.2, 12.0±0.2, 15.0±0.2, 19.2±0.2, and 20.7±0.2. In some embodiments, crystalline Form NP of Compound (II) is characterized by an X-ray powder diffractogram having a signal at at least six two-theta values chosen from 4.7±0.2, 7.8±0.2, 8.1±0.2, 10.1±0.2, 12.0±0.2, 15.0±0.2, 19.2±0.2, and 20.7±0.2. In some embodiments, crystalline Form NP of Compound (II) is characterized by an X-ray powder diffractogram having a signal at at least five two-theta values chosen from 4.7±0.2, 7.8±0.2, 8.1±0.2, 10.1±0.2, 12.0±0.2, 15.0±0.2, 19.2±0.2, and 20.7±0.2. In some embodiments, crystalline Form NP of Compound (II) is characterized by an X-ray powder diffractogram having a signal at at least four two-theta values chosen from 4.7±0.2, 7.8±0.2, 8.1±0.2, 10.1±0.2, 12.0±0.2, 15.0±0.2, 19.2±0.2, and 20.7±0.2. In some embodiments, crystalline Form NP of Compound (II) is characterized by an X-ray powder diffractogram having a signal at at least three two-theta values chosen from 4.7±0.2, 7.8±0.2, 8.1±0.2, 10.1±0.2, 12.0±0.2, 15.0±0.2, 19.2±0.2, and 20.7±0.2. In some embodiments, crystalline Form NP of Compound (II) is characterized by an X-ray powder diffractogram having a signal at at least two two-theta values chosen from 4.7±0.2, 7.8±0.2, 8.1±0.2, 10.1±0.2, 12.0±0.2, 15.0±0.2, 19.2±0.2, and 20.7±0.2. In some embodiments, crystalline Form NP of Compound (II) is characterized by an X-ray powder diffractogram having a signal at at least one two-theta value chosen from 4.7±0.2, 7.8±0.2, 8.1±0.2, 10.1±0.2, 12.0±0.2, 15.0±0.2, 19.2±0.2, and 20.7±0.2.

In some embodiments, crystalline Form NP of Compound (II) is characterized by an X-ray powder diffractogram having a signal at two-theta values of 7.8±0.2, 13.5±0.2, 14.4±0.2, 15.2±0.2, 19.0±0.2, 21.0±0.2, 26.4±0.2. In some embodiments, crystalline Form NP of Compound (II) is characterized by an X-ray powder diffractogram having a signal at at least six two-theta values chosen from 47.8±0.2, 13.5±0.2, 14.4±0.2, 15.2±0.2, 19.0±0.2, 21.0±0.2, 26.4±0.2. In some embodiments, crystalline Form NP of Compound (II) is characterized by an X-ray powder diffractogram having a signal at at least five two-theta values chosen from 47.8±0.2, 13.5±0.2, 14.4±0.2, 15.2±0.2, 19.0±0.2, 21.0±0.2, 26.4±0.2. In some embodiments, crystalline Form NP of Compound (II) is characterized by an X-ray powder diffractogram having a signal at at least four two-theta values chosen from 47.8±0.2, 13.5±0.2, 14.4±0.2, 15.2±0.2, 19.0±0.2, 21.0±0.2, 26.4±0.2. In some embodiments, crystalline Form NP of Compound (II) is characterized by an X-ray powder diffractogram having a signal at at least three two-theta values chosen from 47.8±0.2, 13.5±0.2, 14.4±0.2, 15.2±0.2, 19.0±0.2, 21.0±0.2, 26.4±0.2. In some embodiments, crystalline Form NP of Compound (II) is characterized by an X-ray powder diffractogram having a signal at at least two two-theta values chosen from 47.8±0.2, 13.5±0.2, 14.4±0.2, 15.2±0.2, 19.0±0.2, 21.0±0.2, 26.4±0.2. In some embodiments, crystalline Form NP of Compound (II) is characterized by an X-ray powder diffractogram having a signal at at least one two-theta value chosen from 47.8±0.2, 13.5±0.2, 14.4±0.2, 15.2±0.2, 19.0±0.2, 21.0±0.2, 26.4±0.2.

In some embodiments, crystalline Form NP of Compound (II) is characterized by an X-ray powder diffractogram having a signal at two-theta values of 14.4±0.2, 15.2±0.2, 19.0±0.2, and 21.0±0.2. In some embodiments, crystalline Form NP of Compound (II) is characterized by an X-ray powder diffractogram having a signal at at least three two-theta values chosen from 14.4±0.2, 15.2±0.2, 19.0±0.2, and 21.0±0.2. In some embodiments, crystalline Form NP of Compound (II) is characterized by an X-ray powder diffractogram having a signal at at least two two-theta values chosen from 14.4±0.2, 15.2±0.2, 19.0±0.2, and 21.0±0.2. In some embodiments, crystalline Form NP of Compound (II) is characterized by an X-ray powder diffractogram having a signal at at least one two-theta value chosen from 14.4±0.2, 15.2±0.2, 19.0±0.2, and 21.0±0.2.

In some embodiments, crystalline Form NP of Compound (II) is characterized by an X-ray powder diffractogram substantially similar to that in FIG. 33.

In some embodiments, the present disclosure provides crystalline Form NP of Compound (II) prepared by a process comprising crystallizing Compound (II) from a mixture of n-pentanol and Compound (II). In some embodiments, the crystallizing is carried out for a period of time of more than one week. In some embodiments, the crystallizing is carried out at room temperature.

In some embodiments, the present disclosure provides methods of preparing crystalline Form NP of Compound (II) comprising crystallizing Compound (II) from a mixture of n-pentanol and Compound (II). In some embodiments, the crystallizing is carried out for a period of time of more than one week. In some embodiments, the crystallizing is carried out at room temperature.

### Crystalline Form EE of Compound (II)

In some embodiments, the present disclosure provides crystalline Form EE of Compound (II):

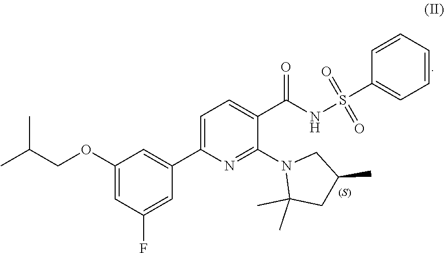

In some embodiments, crystalline Form EE is a 2-ethoxyethanol solvate of Compound (II).

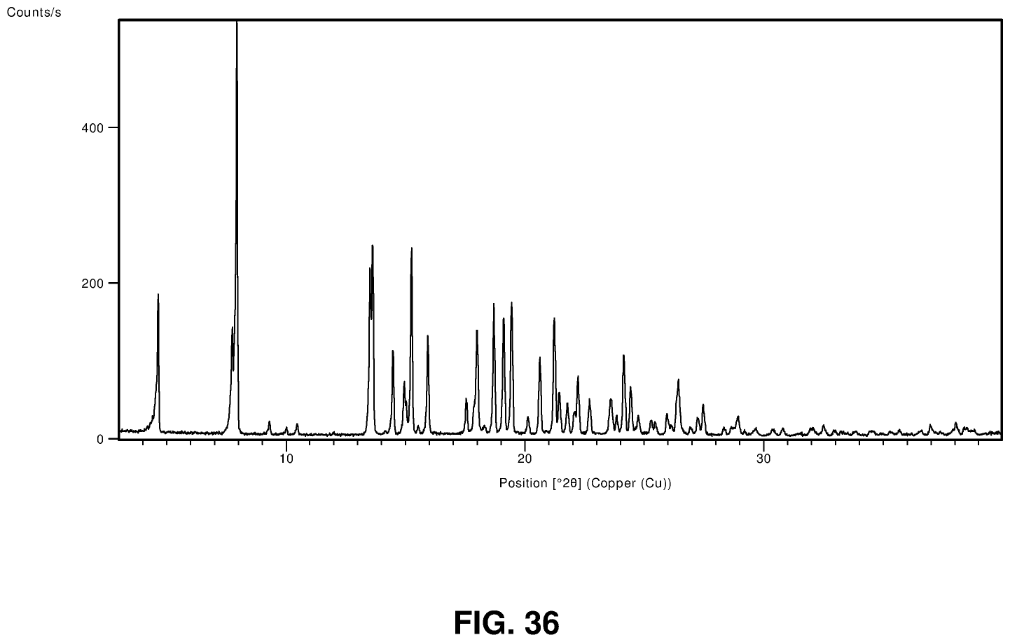

FIG. 36 shows an X-ray powder diffractogram of crystalline Form EE of Compound (II) at ambient conditions.

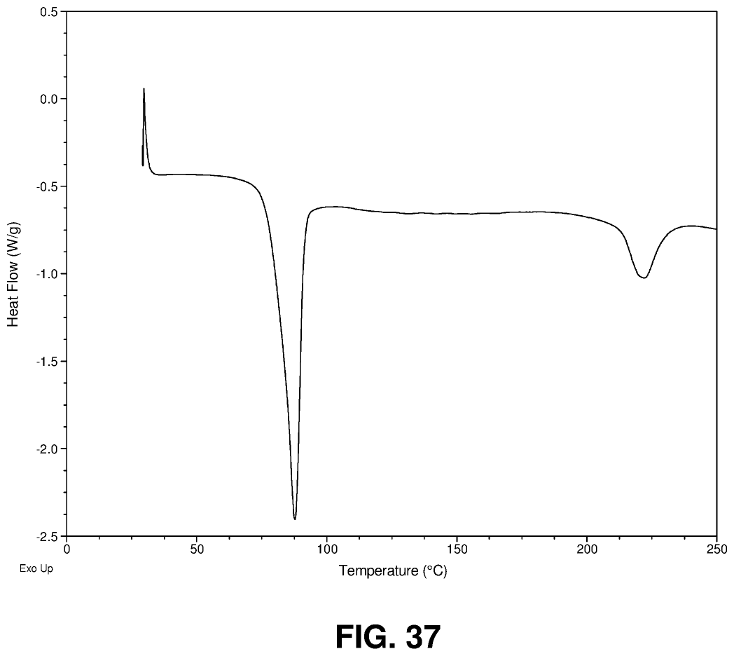

FIG. 37 shows a DSC trace of the crystalline Form EE of Compound (II). In some embodiments, crystalline Form EE of Compound (II) is characterized by a DSC having an onset of desolvation temperature of 81.3° C. and/or a peak temperature of 87.7° C.

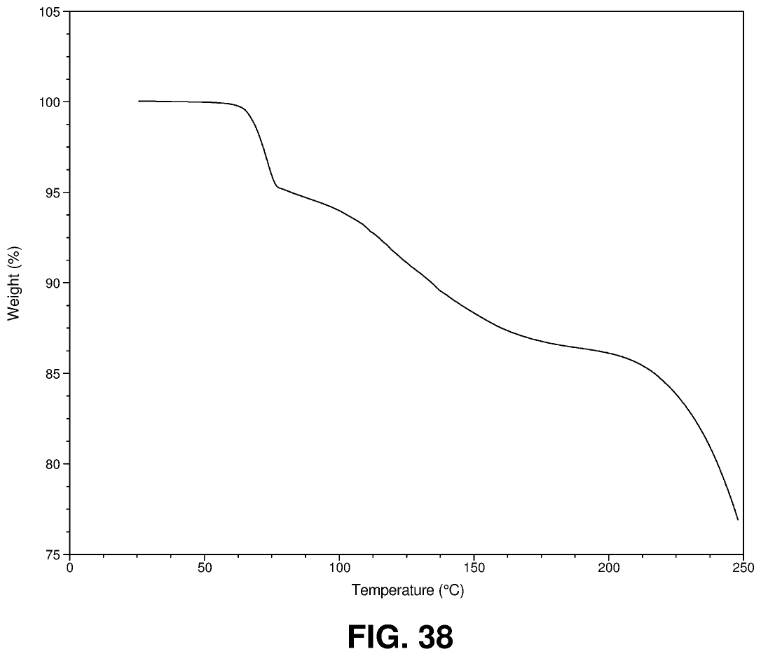

FIG. 38 shows the results of a TGA of crystalline Form EE of Compound (II). In some embodiments, crystalline Form EE of Compound (II) is characterized by a TGA having an onset of decomposition temperature of about 200° C.

In some embodiments, crystalline Form EE of Compound (II) is in substantially pure form. In some embodiments, crystalline Form EE of Compound (II) is characterized by an X-ray powder diffractogram generated by an X-ray powder diffraction analysis with an incident beam of Cu Kα radiation.

In some embodiments, crystalline Form EE of Compound (II) is characterized by an X-ray powder diffractogram having a signal at 4.6±0.2 degrees two-theta. In some embodiments, crystalline Form EE of Compound (II) is characterized by an X-ray powder diffractogram having a signal at 7.9±0.2 degrees two-theta. In some embodiments, crystalline Form EE of Compound (II) is characterized by an X-ray powder diffractogram having a signal at 13.6±0.2 degrees two-theta. In some embodiments, crystalline Form EE of Compound (II) is characterized by an X-ray powder diffractogram having a signal at 14.5±0.2 degrees two-theta. In some embodiments, crystalline Form EE of Compound (II) is characterized by an X-ray powder diffractogram having a signal at 15.3±0.2 degrees two-theta. In some embodiments, crystalline Form EE of Compound (II) is characterized by an X-ray powder diffractogram having a signal at 15.9±0.2 degrees two-theta. In some embodiments, crystalline Form EE of Compound (II) is characterized by an X-ray powder diffractogram having a signal at 18.0±0.2 degrees two-theta. In some embodiments, crystalline Form EE of Compound (II) is characterized by an X-ray powder diffractogram having a signal at 18.7±0.2 degrees two-theta. In some embodiments, crystalline Form EE of Compound (II) is characterized by an X-ray powder diffractogram having a signal at 19.1±0.2 degrees two-theta. In some embodiments, crystalline Form EE of Compound (II) is characterized by an X-ray powder diffractogram having a signal at 19.4±0.2 degrees two-theta. In some embodiments, crystalline Form EE of Compound (II) is characterized by an X-ray powder diffractogram having a signal at 21.2±0.2 degrees two-theta. In some embodiments, crystalline Form EE of Compound (II) is characterized by an X-ray powder diffractogram having a signal at 26.4±0.2 degrees two-theta.

In some embodiments, crystalline Form EE of Compound (II) is characterized by an X-ray powder diffractogram having a signal at two-theta values of 4.6±0.2, 7.9±0.2, 13.6±0.2, 14.5±0.2, 15.3±0.2, 15.9±0.2, 18.0±0.2, 18.7±0.2, 19.1±0.2, 19.4±0.2, 21.2±0.2, and 26.4±0.2. In some embodiments, crystalline Form EE of Compound (II) is characterized by an X-ray powder diffractogram having a signal at at least eleven two-theta values chosen from 4.6±0.2, 7.9±0.2, 13.6±0.2, 14.5±0.2, 15.3±0.2, 15.9±0.2, 18.0±0.2, 18.7±0.2, 19.1±0.2, 19.4±0.2, 21.2±0.2, and 26.4±0.2. In some embodiments, crystalline Form EE of Compound (II) is characterized by an X-ray powder diffractogram having a signal at at least ten two-theta values chosen from 4.6±0.2, 7.9±0.2, 13.6±0.2, 14.5±0.2, 15.3±0.2, 15.9±0.2, 18.0±0.2, 18.7±0.2, 19.1±0.2, 19.4±0.2, 21.2±0.2, and 26.4±0.2. In some embodiments, crystalline Form EE of Compound (II) is characterized by an X-ray powder diffractogram having a signal at at least nine two-theta values chosen from 4.6±0.2, 7.9±0.2, 13.6±0.2, 14.5±0.2, 15.3±0.2, 15.9±0.2, 18.0±0.2, 18.7±0.2, 19.1±0.2, 19.4±0.2, 21.2±0.2, and 26.4±0.2. In some embodiments, crystalline Form EE of Compound (II) is characterized by an X-ray powder diffractogram having a signal at at least eight two-theta values chosen from 4.6±0.2, 7.9±0.2, 13.6±0.2, 14.5±0.2, 15.3±0.2, 15.9±0.2, 18.0±0.2, 18.7±0.2, 19.1±0.2, 19.4±0.2, 21.2±0.2, and 26.4±0.2. In some embodiments, crystalline Form EE of Compound (II) is characterized by an X-ray powder diffractogram having a signal at at least seven two-theta values chosen from 4.6±0.2, 7.9±0.2, 13.6±0.2, 14.5±0.2, 15.3±0.2, 15.9±0.2, 18.0±0.2, 18.7±0.2, 19.1±0.2, 19.4±0.2, 21.2±0.2, and 26.4±0.2. In some embodiments, crystalline Form EE of Compound (II) is characterized by an X-ray powder diffractogram having a signal at at least six two-theta values chosen from 4.6±0.2, 7.9±0.2, 13.6±0.2, 14.5±0.2, 15.3±0.2, 15.9±0.2, 18.0±0.2, 18.7±0.2, 19.1±0.2, 19.4±0.2, 21.2±0.2, and 26.4±0.2. In some embodiments, crystalline Form EE of Compound (II) is characterized by an X-ray powder diffractogram having a signal at at least five two-theta values chosen from 4.6±0.2, 7.9±0.2, 13.6±0.2, 14.5±0.2, 15.3±0.2, 15.9±0.2, 18.0±0.2, 18.7±0.2, 19.1±0.2, 19.4±0.2, 21.2±0.2, and 26.4±0.2. In some embodiments, crystalline Form EE of Compound (II) is characterized by an X-ray powder diffractogram having a signal at at least four two-theta values chosen from 4.6±0.2, 7.9±0.2, 13.6±0.2, 14.5±0.2, 15.3±0.2, 15.9±0.2, 18.0±0.2, 18.7±0.2, 19.1±0.2, 19.4±0.2, 21.2±0.2, and 26.4±0.2. In some embodiments, crystalline Form EE of Compound (II) is characterized by an X-ray powder diffractogram having a signal at at least three two-theta values chosen from 4.6±0.2, 7.9±0.2, 13.6±0.2, 14.5±0.2, 15.3±0.2, 15.9±0.2, 18.0±0.2, 18.7±0.2, 19.1±0.2, 19.4±0.2, 21.2±0.2, and 26.4±0.2. In some embodiments, crystalline Form EE of Compound (II) is characterized by an X-ray powder diffractogram having a signal at at least two two-theta values chosen from 4.6±0.2, 7.9±0.2, 13.6±0.2, 14.5±0.2, 15.3±0.2, 15.9±0.2, 18.0±0.2, 18.7±0.2, 19.1±0.2, 19.4±0.2, 21.2±0.2, and 26.4±0.2. In some embodiments, crystalline Form EE of Compound (II) is characterized by an X-ray powder diffractogram having a signal at at least one two-theta value chosen from 4.6±0.2, 7.9±0.2, 13.6±0.2, 14.5±0.2, 15.3±0.2, 15.9±0.2, 18.0±0.2, 18.7±0.2, 19.1±0.2, 19.4±0.2, 21.2±0.2, and 26.4±0.2.

In some embodiments, crystalline Form EE of Compound (II) is characterized by an X-ray powder diffractogram having a signal at two-theta values of 4.6±0.2, 7.9±0.2, 13.6±0.2, 14.5±0.2, 15.3±0.2, 15.9±0.2, 18.7±0.2, 19.1±0.2, and 26.4±0.2. In some embodiments, crystalline Form EE of Compound (II) is characterized by an X-ray powder diffractogram having a signal at at least eight two-theta values chosen from 4.6±0.2, 7.9±0.2, 13.6±0.2, 14.5±0.2, 15.3±0.2, 15.9±0.2, 18.7±0.2, 19.1±0.2, and 26.4±0.2. In some embodiments, crystalline Form EE of Compound (II) is characterized by an X-ray powder diffractogram having a signal at at least seven two-theta values chosen from 4.6±0.2, 7.9±0.2, 13.6±0.2, 14.5±0.2, 15.3±0.2, 15.9±0.2, 18.7±0.2, 19.1±0.2, and 26.4±0.2. In some embodiments, crystalline Form EE of Compound (II) is characterized by an X-ray powder diffractogram having a signal at at least six two-theta values chosen from 4.6±0.2, 7.9±0.2, 13.6±0.2, 14.5±0.2, 15.3±0.2, 15.9±0.2, 18.7±0.2, 19.1±0.2, and 26.4±0.2. In some embodiments, crystalline Form EE of Compound (II) is characterized by an X-ray powder diffractogram having a signal at at least five two-theta values chosen from 4.6±0.2, 7.9±0.2, 13.6±0.2, 14.5±0.2, 15.3±0.2, 15.9±0.2, 18.7±0.2, 19.1±0.2, and 26.4±0.2. In some embodiments, crystalline Form EE of Compound (II) is characterized by an X-ray powder diffractogram having a signal at at least four two-theta values chosen from 4.6±0.2, 7.9±0.2, 13.6±0.2, 14.5±0.2, 15.3±0.2, 15.9±0.2, 18.7±0.2, 19.1±0.2, and 26.4±0.2. In some embodiments, crystalline Form EE of Compound (II) is characterized by an X-ray powder diffractogram having a signal at at least three two-theta values chosen from 4.6±0.2, 7.9±0.2, 13.6±0.2, 14.5±0.2, 15.3±0.2, 15.9±0.2, 18.7±0.2, 19.1±0.2, and 26.4±0.2. In some embodiments, crystalline Form EE of Compound (II) is characterized by an X-ray powder diffractogram having a signal at at least two two-theta values chosen from 4.6±0.2, 7.9±0.2, 13.6±0.2, 14.5±0.2, 15.3±0.2, 15.9±0.2, 18.7±0.2, 19.1±0.2, and 26.4±0.2. In some embodiments, crystalline Form EE of Compound (II) is characterized by an X-ray powder diffractogram having a signal at at least one two-theta value chosen from 4.6±0.2, 7.9±0.2, 13.6±0.2, 14.5±0.2, 15.3±0.2, 15.9±0.2, 18.7±0.2, 19.1±0.2, and 26.4±0.2.

In some embodiments, crystalline Form EE of Compound (II) is characterized by an X-ray powder diffractogram having a signal at two-theta values of 4.6±0.2, 7.9±0.2, 14.5±0.2, 15.3±0.2, 15.9±0.2, 19.1±0.2, and 26.4±0.2. In some embodiments, crystalline Form EE of Compound (II) is characterized by an X-ray powder diffractogram having a signal at at least six two-theta values chosen from 4.6±0.2, 7.9±0.2, 14.5±0.2, 15.3±0.2, 15.9±0.2, 19.1±0.2, and 26.4±0.2. In some embodiments, crystalline Form EE of Compound (II) is characterized by an X-ray powder diffractogram having a signal at at least five two-theta values chosen from 4.6±0.2, 7.9±0.2, 14.5±0.2, 15.3±0.2, 15.9±0.2, 19.1±0.2, and 26.4±0.2. In some embodiments, crystalline Form EE of Compound (II) is characterized by an X-ray powder diffractogram having a signal at at least four two-theta values chosen from 4.6±0.2, 7.9±0.2, 14.5±0.2, 15.3±0.2, 15.9±0.2, 19.1±0.2, and 26.4±0.2. In some embodiments, crystalline Form EE of Compound (II) is characterized by an X-ray powder diffractogram having a signal at at least three two-theta values chosen from 4.6±0.2, 7.9±0.2, 14.5±0.2, 15.3±0.2, 15.9±0.2, 19.1±0.2, and 26.4±0.2. In some embodiments, crystalline Form EE of Compound (II) is characterized by an X-ray powder diffractogram having a signal at at least two two-theta values chosen from 4.6±0.2, 7.9±0.2, 14.5±0.2, 15.3±0.2, 15.9±0.2, 19.1±0.2, and 26.4±0.2. In some embodiments, crystalline Form EE of Compound (II) is characterized by an X-ray powder diffractogram having a signal at at least one two-theta value chosen from 4.6±0.2, 7.9±0.2, 14.5±0.2, 15.3±0.2, 15.9±0.2, 19.1±0.2, and 26.4±0.2.

In some embodiments, crystalline Form EE of Compound (II) is characterized by an X-ray powder diffractogram having a signal at two-theta values of 14.5±0.2, 15.3±0.2, 19.1±0.2, and 26.4±0.2. In some embodiments, crystalline Form EE of Compound (II) is characterized by an X-ray powder diffractogram having a signal at at least three two-theta values chosen from 14.5±0.2, 15.3±0.2, 19.1±0.2, and 26.4±0.2. In some embodiments, crystalline Form EE of Compound (II) is characterized by an X-ray powder diffractogram having a signal at at least two two-theta values chosen from 14.5±0.2, 15.3±0.2, 19.1±0.2, and 26.4±0.2. In some embodiments, crystalline Form EE of Compound (II) is characterized by an X-ray powder diffractogram having a signal at at least one two-theta value chosen from 14.5±0.2, 15.3±0.2, 19.1±0.2, and 26.4±0.2.

In some embodiments, crystalline Form EE of Compound (II) is characterized by an X-ray powder diffractogram substantially similar to that in FIG. 36.

In some embodiments, the present disclosure provides crystalline Form EE of Compound (II) prepared by a process comprising crystallizing Compound (II) from a mixture of 2-ethoxyethanol and Compound (II). In some embodiments, the crystallizing is carried out for a period of time of more than one week. In some embodiments, the crystallizing is carried out at room temperature.

In some embodiments, the present disclosure provides methods of preparing crystalline Form EE of Compound (II) comprising crystallizing Compound (II) from a mixture of 2-ethoxyethanol and Compound (II). In some embodiments, the crystallizing is carried out for a period of time of more than one week. In some embodiments, the crystallizing is carried out at room temperature.

### Crystalline Form E of Compound (II)

In some embodiments, the present disclosure provides crystalline Form E of Compound (II):

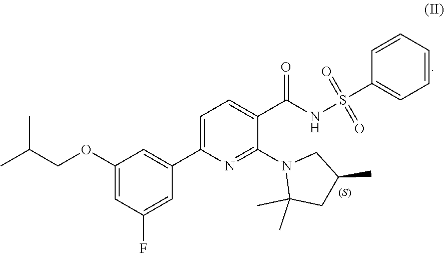

In some embodiments, crystalline Form E is an ethanol solvate of Compound (II).

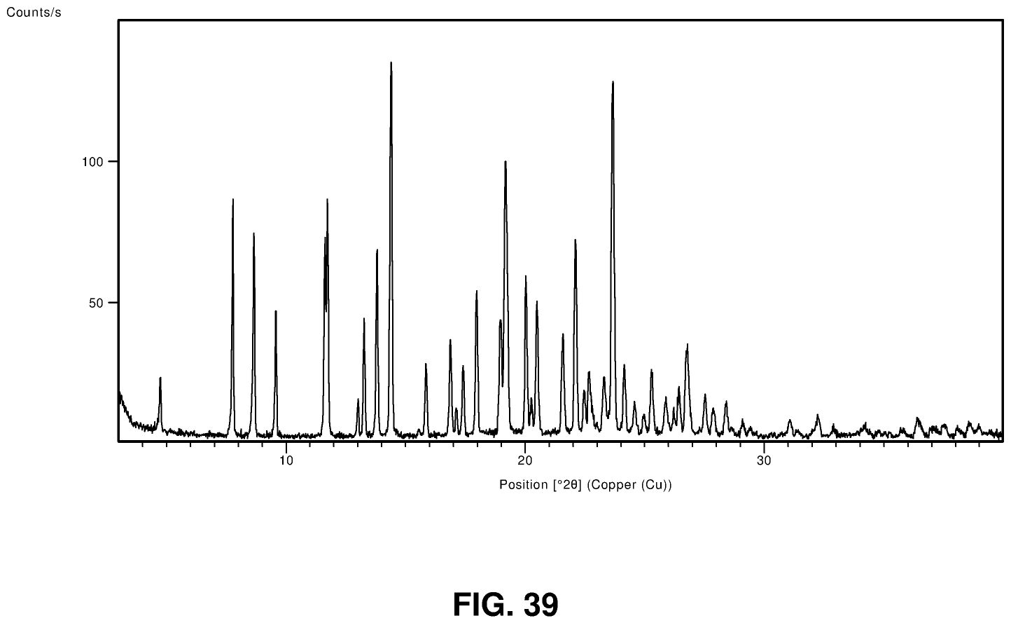

FIG. 39 shows an X-ray powder diffractogram of crystalline Form E of Compound (II) at ambient conditions.

In some embodiments, crystalline Form E of Compound (II) is in substantially pure form. In some embodiments, crystalline Form E of Compound (II) is characterized by an X-ray powder diffractogram generated by an X-ray powder diffraction analysis with an incident beam of Cu Kα radiation.

In some embodiments, crystalline Form E of Compound (II) is characterized by an X-ray powder diffractogram having a signal at 7.8±0.2 degrees two-theta. In some embodiments, crystalline Form E of Compound (II) is characterized by an X-ray powder diffractogram having a signal at 8.7±0.2 degrees two-theta. In some embodiments, crystalline Form E of Compound (II) is characterized by an X-ray powder diffractogram having a signal at 11.7±0.2 degrees two-theta. In some embodiments, crystalline Form E of Compound (II) is characterized by an X-ray powder diffractogram having a signal at 13.8±0.2 degrees two-theta. In some embodiments, crystalline Form E of Compound (II) is characterized by an X-ray powder diffractogram having a signal at 14.4±0.2 degrees two-theta. In some embodiments, crystalline Form E of Compound (II) is characterized by an X-ray powder diffractogram having a signal at 18.0±0.2 degrees two-theta. In some embodiments, crystalline Form E of Compound (II) is characterized by an X-ray powder diffractogram having a signal at 19.2±0.2 degrees two-theta. In some embodiments, crystalline Form E of Compound (II) is characterized by an X-ray powder diffractogram having a signal at 20.0±0.2 degrees two-theta. In some embodiments, crystalline Form E of Compound (II) is characterized by an X-ray powder diffractogram having a signal at 22.1±0.2 degrees two-theta. In some embodiments, crystalline Form E of Compound (II) is characterized by an X-ray powder diffractogram having a signal at 23.7±0.2 degrees two-theta.

In some embodiments, crystalline Form E of Compound (II) is characterized by an X-ray powder diffractogram having a signal at two-theta values of 7.8±0.2, 8.7±0.2, 11.7±0.2, 13.8±0.2, 14.4±0.2, 18.0±0.2, 19.2±0.2, 20.0±0.2, 22.1±0.2, and 23.7±0.2. In some embodiments, crystalline Form E of Compound (II) is characterized by an X-ray powder diffractogram having a signal at at least nine two-theta values chosen from 7.8±0.2, 8.7±0.2, 11.7±0.2, 13.8±0.2, 14.4±0.2, 18.0±0.2, 19.2±0.2, 20.0±0.2, 22.1±0.2, and 23.7±0.2. In some embodiments, crystalline Form E of Compound (II) is characterized by an X-ray powder diffractogram having a signal at at least eight two-theta values chosen from 7.8±0.2, 8.7±0.2, 11.7±0.2, 13.8±0.2, 14.4±0.2, 18.0±0.2, 19.2±0.2, 20.0±0.2, 22.1±0.2, and 23.7±0.2. In some embodiments, crystalline Form E of Compound (II) is characterized by an X-ray powder diffractogram having a signal at at least seven two-theta values chosen from 7.8±0.2, 8.7±0.2, 11.7±0.2, 13.8±0.2, 14.4±0.2, 18.0±0.2, 19.2±0.2, 20.0±0.2, 22.1±0.2, and 23.7±0.2. In some embodiments, crystalline Form E of Compound (II) is characterized by an X-ray powder diffractogram having a signal at at least six two-theta values chosen from 7.8±0.2, 8.7±0.2, 11.7±0.2, 13.8±0.2, 14.4±0.2, 18.0±0.2, 19.2±0.2, 20.0±0.2, 22.1±0.2, and 23.7±0.2. In some embodiments, crystalline Form E of Compound (II) is characterized by an X-ray powder diffractogram having a signal at at least five two-theta values chosen from 7.8±0.2, 8.7±0.2, 11.7±0.2, 13.8±0.2, 14.4±0.2, 18.0±0.2, 19.2±0.2, 20.0±0.2, 22.1±0.2, and 23.7±0.2. In some embodiments, crystalline Form E of Compound (II) is characterized by an X-ray powder diffractogram having a signal at at least four two-theta values chosen from 7.8±0.2, 8.7±0.2, 11.7±0.2, 13.8±0.2, 14.4±0.2, 18.0±0.2, 19.2±0.2, 20.0±0.2, 22.1±0.2, and 23.7±0.2. In some embodiments, crystalline Form E of Compound (II) is characterized by an X-ray powder diffractogram having a signal at at least three two-theta values chosen from 7.8±0.2, 8.7±0.2, 11.7±0.2, 13.8±0.2, 14.4±0.2, 18.0±0.2, 19.2±0.2, 20.0±0.2, 22.1±0.2, and 23.7±0.2. In some embodiments, crystalline Form E of Compound (II) is characterized by an X-ray powder diffractogram having a signal at at least two two-theta values chosen from 7.8±0.2, 8.7±0.2, 11.7±0.2, 13.8±0.2, 14.4±0.2, 18.0±0.2, 19.2±0.2, 20.0±0.2, 22.1±0.2, and 23.7±0.2. In some embodiments, crystalline Form E of Compound (II) is characterized by an X-ray powder diffractogram having a signal at at least one two-theta value chosen from 7.8±0.2, 8.7±0.2, 11.7±0.2, 13.8±0.2, 14.4±0.2, 18.0±0.2, 19.2±0.2, 20.0±0.2, 22.1±0.2, and 23.7±0.2.

In some embodiments, crystalline Form E of Compound (II) is characterized by an X-ray powder diffractogram having a signal at two-theta values of 7.8±0.2, 8.7±0.2, 11.7±0.2, 14.4±0.2, 19.2±0.2, 22.1±0.2, and 23.7±0.2. In some embodiments, crystalline Form E of Compound (II) is characterized by an X-ray powder diffractogram having a signal at at least six two-theta values chosen from 7.8±0.2, 8.7±0.2, 11.7±0.2, 14.4±0.2, 19.2±0.2, 22.1±0.2, and 23.7±0.2. In some embodiments, crystalline Form E of Compound (II) is characterized by an X-ray powder diffractogram having a signal at at least five two-theta values chosen from 7.8±0.2, 8.7±0.2, 11.7±0.2, 14.4±0.2, 19.2±0.2, 22.1±0.2, and 23.7±0.2. In some embodiments, crystalline Form E of Compound (II) is characterized by an X-ray powder diffractogram having a signal at at least four two-theta values chosen from 7.8±0.2, 8.7±0.2, 11.7±0.2, 14.4±0.2, 19.2±0.2, 22.1±0.2, and 23.7±0.2. In some embodiments, crystalline Form E of Compound (II) is characterized by an X-ray powder diffractogram having a signal at at least three two-theta values chosen from 7.8±0.2, 8.7±0.2, 11.7±0.2, 14.4±0.2, 19.2±0.2, 22.1±0.2, and 23.7±0.2. In some embodiments, crystalline Form E of Compound (II) is characterized by an X-ray powder diffractogram having a signal at at least two two-theta values chosen from 7.8±0.2, 8.7±0.2, 11.7±0.2, 14.4±0.2, 19.2±0.2, 22.1±0.2, and 23.7±0.2. In some embodiments, crystalline Form E of Compound (II) is characterized by an X-ray powder diffractogram having a signal at at least one two-theta value chosen from 7.8±0.2, 8.7±0.2, 11.7±0.2, 14.4±0.2, 19.2±0.2, 22.1±0.2, and 23.7±0.2.

In some embodiments, crystalline Form E of Compound (II) is characterized by an X-ray powder diffractogram having a signal at two-theta values of 7.8±0.2, 8.7±0.2, 11.7±0.2, 14.4±0.2, 19.2±0.2, and 23.7±0.2. In some embodiments, crystalline Form E of Compound (II) is characterized by an X-ray powder diffractogram having a signal at at least five two-theta values chosen from 7.8±0.2, 8.7±0.2, 11.7±0.2, 14.4±0.2, 19.2±0.2, and 23.7±0.2. In some embodiments, crystalline Form E of Compound (II) is characterized by an X-ray powder diffractogram having a signal at at least four two-theta values chosen from 7.8±0.2, 8.7±0.2, 11.7±0.2, 14.4±0.2, 19.2±0.2, and 23.7±0.2. In some embodiments, crystalline Form E of Compound (II) is characterized by an X-ray powder diffractogram having a signal at at least three two-theta values chosen from 7.8±0.2, 8.7±0.2, 11.7±0.2, 14.4±0.2, 19.2±0.2, and 23.7±0.2. In some embodiments, crystalline Form E of Compound (II) is characterized by an X-ray powder diffractogram having a signal at at least two two-theta values chosen from 7.8±0.2, 8.7±0.2, 11.7±0.2, 14.4±0.2, 19.2±0.2, and 23.7±0.2. In some embodiments, crystalline Form E of Compound (II) is characterized by an X-ray powder diffractogram having a signal at at least one two-theta value chosen from 7.8±0.2, 8.7±0.2, 11.7±0.2, 14.4±0.2, 19.2±0.2, and 23.7±0.2.

In some embodiments, crystalline Form E of Compound (II) is characterized by an X-ray powder diffractogram having a signal at two-theta values of 7.8±0.2, 8.7±0.2, and 11.7±0.2. In some embodiments, crystalline Form E of Compound (II) is characterized by an X-ray powder diffractogram having a signal at at least two two-theta values chosen from 7.8±0.2, 8.7±0.2, and 11.7±0.2. In some embodiments, crystalline Form E of Compound (II) is characterized by an X-ray powder diffractogram having a signal at at least one two-theta value chosen from 7.8±0.2, 8.7±0.2, and 11.7±0.2.

In some embodiments, crystalline Form E of Compound (II) is characterized by an X-ray powder diffractogram substantially similar to that in FIG. 39.

In some embodiments, the present disclosure provides crystalline Form E of Compound (II) prepared by a process comprising crystallizing Compound (II) from a mixture of ethanol and Compound (II). In some embodiments, the crystallizing is carried out for about 24 hours. In some embodiments, the crystallizing is carried out at room temperature.

In some embodiments, the present disclosure provides methods of preparing crystalline Form E of Compound (II) comprising crystallizing Compound (II) from a mixture of ethanol and Compound (II). In some embodiments, the crystallizing is carried out for about 24 hours. In some embodiments, the crystallizing is carried out at room temperature.

### Crystalline Form T of Compound (II)

In some embodiments, the present disclosure provides crystalline Form T of Compound (II):

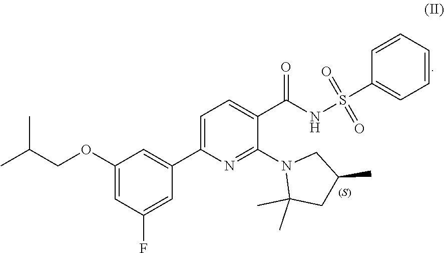

In some embodiments, crystalline Form T is a tetrahydrofuran solvate of Compound (II).

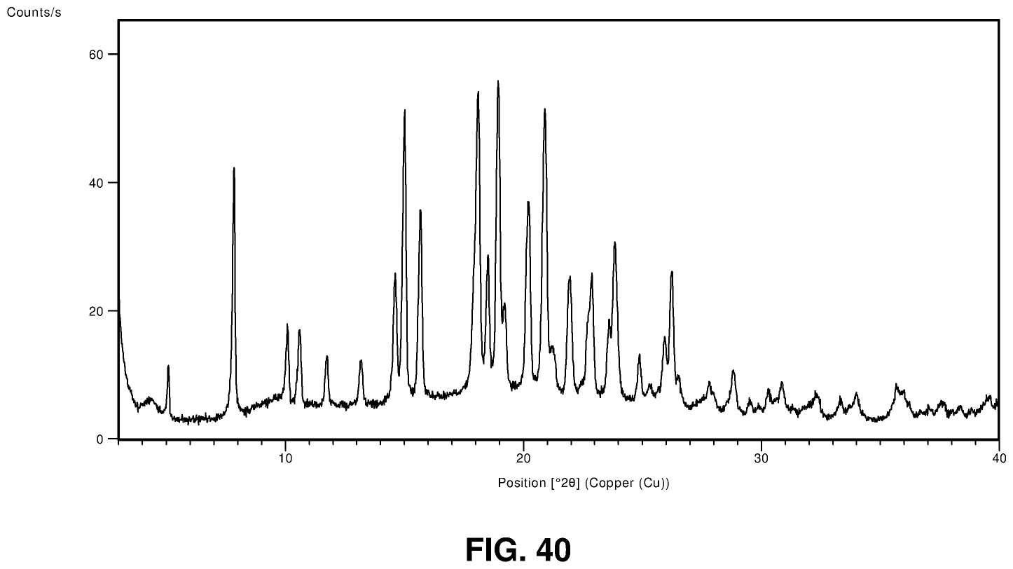

FIG. 40 shows an X-ray powder diffractogram of crystalline Form T of Compound (II) at ambient conditions.

In some embodiments, crystalline Form T of Compound (II) is in substantially pure form. In some embodiments, crystalline Form T of Compound (II) is characterized by an X-ray powder diffractogram generated by an X-ray powder diffraction analysis with an incident beam of Cu Kα radiation.

In some embodiments, crystalline Form T of Compound (II) is characterized by an X-ray powder diffractogram having a signal at 7.9±0.2 degrees two-theta. In some embodiments, crystalline Form T of Compound (II) is characterized by an X-ray powder diffractogram having a signal at 10.6±0.2 degrees two-theta. In some embodiments, crystalline Form T of Compound (II) is characterized by an X-ray powder diffractogram having a signal at 15.0±0.2 degrees two-theta. In some embodiments, crystalline Form T of Compound (II) is characterized by an X-ray powder diffractogram having a signal at 15.7±0.2 degrees two-theta. In some embodiments, crystalline Form T of Compound (II) is characterized by an X-ray powder diffractogram having a signal at 18.1±0.2 degrees two-theta. In some embodiments, crystalline Form T of Compound (II) is characterized by an X-ray powder diffractogram having a signal at 18.5±0.2 degrees two-theta. In some embodiments, crystalline Form T of Compound (II) is characterized by an X-ray powder diffractogram having a signal at 19.0±0.2 degrees two-theta. In some embodiments, crystalline Form T of Compound (II) is characterized by an X-ray powder diffractogram having a signal at 20.2±0.2 degrees two-theta. In some embodiments, crystalline Form T of Compound (II) is characterized by an X-ray powder diffractogram having a signal at 20.9±0.2 degrees two-theta. In some embodiments, crystalline Form T of Compound (II) is characterized by an X-ray powder diffractogram having a signal at 23.8±0.2 degrees two-theta.

In some embodiments, crystalline Form T of Compound (II) is characterized by an X-ray powder diffractogram having a signal at two-theta values of 7.9±0.2, 10.6±0.2, 15.0±0.2, 15.7±0.2, 18.1±0.2, 18.5±0.2, 19.0±0.2, 20.2±0.2, 20.9±0.2, and 23.8±0.2. In some embodiments, crystalline Form T of Compound (II) is characterized by an X-ray powder diffractogram having a signal at at least nine two-theta values chosen from 7.9±0.2, 10.6±0.2, 15.0±0.2, 15.7±0.2, 18.1±0.2, 18.5±0.2, 19.0±0.2, 20.2±0.2, 20.9±0.2, and 23.8±0.2. In some embodiments, crystalline Form T of Compound (II) is characterized by an X-ray powder diffractogram having a signal at at least eight two-theta values chosen from 7.9±0.2, 10.6±0.2, 15.0±0.2, 15.7±0.2, 18.1±0.2, 18.5±0.2, 19.0±0.2, 20.2±0.2, 20.9±0.2, and 23.8±0.2. In some embodiments, crystalline Form T of Compound (II) is characterized by an X-ray powder diffractogram having a signal at at least seven two-theta values chosen from 7.9±0.2, 10.6±0.2, 15.0±0.2, 15.7±0.2, 18.1±0.2, 18.5±0.2, 19.0±0.2, 20.2±0.2, 20.9±0.2, and 23.8±0.2. In some embodiments, crystalline Form T of Compound (II) is characterized by an X-ray powder diffractogram having a signal at at least six two-theta values chosen from 7.9±0.2, 10.6±0.2, 15.0±0.2, 15.7±0.2, 18.1±0.2, 18.5±0.2, 19.0±0.2, 20.2±0.2, 20.9±0.2, and 23.8±0.2. In some embodiments, crystalline Form T of Compound (II) is characterized by an X-ray powder diffractogram having a signal at at least five two-theta values chosen from 7.9±0.2, 10.6±0.2, 15.0±0.2, 15.7±0.2, 18.1±0.2, 18.5±0.2, 19.0±0.2, 20.2±0.2, 20.9±0.2, and 23.8±0.2. In some embodiments, crystalline Form T of Compound (II) is characterized by an X-ray powder diffractogram having a signal at at least four two-theta values chosen from 7.9±0.2, 10.6±0.2, 15.0±0.2, 15.7±0.2, 18.1±0.2, 18.5±0.2, 19.0±0.2, 20.2±0.2, 20.9±0.2, and 23.8±0.2. In some embodiments, crystalline Form T of Compound (II) is characterized by an X-ray powder diffractogram having a signal at at least three two-theta values chosen from 7.9±0.2, 10.6±0.2, 15.0±0.2, 15.7±0.2, 18.1±0.2, 18.5±0.2, 19.0±0.2, 20.2±0.2, 20.9±0.2, and 23.8±0.2. In some embodiments, crystalline Form T of Compound (II) is characterized by an X-ray powder diffractogram having a signal at at least two two-theta values chosen from 7.9±0.2, 10.6±0.2, 15.0±0.2, 15.7±0.2, 18.1±0.2, 18.5±0.2, 19.0±0.2, 20.2±0.2, 20.9±0.2, and 23.8±0.2. In some embodiments, crystalline Form T of Compound (II) is characterized by an X-ray powder diffractogram having a signal at at least one two-theta value chosen from 7.9±0.2, 10.6±0.2, 15.0±0.2, 15.7±0.2, 18.1±0.2, 18.5±0.2, 19.0±0.2, 20.2±0.2, 20.9±0.2, and 23.8±0.2.

In some embodiments, crystalline Form T of Compound (II) is characterized by an X-ray powder diffractogram having a signal at two-theta values of 7.9±0.2, 10.6±0.2, 15.0±0.2, 15.7±0.2, 18.1±0.2, 18.5±0.2, and 19.0±0.2. In some embodiments, crystalline Form T of Compound (II) is characterized by an X-ray powder diffractogram having a signal at at least six two-theta values chosen from 7.9±0.2, 10.6±0.2, 15.0±0.2, 15.7±0.2, 18.1±0.2, 18.5±0.2, and 19.0±0.2. In some embodiments, crystalline Form T of Compound (II) is characterized by an X-ray powder diffractogram having a signal at at least five two-theta values chosen from 7.9±0.2, 10.6±0.2, 15.0±0.2, 15.7±0.2, 18.1±0.2, 18.5±0.2, and 19.0±0.2. In some embodiments, crystalline Form T of Compound (II) is characterized by an X-ray powder diffractogram having a signal at at least four two-theta values chosen from 7.9±0.2, 10.6±0.2, 15.0±0.2, 15.7±0.2, 18.1±0.2, 18.5±0.2, and 19.0±0.2. In some embodiments, crystalline Form T of Compound (II) is characterized by an X-ray powder diffractogram having a signal at at least three two-theta values chosen from 7.9±0.2, 10.6±0.2, 15.0±0.2, 15.7±0.2, 18.1±0.2, 18.5±0.2, and 19.0±0.2. In some embodiments, crystalline Form T of Compound (II) is characterized by an X-ray powder diffractogram having a signal at at least two two-theta values chosen from 7.9±0.2, 10.6±0.2, 15.0±0.2, 15.7±0.2, 18.1±0.2, 18.5±0.2, and 19.0±0.2. In some embodiments, crystalline Form T of Compound (II) is characterized by an X-ray powder diffractogram having a signal at at least one two-theta value chosen from 7.9±0.2, 10.6±0.2, 15.0±0.2, 15.7±0.2, 18.1±0.2, 18.5±0.2, and 19.0±0.2.

In some embodiments, crystalline Form T of Compound (II) is characterized by an X-ray powder diffractogram having a signal at two-theta values of 10.6±0.2, 18.1±0.2, and 18.5±0.2. In some embodiments, crystalline Form T of Compound (II) is characterized by an X-ray powder diffractogram having a signal at at least two two-theta values chosen from 10.6±0.2, 18.1±0.2, and 18.5±0.2. In some embodiments, crystalline Form T of Compound (II) is characterized by an X-ray powder diffractogram having a signal at at least one two-theta value chosen from 10.6±0.2, 18.1±0.2, and 18.5±0.2.

In some embodiments, crystalline Form T of Compound (II) is characterized by an X-ray powder diffractogram substantially similar to that in FIG. 40.

In some embodiments, the present disclosure provides crystalline Form T of Compound (II) prepared by a process comprising crystallizing Compound (II) from a mixture of tetrahydrofuran and Compound (II). In some embodiments, the crystallizing is carried out for about 24 hours. In some embodiments, the crystallizing is carried out for more than one week. In some embodiments, the crystallizing is carried out at room temperature.

In some embodiments, the present disclosure provides methods of preparing crystalline Form T of Compound (II) comprising crystallizing Compound (II) from a mixture of tetrahydrofuran and Compound (II). In some embodiments, the crystallizing is carried out for about 24 hours. In some embodiments, the crystallizing is carried out for more than one week. In some embodiments, the crystallizing is carried out at room temperature.

Crystalline Form AC of Compound (II)

In some embodiments, the present disclosure provides crystalline Form AC of Compound (II):

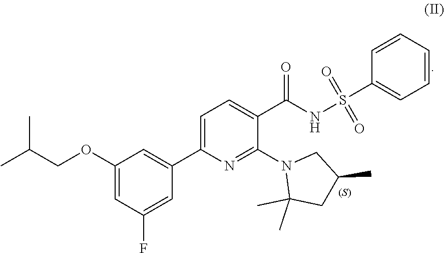

In some embodiments, crystalline Form AC is an acetonitrile solvate of Compound (II).

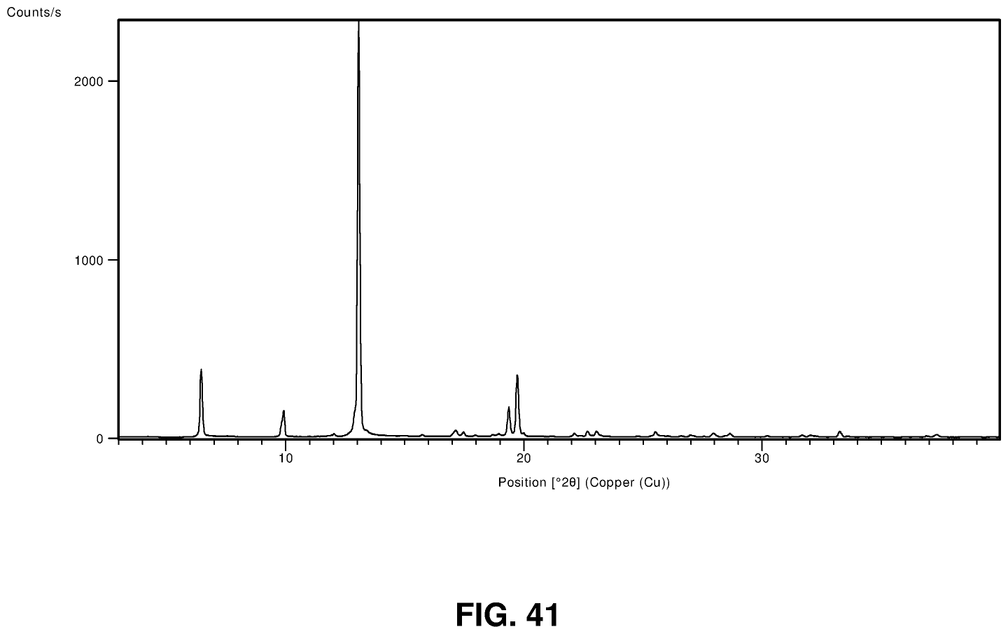

FIG. 41 shows an X-ray powder diffractogram of crystalline Form AC of Compound (II) at ambient conditions.

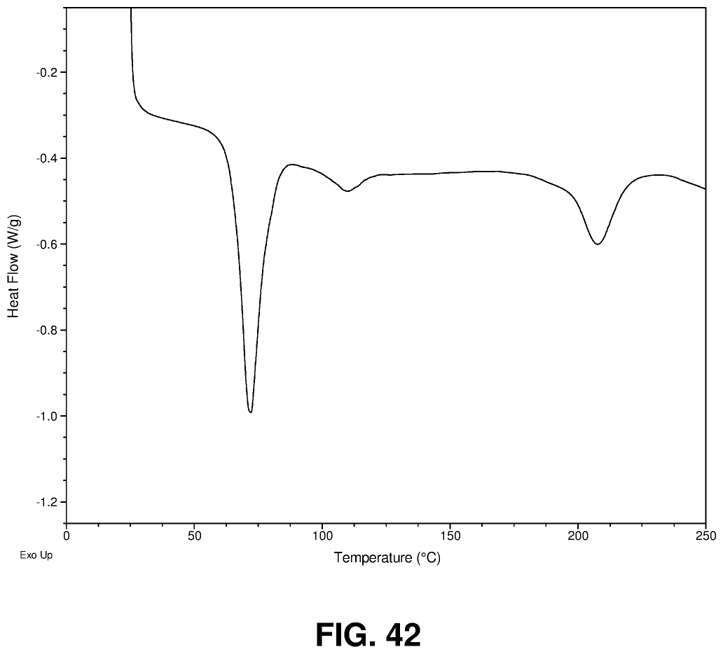

FIG. 42 shows a DSC trace of the crystalline Form AC of Compound (II). In some embodiments, crystalline Form AC of Compound (II) is characterized by a DSC having an onset of desolvation temperature of 65.0° C. and/or a peak temperature of 71.6° C. In some embodiments, crystalline Form AC of Compound (II) is characterized by a DSC having an onset of desolvation temperature of 98.8° C. and/or a peak temperature of 109.7° C.

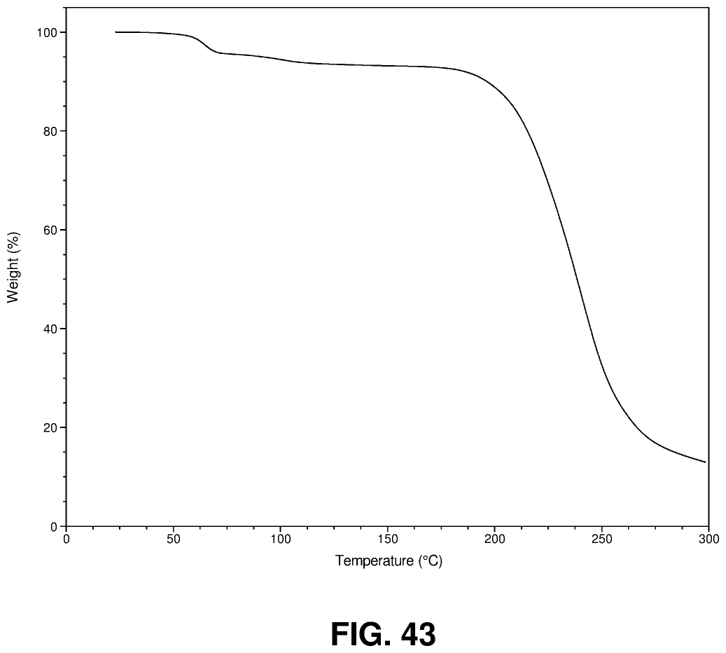

FIG. 43 shows the results of a TGA of crystalline Form AC of Compound (II). In some embodiments, crystalline Form AC of Compound (II) is characterized by a TGA having an onset of decomposition temperature of about 175° C.

In some embodiments, crystalline Form AC of Compound (II) is in substantially pure form. In some embodiments, crystalline Form AC of Compound (II) is characterized by an X-ray powder diffractogram generated by an X-ray powder diffraction analysis with an incident beam of Cu Kα radiation.

In some embodiments, crystalline Form AC of Compound (II) is characterized by an X-ray powder diffractogram having a signal at 6.5±0.2 degrees two-theta. In some embodiments, crystalline Form AC of Compound (II) is characterized by an X-ray powder diffractogram having a signal at 13.1±0.2 degrees two-theta. In some embodiments, crystalline Form AC of Compound (II) is characterized by an X-ray powder diffractogram having a signal at 19.4±0.2 degrees two-theta. In some embodiments, crystalline Form AC of Compound (II) is characterized by an X-ray powder diffractogram having a signal at 19.7±0.2 degrees two-theta.

In some embodiments, crystalline Form AC of Compound (II) is characterized by an X-ray powder diffractogram having a signal at two-theta values of 6.5±0.2, 13.1±0.2, 19.4±0.2, and 19.7±0.2. In some embodiments, crystalline Form AC of Compound (II) is characterized by an X-ray powder diffractogram having a signal at at least three two-theta values chosen from 6.5±0.2, 13.1±0.2, 19.4±0.2, and 19.7±0.2. In some embodiments, crystalline Form AC of Compound (II) is characterized by an X-ray powder diffractogram having a signal at at least two two-theta values chosen from 6.5±0.2, 13.1±0.2, 19.4±0.2, and 19.7±0.2. In some embodiments, crystalline Form AC of Compound (II) is characterized by an X-ray powder diffractogram having a signal at at least one two-theta values chosen from 6.5±0.2, 13.1±0.2, 19.4±0.2, and 19.7±0.2.

In some embodiments, crystalline Form AC of Compound (II) is characterized by an X-ray powder diffractogram having a signal at two-theta values of 6.5±0.2, 13.1±0.2, and 19.4±0.2. In some embodiments, crystalline Form AC of Compound (II) is characterized by an X-ray powder diffractogram having a signal at at least two two-theta values chosen from 6.5±0.2, 13.1±0.2, and 19.4±0.2. In some embodiments, crystalline Form AC of Compound (II) is characterized by an X-ray powder diffractogram having a signal at at least one two-theta values chosen from 6.5±0.2, 13.1±0.2, and 19.4±0.2.

In some embodiments, crystalline Form AC of Compound (II) is characterized by an X-ray powder diffractogram substantially similar to that in FIG. 41.

In some embodiments, the present disclosure provides crystalline Form AC of Compound (II) prepared by a process comprising crystallizing Compound (II) from a mixture of acetonitrile and Compound (II). In some embodiments, the crystallizing is carried out for about four days. In some embodiments, the crystallizing is carried out at room temperature. In some embodiments, the crystallizing is carried out below room temperature.

In some embodiments, the present disclosure provides methods of preparing crystalline Form AC of Compound (II) comprising crystallizing Compound (II) from a mixture of acetonitrile and Compound (II). In some embodiments, the crystallizing is carried out for about four days. In some embodiments, the crystallizing is carried out at room temperature. In some embodiments, the crystallizing is carried out below room temperature.

### Solvates

In some embodiments, the present disclosure provides at least one solvate of Compound (I) chosen from methanol solvates and dioxane/heptane solvates. Such solvates of Compound (I) can be prepared by stirring Compound (I) in a relevant solvent. In some embodiments, the present disclosure provides at least one solvate of Compound (II) chosen from iso-propanol solvates, n-propanol solvates, butanol solvates, and 2-methyl-1-propanol solvates, pentanol solvates, tetrahydrofuran solvates, ethanol solvates, acetonitrile solvates, and 2-ethoxyethanol solvates of Compound (II). Such solvates of Compound (II) can be prepared by stirring Compound (II) in a relevant solvent.

### Compositions and Methods

In some embodiments, the present disclosure provides compositions comprising at least one crystalline form disclosed herein chosen from crystalline forms of Compound (I), pharmaceutically acceptable salts thereof, and deuterated analogs of any of the foregoing and a pharmaceutically acceptable carrier. In some embodiments, the present disclosure provides compositions comprising at least one crystalline form chosen from crystalline forms disclosed herein of Compound (II), pharmaceutically acceptable salts thereof, and deuterated analogs of any of the foregoing and a pharmaceutically acceptable carrier.

In some embodiments, the present disclosure provides methods of treating cystic fibrosis comprising administering to a patient in need thereof at least one crystalline form chosen from crystalline forms disclosed herein of Compound (I), pharmaceutically acceptable salts thereof, and deuterated analogs of any of the foregoing. In some embodiments, the present disclosure provides methods of treating cystic fibrosis comprising administering to a patient in need thereof at least one crystalline form chosen from crystalline forms disclosed herein of Compound (II), pharmaceutically acceptable salts thereof, and deuterated analogs of any of the foregoing.

In some embodiments, disclosed herein is a method of treating, lessening the severity of, or symptomatically treating cystic fibrosis in a patient comprising administering an effective amount of at least one pharmaceutical composition of this disclosure to the patient, such as a human, wherein said patient has cystic fibrosis and is chosen from patients with F508del/minimal function (MF) genotypes, patients with F508del/F508del genotypes, patients with F508del/gating genotypes, and patients with F508del/residual function (RF) genotypes.

In some embodiments, the patient is heterozygous for F508del, and the other CFTR genetic mutation is any CF-causing mutation, and is expected to be and/or is responsive to any combinations of (i) the novel compounds disclosed herein, such as crystalline forms chosen from crystalline forms of Compound (I), pharmaceutically acceptable salts thereof, and deuterated analogs of any of the foregoing, or crystalline forms chosen from crystalline forms of Compound (II), pharmaceutically acceptable salts thereof, and deuterated analogs of any of the foregoing; and (ii) Compound (III), and/or Compound (IV) and/or Compound (V) genotypes based on in vitro and/or clinical data.

Compound (III) has the following structure:

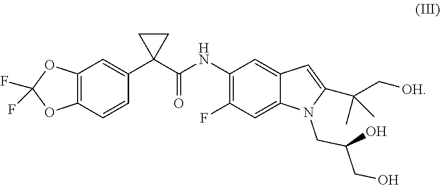 [SMILES: CC(C)(CO)c1cc2cc(NC(=O)C3(c4ccc5c(c4)OC(F)(F)O5)CC3)c(F)cc2n1C[C@@H](O)CO]

A chemical name for Compound (III) is (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide.

Compound (IV) has the following structure:

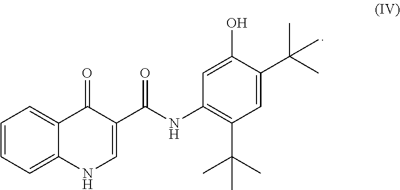 [SMILES: CC(C)(C)c1cc(C(C)(C)C)c(NC(=O)c2c[nH]c3ccccc3c2=O)cc1O]

A chemical name for Compound (IV) is N-(5-hydroxy-2,4-di-tert-butyl-phenyl)-4-oxo-1H-quinoline-3-carboxamide.

Compound (V) is depicted as having the following structure:

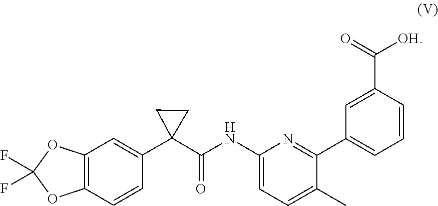 [SMILES: Cc1ccc(NC(=O)C2(c3ccc4c(c3)OC(F)(F)O4)CC2)nc1-c1cccc(C(=O)O)c1]

A chemical name for Compound (V) is 3-(6-(1-(2,2-difluorobenzo[d][1,3]dioxol-5-yl)cyclopropanecarboxamido)-3-methylpyridin-2-yl)benzoic acid.

The compounds, pharmaceutically acceptable salts thereof, and deuterated analogs of any of the foregoing, and the pharmaceutical compositions can be used for treating cystic fibrosis.

A CFTR mutation may affect the CFTR quantity, i.e., the number of CFTR channels at the cell surface, or it may impact CFTR function, i.e., the functional ability of each channel to open and transport ions. Mutations affecting CFTR quantity include mutations that cause defective synthesis (Class I defect), mutations that cause defective processing and trafficking (Class II defect), mutations that cause reduced synthesis of CFTR (Class V defect), and mutations that reduce the surface stability of CFTR (Class VI defect). Mutations that affect CFTR function include mutations that cause defective gating (Class III defect) and mutations that cause defective conductance (Class IV defect).

In some embodiments, disclosed herein methods of treating, lessening the severity of, or symptomatically treating cystic fibrosis in a patient comprising administering an effective amount of a compound, pharmaceutically acceptable salt thereof, or a deuterated analog of any of the foregoing; or a pharmaceutical composition, of this disclosure to a patient, such as a human, wherein said patient has cystic fibrosis. In some embodiments, the patient has F508del/minimal function (MF) genotypes, F508del/F508del genotypes, F508del/gating genotypes, or F508del/residual function (RF) genotypes.

As used herein, “minimal function (MF) mutations” refer to CFTR gene mutations associated with minimal CFTR function (little-to-no functioning CFTR protein) and include, for example, mutations associated with severe defects in ability of the CFTR channel to open and close, known as defective channel gating or “gating mutations”; mutations associated with severe defects in the cellular processing of CFTR and its delivery to the cell surface; mutations associated with no (or minimal) CFTR synthesis; and mutations associated with severe defects in channel conductance. Table C below includes a non-exclusive list of CFTR minimal function mutations, which are detectable by an FDA-cleared genotyping assay. In some embodiments, a mutation is considered a MF mutation if it meets at least 1 of the following 2 criteria:
(1) biological plausibility of no translated protein (genetic sequence predicts the complete absence of CFTR protein), or
(2) in vitro testing that supports lack of responsiveness to Compound (III), Compound (IV) or the combination of Compound (III) and Compound (IV), and evidence of clinical severity on a population basis (as reported in large patient registries).

In some embodiments, the minimal function mutations are those that result in little-to-no functioning CFTR protein and are not responsive in vitro to Compound (III), Compound (IV), or the combination of Compound (III) and Compound (IV).

In some embodiments, the minimal function mutations are those that are not responsive in vitro to Compound (III), Compound (IV) or the combination of Compound (III) and Compound (IV). In some embodiments, the minimal function mutations are mutations based on in vitro testing met the following criteria in in vitro experiments:
baseline chloride transport that was <10% of wildtype CFTR, and
an increase in chloride transport of <10% over baseline following the addition of TEZ, IVA, or TEZ/IVA in the assay.
   In some embodiments, patients with at least one minimal function mutation exhibit evidence of clinical severity as defined as:
average sweat chloride >86 mmol/L, and
prevalence of pancreatic insufficiency (PI) >50%.

Patients with an F508del/minimal function genotype are defined as patients that are heterozygous F508del-CFTR with a second CFTR allele containing a minimal function mutation. In some embodiments, patients with an F508del/minimal function genotype are patients that are heterozygous F508del-CFTR with a second CFTR allele containing a mutation that results in a CFTR protein with minimal CFTR function (little-to-no functioning CFTR protein) and that is not responsive in vitro to Compound (III), Compound (IV) or the combination of Compound (III) and Compound (IV).

In some embodiments, minimal function mutations can be determined using 3 major sources:
biological plausibility for the mutation to respond (i.e., mutation class)
evidence of clinical severity on a population basis (per CFTR2 patient registry; accessed on 15 Feb. 2016)
   average sweat chloride >86 mmol/L, and
   prevalence of pancreatic insufficiency (PI) >50%
in vitro testing
   mutations resulting in baseline chloride transport <10% of wild-type CFTR were considered minimal function
   mutations resulting in chloride transport <10% of wild-type CFTR following the addition of Compound II and/or Compound III were considered nonresponsive.

As used herein, a “residual function mutations” refer to are Class II through V mutations that have some residual chloride transport and result in a less severe clinical phenotype. Residual function mutations are mutation in the CFTR gene that result in reduced protein quantity or function at the cell surface which can produce partial CFTR activity.

Non-limiting examples of CFTR gene mutations known to result in a residual function phenotype include a CFTR residual function mutation selected from 2789+5G→A, 3849+1 OkbC→T, 3272-26A→G, 711+3A→G, E56K, P67L, R74W, D11OE, D11OH, R117C, L206W, R347H, R352Q, A455E, D579G, E831X, S945L, S977F, F1052V, R1070W, F1074L, D1 152H, D1270N, E193K, and K1060T. For example, CFTR mutations that cause defective mRNA splicing, such as 2789+507 A, result in reduced protein synthesis, but deliver some functional CFTR to the surface of the cell to provide residual function. Other CFTR mutations that reduce conductance and/or gating, such as R1 17H, result in a normal quantity of CFTR channels at the surface of the cell, but the functional level is low, resulting in residual function. In some embodiments, the CFTR residual function mutation is selected from R117H, S1235R, I1027T, R668C, G576A, M470V, L997F, R75Q, R1070Q, R31C, D614G, G1069R, R1162L, E56K, A1067T, E193K, and K1060T. In some embodiments, the CFTR residual function mutation is selected from R117H, S1235R, I1027T, R668C, G576A, M470V, L997F, R75Q, R1070Q, R31C, D614G, G1069R, R1162L, E56K, and A1067T.

Residual CFTR function can be characterized at the cellular (in vitro) level using cell based assays, such as an FRT assay (Van Goar, F. et al. (2009) PNAS Vol. 106, No. 44, 18825-18830; and Van Goor, F. et al. (2011) PNAS Vol. 108, No. 46, 18843-18846), to measure the amount of chloride transport through the mutated CFTR channels. Residual function mutations result in a reduction but not complete elimination of CFTR dependent ion transport. In some embodiments, residual function mutations result in at least about 10% reduction of CFTR activity in an FRT assay. In some embodiments, the residual function mutations result in up to about 90% reduction in CFTR activity in an FRT assay.

Patients with an F508del/residual function genotype are defined as patients that are heterozygous F508del-CFTR with a second CFTR allele that contains a mutation that results in reduced protein quantity or function at the cell surface which can produce partial CFTR activity.

Patients with an F508del/gating mutation genotype are defined as patients that are heterozygous F508del-CFTR with a second CFTR allele that contains a mutation associated with a gating defect and clinically demonstrated to be responsive to Compound III. Examples of such mutations include: G178R, S549N, S549R, G551D, G551S, G1244E, S1251N, S1255P, and G1349D.

In some embodiments, the methods of treating, lessening the severity of, or symptomatically treating cystic fibrosis disclosed herein are each independently produces an increase in chloride transport above the baseline chloride transport of the patient.

In some embodiments, in the methods of treating, lessening the severity of, or symptomatically treating cystic fibrosis disclosed herein, the patient is heterozygous for F508del, and the other CFTR genetic mutation is any CF-causing mutation. In some embodiments, the patient is heterozygous for F508del, and the other CFTR genetic mutation is any CF-causing mutation, and is expected to be and/or is responsive to any of the novel compounds disclosed herein, such as Compound (I), Compound (II), Compound (III) and/or Compound (IV) genotypes based on in vitro and/or clinical data. In some embodiments, the patient is heterozygous for F508del, and the other CFTR genetic mutation is any CF-causing mutation, and is expected to be and/or is responsive to any combinations of (i) the novel compounds disclosed herein, such as Compound (I) and Compound (II), and (ii) Compound (III), and/or Compound (IV) and/or Compound (V) genotypes based on in vitro and/or clinical data.

In some embodiments, in the methods of treating, lessening the severity of, or symptomatically treating cystic fibrosis disclosed herein, the patient possesses a CFTR genetic mutation selected from any of the mutations listed in Table A.

TABLE A
CF Mutations

078delT
1078delT
11234V
1154insTC
1161delC
1213delT
1248 + 1G→A
1249 − 1G→A
124del23bp
1259insA
1288insTA
1341 + 1G -> A
1342 − 2A -> C
1461ins4
1471delA
1497delGG
1507del
1525 − 1G→A
1525 − 2A→G
1548delG
1577delTA
1609delCA
1677delTA
1716G/A
1717 − 1G→A
1717 − 8G→A
1782delA
1811 + 1.6kbA -> G
1811 + 1G -> C
1811 + 1.6kbA→G
1811 + 1G→C
1812 − 1G -> A
1898 + 1G -> A
1812-1G→A
1824delA
182delT
1119delA
185 + 1G→T
1898 + 1G -> T
1898 + 1G→A
1898 + 1G→C
1898 + 3A -> G
1898 + 5G -> T
1924del7
1949del84
2043delG
2055del9→A
2105 −
2117del13insAGAAA
2118del14
2143delT
2183AA -> G+
2183AA→G
2183AA→G^{a}
2183delAA -> G#
2183delAA→G
2184delA
2184insA
2307insA
2347delG
2556insAT
2585delT
2594delGT
2622 + 1G -> A
2622 + IG -> A
2659delC
2711delT
271delT
2721del11
2732insA
2789 + 2insA
2789 + 5G→A
2790 − 1G→C
2790 − IG -> C
2869insG
2896insAG
2942insT
2957delT
296 + 1G→A
2991del32
3007delG
3028delA
3040G→C
306insA
306insA 1138insG
3120G→A
3121 − 1G→A
3121 − 2A→G
3121-977_3499 + 248
del2515
3132delTG
3141del9
3171delC
3195del6
3199del6
3272 − 26A -> G
3500 − 2A→G
3600 + 2insT
365 − 366insT
3659delC
3667ins4
3737delA
3791delC
3821delT
3849 + 10kbC→T
3849 + IOkbC -> T
3850 − 1G→A
3850 − 3T -> G
3850 − IG -> A
3876delA
3878delG
3905InsT
3905insT
394delTT
4005 + 1G -> A
4005 + 2T -> C
4005 + 1G→A
4005 + IG -> A
4010del4
4015delA
4016insT
4021dupT
4040delA
405 + 1G→A
405 + 3A→C
405 + IG -> A
406 − 1G→A
406 − IG -> A
4209TGTT -> A
4209TGTT→AA
4279insA
4326delTC
4374 + 1G→T
4374 + IG -> T
4382delA
4428insGA
442delA
457TAT→G
541delC
574delA
5T
621 + 1G→T
621 + 3A -> G
663delT
663delT
1548delG
675del4
711 + 1G -> T
711 + 3A -> G
711 + 1G→T
711 + 3A→G
711 + 5G→A
712 − 1G -> T
7T
852del22
935delA
991del5
A1006E
A120T
A234D
A349V
A455E
A613T
A46D
A46Db
A559T
A559Tb
A561E
C276X
C524R
C524X
CFTRdel2,3
CFTRdele22-23
D110E
D110H
D1152H
D1270N
D192G
D443Y
D513G
D579G
D614G
D836Y
D924N
D979V
E1104X
E116K
E1371X
E193K
E193X
E403D
E474K
E56K
E585X
E588V
E60K
E822K
E822X
E831X
E92K
E92X
F1016S
F1052V
F1074L
F1099L
F191V
F311del
F311L
F508C
F508del
F575Y
G1061R
G1069R
G1244E
G1249R
G126D
G1349D
G149R
G178R
G194R
G194V
G27R
G27X
G314E
G330X
G458V
G463V
G480C
G542X
G550X
G551D
G551S
G576A
G622D
G628R
G628R(G -> A)
G970D
G673X
G85E
G91R
G970R
G970R
H1054D
H1085P
H1085R
H1375P
H139R
H199R
H199Y
H609R
H939R
I1005R
I1027T
I1234V
I1269N
I1366N
I148T
I175V
I3336K
I502T
I506S
I506T
I507del
I507del
I601F
I618T
I807M
I980K
IVS14b + 5G -> A
K710X
K710X
K710X
L102R
L1065P
L1077P
L1077Pb
L1254X
L1324P
L1335P
L138ins
L1480P
L15P
L165S
L206W
L218X
L227R
L320V
L346P
L453S
L467P
L467Pb
L558S
L571S
L732X
L927P
L967S
L997F
M1101K
M1101R
M152V
M1T
M1V
M265R
M470V
M952I
M952T
N1303K
P205S
P574H
P5L
P67L
P750L
P99L
Q1100P
Q1291H
Q1291R
Q1313X
Q1382X
Q1411X
Q1412X
Q220X
Q237E
Q237H
Q452P
Q290X
Q359K/T360K
Q39X
Q414
Q414X
E585X
Q493X
Q525X
Q552X
Q685X
Q890X
Q890X
Q98R
Q98X
R1066C
R1066H
R1066M
R1070Q
R1070W
R1102X
R1158X
R1162L
R1162X
R117C
R117G
R117H
R117L
R117P
R1283M
R1283S
R170H
R258G
R31C
R31L
R334L
R334Q
R334W
R347H
R347L
R347P
R352Q
R352W
R516G
R553Q
R553X
R560K
R560S
R560T
R668C
R709X
R74W
R751L
R75Q
R75X
R764X
R792G
R792X
R851X
R933G
S1118F
S1159F
S1159P
S1196X
S1235R
S1251N
S1255P
S1255X
S13F
S341P
S434X
S466X
S489X
S492F
S4X
S549N
S549R
S549R(A -> C)
S549R(T -> G)
S589N
S737F
S912L
S912X
S945L
S977F
T1036N
T1053I
T1246I
T338I
T604I
V1153E
V1240G
V1293G
V201M
V232D
V456A
V456F
V520F
V562I
V754M
W1089X
W1098C
W1098R
W1098X
W1204X
W1282R
W1282X
W361R
W401X
W496X
W57G
W57R
W57X
W846X
Y1014C
Y1032C
Y1092X
Y109N
Y122X
Y161D
Y161S
Y563D
Y563N
Y569C
Y569D
Y569Db
Y849X
Y913C
Y913X

In some embodiments, in the methods of treating, lessening the severity of, or symptomatically treating cystic fibrosis disclosed herein, the patient possesses a CFTR genetic mutation selected from G178R, G551S, G970R, G1244E, S1255P, G1349D, S549N, S549R, S1251N, E193K, F1052V, G1069R, R117C, D110H, R347H, R352Q, E56K, P67L, L206W, A455E, D579G, S1235R, S945L, R1070W, F1074L, D110E, D1270N, D1152H, 1717-1G->A, 621+1G->T, 3120+1G->A, 1898+1G->A, 711+1G->T, 2622+1G->A, 405+1G->A, 406-1G->A, 4005+1G->A, 1812-1G->A, 1525-1G->A, 712-1G->T, 1248+1G->A, 1341+1G->A, 3121-1G->A, 4374+1G->T, 3850-1G->A, 2789+5G->A, 3849+10kbC->T, 3272-26A->G, 711+5G->A, 3120G->A, 1811+1.6kbA->G, 711+3A->G, 1898+3A->G, 1717-8G->A, 1342-2A->C, 405+3A->C, 1716G/A, 1811+1G->C, 1898+5G->T, 3850-3T->G, IVS14b+5G->A, 1898+1G->T, 4005+2T->C, 621+3A->G, 1949del84, 3141del9, 3195del6, 3199del6, 3905InsT, 4209TGTT->A, A1006E, A120T, A234D, A349V, A613T, C524R, D192G, D443Y, D513G, D836Y, D924N, D979V, E116K, E403D, E474K, E588V, E60K, E822K, F1016S, F1099L, F191V, F311del, F311L, F508C, F575Y, G1061R, G1249R, G126D, G149R, G194R, G194V, G27R, G314E, G458V, G463V, G480C, G622D, G628R, G628R(G->A), G91R, G970D, H1054D, H1085P, H1085R, H1375P, H139R, H199R, H609R, H939R, 11005R, I1234V, I1269N, I1366N, I175V, 1502T, I506S, I506T, I601F, I618T, I807M, 1980K, L102R, L1324P, L1335P, L138ins, L1480P, L15P, L165S, L320V, L346P, L453S, L571S, L967S, M1101R, M152V, M1T, M1V, M265R, M952I, M952T, P574H, PSL, P750L, P99L, Q1100P, Q1291H, Q1291R, Q237E, Q237H, Q452P, Q98R, R1066C, R1066H, R117G, R117L, R117P, R1283M, R1283S, R170H, R258G, R31L, R334L, R334Q, R347L, R352W, R516G, R553Q, R751L, R792G, R933G, S1118F, S1159F, S1159P, S13F, S549R(A->C), S549R(T->G), S589N, S737F, S912L, T1036N, T10531, T12461, T6041, V1153E, V1240G, V1293G, V201M, V232D, V456A, V456F, V562I, W1098C, W1098R, W1282R, W361R, W57G, W57R, Y1014C, Y1032C, Y109N, Y161D, Y161S, Y563D, Y563N, Y569C, and Y913C.

In some embodiments, the patient has at least one combination mutation chosen from: G178R, G551S, G970R, G1244E, S1255P, G1349D, S549N, S549R, S1251N, E193K, F1052V, G1069R, R117C, D110H, R347H, R352Q, E56K, P67L, L206W, A455E, D579G, S1235R, S945L, R1070W, F1074L, D110E, D1270N, D1152H, 1717-1G->A, 621+1G->T, 3120+1G->A, 1898+1G->A, 711+1G->T, 2622+1G->A, 405+1G->A, 406-1G->A, 4005+1G->A, 1812-1G->A, 1525-1G->A, 712-1G->T, 1248+1G->A, 1341+1G->A, 3121-1G->A, 4374+1G->T, 3850-1G->A, 2789+5G->A, 3849+10kbC->T, 3272-26A->G, 711+5G->A, 3120G->A, 1811+1.6kbA->G, 711+3A->G, 1898+3A->G, 1717-8G->A, 1342-2A->C, 405+3A->C, 1716G/A, 1811+1G->C, 1898+5G->T, 3850-3T->G, IVS14b+5G->A, 1898+1G->T, 4005+2T->C, and 621+3A->G.

In some embodiments, the patient has at least one combination mutation chosen from: 1949del84, 3141del9, 3195del6, 3199del6, 3905InsT, 4209TGTT->A, A1006E, A120T, A234D, A349V, A613T, C524R, D192G, D443Y, D513G, D836Y, D924N, D979V, E116K, E403D, E474K, E588V, E60K, E822K, F1016S, F1099L, F191V, F311del, F311L, F508C, F575Y, G1061R, G1249R, G126D, G149R, G194R, G194V, G27R, G314E, G458V, G463V, G480C, G622D, G628R, G628R(G->A), G91R, G970D, H1054D, H1085P, H1085R, H1375P, H139R, H199R, H609R, H939R, 11005R, I1234V, I1269N, I1366N, I175V, 1502T, I506S, I506T, I601F, I618T, I807M, 1980K, L102R, L1324P, L1335P, L138ins, L1480P, L15P, L165S, L320V, L346P, L453S, L571S, L967S, M1101R, M152V, M1T, M1V, M265R, M952I, M952T, P574H, PSL, P750L, P99L, Q1100P, Q1291H, Q1291R, Q237E, Q237H, Q452P, Q98R, R1066C, R1066H, R117G, R117L, R117P, R1283M, R1283S, R170H, R258G, R31L, R334L, R334Q, R347L, R352W, R516G, R553Q, R751L, R792G, R933G, S1118F, S1159F, S1159P, S13F, S549R(A->C), S549R(T->G), S589N, S737F, S912L, T1036N, T10531, T12461, T6041, V1153E, V1240G, V1293G, V201M, V232D, V456A, V456F, V562I, W1098C, W1098R, W1282R, W361R, W57G, W57R, Y1014C, Y1032C, Y109N, Y161D, Y161S, Y563D, Y563N, Y569C, and Y913C.

In some embodiments, in the methods of treating, lessening the severity of, or symptomatically treating cystic fibrosis disclosed herein, the patient possesses a CFTR genetic mutation G551D. In some embodiments, the patient is homozygous for the G551D genetic mutation. In some embodiments, the patient is heterozygous for the G551D genetic mutation. In some embodiments, the patient is heterozygous for the G551D genetic mutation, having the G551D mutation on one allele and any other CF-causing mutation on the other allele. In some embodiments, the patient is heterozygous for the G551D genetic mutation on one allele and the other CF-causing genetic mutation on the other allele is any one of F508del, G542X, N1303K, W1282X, R117H, R553X, 1717-1G->A, 621+1G->T, 2789+5G->A, 3849+10kbC->T, R1162X, G85E, 3120+1G->A, 41507, 1898+1G->A, 3659delC, R347P, R560T, R334W, A455E, 2184delA, or 711+1G->T. In some embodiments, the patient is heterozygous for the G551D genetic mutation, and the other CFTR genetic mutation is F508del. In some embodiments, the patient is heterozygous for the G551D genetic mutation, and the other CFTR genetic mutation is R117H.

In some embodiments, in the methods of treating, lessening the severity of, or symptomatically treating cystic fibrosis disclosed herein, the patient possesses a CFTR genetic mutation F508del. In some embodiments, the patient is homozygous for the F508del genetic mutation. In some embodiments, the patient is heterozygous for the F508del genetic mutation wherein the patient has the F508del genetic mutation on one allele and any CF-causing genetic mutation on the other allele. In some embodiments, the patient is heterozygous for F508del, and the other CFTR genetic mutation is any CF-causing mutation, including, but not limited to G551D, G542X, N1303K, W1282X, R117H, R553X, 1717-1G->A, 621+1G->T, 2789+5G->A, 3849+10kbC->T, R1162X, G85E, 3120+1G->A, 41507, 1898+1G->A, 3659delC, R347P, R560T, R334W, A455E, 2184delA, or 711+1G->T. In some embodiments, the patient is heterozygous for F508del, and the other CFTR genetic mutation is G551D. In some embodiments, the patient is heterozygous for F508del, and the other CFTR genetic mutation is R117H.

In some embodiments, the patient has at least one combination mutation chosen from:

### D443Y; G576A; R668C,

### F508C; S1251N,

### G576A; R668C,

### G970R; M470V,

### R74W; D1270N,

### R74W; V201M, and

### R74W; V201M; D1270N.

In some embodiments, in the methods of treating, lessening the severity of, or symptomatically treating cystic fibrosis disclosed herein, the patient possesses a CFTR genetic mutation selected from G178R, G551S, G970R, G1244E, S1255P, G1349D, S549N, S549R, S1251N, E193K, F1052V and G1069R. In some embodiments, the patient possesses a CFTR genetic mutation selected from G178R, G551S, G970R, G1244E, S1255P, G1349D, S549N, S549R and S1251N. In some embodiments, the patient possesses a CFTR genetic mutation selected from E193K, F1052V and G1069R. In some embodiments, the method produces an increase in chloride transport relative to baseline chloride transport of the patient of the patient.

In some embodiments, in the methods of treating, lessening the severity of, or symptomatically treating cystic fibrosis disclosed herein, the patient possesses a CFTR genetic mutation selected from R117C, D110H, R347H, R352Q, E56K, P67L, L206W, A455E, D579G, S1235R, S945L, R1070W, F1074L, D110E, D1270N and D1152H.

In some embodiments, the patient possesses a CFTR genetic mutation selected from 1717-1G->A, 621+1G->T, 3120+1G->A, 1898+1G->A, 711+1G->T, 2622+1G->A, 405+1G->A, 406-1G->A, 4005+1G->A, 1812-1G->A, 1525-1G->A, 712-1G->T, 1248+1G->A, 1341+1G->A, 3121-1G->A, 4374+1G->T, 3850-1G->A, 2789+5G->A, 3849+10kbC->T, 3272-26A->G, 711+5G->A, 3120G->A, 1811+1.6kbA->G, 711+3A->G, 1898+3A->G, 1717-8G->A, 1342-2A->C, 405+3A->C, 1716G/A, 1811+1G->C, 1898+5G->T, 3850-3T->G, IVS14b+5G->A, 1898+1G->T, 4005+2T->C and 621+3A->G. In some embodiments, the patient possesses a CFTR genetic mutation selected from 1717-1G->A, 1811+1.6kbA->G, 2789+5G->A, 3272-26A->G and 3849+10kbC->T. In some embodiments, the patient possesses a CFTR genetic mutation selected from 2789+5G->A and 3272-26A->G.

In some embodiments, in the methods of treating, lessening the severity of, or symptomatically treating cystic fibrosis disclosed herein, the patient possesses a CFTR genetic mutation selected from G178R, G551S, G970R, G1244E, S1255P, G1349D, S549N, S549R, S1251N, E193K, F1052V, G1069R, R117C, D110H, R347H, R352Q, E56K, P67L, L206W, A455E, D579G, S1235R, S945L, R1070W, F1074L, D110E, D1270N, D1152H, 1717-1G->A, 621+1G->T, 3120+1G->A, 1898+1G->A, 711+1G->T, 2622+1G->A, 405+1G->A, 406-1G->A, 4005+1G->A, 1812-1G->A, 1525-1G->A, 712-1G->T, 1248+1G->A, 1341+1G->A, 3121-1G->A, 4374+1G->T, 3850-1G->A, 2789+5G->A, 3849+10kbC->T, 3272-26A->G, 711+5G->A, 3120G->A, 1811+1.6kbA->G, 711+3A->G, 1898+3A->G, 1717-8G->A, 1342-2A->C, 405+3A->C, 1716G/A, 1811+1G->C, 1898+5G->T, 3850-3T->G, IVS14b+5G->A, 1898+1G->T, 4005+2T->C and 621+3A->G, and human CFTR mutations selected from F508del, R117H, and G551D.

In some embodiments, in the methods of treating, lessening the severity of, or symptomatically treating cystic fibrosis disclosed herein, the patient possesses a CFTR genetic mutation selected from G178R, G551S, G970R, G1244E, S1255P, G1349D, S549N, S549R, S1251N, E193K, F1052V, G1069R, R117C, D110H, R347H, R352Q, E56K, P67L, L206W, A455E, D579G, S1235R, S945L, R1070W, F1074L, D110E, D1270N, D1152H, 1717-1G->A, 621+1G->T, 3120+1G->A, 1898+1G->A, 711+1G->T, 2622+1G->A, 405+1G->A, 406-1G->A, 4005+1G->A, 1812-1G->A, 1525-1G->A, 712-1G->T, 1248+1G->A, 1341+1G->A, 3121-1G->A, 4374+1G->T, 3850-1G->A, 2789+5G->A, 3849+10kbC->T, 3272-26A->G, 711+5G->A, 3120G->A, 1811+1.6kbA->G, 711+3A->G, 1898+3A->G, 1717-8G->A, 1342-2A->C, 405+3A->C, 1716G/A, 1811+1G->C, 1898+5G->T, 3850-3T->G, IVS14b+5G->A, 1898+1G->T, 4005+2T->C, 621+3A->G, and a CFTR mutation selected from F508del, R117H, and G551D; and a CFTR mutations selected from F508del, R117H, and G551D.

In some embodiments, the patient possesses a CFTR genetic mutation selected from G178R, G551S, G970R, G1244E, S1255P, G1349D, S549N, S549R, S1251N, E193K, F1052V and G1069R, and a human CFTR mutation selected from F508del, R117H, and G551D. In some embodiments, the patient possesses a CFTR genetic mutation selected from G178R, G551S, G970R, G1244E, S1255P, G1349D, S549N, S549R and S1251N, and a human CFTR mutation selected from F508del, R117H, and G551D. In some embodiments, the patient possesses a CFTR genetic mutation selected from E193K, F1052V and G1069R, and a human CFTR mutation selected from F508del, R117H, and G551D.

In some embodiments, the patient possesses a CFTR genetic mutation selected from R117C, D110H, R347H, R352Q, E56K, P67L, L206W, A455E, D579G, S1235R, S945L, R1070W, F1074L, D110E, D1270N and D1152H, and a human CFTR mutation selected from F508del, R117H, and G551D.

In some embodiments, the patient possesses a CFTR genetic mutation selected from 1717-1G->A, 621+1G->T, 3120+1G->A, 1898+1G->A, 711+1G->T, 2622+1G->A, 405+1G->A, 406-1G->A, 4005+1G->A, 1812-1G->A, 1525-1G->A, 712-1G->T, 1248+1G->A, 1341+1G->A, 3121-1G->A, 4374+1G->T, 3850-1G->A, 2789+5G->A, 3849+10kbC->T, 3272-26A->G, 711+5G->A, 3120G->A, 1811+1.6kbA->G, 711+3A->G, 1898+3A->G, 1717-8G->A, 1342-2A->C, 405+3A->C, 1716G/A, 1811+1G->C, 1898+5G->T, 3850-3T->G, IVS14b+5G->A, 1898+1G->T, 4005+2T->C and 621+3A->G, and a human CFTR mutation selected from F508del, R117H, and G551D. In some embodiments, the patient possesses a CFTR genetic mutation selected from 1717-1G->A, 1811+1.6kbA->G, 2789+5G->A, 3272-26A->G and 3849+10kbC->T, and a human CFTR mutation selected from F508del, R117H, and G551D. In some embodiments, the patient possesses a CFTR genetic mutation selected from 2789+5G->A and 3272-26A->G, and a human CFTR mutation selected from F508del, R117H.

In some embodiments, the patient is heterozygous having a CF-causing mutation on one allele and a CF-causing mutation on the other allele. In some embodiments, the patient is heterozygous for F508del, and the other CFTR genetic mutation is any CF-causing mutation, including, but not limited to F508del on one CFTR allele and a CFTR mutation on the second CFTR allele that is associated with minimal CFTR function, residual CFTR function, or a defect in CFTR channel gating activity.

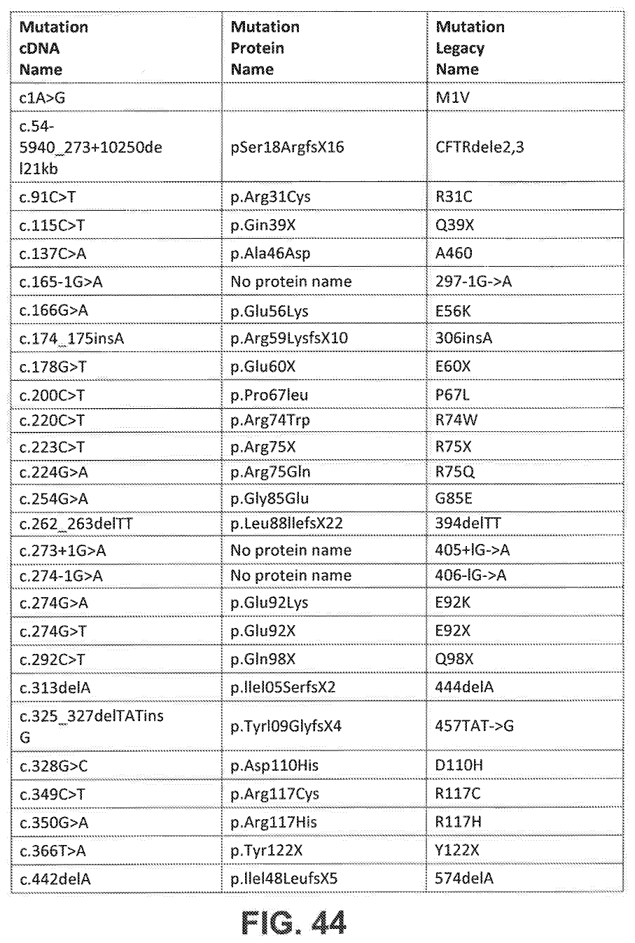

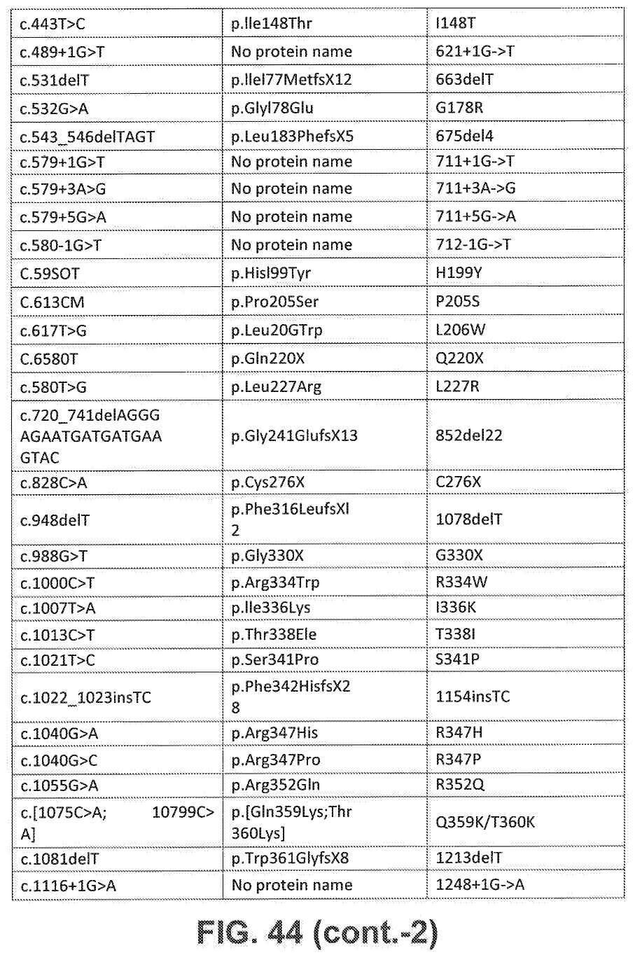

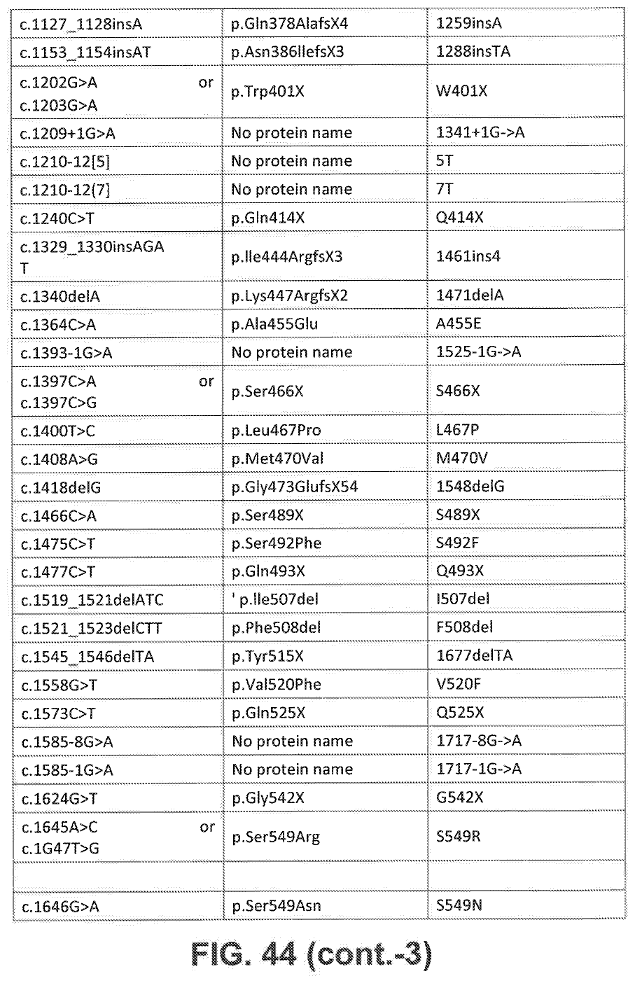

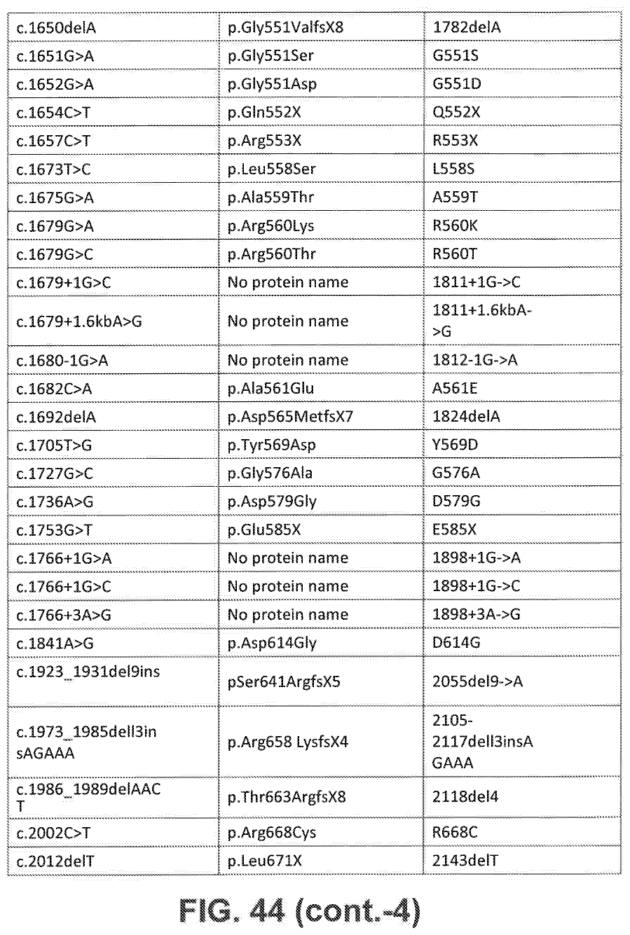

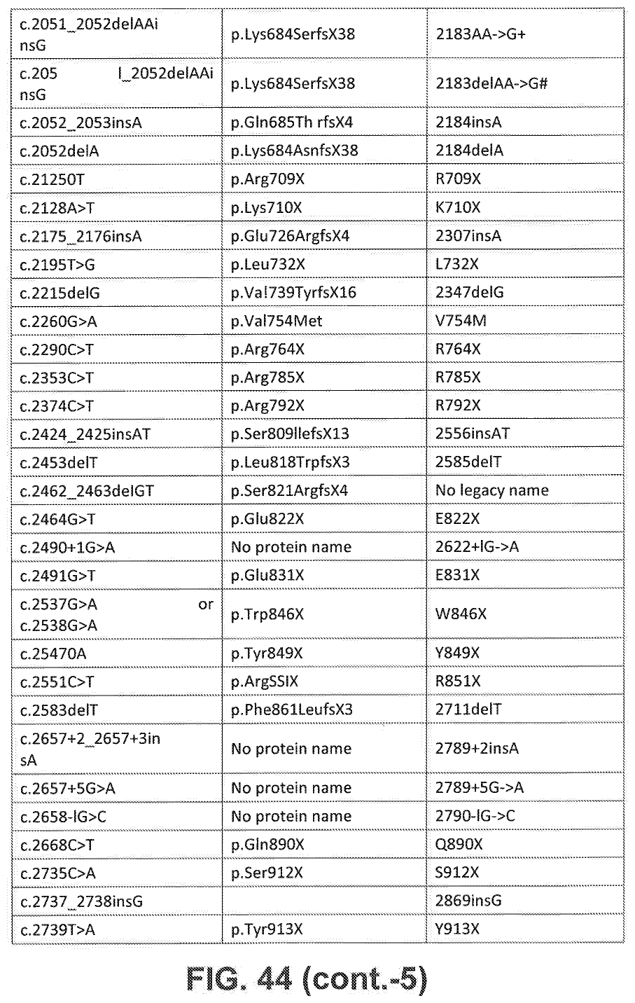

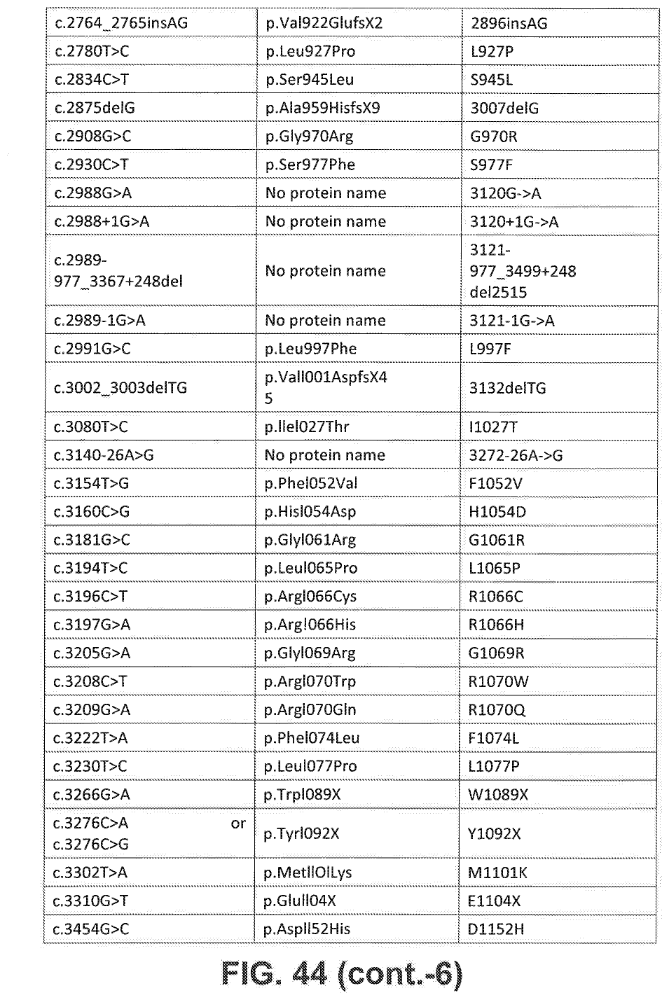

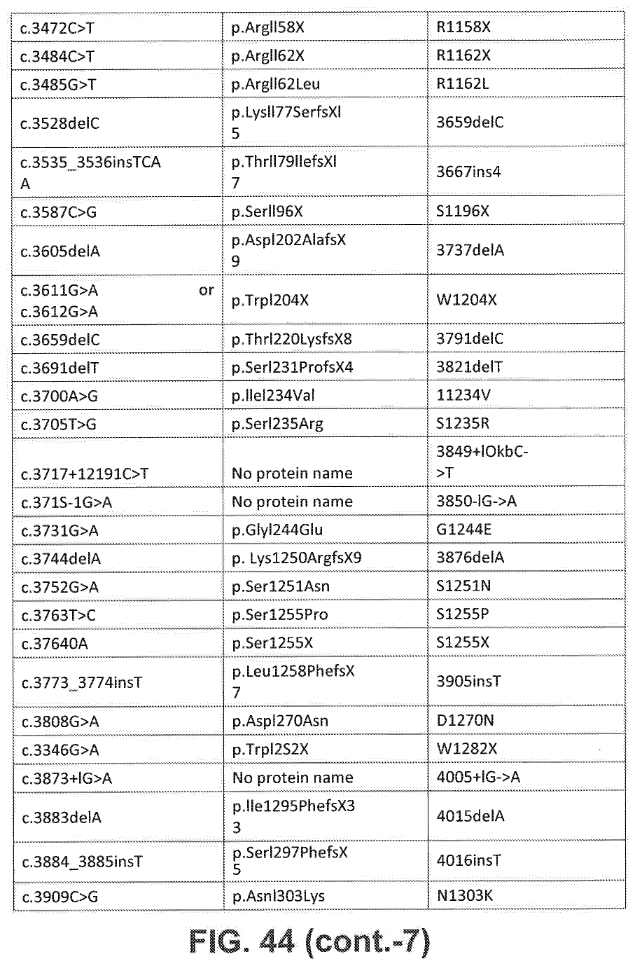

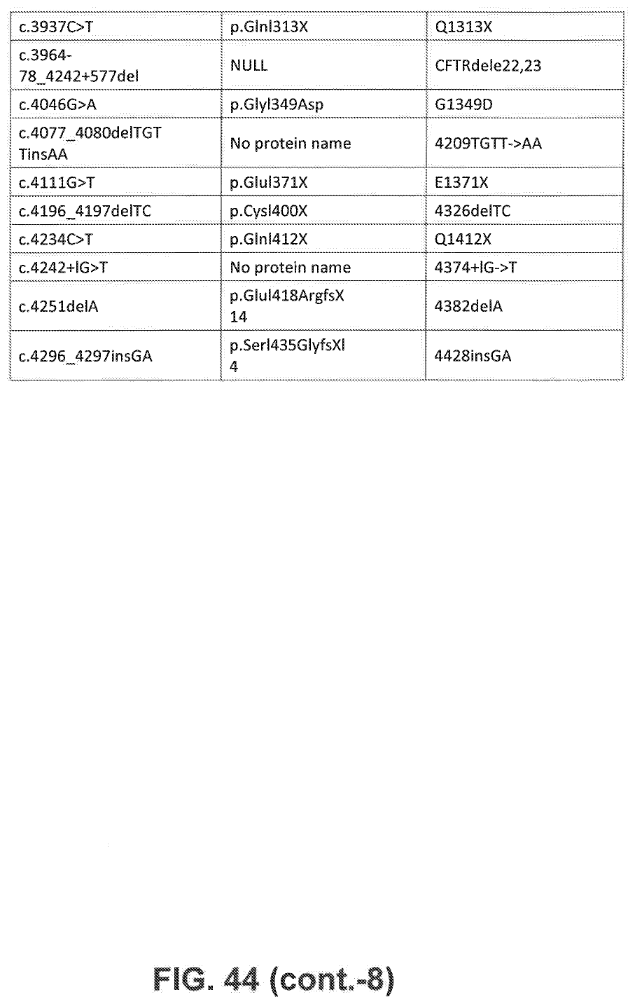

In some embodiments, the CF-causing mutation is chosen from Table A. In some embodiments, the CF-causing mutation is selected from Table B. In some embodiments, the CF-causing mutation is chosen from Table C. In some embodiments, the CF-causing mutation is chosen from FIG. 44. In some embodiments, the patient is heterozygous having a CF-causing mutation on one CFTR allele chosen from the mutations listed in the table from FIG. 44 and a CF-causing mutation on the other CFTR allele is chosen from the CFTR mutations listed in Table B:

TABLE B
CFTR Mutations

| | | |
|---|---|---|
| Q39X | 621 + 1G→T | A559T |
| W57X | 1248 + 1G→A | R560T |
| E60X | 1341 + 1G→A | R560S |
| R75X | 1717 − 1G→A | A561E |
| E92X | 1811 + 1.6kbA→G | Y569D |
| Q98X | 1811 + 1G→C | L1065P |
| Y122X | 1812 − 1G→A | R1066C |
| L218X | 1898 + 1G→A | R1066M |
| Q220X | 2622 + 1G→A | L1077P |
| C276X | 3120 + 1G→A | H1085R |
| Q290X | 3120G→A | M1101K |
| G330X | 3850 − 1G→A | N1303K |
| W401X | 4005 + 1G→A | 3849 + 10kbC→T |
| Q414X | 4374 + 1G→T | 3272 − 26A→G |
| S434X | 663delT | 711 + 3A→G |
| S466X | 2183AA→G | E56K |
| S489X | CFTRdel2,3 | P67L |
| Q493X | 3659delC | R74W |
| W496X | 394delTT | D110E |
| Q525X | 2184insA | D110H |
| G542X | 3905insT | R117C |
| Q552X | 2184delA | L206W |
| R553X | 1078delT | R347H |
| E585X | 1154insTC | R352Q |
| G673X | 2183delAA→G | A455E |
| R709X | 2143delT | D579G |
| K710X | 1677delTA | E831X |
| L732X | 3876delA | S945L |
| R764X | 2307insA | S977F |
| R785X | 4382delA | F1052V |
| R792X | 4016insT | R1070W |
| E822X | 2347delG | F1074L |
| W846X | 3007delG | D1152H |
| R851X | 574delA | D1270N |
| Q890X | 2711delT | G178R |
| S912X | 3791delC | S549N |
| W1089X | CFTRdele22-23 | S549R |
| Y1092X | 457TAT→G | G551D |
| E1104X | 2043delG | G551S |
| R1158X | 2869insG | G1244E |
| R1162X | 3600 + 2insT | S1251N |
| S1196X | 3737delA | S1255P |
| W1204X | 4040delA | G1349D |
| S1255X | 541delC | |
| W1282X | A46D | |
| Q1313X | T338I | |
| 621 + 1G→T | R347P | |
| 711 + 1G→T | L927P | |
| 711 + 5G→A | G85E | |
| 712 − 1G→T | S341P | |
| 405 + 1G→A | L467P | |
| 405 + 3A→C | I507del | |
| 406 − 1G→A | V520F | |

### Table C: CFTR Mutations

| Criteria | Mutation | | | | |
|---|---|---|---|---|---|
| Truncation mutations | Q2X | L218X | Q525X | R792X | E1104X |
| % PI >50% and/or | S4X | Q220X | G542X | E822X | W1145X |
| SwCl⁻ >86 mmol/L | W19X | Y275X | G550X | W882X | R1158X |
| No full-length | G27X | C276X | Q552X | W846X | R1162X |
| protein | Q39X | Q290X | R553X | Y849X | S1196X |
| | W57X | G330X | E585X | R851X | W1204X |
| | E60X | W401X | G673X | Q890X | L1254X |
| | R75X | Q414X | Q685X | S912X | S1255X |
| | L88X | S434X | R709X | Y913X | W1282X |
| | E92X | S466X | K710X | Q1042X | Q1313X |
| | Q98X | S489X | Q715X | W1089X | Q1330X |
| | Y122X | Q493X | L732X | Y1092X | E1371X |
| | E193X | W496X | R764X | W1098X | Q1382X |
| | W216X | C524X | R785X | R1102X | Q1411X |
| Splice mutations | 185 + 1G→T | 711 + 5G→A | 1717 − 8G→A | 2622 + 1G→A | 3121 − 1G→A |
| %PI >50% and/or | 296 + 1G→A | 712 − 1G→T | 1717 − 1G→A | 2790 − 1G→C | 3500 − 2A→G |
| SwCl⁻ >86 mmol/L | 296 + 1G→T | 1248 + 1G→A | 1811 + 1G→C | 3040G→C | 3600 + 2insT |
| No or little mature | 405 + 1G→A | 1249 − 1G→A | 1811 + 1.6kbA→G | (G970R) | 3850 − 1G→A |
| mRNA | 405 + 3A→C | 1341 + 1G→A | 1811 + 1643G→T | 3120G→A | 4005 + 1G→A |
| | 406 − 1G→A | 1525 − 2A→G | 1812 − 1G→A | 3120 + 1G→A | 4374 + 1G→T |
| | 621 + 1G→T | 1525 − 1G→A | 1898 + 1G→A | 3121 − 2A→G | |
| | 711 + 1G→T | | 1898 + 1G→C | | |
| Small (≤3 nucleotide) | 182delT | 1078delT | 1677delTA | 2711delT | 3737delA |
| insertion/deletion | 306insA | 1119delA | 1782delA | 2732insA | 3791delC |
| (ins/del) frameshift | 306delTAGA | 1138insG | 1824delA | 2869insG | 3821delT |
| mutations | 365-366insT | 1154insTC | 1833delT | 2896insAG | 3876delA |
| % PI >50% and/or | 394delTT | 1161delC | 2043delG | 2942insT | 3878delG |
| SwCl⁻ >86 mmol/L | 442delA | 1213delT | 2143delT | 2957delT | 3905insT |
| Garbled and/or | 444delA | 1259insA | 2183AA→G ^{a} | 3007delG | 4016insT |
| truncated protein | 457TAT→G | 1288insTA | 2184delA | 3028delA | 4021dupT |
| | 541delC | 1343delG | 2184insA | 3171delC | 4022insT |
| | 574delA | 1471delA | 2307insA | 3171insC | 4040delA |
| | 663delT | 1497delGG | 2347delG | 3271delGG | 4279insA |
| | 849delG | 1548delG | 2585delT | 3349insT | 4326delTC |
| | 935delA | 1609delCA | 2594delGT | 3659delC | |
| Non-small (>3 | CFTRdele1 | CFTRdele16-17b | 1461ins4 | | |
| nucleotide) | CFTRdele2 | CFTRdele17a,17b | 1924del7 | | |
| insertion/deletion | CFTRdele2,3 | CFTRdele17a-18 | 2055del9→A | | |
| (ins/del) frameshift | CFTRdele2-4 | CFTRdele19 | 2105-2117del13insAGAAA | | |
| mutations | CFTRdele3-10,14b-16 | CFTRdele19-21 | 2372del8 | | |
| % PI >50% and/or | CFTRdele4-7 | CFTRdele21 | 2721del11 | | |
| SwCl⁻ >86 mmol/L | CFTRdele4-11 | CFTRdele22-24 | 2991del32 | | |
| Garbled and/or | CFTR50kbdel | CFTRdele22,23 | 3121-977_3499 + 248del2515 | | |
| truncated protein | CFTRdup6b-10 | 124del23bp | 3667ins4 | | |
| | CFTRdele11 | 602del14 | 4010del4 | | |
| | CFTRdele13,14a | 852del22 | 4209TGTT→AA | | |
| | CFTRdelel4b-17b | 991del5 | | | |
| Class II, III, IV | A46D^{b} | V520F | Y569D^{b} | N1303K | |
| mutations not | G85E | A559T^{b} | L1065P | | |
| responsive to | R347P | R560T | R1066C | | |
| Compound III, | L467P^{b} | R560S | L1077P^{b} | | |
| Compound IV, or | I507del | A561E | M1101K | | |
| Compound III/ | | | | | |
| Compound IV | | | | | |
| % PI >50% and/or | | | | | |
| SwCl⁻ >86 mmol/L | | | | | |
| AND | | | | | |
| Not responsive in | | | | | |
| vitro to Compound | | | | | |
| III, Compound IV, | | | | | |
| or Compound | | | | | |
| III/Compound IV | | | | | |

CFTR: cystic fibrosis transmembrane conductance regulator; SwCl: sweat chloride
Source: CFTR2.org [Internet]. Baltimore (MD): Clinical and functional translation of CFTR. The Clinical and Functional Translation of CFTR (CFTR2), US Cystic Fibrosis Foundation, Johns Hopkins University, the Hospital for Sick Children. Available at: http://www.cftr2.org/. Accessed 15 Feb. 2016.
Notes:
% PI: percentage of F508del-CFTR heterozygous patients in the CFTR2 patient registry who are pancreatic insufficient; SwCl: mean sweat chloride of F508del-CFTR heterozygous patients in the CFTR2 patient registry.
^{a} Also known as 2183delAA→G.
^{b}Unpublished data.

In some embodiments, the patient is: with F508del/MF (F/MF) genotypes (heterozygous for F508del and an MF mutation not expected to respond to CFTR modulators, such as Compound IV); with F508del/F508del (F/F) genotype (homozygous for F508del); and/or with F508del/gating (F/G) genotypes (heterozygous for F508del and a gating mutation known to be CFTR modulator-responsive (e.g., Compound IV-responsive). In some embodiments, the patient with F508del/MF (F/MF) genotypes has a MF mutation that is not expected to respond to Compound III, Compound IV, and both of Compound III and Compound IV. In some embodiments, the patient with F508del/MF (F/MF) genotypes has any one of the MF mutations in Table C.

In some embodiments, the patient is heterozygous for F508del, and the other CFTR genetic mutation is any CF-causing mutation, including truncation mutations, splice mutations, small (<3 nucleotide) insertion or deletion (ins/del) frameshift mutations; non-small (>3 nucleotide) insertion or deletion (ins/del) frameshift mutations; and Class II, III, IV mutations not responsive to Compound IV alone or in combination with Compound III or Compound V.

In some embodiments, the patient is heterozygous for F508del, and the other CFTR genetic mutation is a truncation mutation. In some specific embodiments, the truncation mutation is a truncation mutation listed in Table C.

In some embodiments, the patient is heterozygous for F508del, and the other CFTR genetic mutation is a splice mutation. In some specific embodiments, the splice mutation is a splice mutation listed in Table C.

In some embodiments, the patient is heterozygous for F508del, and the other CFTR genetic mutation is a small (<3 nucleotide) insertion or deletion (ins/del) frameshift mutation. In some specific embodiments, the small (<3 nucleotide) insertion or deletion (ins/del) frameshift mutation is a small (<3 nucleotide) insertion or deletion (ins/del) frameshift mutation listed in Table C.

In some embodiments, the patient is heterozygous for F508del, and the other CFTR genetic mutation is any CF-causing mutation expected to be and/or is responsive to, based on in vitro and/or clinical data, any combination of (i) a novel compound chosen from those disclosed herein (e.g., Compound (I), (II), and pharmaceutically acceptable salts thereof, and their deuterated derivatives), and (ii) Compound III, and/or Compound IV, and/or Compound V.

In some embodiments, the patient is heterozygous for F508del, and the other CFTR genetic mutation is any CF-causing mutation expected to be and/or is responsive, based on in vitro and/or clinical data, to the triple combination of a novel compound chosen from those disclosed herein (e.g., compounds of Formula (I), (II), (III), (IV), or (V), and pharmaceutically acceptable salts thereof, and their deuterated derivatives), and Compound III, and Compound IV.

In some embodiments, the patient is heterozygous for F508del, and the other CFTR genetic mutation is a non-small (>3 nucleotide) insertion or deletion (ins/del) frameshift mutation. In some specific embodiments, the non-small (>3 nucleotide) insertion or deletion (ins/del) frameshift mutation is a non-small (>3 nucleotide) insertion or deletion (ins/del) frameshift mutation listed in Table 5B.

In some embodiments, the patient is heterozygous for F508del, and the other CFTR genetic mutation is a Class II, III, IV mutations not responsive to Compound IV alone or in combination with Compound III or Compound V. In some specific embodiments, the Class II, III, IV mutations not responsive to Compound IV alone or in combination with Compound III or Compound V is a Class II, III, IV mutations not responsive to Compound IV alone or in combination with Compound III or Compound V listed in Table C.

In some embodiments, the patient is heterozygous for F508del, and the other CFTR genetic mutation is any mutation listed in Table C.

In some embodiments, the patient is heterozygous for F508del, and the other CFTR genetic mutation is any mutation, but other than F508del, listed in Table A, B, C, and FIG. 44.

In some embodiments, the patient is heterozygous for F508del, and the other CFTR genetic mutation is any mutation listed in Table A. In some embodiments, the patient is heterozygous for F508del, and the other CFTR genetic mutation is any mutation listed in Table B. In some embodiments, the patient is heterozygous for F508del, and the other CFTR genetic mutation is any mutation listed in Table C. In some embodiments, the patient is heterozygous for F508del, and the other CFTR genetic mutation is any mutation listed in FIG. 44.

In some embodiments, the patient is homozygous for F508del.

In some embodiments, the patient is heterozygous having one CF-causing mutation on one CFTR allele selected from the mutations listed in the table from FIG. 44 and another CF-causing mutation on the other CFTR allele is selected from the CFTR mutations listed in Table C.

In some embodiments, the composition disclosed herein is useful for treating, lessening the severity of, or symptomatically treating cystic fibrosis in patients who exhibit residual CFTR activity in the apical membrane of respiratory and non-respiratory epithelia. The presence of residual CFTR activity at the epithelial surface can be readily detected using methods known in the art, e.g., standard electrophysiological, biochemical, or histochemical techniques. Such methods identify CFTR activity using in vivo or ex vivo electrophysiological techniques, measurement of sweat or salivary Cl⁻ concentrations, or ex vivo biochemical or histochemical techniques to monitor cell surface density. Using such methods, residual CFTR activity can be readily detected for patients that are heterozygous or homozygous for a variety of different mutations, including patients heterozygous for the most common mutation, F508del, as well as other mutations such as the G551D mutation, or the R117H mutation. In some embodiments, compositions disclosed herein are useful for treating, lessening the severity of, or symptomatically treating cystic fibrosis in patients who exhibit little to no residual CFTR activity. In some embodiments, compositions disclosed herein are useful for treating, lessening the severity of, or symptomatically treating cystic fibrosis in patients who exhibit little to no residual CFTR activity in the apical membrane of respiratory epithelia.

In some embodiments, the compositions disclosed herein are useful for treating or lessening the severity of cystic fibrosis in patients who exhibit residual CFTR activity using pharmacological methods. Such methods increase the amount of CFTR present at the cell surface, thereby inducing a hitherto absent CFTR activity in a patient or augmenting the existing level of residual CFTR activity in a patient.

In some embodiments, the compositions disclosed herein are useful for treating or lessening the severity of cystic fibrosis in patients with certain genotypes exhibiting residual CFTR activity.

In some embodiments, compositions disclosed herein are useful for treating, lessening the severity of, or symptomatically treating cystic fibrosis in patients within certain clinical phenotypes, e.g., a mild to moderate clinical phenotype that typically correlates with the amount of residual CFTR activity in the apical membrane of epithelia. Such phenotypes include patients exhibiting pancreatic sufficiency.

In some embodiments, the compositions disclosed herein are useful for treating, lessening the severity of, or symptomatically treating patients diagnosed with pancreatic sufficiency, idiopathic pancreatitis and congenital bilateral absence of the vas deferens, or mild lung disease wherein the patient exhibits residual CFTR activity.

In some embodiments, this disclosure relates to a method of augmenting or inducing anion channel activity in vitro or in vivo, comprising contacting the channel with a composition disclosed herein. In some embodiments, the anion channel is a chloride channel or a bicarbonate channel. In some embodiments, the anion channel is a chloride channel.

The exact amount of a pharmaceutical composition required will vary from subject to subject, depending on the species, age, and general condition of the subject, the severity of the disease, the particular agent, its mode of administration, and the like. The compounds of this disclosure may be formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of this disclosure will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient”, as used herein, means an animal, such as a mammal, and even further such as a human.

In some embodiments, the disclosure also is directed to methods of treatment using isotope-labelled compounds of the afore-mentioned compounds, which, in some embodiments, are referred to as Compound I′, Compound II′, Compound III′, Compound IV, Compound IV-d or Compound V′. In some embodiments, Compound I′, Compound II′, Compound III′, Compound IV, Compound IV-d, Compound V′, or pharmaceutically acceptable salts thereof, wherein the formula and variables of such compounds and salts are each and independently as described above or any other embodiments described above, provided that one or more atoms therein have been replaced by an atom or atoms having an atomic mass or mass number which differs from the atomic mass or mass number of the atom which usually occurs naturally (isotope labelled). Examples of isotopes which are commercially available and suitable for the disclosure include isotopes of hydrogen, carbon, nitrogen, oxygen, phosphorus, fluorine and chlorine, for example ²H, ³H, ¹³C, ¹⁴C, ¹⁵N, ¹⁸O, ¹⁷O, ³¹P, ³²P, ³⁵S, ¹⁸F and ³⁶Cl, respectively.

The isotope-labelled compounds and salts can be used in a number of beneficial ways. They can be suitable for medicaments and/or various types of assays, such as substrate tissue distribution assays. For example, tritium (³H)- and/or carbon-14 (¹⁴C)-labelled compounds are particularly useful for various types of assays, such as substrate tissue distribution assays, due to relatively simple preparation and excellent detectability. For example, deuterium (²H)-labelled ones are therapeutically useful with potential therapeutic advantages over the non-²H-labelled compounds. In general, deuterium (²H)-labelled compounds and salts can have higher metabolic stability as compared to those that are not isotope-labelled owing to the kinetic isotope effect described below. Higher metabolic stability translates directly into an increased in vivo half-life or lower dosages, which could be desired. The isotope-labelled compounds and salts can usually be prepared by carrying out the procedures disclosed in the synthesis schemes and the related description, in the example part and in the preparation part in the present text, replacing a non-isotope-labelled reactant by a readily available isotope-labelled reactant.

In some embodiments, the isotope-labelled compounds and salts are deuterium (²H)-labelled ones. In some specific embodiments, the isotope-labelled compounds and salts are deuterium (²H)-labelled, wherein one or more hydrogen atoms therein have been replaced by deuterium. In chemical structures, deuterium is represented as “D.”

The deuterium (²H)-labelled compounds and salts can manipulate the oxidative metabolism of the compound by way of the primary kinetic isotope effect. The primary kinetic isotope effect is a change of the rate for a chemical reaction that results from exchange of isotopic nuclei, which in turn is caused by the change in ground state energies necessary for covalent bond formation after this isotopic exchange. Exchange of a heavier isotope usually results in a lowering of the ground state energy for a chemical bond and thus causes a reduction in the rate-limiting bond breakage. If the bond breakage occurs in or in the vicinity of a saddle-point region along the coordinate of a multi-product reaction, the product distribution ratios can be altered substantially. For explanation: if deuterium is bonded to a carbon atom at a non-exchangeable position, rate differences of k_{M}/k_{D}=2-7 are typical. For a further discussion, see S. L. Harbeson and R. D. Tung, Deuterium In Drug Discovery and Development, Ann. Rep. Med. Chem. 2011, 46, 403-417, incorporated in its entirety herein by reference.

The concentration of the isotope(s) (e.g., deuterium) incorporated into the isotope-labelled compounds and salt of the disclosure may be defined by the isotopic enrichment factor. The term “isotopic enrichment factor” as used herein means the ratio between the isotopic abundance and the natural abundance of a specified isotope. In some embodiments, if a substituent in a compound of the disclosure is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

When discovering and developing therapeutic agents, the person skilled in the art attempts to optimize pharmacokinetic parameters while retaining desirable in vitro properties. It may be reasonable to assume that many compounds with poor pharmacokinetic profiles are susceptible to oxidative metabolism.

In some embodiments, Compound IV′ as used herein includes the deuterated compound disclosed in U.S. Pat. No. 8,865,902 (which is incorporated herein by reference), and CTP-656.

In some embodiments, Compound IV′ is:

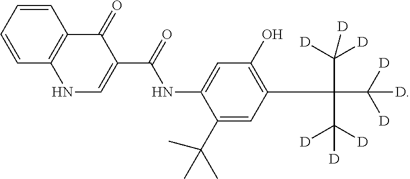

Additional embodiments include:

### 1. Crystalline Form A of Compound (I):

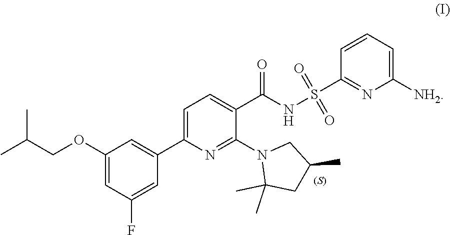

2. Crystalline Form A according to embodiment 1 in substantially pure form.
3. Crystalline Form A according to embodiment 1, characterized by an X-ray powder diffractogram having a signal at at least three two-theta values chosen from 4.3±0.2, 12.8±0.2, 17.1±0.2, 20.5±0.2, 24.2±0.2, and 28.1±0.2.
4. Crystalline Form A according to embodiment 1, characterized by an X-ray powder diffractogram having a signal at at least three two-theta values chosen from 4.3±0.2, 17.1±0.2, 20.5±0.2, 24.2±0.2, and 28.1±0.2.
5. Crystalline Form A according to embodiment 1, characterized by an X-ray powder diffractogram having a signal at three two-theta values of 4.3±0.2, 17.1±0.2, and 24.2±0.2.
6. Crystalline Form A according to embodiment 1, characterized by an X-ray powder diffractogram having a signal at six two-theta values of 4.3±0.2, 12.8±0.2, 17.1±0.2, 20.5±0.2, 24.2±0.2, and 28.1±0.2.
7. Crystalline Form A of embodiment 1, characterized by an X-ray powder diffractogram substantially similar to that in FIG. 1.
8. Crystalline Form A of Compound (I) prepared by a process comprising crystallizing Compound (I)

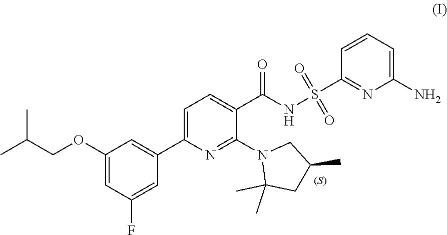

from a mixture of ethanol, water, and Compound (I).
9. A method of preparing Crystalline Form A of Compound (I), comprising crystallizing Compound (I)

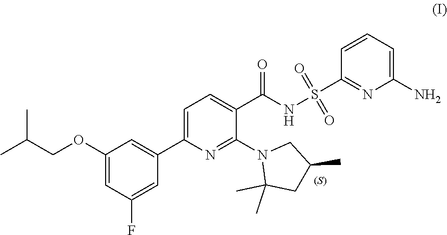

from a mixture of ethanol, water, and Compound (I).

### 10. Crystalline Form B of Compound (I):

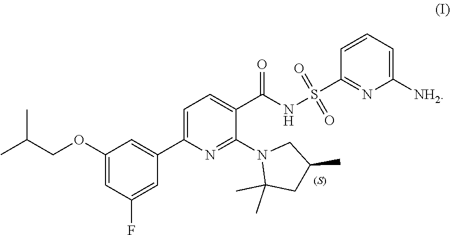

11. Crystalline Form B according to embodiment 10 in substantially pure form.
12. Crystalline Form B according to embodiment 10, characterized by an X-ray powder diffractogram having a signal at at least three two-theta values chosen from 12.4±0.2, 14.5±0.2, 16.9±0.2, 17.5±0.2, 19.8±0.2, 23.1±0.2, 23.9±0.2, 26.1±0.2, and 28.3±0.2.
13. Crystalline Form B according to embodiment 10, characterized by an X-ray powder diffractogram having a signal at at least three two-theta values chosen from 12.4±0.2, 16.9±0.2, 17.5±0.2, 19.8±0.2, 23.1±0.2, and 26.1±0.2.
14. Crystalline Form B according to embodiment 10, characterized by an X-ray powder diffractogram having a signal at three two-theta values of 12.4±0.2, 19.8±0.2, and 23.1±0.2.
15. Crystalline Form B according to embodiment 10, characterized by an X-ray powder diffractogram having a signal at six two-theta values of 12.4±0.2, 16.9±0.2, 17.5±0.2, 19.8±0.2, 23.1±0.2, and 26.1±0.2.
16. Crystalline Form B of embodiment 10, characterized by an X-ray powder diffractogram substantially similar to that in FIG. 5.
17. Crystalline Form B of Compound (I) prepared by a process comprising crystallizing Compound (I)

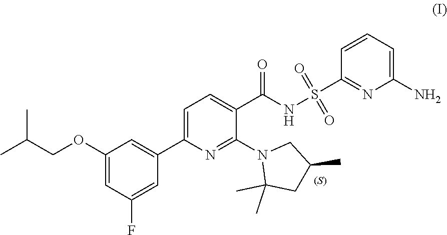

from a mixture of isopropylacetate and Compound (I).
18. A method of preparing Crystalline Form B of Compound (I), comprising crystallizing Compound (I)

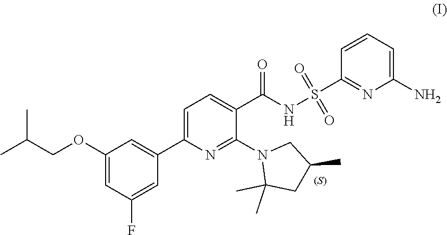

from a mixture of isopropylacetate and Compound (I).

### 19. Crystalline Form H of Compound (I):

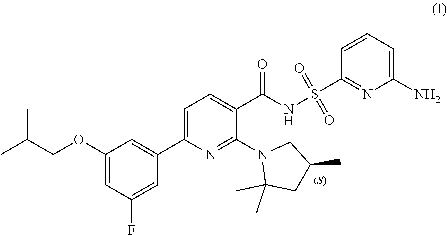

20. Crystalline Form H according to embodiment 19 in substantially pure form.
21. Crystalline Form H according to embodiment 19, characterized by an X-ray powder diffractogram having a signal at at least three two-theta values chosen from 13.2±0.2, 14.8±0.2, 15.5±0.2, 16.8±0.2, 17.2±0.2, 17.8±0.2, 19.2±0.2, 22.1±0.2, 25.1±0.2, and 28.5±0.2.
22. Crystalline Form H according to embodiment 19, characterized by an X-ray powder diffractogram having a signal at at least three two-theta values chosen from 13.2±0.2, 14.8±0.2, 16.8±0.2, 17.8±0.2, 19.2±0.2, and 28.5±0.2.
23. Crystalline Form H according to embodiment 19, characterized by an X-ray powder diffractogram having a signal at three two-theta values of 13.2±0.2, 17.8±0.2, and 19.2±0.2.
24. Crystalline Form H according to embodiment 19, characterized by an X-ray powder diffractogram having a signal at six two-theta values of 13.2±0.2, 14.8±0.2, 16.8±0.2, 17.8±0.2, 19.2±0.2, and 28.5±0.2.
25. Crystalline Form H of embodiment 19, characterized by an X-ray powder diffractogram substantially similar to that in FIG. 8.
26. Crystalline Form H of Compound (I) prepared by a process comprising crystallizing Compound (I)

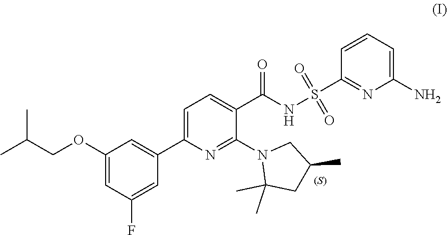

from a mixture of methanol, water, and Compound (I).
27. A method of preparing Crystalline Form H of Compound (I), comprising crystallizing Compound (I)

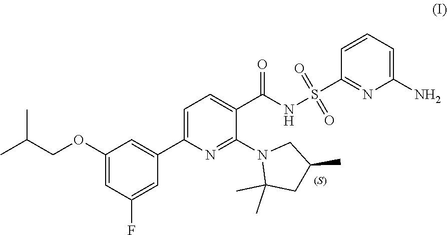

from a mixture of methanol, water, and Compound (I).

### 28. Crystalline Form S of Compound (I):

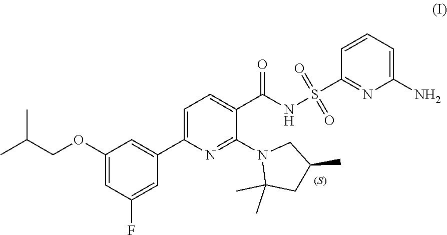

29. Crystalline Form S according to embodiment 28 in substantially pure form.
30. Crystalline Form S according to embodiment 28, characterized by an X-ray powder diffractogram having a signal at at least three two-theta values chosen from 11.5±0.2, 14.9±0.2, 16.6±0.2, 17.0±0.2, 18.8±0.2, 20.5±0.2, 21.2±0.2, 22.6±0.2, 26.4±0.2, and 25.3±0.2.
31. Crystalline Form S according to embodiment 28, characterized by an X-ray powder diffractogram having a signal at at least three two-theta values chosen from 11.5±0.2, 14.9±0.2, 17.0±0.2, 18.8±0.2, 21.2±0.2, and 22.6±0.2.
32. Crystalline Form S according to embodiment 28, characterized by an X-ray powder diffractogram having a signal at three two-theta values of 14.9±0.2, 18.8±0.2, and 21.2±0.2.
33. Crystalline Form S according to embodiment 28, characterized by an X-ray powder diffractogram having a signal at six two-theta values of 11.5±0.2, 14.9±0.2, 17.0±0.2, 18.8±0.2, 21.2±0.2, and 22.6±0.2.
34. Crystalline Form S according to embodiment 28, characterized by an X-ray powder diffractogram substantially similar to that in FIG. 12.
35. Crystalline Form S of Compound (I) prepared by a process comprising crystallizing Compound (I)

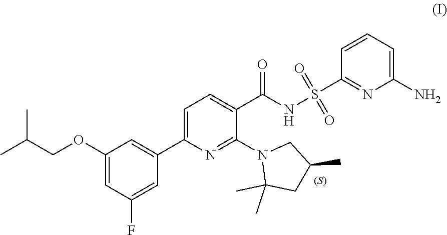

from a mixture of 1,4-dioxane, heptane, and Compound (I).
36. A method of preparing Crystalline Form S of Compound (I), comprising crystallizing Compound (I)

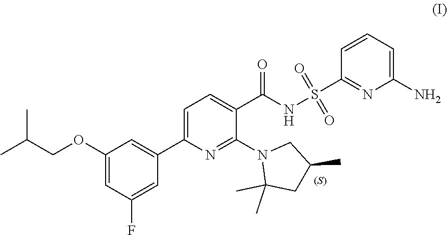

from a mixture of 1,4-dioxane, heptane, and Compound (I).

### 37. Crystalline Form A2 of Compound (II):

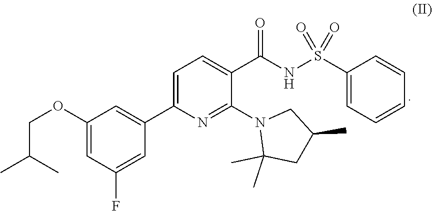 [SMILES: CC(C)COc1cc(F)cc(-c2ccc(C(=O)NS(=O)(=O)c3ccccc3)c(N3C[C@@H](C)CC3(C)C)n2)c1]

38. Crystalline Form A2 according to embodiment 37 in substantially pure form.
39. Crystalline Form A2 according to embodiment 37, characterized by an X-ray powder diffractogram having a signal at at least five two-theta values chosen from 8.4±0.2, 9.0±0.2, 11.2±0.2, 13.8±0.2, 15.3±0.2, 15.7±0.2, 19.3±0.2, 20.0±0.2, 20.4±0.2, 23.7±0.2, 24.9±0.2, and 27.8±0.2.
40. Crystalline Form A2 according to embodiment 37, characterized by an X-ray powder diffractogram having a signal at at least three two-theta values chosen from 9.0±0.2, 11.2±0.2, 13.8±0.2, 15.3±0.2, 15.7±0.2, 19.3±0.2, 20.0±0.2, 20.4±0.2, 23.7±0.2, and 24.9±0.2.
41. Crystalline Form A2 according to embodiment 37, characterized by an X-ray powder diffractogram having a signal at least three two-theta values of 9.0±0.2, 11.2±0.2, 13.8±0.2, 15.3±0.2, 15.7±0.2, 19.3±0.2, and 20.0±0.2.
42. Crystalline Form A2 according to embodiment 37, characterized by an X-ray powder diffractogram having a signal at least three two-theta values of 9.0±0.2, 11.2±0.2, 13.8±0.2, 15.3±0.2, 15.7±0.2, and 19.3±0.2.
43. Crystalline Form A2 according to embodiment 37, characterized by an X-ray powder diffractogram having a signal at at least three two-theta values chosen from 8.4±0.2, 9.0±0.2, 11.2±0.2, 13.8±0.2, 15.3±0.2, 15.7±0.2, and 19.3±0.2.
44. Crystalline Form A2 according to embodiment 37, characterized by an X-ray powder diffractogram having a signal at five two-theta values of 9.0±0.2, 11.2±0.2, 13.8±0.2, 15.7±0.2, and 20.0±0.2.
45. Crystalline Form A2 according to embodiment 37, characterized by an X-ray powder diffractogram substantially similar to that in FIG. 18.
46. Crystalline Form A2 of Compound (II) prepared by a process comprising crystallizing Compound (II)

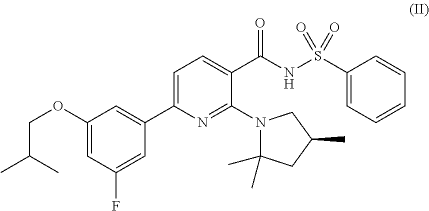 [SMILES: CC(C)COc1cc(F)cc(-c2ccc(C(=O)NS(=O)(=O)c3ccccc3)c(N3C[C@@H](C)CC3(C)C)n2)c1]

from a mixture of ethanol, water, and Compound (II).
47. A method of preparing Crystalline Form A2 of Compound (II), comprising crystallizing Compound (II)

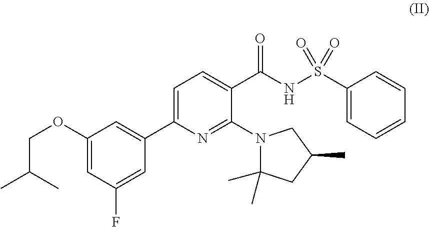 [SMILES: CC(C)COc1cc(F)cc(-c2ccc(C(=O)NS(=O)(=O)c3ccccc3)c(N3C[C@@H](C)CC3(C)C)n2)c1]

from a mixture of ethanol, water, and Compound (II).
48. At least one solvate of Compound (II):

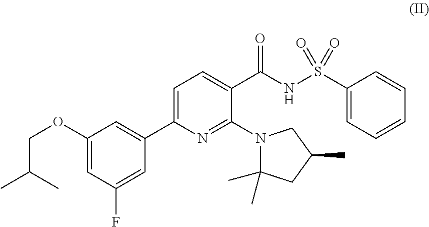 [SMILES: CC(C)COc1cc(F)cc(-c2ccc(C(=O)NS(=O)(=O)c3ccccc3)c(N3C[C@@H](C)CC3(C)C)n2)c1]

chosen from iso-propanol solvates, n-propanol solvates, butanol solvates, and 2-methyl-1-propanol solvates, pentanol solvates, tetrahydrofuran solvates, ethanol solvates, acetonitrile solvates, and 2-ethoxyethanol solvates of Compound (II).

### 49. Crystalline Form IP of Compound (II):

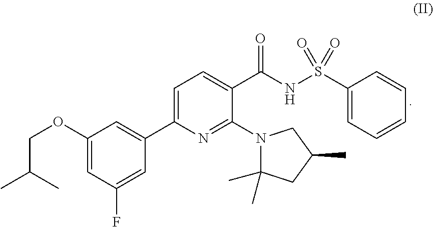 [SMILES: CC(C)COc1cc(F)cc(-c2ccc(C(=O)NS(=O)(=O)c3ccccc3)c(N3C[C@@H](C)CC3(C)C)n2)c1]

50. Crystalline Form IP according to embodiment 49 in substantially pure form.
51. Crystalline Form IP according to embodiment 49, characterized by an X-ray powder diffractogram having a signal at at least five two-theta values chosen from 9.7±0.2, 10.1±0.2, 12.0±0.2, 14.6±0.2, 15.0±0.2, 17.7±0.2, 18.2±0.2, 19.0±0.2, 19.4±0.2, and 20.6±0.2.
52. Crystalline Form IP according to embodiment 49, characterized by an X-ray powder diffractogram having a signal at at least three two-theta values chosen from 10.1±0.2, 12.0±0.2, 14.6±0.2, 15.0±0.2, 19.0±0.2, 19.4±0.2, and 20.6±0.2.
53. Crystalline Form IP according to embodiment 49, characterized by an X-ray powder diffractogram having a signal at four two-theta values of 10.1±0.2, 12.0±0.2, 14.6±0.2, and 15.0±0.2.
54. Crystalline Form IP according to embodiment 49, characterized by an X-ray powder diffractogram substantially similar to that in FIG. 22.
55. Crystalline Form IP of Compound (II) prepared by a process comprising crystallizing Compound (II)

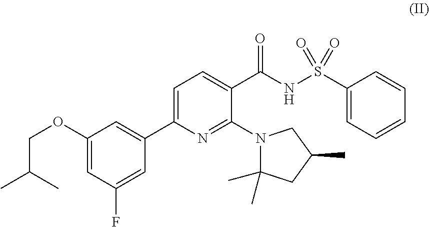 [SMILES: CC(C)COc1cc(F)cc(-c2ccc(C(=O)NS(=O)(=O)c3ccccc3)c(N3C[C@@H](C)CC3(C)C)n2)c1]

from a mixture of isopropanol and Compound (II).
56. A method of preparing Crystalline Form IP of Compound (II), comprising crystallizing Compound (II)

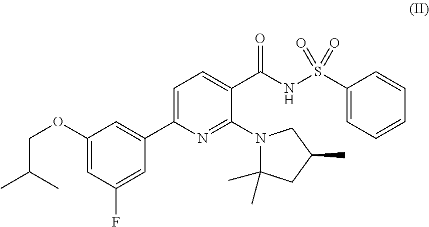 [SMILES: CC(C)COc1cc(F)cc(-c2ccc(C(=O)NS(=O)(=O)c3ccccc3)c(N3C[C@@H](C)CC3(C)C)n2)c1]

from a mixture of isopropanol and Compound (II).

### 57. Crystalline Form NPR of Compound (II):

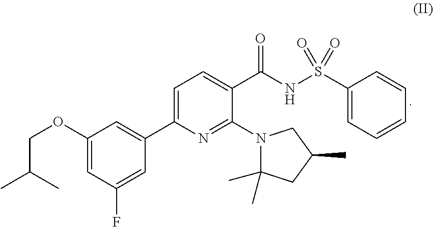 [SMILES: CC(C)COc1cc(F)cc(-c2ccc(C(=O)NS(=O)(=O)c3ccccc3)c(N3C[C@@H](C)CC3(C)C)n2)c1]

58. Crystalline Form NPR according to embodiment 57 in substantially pure form.
59. Crystalline Form NPR according to embodiment 57, characterized by an X-ray powder diffractogram having a signal at at least five two-theta values chosen from 4.8±0.2, 7.7±0.2, 8.0±0.2, 10.1±0.2, 12.1±0.2, 14.7±0.2, 15.0±0.2, 15.5±0.2, 17.7±0.2, 19.1±0.2, and 20.6±0.2.
60. Crystalline Form NPR according to embodiment 57, characterized by an X-ray powder diffractogram having a signal at at least three two-theta values chosen from 4.8±0.2, 7.7±0.2, 8.0±0.2, 10.1±0.2, 12.1±0.2, 15.0±0.2, 15.5±0.2, 19.1±0.2, and 20.6±0.2.
61. Crystalline Form NPR according to embodiment 57, characterized by an X-ray powder diffractogram having a signal at five two-theta values chosen from 4.8±0.2, 7.7±0.2, 8.0±0.2, 10.1±0.2, 12.1±0.2, 15.0±0.2, 19.1±0.2, and 20.6±0.2.
62. Crystalline Form NPR according to embodiment 57, characterized by an X-ray powder diffractogram having a signal at three two-theta values of 10.1±0.2, 12.1±0.2, and 15.0±0.2.
63. Crystalline Form NPR according to embodiment 57, characterized by an X-ray powder diffractogram substantially similar to that in FIG. 26.
64. Crystalline Form NPR of Compound (II) prepared by a process comprising crystallizing Compound (II)

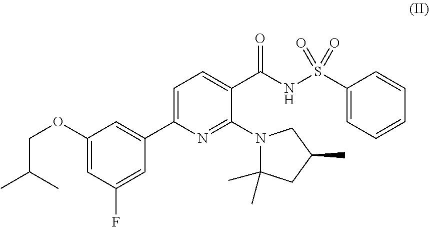 [SMILES: CC(C)COc1cc(F)cc(-c2ccc(C(=O)NS(=O)(=O)c3ccccc3)c(N3C[C@@H](C)CC3(C)C)n2)c1]

from a mixture of n-propanol and Compound (II).
65. A method of preparing Crystalline Form NPR of Compound (II), comprising crystallizing Compound (II)

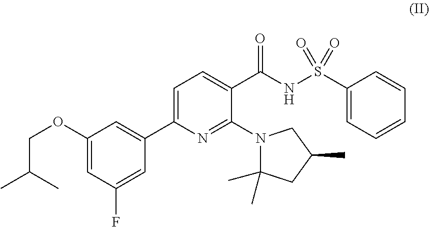 [SMILES: CC(C)COc1cc(F)cc(-c2ccc(C(=O)NS(=O)(=O)c3ccccc3)c(N3C[C@@H](C)CC3(C)C)n2)c1]

from a mixture of n-propanol and Compound (II).

### 66. Crystalline Form 2B of Compound (II):

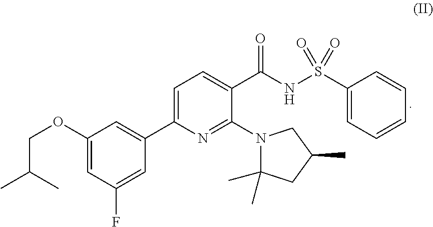 [SMILES: CC(C)COc1cc(F)cc(-c2ccc(C(=O)NS(=O)(=O)c3ccccc3)c(N3C[C@@H](C)CC3(C)C)n2)c1]

67. Crystalline Form 2B according to embodiment 66 in substantially pure form.
68. Crystalline Form 2B according to embodiment 66, characterized by an X-ray powder diffractogram having a signal at at least five two-theta values chosen from 4.7±0.2, 7.8±0.2, 10.2±0.2, 12.0±0.2, 13.6±0.2, 14.1±0.2, 14.5±0.2, 15.0±0.2, 15.6±0.2, 17.9±0.2, 18.8±0.2, 19.2±0.2, and 20.8±0.2.
69. Crystalline Form 2B according to embodiment 66, characterized by an X-ray powder diffractogram having a signal at at least five two-theta values chosen from 4.7±0.2, 7.8±0.2, 10.2±0.2, 12.0±0.2, 14.1±0.2, 15.0±0.2, 15.6±0.2, 17.9±0.2, 19.2±0.2, and 20.8±0.2.
70. Crystalline Form 2B according to embodiment 66, characterized by an X-ray powder diffractogram having a signal at at least three two-theta values chosen from 4.7±0.2, 7.8±0.2, 10.2±0.2, 12.0±0.2, 14.1±0.2, 15.0±0.2, 19.2±0.2, and 20.8±0.2.
71. Crystalline Form 2B according to embodiment 66, characterized by an X-ray powder diffractogram having a signal at four two-theta values of 10.2±0.2, 12.0±0.2, 14.1±0.2, and 15.0±0.2.
72. Crystalline Form 2B according to embodiment 66, characterized by an X-ray powder diffractogram substantially similar to that in FIG. 27.
73. Crystalline Form 2B of Compound (II) prepared by a process comprising crystallizing Compound (II)

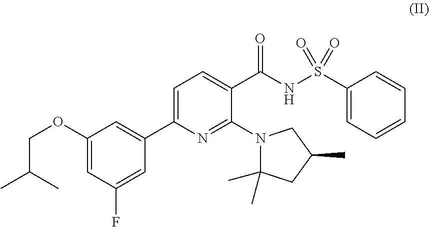 [SMILES: CC(C)COc1cc(F)cc(-c2ccc(C(=O)NS(=O)(=O)c3ccccc3)c(N3C[C@@H](C)CC3(C)C)n2)c1]

from a mixture of 2-butanol and Compound (II).
74. A method of preparing Crystalline Form 2B of Compound (II), comprising crystallizing Compound (II)

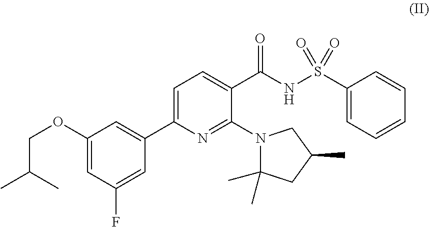 [SMILES: CC(C)COc1cc(F)cc(-c2ccc(C(=O)NS(=O)(=O)c3ccccc3)c(N3C[C@@H](C)CC3(C)C)n2)c1]

from a mixture of 2-butanol and Compound (II).

### 75. Crystalline Form MP of Compound (II):

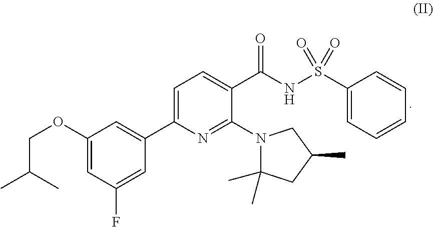 [SMILES: CC(C)COc1cc(F)cc(-c2ccc(C(=O)NS(=O)(=O)c3ccccc3)c(N3C[C@@H](C)CC3(C)C)n2)c1]

76. Crystalline Form MP according to embodiment 75 in substantially pure form.
77. Crystalline Form MP according to embodiment 75, characterized by an X-ray powder diffractogram having a signal at at least five two-theta values chosen from 4.7±0.2, 7.8±0.2, 8.1±0.2, 10.1±0.2, 12.0±0.2, 15.0±0.2, 15.4±0.2, 15.7±0.2, 17.6±0.2, 18.0±0.2, 19.2±0.2, and 20.7±0.2.
78. Crystalline Form MP according to embodiment 75, characterized by an X-ray powder diffractogram having a signal at at least five two-theta values chosen from 4.7±0.2, 7.8±0.2, 8.1±0.2, 10.1±0.2, 12.0±0.2, 15.0±0.2, 19.2±0.2, and 20.7±0.2.
79. Crystalline Form MP according to embodiment 75, characterized by an X-ray powder diffractogram having a signal at at least three two-theta values chosen from 4.7±0.2, 7.8±0.2, 10.1±0.2, 12.0±0.2, 15.0±0.2, 19.2±0.2, and 20.7±0.2.
80. Crystalline Form MP according to embodiment 75, characterized by an X-ray powder diffractogram having a signal at three two-theta values of 10.1±0.2, 12.0±0.2, and 15.0±0.2.
81. Crystalline Form MP according to embodiment 75, characterized by an X-ray powder diffractogram substantially similar to that in FIG. 30.
82. Crystalline Form MP of Compound (II) prepared by a process comprising crystallizing Compound (II)

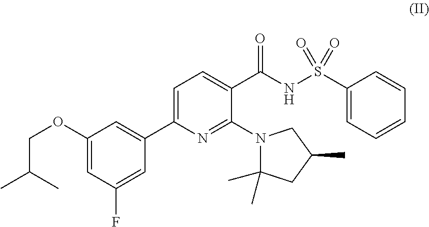 [SMILES: CC(C)COc1cc(F)cc(-c2ccc(C(=O)NS(=O)(=O)c3ccccc3)c(N3C[C@@H](C)CC3(C)C)n2)c1]

from a mixture of 2-methyl-1-propanol and Compound (II).
83. A method of preparing Crystalline Form MP of Compound (II), comprising crystallizing Compound (II)

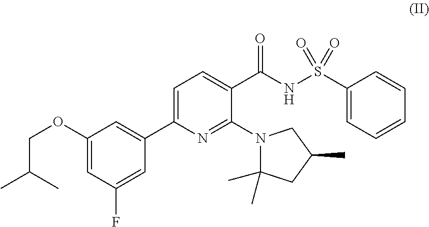 [SMILES: CC(C)COc1cc(F)cc(-c2ccc(C(=O)NS(=O)(=O)c3ccccc3)c(N3C[C@@H](C)CC3(C)C)n2)c1]

from a mixture of 2-methyl-1-propanol and Compound (II).

### 84. Crystalline Form NP of Compound (II):

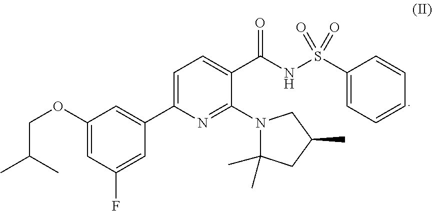 [SMILES: CC(C)COc1cc(F)cc(-c2ccc(C(=O)NS(=O)(=O)c3ccccc3)c(N3C[C@@H](C)CC3(C)C)n2)c1]

85. Crystalline Form NP according to embodiment 84 in substantially pure form.
86. Crystalline Form NP according to embodiment 84, characterized by an X-ray powder diffractogram having a signal at at least five two-theta values chosen from 7.8±0.2, 13.5±0.2, 14.4±0.2, 15.2±0.2, 18.6±0.2, 19.0±0.2, 19.3±0.2, 21.0±0.2, and 26.4±0.2.
87. Crystalline Form NP according to embodiment 84, characterized by an X-ray powder diffractogram having a signal at at least five two-theta values chosen from 7.8±0.2, 13.5±0.2, 14.4±0.2, 15.2±0.2, 19.0±0.2, 21.0±0.2, and 26.4±0.2.
88. Crystalline Form NP according to embodiment 84, characterized by an X-ray powder diffractogram having a signal at at least three two-theta values chosen from 7.8±0.2, 13.5±0.2, 14.4±0.2, 15.2±0.2, 19.0±0.2, 21.0±0.2, and 26.4±0.2.
89. Crystalline Form NP according to embodiment 84, characterized by an X-ray powder diffractogram having a signal at four two-theta values 14.4±0.2, 15.2±0.2, 19.0±0.2, and 26.4±0.2.
90. Crystalline Form NP according to embodiment 84, characterized by an X-ray powder diffractogram substantially similar to that in FIG. 33.
91. Crystalline Form NP of Compound (II) prepared by a process comprising crystallizing Compound (II)

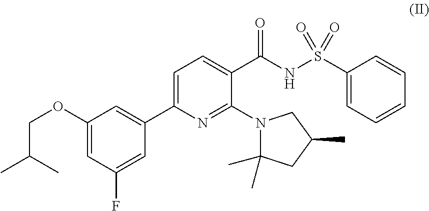 [SMILES: CC(C)COc1cc(F)cc(-c2ccc(C(=O)NS(=O)(=O)c3ccccc3)c(N3C[C@@H](C)CC3(C)C)n2)c1]

from a mixture of n-pentanol and Compound (II).
92. A method of preparing Crystalline Form NP of Compound (II), comprising crystallizing Compound (II)

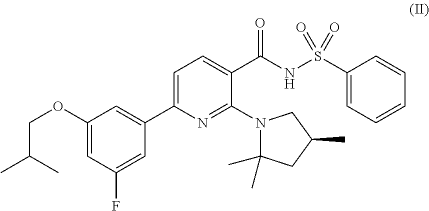 [SMILES: CC(C)COc1cc(F)cc(-c2ccc(C(=O)NS(=O)(=O)c3ccccc3)c(N3C[C@@H](C)CC3(C)C)n2)c1]

from a mixture of n-pentanol and Compound (II).

### 93. Crystalline Form EE of Compound (II):

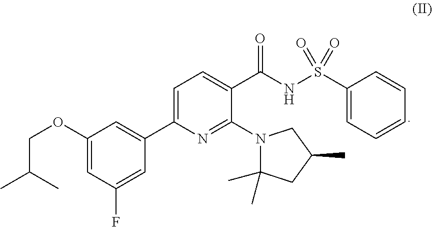 [SMILES: CC(C)COc1cc(F)cc(-c2ccc(C(=O)NS(=O)(=O)c3ccccc3)c(N3C[C@@H](C)CC3(C)C)n2)c1]

94. Crystalline Form EE according to embodiment 93 in substantially pure form.
95. Crystalline Form EE according to embodiment 93, characterized by an X-ray powder diffractogram having a signal at at least five two-theta values chosen from 4.6±0.2, 7.9±0.2, 13.6±0.2, 14.5±0.2, 15.3±0.2, 15.9±0.2, 18.0±0.2, 18.7±0.2, 19.1±0.2, 19.4±0.2, 21.2±0.2, and 26.4±0.2.
96. Crystalline Form EE according to embodiment 93, characterized by an X-ray powder diffractogram having a signal at at least five two-theta values chosen from 4.6±0.2, 7.9±0.2, 13.5±0.2, 13.6±0.2, 14.5±0.2, 15.3±0.2, 15.9±0.2, 18.7±0.2, 19.1±0.2, and 26.4±0.2.
97. Crystalline Form EE according to embodiment 93, characterized by an X-ray powder diffractogram having a signal at at least three two-theta values chosen from 4.6±0.2, 7.9±0.2, 14.5±0.2, 15.3±0.2, 15.9±0.2, 19.1±0.2, and 26.4±0.2.
98. Crystalline Form EE according to embodiment 93, characterized by an X-ray powder diffractogram having a signal at three two-theta values of 14.5±0.2, 15.3±0.2, 19.1±0.2, and 26.4±0.2.
99. Crystalline Form EE according to embodiment 93, characterized by an X-ray powder diffractogram substantially similar to that in FIG. 36.
100. Crystalline Form EE of Compound (II) prepared by a process comprising crystallizing Compound (II)

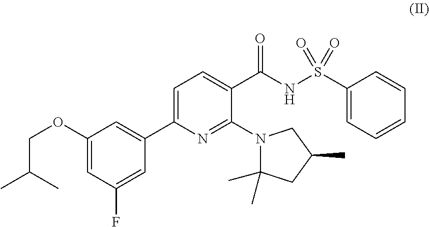 [SMILES: CC(C)COc1cc(F)cc(-c2ccc(C(=O)NS(=O)(=O)c3ccccc3)c(N3C[C@@H](C)CC3(C)C)n2)c1]

from a mixture of 2-ethoxyethanol and Compound (II).
101. A method of preparing Crystalline Form EE of Compound (II), comprising crystallizing Compound (II)

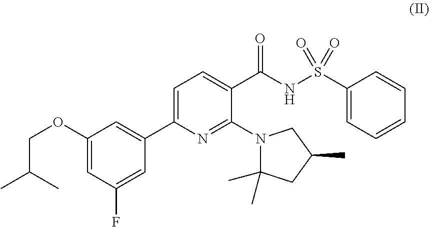 [SMILES: CC(C)COc1cc(F)cc(-c2ccc(C(=O)NS(=O)(=O)c3ccccc3)c(N3C[C@@H](C)CC3(C)C)n2)c1]

from a mixture of 2-ethoxyethanol and Compound (II).

### 102. Crystalline Form E of Compound (II):

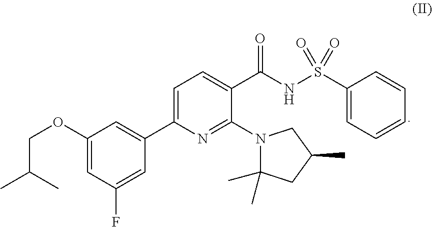 [SMILES: CC(C)COc1cc(F)cc(-c2ccc(C(=O)NS(=O)(=O)c3ccccc3)c(N3C[C@@H](C)CC3(C)C)n2)c1]

103. Crystalline Form E according to embodiment 102 in substantially pure form.
104. Crystalline Form E according to embodiment 102, characterized by an X-ray powder diffractogram having a signal at at least five two-theta values chosen from 7.8±0.2, 8.7±0.2, 11.7±0.2, 13.8±0.2, 14.4±0.2, 18.0±0.2, 19.2±0.2, 20.0±0.2, 22.1±0.2, and 23.7±0.2.
105. Crystalline Form E according to embodiment 102, characterized by an X-ray powder diffractogram having a signal at at least five two-theta values chosen from 7.8±0.2, 8.7±0.2, 11.7±0.2, 14.4±0.2, 19.2±0.2, 22.1±0.2, and 23.7±0.2.
106. Crystalline Form E according to embodiment 102, characterized by an X-ray powder diffractogram having a signal at at least three two-theta values chosen from 7.8±0.2, 8.7±0.2, 11.7±0.2, 14.4±0.2, 19.2±0.2, and 23.7±0.2.
107. Crystalline Form E according to embodiment 102, characterized by an X-ray powder diffractogram having a signal at three two-theta values of 7.8±0.2, 8.7±0.2, and 11.7±0.2.
108. Crystalline Form E according to embodiment 102, characterized by an X-ray powder diffractogram substantially similar to that in FIG. 39.
109. Crystalline Form E of Compound (II) prepared by a process comprising crystallizing Compound (II)

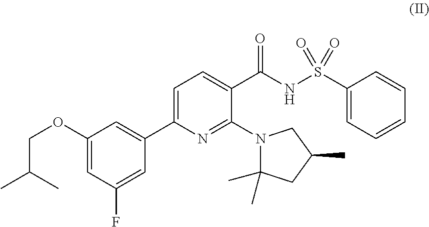 [SMILES: CC(C)COc1cc(F)cc(-c2ccc(C(=O)NS(=O)(=O)c3ccccc3)c(N3C[C@@H](C)CC3(C)C)n2)c1]

from a mixture of ethanol and Compound (II).
110. A method of preparing Crystalline Form E of Compound (II), comprising crystallizing Compound (II)

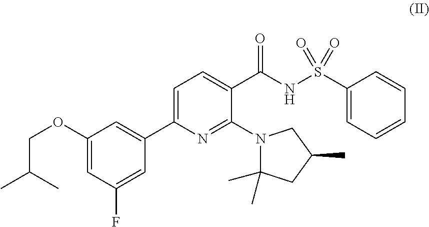 [SMILES: CC(C)COc1cc(F)cc(-c2ccc(C(=O)NS(=O)(=O)c3ccccc3)c(N3C[C@@H](C)CC3(C)C)n2)c1]

from a mixture of ethanol and Compound (II).

### 111. Crystalline Form T of Compound (II):

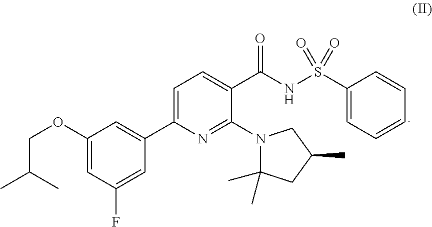 [SMILES: CC(C)COc1cc(F)cc(-c2ccc(C(=O)NS(=O)(=O)c3ccccc3)c(N3C[C@@H](C)CC3(C)C)n2)c1]

112. Crystalline Form T according to embodiment 111 in substantially pure form.
113. Crystalline Form T according to embodiment 111, characterized by an X-ray powder diffractogram having a signal at at least five two-theta values chosen from 7.9±0.2, 10.6±0.2, 15.0±0.2, 15.7±0.2, 18.1±0.2, 18.5±0.2, 19.0±0.2, 20.2±0.2, 20.9±0.2, and 23.8±0.2.
114. Crystalline Form T according to embodiment 111, characterized by an X-ray powder diffractogram having a signal at at least five two-theta values chosen from 7.9±0.2, 10.6±0.2, 15.0±0.2, 15.7±0.2, 18.1±0.2, 18.5±0.2, and 19.0±0.2.
115. Crystalline Form T according to embodiment 111, characterized by an X-ray powder diffractogram having a signal at at least three two-theta values chosen from 7.9±0.2, 10.6±0.2, 15.0±0.2, 15.7±0.2, 18.1±0.2, 18.5±0.2, and 19.0±0.2.
116. Crystalline Form T according to embodiment 111, characterized by an X-ray powder diffractogram having a signal at three two-theta values of 10.6±0.2, 18.1±0.2, and 18.5±0.2.
117. Crystalline Form T according to embodiment 111, characterized by an X-ray powder diffractogram substantially similar to that in FIG. 40.
118. Crystalline Form T of Compound (II) prepared by a process comprising crystallizing Compound (II)

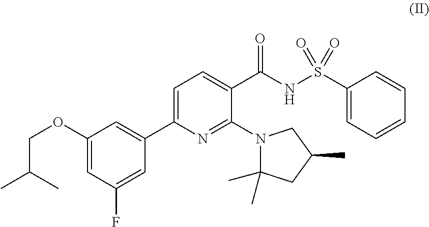 [SMILES: CC(C)COc1cc(F)cc(-c2ccc(C(=O)NS(=O)(=O)c3ccccc3)c(N3C[C@@H](C)CC3(C)C)n2)c1]

from a mixture of tetrahydrofuran and Compound (II).
119. A method of preparing Crystalline Form T of Compound (II), comprising crystallizing Compound (II)

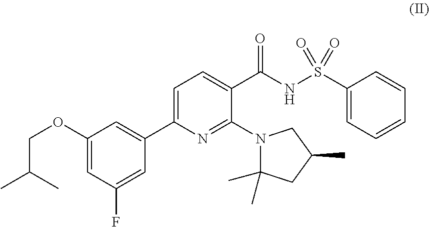 [SMILES: CC(C)COc1cc(F)cc(-c2ccc(C(=O)NS(=O)(=O)c3ccccc3)c(N3C[C@@H](C)CC3(C)C)n2)c1]

from a mixture of tetrahydrofuran and Compound (II).

### 120. Crystalline Form AC of Compound (II):

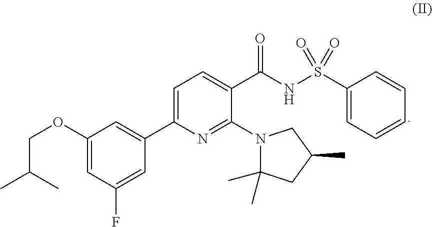 [SMILES: CC(C)COc1cc(F)cc(-c2ccc(C(=O)NS(=O)(=O)c3ccccc3)c(N3C[C@@H](C)CC3(C)C)n2)c1]

121. Crystalline Form AC according to embodiment 120 in substantially pure form.
122. Crystalline Form AC according to embodiment 120, characterized by an X-ray powder diffractogram having a signal at at least three two-theta values chosen from 6.5±0.2, 13.1±0.2, 19.4±0.2, and 19.7±0.2.
123. Crystalline Form AC according to embodiment 120, characterized by an X-ray powder diffractogram having a signal at at least two two-theta values chosen from 6.5±0.2, 13.1±0.2, 19.4±0.2, and 19.7±0.2.
124. Crystalline Form AC according to embodiment 120, characterized by an X-ray powder diffractogram having a signal at two-theta values of 6.5±0.2, 13.1±0.2, 19.4±0.2, and 19.7±0.2.
125. Crystalline Form AC according to embodiment 120, characterized by an X-ray powder diffractogram having a signal at three two-theta values of 6.5±0.2, 13.1±0.2, and 19.4±0.2.
126. Crystalline Form AC according to embodiment 120, characterized by an X-ray powder diffractogram substantially similar to that in FIG. 41.
127. Crystalline Form AC of Compound (II) prepared by a process comprising crystallizing Compound (II)

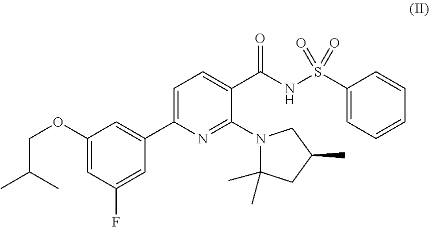 [SMILES: CC(C)COc1cc(F)cc(-c2ccc(C(=O)NS(=O)(=O)c3ccccc3)c(N3C[C@@H](C)CC3(C)C)n2)c1]

from a mixture of acetonitrile and Compound (II).
128. A method of preparing Crystalline Form AC of Compound (II), comprising crystallizing Compound (II)

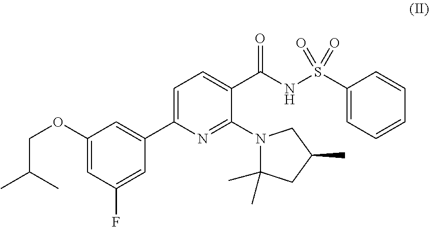 [SMILES: CC(C)COc1cc(F)cc(-c2ccc(C(=O)NS(=O)(=O)c3ccccc3)c(N3C[C@@H](C)CC3(C)C)n2)c1]

from a mixture of acetonitrile and Compound (II).
129. A pharmaceutical formulation comprising at least one crystalline form according to any one of embodiments 1-128 and a pharmaceutically acceptable carrier.
130. A method of treating cystic fibrosis comprising administering to a patient in need thereof at least one crystalline form according to any one of embodiments 1-128 or the pharmaceutical formulation according to embodiment 129.

### EXAMPLES

### General Experimental Procedures

Reagents and starting materials were obtained by commercial sources unless otherwise stated and were used without purification. Proton and carbon NMR spectra were acquired on either of a Bruker Biospin DRX 400 MHz FTNMR spectrometer operating at a ¹H and ¹³C resonant frequency of 400 and 100 MHz respectively, or on a 300 MHz NMR spectrometer. One dimensional proton and carbon spectra were acquired using a broadband observe (BBFO) probe with 20 Hz sample rotation at 0.1834 and 0.9083 Hz/Pt digital resolution respectively. All proton and carbon spectra were acquired with temperature control at 30° C. using standard, previously published pulse sequences and routine processing parameters.

Solid state ¹³C and ¹⁹F NMR data was obtained using Bruker-Biospin 400 MHz wide-bore spectrometer equipped with Bruker-Biospin 4 mm HFX probe was used. Samples were packed into 4 mm rotors and spun under Magic Angle Spinning (MAS) condition with typical spinning speed of 12.5 kHz. The proton relaxation time was estimated from ¹H MAS T₁ saturation recovery relaxation experiment and used to set up proper recycle delay of the ¹³C cross-polarization (CP) MAS experiment. The fluorine relaxation time was estimated from ¹⁹F MAS T₁ saturation recovery relaxation experiment and used to set up proper recycle delay of the ¹⁹F MAS experiment. The CP contact time of CPMAS experiments was set to 2 ms. A CP proton pulse with linear ramp (from 50% to 100%) was employed. All spectra were externally referenced by adjusting the magnetic field to set carbon resonance of adamantane to 29.5 ppm. TPPM15 proton decoupling sequence was used with the field strength of approximately 100 kHz for both ¹³C and ¹⁹F acquisitions.

Final purity of compounds was determined by reversed phase UPLC using an Acquity UPLC BEH C₁₈ column (50×2.1 mm, 1.7 μm particle) made by Waters (pn: 186002350), and a dual gradient run from 1-99% mobile phase B over 3.0 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C. Final purity was calculated by averaging the area under the curve (AUC) of two UV traces (220 nm, 254 nm). Low-resolution mass spectra were reported as [M+H]⁺ species obtained using a single quadrupole mass spectrometer equipped with an electrospray ionization (ESI) source capable of achieving a mass accuracy of 0.1 Da and a minimum resolution of 1000 (no units on resolution) across the detection range. Optical purity of methyl (2S)-2,4-dimethyl-4-nitro-pentanoate was determined using chiral gas chromatography (GC) analysis on an Agilent 7890A/MSD 5975C instrument, using a Restek Rt-βDEXcst (30m×0.25 mm×0.25 um_df) column, with a 2.0 mL/min flow rate (H2 carrier gas), at an injection temperature of 220° C. and an oven temperature of 120° C., 15 minutes.

### Powder X-Ray Diffraction

Powder x-ray diffraction patterns were acquired at room temperature in reflection or in transmission mode using a Bruker D8 Advance or PANalytical Empyrean diffractometer with a copper radiation source. Samples were analyzed on a silicon sample holder or in a 96-well plate sample holder from 3-40° 2-theta on continuous mode with a step size of around 0.013° and with total scan time of approximately 2-15 minutes. Samples were analyzed while spinning in the reflection mode.

### Single Crystal X-Ray Diffraction

X-ray diffraction data were acquired at 100 K or 296.15 Kon a Bruker diffractometer equipped with Mo K_{α} radiation (X, =0.71073 A) and a CCD detector. The structure was solved and refined using the SHELX program suite (Sheldrick, G. M., Acta Cryst., (2008) A64, 112-122).

### Thermogravimetric Analysis (TGA)

TGA was used to investigate the presence of residual solvents in the lots characterized, and identify the temperature at which decomposition of the sample occurs. TGA data were collected on a TA Discovery Thermogravimetric Analyzer or equivalent instrumentation. A sample with weight of approximately 1-20 mg was scanned from approximately 25° C. to 300° C. at a heating rate of 5° C./min or 10° C./min. Data were collected and analyzed by Trios software (TA Instruments, New Castle, Del.) or collected by Thermal Advantage Q Series™ software and analyzed by Universal Analysis software (TA Instruments, New Castle, Del.).

Differential Scanning calorimetry (DSC)

DSC data were acquired using a TA Instruments Q2000 or equivalent instrumentation. A sample with a weight between 1 and 10 mg was weighed into an aluminum pan with either a solid lid or pinhole lid. For a straight ramp, sample was heated from the starting point of approximate 25° C. to 250-300° C. For modulated heating, samples were modulated at 0.32° C./min or 1° C./min every 60 s. Data were collected and analyzed by Trios software (TA Instruments, New Castle, Del.) or collected by Thermal Advantage Q Series™ software and analyzed by Universal Analysis software (TA Instruments, New Castle, Del.). The observed endo- and exotherms were integrated between baseline temperature points that were above and below the temperature range over which the endotherm was observed.

### Example 1: Synthesis of Compound (I)

### Part A: Synthesis of (4S)-2,2,4-trimethylpyrrolidine hydrochloride

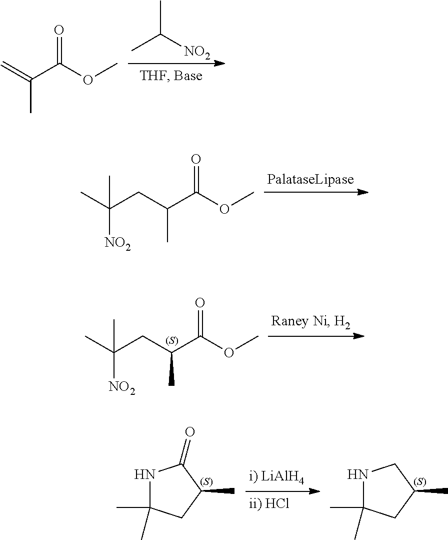

### Step 1: Synthesis of methyl-2,4-dimethyl-4-nitro-pentanoate

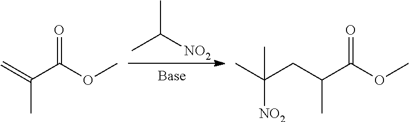

Tetrahydrofuran (THF, 4.5 L) was added to a 20 L glass reactor and stirred under N2 at room temperature. 2-Nitropropane (1.5 kg, 16.83 mol) and 1,8-Diazabicyclo[5.4.0]undec-7-ene (DBU) (1.282 kg, 8.42 mol) were then charged to the reactor, and the jacket temperature was increased to 50° C. Once the reactor contents were close to 50° C., methyl methacrylate (1.854 kg, 18.52 mol) was added slowly over 100 minutes. The reaction temperature was maintained at or close to 50° C. for 21 hours. The reaction mixture was concentrated in vacuo then transferred back to the reactor and diluted with methyl tert-butyl ether (MTBE) (14 L). 2 M HCl (7.5 L) was added, and this mixture was stirred for 5 minutes then allowed to settle. Two clear layers were visible—a lower yellow aqueous phase and an upper green organic phase. The aqueous layer was removed, and the organic layer was stirred again with 2 M HCl (3 L). After separation, the HCl washes were recombined and stirred with MTBE (3 L) for 5 minutes. The aqueous layer was removed, and all of the organic layers were combined in the reactor and stirred with water (3 L) for 5 minutes. After separation, the organic layers were concentrated in vacuo to afford a cloudy green oil. Crude product was treated with MgSO₄ and filtered to afford methyl-2,4-dimethyl-4-nitro-pentanoate as a clear green oil (3.16 kg, 99% yield). ¹H NMR (400 MHz, Chloroform-d) δ 3.68 (s, 3H), 2.56-2.35 (m, 2H), 2.11-2.00 (m, 1H), 1.57 (s, 3H), 1.55 (s, 3H), 1.19 (d, J=6.8 Hz, 3H).

### Step 2: Synthesis of methyl (25)-2,4-dimethyl-4-nitro-pentanoate

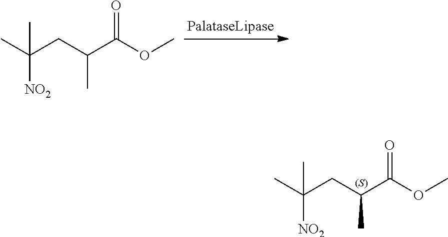

A reactor was charged with purified water (2090 L; 10 vol) and then potassium phosphate monobasic (27 kg, 198.4 moles; 13 g/L for water charge). The pH of the reactor contents was adjusted to pH 6.5 (±0.2) with 20% (w/v) potassium carbonate solution. The reactor was charged with racemic methyl-2,4-dimethyl-4-nitro-pentanoate (209 kg; 1104.6 moles), and Palatase 20000L lipase (13 L, 15.8 kg; 0.06 vol).

The reaction mixture was adjusted to 32±2° C. and stirred for 15-21 hours, and pH 6.5 was maintained using a pH stat with the automatic addition of 20% potassium carbonate solution. When the racemic starting material was converted to >98% ee of the S-enantiomer, as determined by chiral GC, external heating was switched off. The reactor was then charged with MTBE (35 L; 5 vol), and the aqueous layer was extracted with MTBE (3 times, 400-1000 L). The combined organic extracts were washed with aqueous Na₂CO₃ (4 times, 522 L, 18% w/w 2.5 vol), water (523 L; 2.5 vol), and 10% aqueous NaCl (314 L, 1.5 vol). The organic layer was concentrated in vacuo to afford methyl (2S)-2,4-dimethyl-4-nitro-pentanoate as a mobile yellow oil (>98% ee, 94.4 kg; 45% yield).

### Step 3: Synthesis of (3S)-3,5,5-trimethylpyrrolidin-2-one

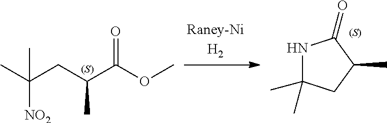

A 20 L reactor was purged with N2. The vessel was charged sequentially with DI water-rinsed, damp Raney® Ni (2800 grade, 250 g), methyl (2S)-2,4-dimethyl-4-nitro-pentanoate (1741 g, 9.2 mol), and ethanol (13.9 L, 8 vol). The reaction was stirred at 900 rpm, and the reactor was flushed with H2 and maintained at ˜2.5 bar. The reaction mixture was then warmed to 60° C. for 5 hours. The reaction mixture was cooled and filtered to remove Raney nickel, and the solid cake was rinsed with ethanol (3.5 L, 2 vol). The ethanolic solution of the product was combined with a second equal sized batch and concentrated in vacuo to reduce to a minimum volume of ethanol (˜1.5 volumes). Heptane (2.5 L) was added, and the suspension was concentrated again to ˜1.5 volumes. This was repeated 3 times; the resulting suspension was cooled to 0-5° C., filtered under suction, and washed with heptane (2.5 L). The product was dried under vacuum for 20 minutes then transferred to drying trays and dried in a vacuum oven at 40° C. overnight to afford (3S)-3,5,5-trimethylpyrrolidin-2-one as a white crystalline solid (2.042 kg, 16.1 mol, 87%). ¹H NMR (400 MHz, Chloroform-d) δ 6.39 (s, 1H), 2.62 (ddq, J=9.9, 8.6, 7.1 Hz, 1H), 2.17 (dd, J=12.4, 8.6 Hz, 1H), 1.56 (dd, J=12.5, 9.9 Hz, 1H), 1.31 (s, 3H), 1.25 (s, 3H), 1.20 (d, J=7.1 Hz, 3H).

### Step 4: Synthesis of (4S)-2,2,4-trimethylpyrrolidine hydrochloride

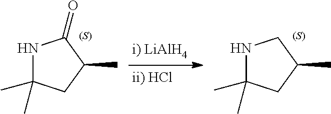

A glass lined 120 L reactor was charged with lithium aluminium hydride pellets (2.5 kg, 66 mol) and dry THF (60 L) and warmed to 30° C. The resulting suspension was charged with (S)-3,5,5-trimethylpyrrolidin-2-one (7.0 kg, 54 mol) in THF (25 L) over 2 hours while maintaining the reaction temperature at 30 to 40° C. After complete addition, the reaction temperature was increased to 60-63° C. and maintained overnight. The reaction mixture was cooled to 22° C., then cautiously quenched with the addition of ethyl acetate (EtOAc) (1.0 L, 10 moles), followed by a mixture of THF (3.4 L) and water (2.5 kg, 2.0 eq), and then a mixture of water (1.75 kg) with 50% aqueous sodium hydroxide (750 g, 2 equiv water with 1.4 equiv sodium hydroxide relative to aluminum), followed by 7.5 L water. After the addition was complete, the reaction mixture was cooled to room temperature, and the solid was removed by filtration and washed with THF (3×25 L). The filtrate and washings were combined and treated with 5.0 L (58 moles) of aqueous 37% HCl (1.05 equiv.) while maintaining the temperature below 30° C. The resultant solution was concentrated by vacuum distillation to a slurry. Isopropanol (8 L) was added and the solution was concentrated to near dryness by vacuum distillation. Isopropanol (4 L) was added, and the product was slurried by warming to 50° C. MTBE (6 L) was added, and the slurry was cooled to 2-5° C. The product was collected by filtration and rinsed with 12 L MTBE and dried in a vacuum oven (55° C./300 torr/N2 bleed) to afford (4S)-2,2,4-trimethylpyrrolidine.HCl as a white, crystalline solid (6.21 kg, 75% yield). ¹H NMR (400 MHz, DMSO-d6) δ 9.34 (br d, 2H), 3.33 (dd, J=11.4, 8.4 Hz, 1H), 2.75 (dd, J=11.4, 8.6 Hz, 1H), 2.50-2.39 (m, 1H), 1.97 (dd, J=12.7, 7.7 Hz, 1H), 1.42 (s, 3H), 1.38 (dd, J=12.8, 10.1 Hz, 1H), 1.31 (s, 3H), 1.05 (d, J=6.6 Hz, 3H).

### Part B: N-[(6-amino-2-pyridyl)sulfonyl]-6-(3-fluoro-5-isobutoxy-phenyl)-2-[(4S)-2,2,4-trimethylpyrrolidin-1-yl]pyridine-3-carboxamide (Compound (I))

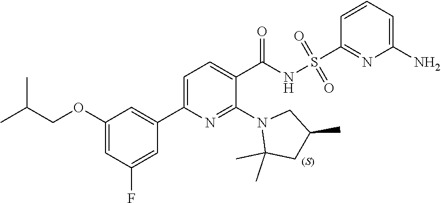

### Step 1: tert-butyl 2-chloro-6-(3-fluoro-5-isobutoxy-phenyl)pyridine-3-carboxylate

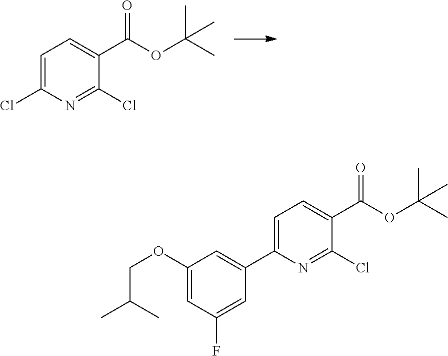 [SMILES: CC(C)(C)OC(=O)c1ccc(Cl)nc1Cl ; CC(C)COc1cc(F)cc(-c2ccc(C(=O)OC(C)(C)C)c(Cl)n2)c1]

tert-Butyl 2,6-dichloropyridine-3-carboxylate (15.0 g, 60.5 mmol) and (3-fluoro-5-isobutoxy-phenyl)boronic acid (13.46 g, 63.48 mmol) were combined and fully dissolved in ethanol (150 mL) and toluene (150 mL). A suspension of sodium carbonate (19.23 g, 181.4 mmol) in water (30 mL) was added. Tetrakis(triphenylphosphine)palladium (0) (2.096 g, 1.814 mmol) was added under nitrogen. The reaction mixture was allowed to stir at 60° C. for 16 hours. Volatiles were removed under reduced pressure. The remaining solids were partitioned between water (100 mL) and ethyl acetate (100 mL). The organic layer was washed with brine (1×100 mL), dried over sodium sulfate, filtered, and concentrated under reduced pressure. The material was subjected silica gel column chromatography on a 330 gram silica gel column, 0 to 20% ethyl acetate in hexanes gradient. The material was repurified on a 220 gram silica gel column, isocratic 100% hexane for 10 minutes, then a 0 to 5% ethyl acetate in hexanes gradient to yield tert-butyl 2-chloro-6-(3-fluoro-5-isobutoxy-phenyl)pyridine-3-carboxylate (18.87 g, 49.68 mmol, 82.2%) as a colorless oil. ¹H NMR (400 MHz, DMSO-d₆) δ 8.24 (d, J=8.0 Hz, 1H), 8.16 (d, J=8.1 Hz, 1H), 7.48 (dd, J=9.4, 2.0 Hz, 2H), 6.99 (dt, J=10.8, 2.2 Hz, 1H), 3.86 (d, J=6.5 Hz, 2H), 2.05 (dt, J=13.3, 6.6 Hz, 1H), 1.57 (d, J=9.3 Hz, 9H), 1.00 (t, J=5.5 Hz, 6H). ESI-MS m/z calc. 379.13504, found 380.2 (M+1)±; Retention time: 2.57 minutes.

### Step 2: 2-chloro-6-(3-fluoro-5-isobutoxy-phenyl)pyridine-3-carboxylic acid

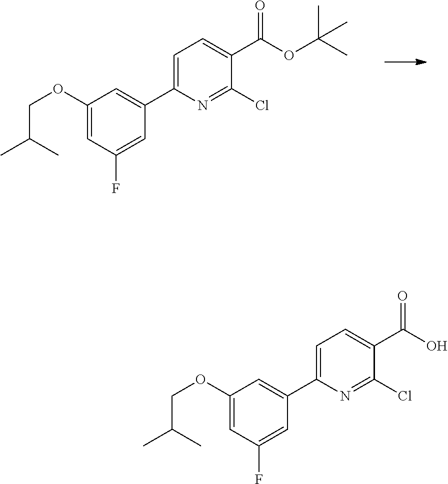

tert-Butyl 2-chloro-6-(3-fluoro-5-isobutoxy-phenyl)pyridine-3-carboxylate (18.57 g, 48.89 mmol) was dissolved in dichloromethane (200 mL). Trifluoroacetic acid (60 mL, 780 mmol) was added and the reaction mixture was allowed to stir at room temperature for 1 hour. The reaction mixture was stirred at 40° C. for 2 hours. The reaction mixture was concentrated under reduced pressure and taken up in ethyl acetate (100 mL). It was washed with a saturated aqueous sodium bicarbonate solution (1×100 mL) and brine (1×100 mL), dried over sodium sulfate, filtered, and concentrated under reduced pressure. The crude product was suspended in ethyl acetate (75 mL) and washed with aqueous HCl (1 N, 1×75 mL). The organic layer was dried over sodium sulfate, filtered and concentrated under reduced pressure. The remaining solid (17.7 g) was stirred as a slurry in dichloromethane (35 mL) at 40° C. for 30 minutes. After cooling to room temperature, the remaining slurry was filtered, and then rinsed with cold dichloromethane to give 2-chloro-6-(3-fluoro-5-isobutoxy-phenyl)pyridine-3-carboxylic acid (11.35 g, 35.06 mmol, 72%) as a white solid. ¹H NMR (400 MHz, DMSO-d₆) δ 13.76 (s, 1H), 8.31 (d, J=8.0 Hz, 1H), 8.17 (d, J=8.1 Hz, 1H), 7.54-7.47 (m, 2H), 7.00 (dt, J=10.8, 2.3 Hz, 1H), 3.87 (d, J=6.5 Hz, 2H), 2.05 (dt, J=13.3, 6.6 Hz, 1H), 1.01 (d, J=6.7 Hz, 6H). ESI-MS m/z calc. 323.1, found 324.1 (M+1)±; Retention time: 1.96 minutes.

### Step 3: N-[(6-amino-2-pyridyl)sulfonyl]-2-chloro-6-(3-fluoro-5-isobutoxy-phenyl)pyridine-3-carboxamide

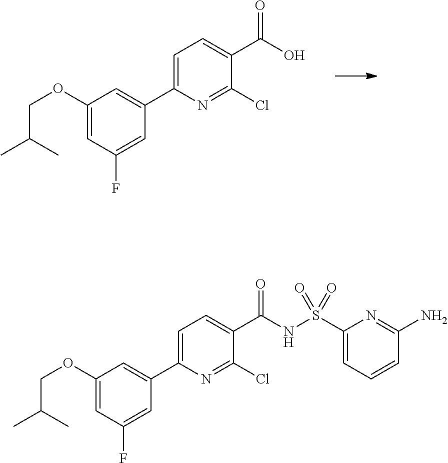 [SMILES: CC(C)COc1cc(F)cc(-c2ccc(C(=O)O)c(Cl)n2)c1 ; CC(C)COc1cc(F)cc(-c2ccc(C(=O)NS(=O)(=O)c3cccc(N)n3)c(Cl)n2)c1]

2-Chloro-6-(3-fluoro-5-isobutoxy-phenyl)pyridine-3-carboxylic acid (3.00 g, 9.27 mmol) was dissolved in N,N-dimethylformamide (30.00 mL), and 1,1′-carbonyldiimidazole (2.254 g, 13.90 mmol) was added to the solution. The solution was allowed to stir at 65° C. for 1 hour. In a separate flask, sodium hydride (444.8 mg, 11.12 mmol) was added to a solution of 6-aminopyridine-2-sulfonamide (1.926 g, 11.12 mmol) in N,N-dimethylformamide (15.00 mL). This mixture was stirred for one hour before being added to the prior reaction mixture. The final reaction mixture was stirred at 65° C. for 15 minutes. Volatiles were removed under reduced pressure. The remaining oil was taken up in ethyl acetate and washed with aqueous HCl (1 N, 1×75 mL) and brine (3×75 mL). The organic layer was dried over sodium sulfate, filtered, and concentrated under reduced pressure. The remaining white solid (4.7 g) was fully dissolved in isopropanol (120 mL) in an 85° C. water bath. The colorless solution was allowed to slowly cool to room temperature with slow stirring over 16 hours. The crystalline solids that had formed were collected by vacuum filtration, and then rinsed with cold isopropanol (50 mL). Upon drying, N-[(6-amino-2-pyridyl)sulfonyl]-2-chloro-6-(3-fluoro-5-isobutoxy-phenyl)pyridine-3-carboxamide (3.24 g, 6.765 mmol, 73%) was obtained as a white solid. ¹H NMR (400 MHz, DMSO-d₆) δ 12.78 (s, 1H), 8.15 (d, J=8.0 Hz, 1H), 8.09 (d, J=7.9 Hz, 1H), 7.73-7.63 (m, 1H), 7.49 (dd, J=8.6, 1.9 Hz, 2H), 7.21 (d, J=7.3 Hz, 1H), 6.99 (dt, J=10.7, 2.2 Hz, 1H), 6.74 (d, J=8.4 Hz, 1H), 6.64 (s, 2H), 3.86 (d, J=6.5 Hz, 2H), 2.05 (dp, J=13.3, 6.5 Hz, 1H), 1.02 (dd, J=12.7, 6.4 Hz, 6H).

### Step 4: N-[(6-amino-2-pyridyl)sulfonyl]-6-(3-fluoro-5-isobutoxy-phenyl)-2-[(4S)-2,2,4-trimethylpyrrolidin-1-yl]pyridine-3-carboxamide (Compound (I)) and N-[(6-amino-2-pyridyl)sulfonyl]-6-(3-fluoro-5-isobutoxy-phenyl)-2-[(4R)-2,2,4-trimethylpyrrolidin-1-yl]pyridine-3-carboxamide

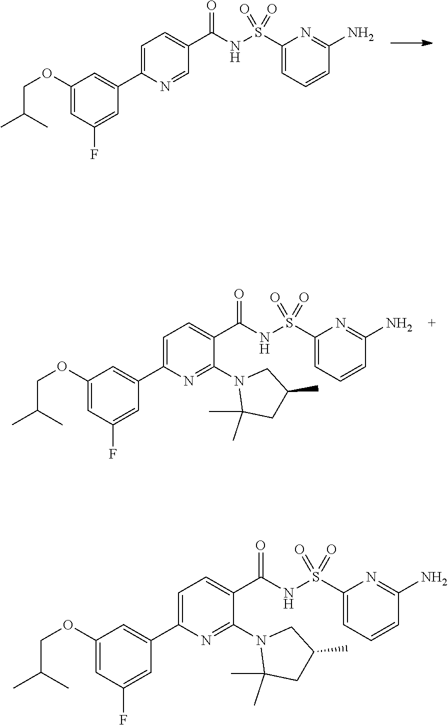 [SMILES: CC(C)COc1cc(F)cc(-c2ccc(C(=O)NS(=O)(=O)c3cccc(N)n3)cn2)c1 ; CC(C)COc1cc(F)cc(-c2ccc(C(=O)NS(=O)(=O)c3cccc(N)n3)c(N3C[C@@H](C)CC3(C)C)n2)c1 ; CC(C)COc1cc(F)cc(-c2ccc(C(=O)NS(=O)(=O)c3cccc(N)n3)c(N3C[C@H](C)CC3(C)C)n2)c1]

N-[(6-Amino-2-pyridyl)sulfonyl]-2-chloro-6-(3-fluoro-5-isobutoxy-phenyl)pyridine-3-carboxamide (309 mg, 0.645 mmol) was dissolved in dimethylsulfoxide (3.708 mL) and potassium carbonate (445.9 mg, 3.226 mmol) was slowly added, followed by 2,2,4-trimethylpyrrolidine (146.0 mg, 1.290 mmol). The reaction mixture was sealed and heated at 150° C. for 72 hours. The reaction was cooled down, diluted with water (50 mL), extracted 3 times with 50 mL portions of ethyl acetate, washed with brine, dried over sodium sulfate, filtered and evaporated to dryness. The crude material was dissolved in 2 mL of dichloromethane and purified by on silica gel using a gradient of 0 to 80% ethyl acetate in hexanes. The stereoisomers were separated using supercritical fluid chromatography on a ChiralPak AD-H (250×4.6 mm), 5 μm column using 25% isopropanol with 1.0% diethylamine in CO₂ at a flow rate of 3.0 mL/min. The separated enationmers were separately concentrated, diluted with ethyl acetate (3 mL) and washed with 1N aqueous hydrochloric acid. The organic layers were dried over sodium sulfate, filtered, and evaporated to dryness to give the pure compounds as pale yellow solids.

The first compound to elute from the SFC conditions given above gave N-[(6-amino-2-pyridyl)sulfonyl]-6-(3-fluoro-5-isobutoxy-phenyl)-2-[(4R)-2,2,4-trimethylpyrrolidin-1-yl]pyridine-3-carboxamide (Hydrochloric Acid)¹H NMR (400 MHz, DMSO-d6) δ 12.47 (s, 1H), 7.78 (d, J=8.0 Hz, 1H), 7.69-7.57 (m, 1H), 7.56-7.46 (m, 1H), 7.41 (dt, J=10.1, 1.8 Hz, 1H), 7.26 (d, J=8.0 Hz, 1H), 7.21 (d, J=7.2 Hz, 1H), 6.89 (dt, J=10.7, 2.3 Hz, 1H), 6.69 (d, J=8.3 Hz, 1H), 3.83 (d, J=6.7 Hz, 2H), 2.61 (dq, J=9.7, 4.9 Hz, 2H), 2.24 (d, J=15.8 Hz, 1H), 2.06 (dq, J=13.3, 6.7 Hz, 1H), 1.93-1.82 (m, 1H), 1.61 (s, 3H), 1.59 (s, 3H), 1.48-1.33 (m, 1H), 1.32-1.20 (m, 2H), 0.99 (d, J=6.6 Hz, 6H), 0.88 (d, J=6.2 Hz, 3H). ESI-MS m/z calc. 555.2, found 556.4 (M+1)±; Retention time: 2.76 minutes.

The second compound to elute from the SFC conditions described above gave N-[(6-amino-2-pyridyl)sulfonyl]-6-(3-fluoro-5-isobutoxy-phenyl)-2-[(4S)-2,2,4-trimethylpyrrolidin-1-yl]pyridine-3-carboxamide (Compound I) (Hydrochloric Acid (1)) ¹H NMR (400 MHz, Chloroform-d) δ 15.49 (s, 1H), 8.49 (d, J=8.2 Hz, 1H), 7.75-7.56 (m, 3H), 7.34 (t, J=1.8 Hz, 1H), 7.30 (dt, J=9.4, 1.9 Hz, 1H), 6.75-6.66 (m, 2H), 3.95 (s, 1H), 3.78 (d, J=6.5 Hz, 2H), 3.42 (s, 1H), 2.88-2.74 (m, 1H), 2.23 (dd, J=12.5, 8.0 Hz, 1H), 2.17-2.08 (m, 1H), 1.98-1.87 (m, 1H), 1.55 (s, 3H), 1.39 (s, 3H), 1.31 (d, J=6.7 Hz, 3H), 1.05 (d, J=6.7 Hz, 6H). ESI-MS m/z calc. 555.2, found 556.4 (M+1)±; Retention time: 2.77 minutes. Absolute stereochemistry was confirmed by X-ray crystallography.

### Example 2: Synthesis of Compound (II): Synthesis of N-(benzenesulfonyl)-6-(3-fluoro-5-isobutoxy-phenyl)-2-[(4S)-2,2,4-trimethyl-pyrrolidin-1-yl]pyridine-3-carboxamide

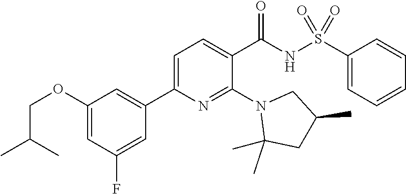 [SMILES: CC(C)COc1cc(F)cc(-c2ccc(C(=O)NS(=O)(=O)c3ccccc3)c(N3C[C@@H](C)CC3(C)C)n2)c1]

### Step 1: Synthesis of tert-Butyl 2,6-dichloropyridine-3-carboxylate

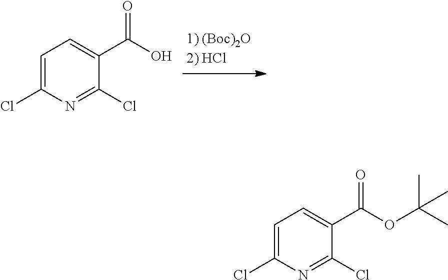

A solution of 2,6-dichloropyridine-3-carboxylic acid (10 g, 52.08 mmol) in THF (210 mL) was treated successively with di-tert-butyl dicarbonate (17 g, 77.89 mmol) and 4-(dimethylamino)pyridine (3.2 g, 26.19 mmol) and stirred overnight at room temperature. At this point, HCl 1N (400 mL) was added, and the mixture was stirred vigorously for 10 minutes. The product was extracted with ethyl acetate (2×300 mL), and the combined organic layers were washed with water (300 mL) and brine (150 mL) and dried over sodium sulfate and concentrated under reduced pressure to give 12.94 g (96% yield) of tert-butyl 2,6-dichloropyridine-3-carboxylate as a colorless oil. ESI-MS m/z calc. 247.02, found 248.1 (M+1)⁺; Retention time: 2.27 minutes. ¹H NMR (300 MHz, CDCl₃) ppm 1.60 (s, 9H), 7.30 (d, J=7.9 Hz, 1H), 8.05 (d, J=8.2 Hz, 1H).

### Step 2: tert-butyl 2-chloro-6-(3-fluoro-5-isobutoxy-phenyl)pyridine-3-carboxylate

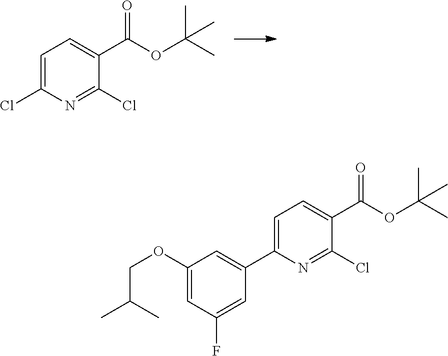 [SMILES: CC(C)(C)OC(=O)c1ccc(Cl)nc1Cl ; CC(C)COc1cc(F)cc(-c2ccc(C(=O)OC(C)(C)C)c(Cl)n2)c1]

tert-Butyl 2,6-dichloropyridine-3-carboxylate (15.0 g, 60.5 mmol) and (3-fluoro-5-isobutoxy-phenyl)boronic acid (13.46 g, 63.48 mmol) were combined and fully dissolved in ethanol (150 mL) and toluene (150 mL). A suspension of sodium carbonate (19.23 g, 181.4 mmol) in water (30 mL) was added. Tetrakis(triphenylphosphine)palladium (0) (2.096 g, 1.814 mmol) was added under nitrogen. The reaction mixture was allowed to stir at 60° C. for 16 hours. Volatiles were removed under reduced pressure. The remaining solids were partitioned between water (100 mL) and ethyl acetate (100 mL). The organic layer was washed with brine (1×100 mL), dried over sodium sulfate, filtered and concentrated under reduced pressure. The material was subjected silica gel column chromatography on a 330 gram silica gel column, 0 to 20% ethyl acetate in hexanes gradient. The material was repurified on a 220 gram silica gel column, isocratic 100% hexane for 10 minutes, then a 0 to 5% ethyl acetate in hexanes gradient to yield tert-butyl 2-chloro-6-(3-fluoro-5-isobutoxy-phenyl)pyridine-3-carboxylate (18.87 g, 49.68 mmol, 82.2%) was obtained as a colorless oil. ¹H NMR (400 MHz, DMSO-d₆) δ 8.24 (d, J=8.0 Hz, 1H), 8.16 (d, J=8.1 Hz, 1H), 7.48 (dd, J=9.4, 2.0 Hz, 2H), 6.99 (dt, J=10.8, 2.2 Hz, 1H), 3.86 (d, J=6.5 Hz, 2H), 2.05 (dt, J=13.3, 6.6 Hz, 1H), 1.57 (d, J=9.3 Hz, 9H), 1.00 (t, J=5.5 Hz, 6H). ESI-MS m/z calc. 379.13504, found 380.2 (M+1)±; Retention time: 2.57 minutes.

### Step 3: 2-chloro-6-(3-fluoro-5-isobutoxy-phenyl)pyridine-3-carboxylic acid

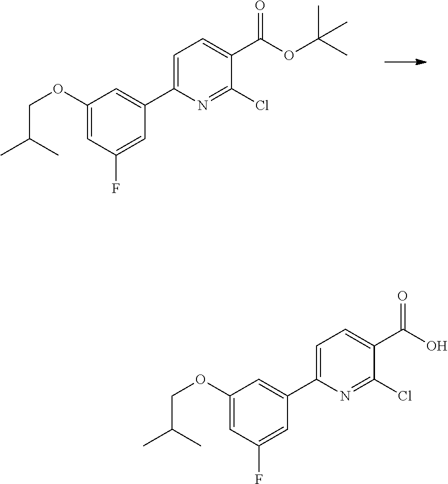

tert-Butyl 2-chloro-6-(3-fluoro-5-isobutoxy-phenyl)pyridine-3-carboxylate (18.57 g, 48.89 mmol) was dissolved in dichloromethane (200 mL). Trifluoroacetic acid (60 mL, 780 mmol) was added and the reaction mixture was allowed to stir at room temperature for 1 hour. The reaction mixture was stirred at 40° C. for 2 hours. The reaction mixture was concentrated under reduced pressure and taken up in ethyl acetate (100 mL). It was washed with a saturated aqueous sodium bicarbonate solution (1×100 mL) and brine (1×100 mL), dried over sodium sulfate, filtered and concentrated under reduced pressure. The crude product was suspended in ethyl acetate (75 mL) and washed with aqueous HCl (1 N, 1×75 mL). The organic layer was dried over sodium sulfate, filtered and concentrated under reduced pressure. The remaining solid (17.7 g) was stirred as a slurry in dichloromethane (35 mL) at 40° C. for 30 minutes. After cooling to room temperature, the remaining slurry was filtered, and then rinsed with cold dichloromethane to give 2-chloro-6-(3-fluoro-5-isobutoxy-phenyl)pyridine-3-carboxylic acid (11.35 g, 35.06 mmol, 72%) as a white solid. ¹H NMR (400 MHz, DMSO-d₆) δ 13.76 (s, 1H), 8.31 (d, J=8.0 Hz, 1H), 8.17 (d, J=8.1 Hz, 1H), 7.54-7.47 (m, 2H), 7.00 (dt, J=10.8, 2.3 Hz, 1H), 3.87 (d, J=6.5 Hz, 2H), 2.05 (dt, J=13.3, 6.6 Hz, 1H), 1.01 (d, J=6.7 Hz, 6H). ESI-MS m/z calc. 323.1, found 324.1 (M+1)⁺; Retention time: 1.96 minutes.

### Step 4: N-(benzenesulfonyl)-2-chloro-6-(3-fluoro-5-isobutoxy-phenyl)pyridine-3-carboxamide

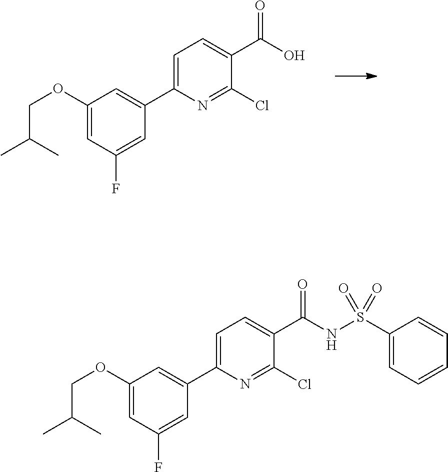 [SMILES: CC(C)COc1cc(F)cc(-c2ccc(C(=O)O)c(Cl)n2)c1 ; CC(C)COc1cc(F)cc(-c2ccc(C(=O)NS(=O)(=O)c3ccccc3)c(Cl)n2)c1]

To a solution of the 2-chloro-6-(3-fluoro-5-isobutoxy-phenyl)pyridine-3-carboxylic acid (10.0 g, 30.89 mmol) in DMF (30.0 mL) at ambient temperature in a round bottom flask was slowly added carbonyldiimidazole (5.510 g, 33.98 mmol) portionwise and the mixture stirred for 100 min. Meanwhile to benzenesulfonamide (6.069 g, 38.61 mmol) in DMF (30.0 mL) (homogenous solution) in another round bottom flask was added NaHMDS in THF (38.61 mL of 1.0 M, 38.61 mmol) portionwise via syringe over 30-45 min and on completion of addition the mixture was stirred a further 30 min. The mixture containing the activated acid was then added to the mixture containing the deprotonated sulfonamide and the combined mixture was stirred 1 h. The reaction was cooled with a 0° C. bath and quenched by addition of 12N aqueous HCl (11.58 mL) in portions over 2-3 minutes resulting in precipitated solids. Transferred the reaction mixture to a separatory funnel and ethyl acetate (100.0 mL) was added. Added 1N aqueous HCl (20.0 mL) giving a pH=2-3 then separated the layers and washed the organic layer with 5:1 water/saturated aqueous brine (120.0 mL), saturated aqueous brine (1×50 mL, 1×30 mL), dried (sodium sulfate), filtered and concentrated under reduced pressure to a clear light yellow oil that was concentrated from isopropanol several more times resulting in precipitation of a solid. The solid was slurried overnight in isopropanol then filtered and washed the solid with heptane (50 mL) and dried in vacuo giving N-(benzenesulfonyl)-2-chloro-6-(3-fluoro-5-isobutoxy-phenyl)pyridine-3-carboxamide (10.22 g, 22.08 mmol, 71.47%) as a white solid. ¹H NMR (400 MHz, DMSO) δ 12.85 (s, 1H), 8.15 (d, J=8.0 Hz, 1H), 8.09 (d, J=8.0 Hz, 1H), 8.02 (dd, J=5.3, 3.3 Hz, 2H), 7.76 (d, J=7.4 Hz, 1H), 7.69 (t, J=7.6 Hz, 2H), 7.51-7.43 (m, 2H), 6.99 (dd, J=10.8, 2.3 Hz, 1H), 3.85 (d, J=6.5 Hz, 2H), 2.04 (dt, J=13.3, 6.6 Hz, 1H), 1.00 (d, J=6.7 Hz, 6H). ESI-MS m/z calc. 462.08163, found 463.19 (M+1)±; Retention time: 2.93 minutes [5 minute method].

### Step 5: N-(benzenesulfonyl)-6-(3-fluoro-5-isobutoxy-phenyl)-2-[(4S)-2,2,4-trimethylpyrrolidin-1-yl]pyridine-3-carboxamide (Compound (II))

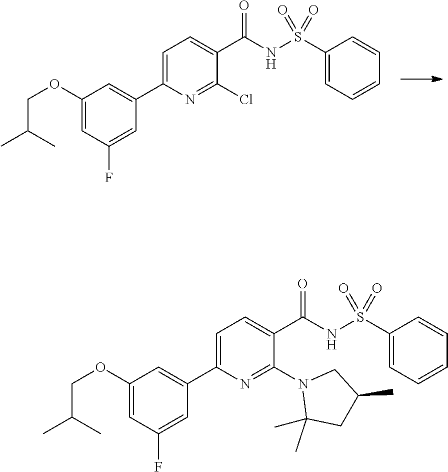 [SMILES: CC(C)COc1cc(F)cc(-c2ccc(C(=O)NS(=O)(=O)c3ccccc3)c(Cl)n2)c1 ; CC(C)COc1cc(F)cc(-c2ccc(C(=O)NS(=O)(=O)c3ccccc3)c(N3C[C@@H](C)CC3(C)C)n2)c1]

To a round bottom flask outfitted with a reflux condenser was added N-(benzenesulfonyl)-2-chloro-6-(3-fluoro-5-isobutoxy-phenyl)pyridine-3-carboxamide (10.0 g, 21.60 mmol) and NMP (40 mL) and stirring was commenced. Warmed to 50° C. and began portionwise addition of potassium carbonate (5.970 g, 43.20 mmol) followed by (4S)-2,2,4-trimethylpyrrolidine (4.890 g, 43.20 mmol) in one portion. After stirring for 10 min, heated the mixture to 125° C. for 65 h, then cooled to 10° C. and added 1N aqueous HCl (50.0 mL, 50.00 mmol) in portions to give pH 1-2 and a precipitated solid. Added ethyl acetate (100.0 mL) to dissolve solid and diluted the aqueous layer with water (50.0 mL) and stirred for 10 min. The mixture was transferred to a separatory funnel and layers were allowed to separate. Added aqueous 1N HCl dropwise until all solids were dissolved. Separated the layers and the aqueous layer was back extracted with ethyl acetate (50.00 mL) followed by combination of the organic layers. To the combined organic layers was added water (50.00 mL) giving an emulsion which was clarified by the addition of 1N aqueous HCl (25.00 mL). Separated the layers then the organic layer was washed with saturated aqueous brine (50.00 mL), dried over Na₂SO₄, filtered through celite and rinsed with ethyl acetate (30.00 mL). The filtrate was concentrated under reduced pressure and the residue was purified by silica gel chromatography using a gradient from 100% hexanes to 50% EtOAc giving a light amber oil which was evaporated from isopropanol several times under reduced pressure providing N-(benzenesulfonyl)-6-(3-fluoro-5-isobutoxy-phenyl)-2-[(4S)-2,2,4-trimethylpyrrolidin-1-yl]pyridine-3-carboxamide (9.73 g, 18.03 mmol, 83.5%) as a yellow solid. ¹H NMR (400 MHz, DMSO-d6) δ 12.57 (s, 1H), 8.16-7.88 (m, 2H), 7.82-7.57 (m, 4H), 7.47 (t, J=1.8 Hz, 1H), 7.40 (dt, J=9.9, 2.0 Hz, 1H), 7.27 (d, J=8.1 Hz, 1H), 6.89 (dt, J=10.8, 2.3 Hz, 1H), 3.83 (d, J=6.6 Hz, 2H), 2.48-2.28 (m, 2H), 2.07 (dtt, J=26.6, 13.4, 6.4 Hz, 2H), 1.83 (dd, J=11.9, 5.5 Hz, 1H), 1.57 (d, J=17.3 Hz, 6H), 1.38 (t, J=12.1 Hz, 1H), 1.04 (d, J=6.1 Hz, 1H), 0.98 (d, J=6.7 Hz, 6H), 0.66 (d, J=6.3 Hz, 3H). ESI-MS m/z calc. 539.2254, found 540.0 (M+1)±; Retention time: 3.25 minutes [5 minute method].

### Example 3: Synthesis of Compound III: (R)-1-(2,2-Difluorobenzo[d]-[1,3]dioxol-5-yl)-N-(1-(2,3-dihydroxypropyl)-6-fluoro-2-(1-hydroxy-2-methyl-propan-2-yl)-1H-indol-5-yl)cyclopropanecarboxamide

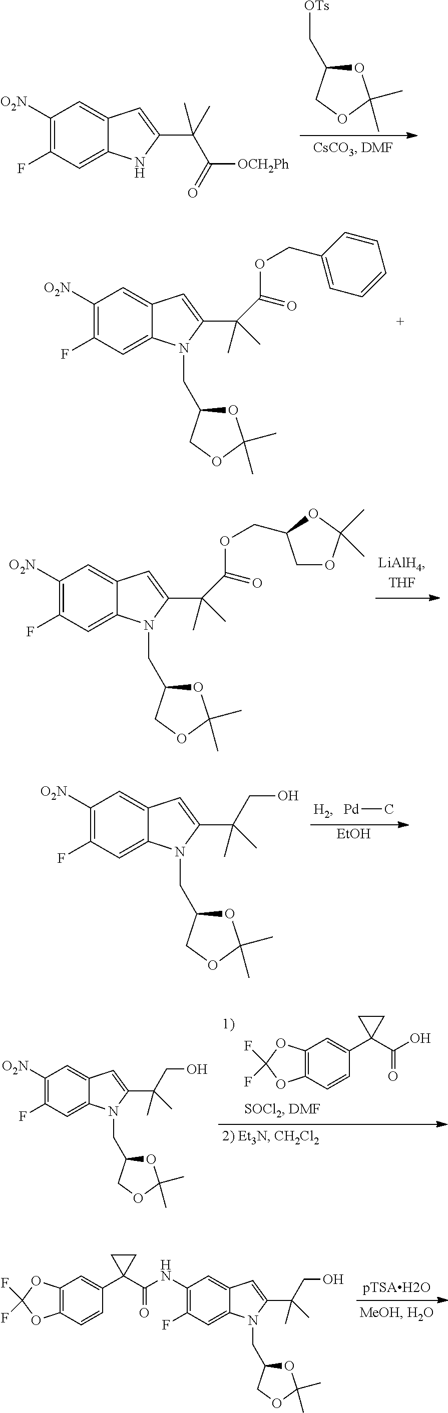

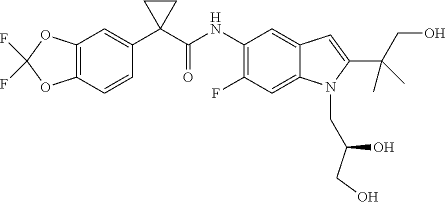 [SMILES: CC(C)(CO)c1cc2cc(NC(=O)C3(c4ccc5c(c4)OC(F)(F)O5)CC3)c(F)cc2n1C[C@@H](O)CO]

### Step 1: (R)-Benzyl 2-(1-((2,2-dimethyl-1,3-dioxolan-4-yl)methyl)-6-fluoro-5-nitro-1H-indol-2-yl)-2-methylpropanoate and ((S)-2,2-Dimethyl-1,3-dioxolan-4-yl)methyl 2-(1-4(R)-2,2-dimethyl-1,3-dioxolan-4-yl)methyl)-6-fluoro-5-nitro-1H-indol-2-yl)-2-methylpropanoate

Cesium carbonate (8.23 g, 25.3 mmol) was added to a mixture of benzyl 2-(6-fluoro-5-nitro-1H-indol-2-yl)-2-methylpropanoate (3.0 g, 8.4 mmol) and (S)-(2,2-dimethyl-1,3-dioxolan-4-yl)methyl 4-methylbenzenesulfonate (7.23 g, 25.3 mmol) in DMF (N,N-dimethylformamide) (17 mL). The reaction was stirred at 80° C. for 46 hours under a nitrogen atmosphere. The mixture was then partitioned between ethyl acetate and water. The aqueous layer was extracted with ethyl acetate. The combined ethyl acetate layers were washed with brine, dried over MgSO₄, filtered and concentrated. The crude product, a viscous brown oil which contains both of the products shown above, was taken directly to the next step without further purification. (R)-Benzyl 2-(1-((2,2-dimethyl-1,3-dioxolan-4-yl)methyl)-6-fluoro-5-nitro-1H-indol-2-yl)-2-methylpropanoate, ESI-MS m/z calc. 470.2, found 471.5 (M+1)±. Retention time 2.20 minutes. ((S)-2,2-Dimethyl-1,3-dioxolan-4-yl)methyl 2-(1-(((R)-2,2-dimethyl-1,3-dioxolan-4-yl)methyl)-6-fluoro-5-nitro-1H-indol-2-yl)-2-methylpropanoate, ESI-MS m/z calc. 494.5, found 495.7 (M+1)±. Retention time 2.01 minutes.

### Step 2: (R)-2-(1-((2,2-dimethyl-1,3-dioxolan-4-yl)methyl)-6-fluoro-5-nitro-1H-indol-2-yl)-2-methylpropan-1-ol

The crude reaction mixture obtained in step (A) was dissolved in THF (tetrahydrofuran) (42 mL) and cooled in an ice-water bath. LiAlH₄ (16.8 mL of 1 M solution, 16.8 mmol) was added drop-wise. After the addition was complete, the mixture was stirred for an additional 5 minutes. The reaction was quenched by adding water (1 mL), 15% NaOH solution (1 mL) and then water (3 mL). The mixture was filtered over Celite, and the solids were washed with THF and ethyl acetate. The filtrate was concentrated and purified by column chromatography (30-60% ethyl acetate-hexanes) to obtain (R)-2-(1-((2,2-dimethyl-1,3-dioxolan-4-yl)methyl)-6-fluoro-5-nitro-1H-indol-2-yl)-2-methylpropan-1-ol as a brown oil (2.68 g, 87% over 2 steps). ESI-MS m/z calc. 366.4, found 367.3 (M+1)±. Retention time 1.68 minutes. ¹H NMR (400 MHz, DMSO-d6) δ 8.34 (d, J=7.6 Hz, 1H), 7.65 (d, J=13.4 Hz, 1H), 6.57 (s, 1H), 4.94 (t, J=5.4 Hz, 1H), 4.64-4.60 (m, 1H), 4.52-4.42 (m, 2H), 4.16-4.14 (m, 1H), 3.76-3.74 (m, 1H), 3.63-3.53 (m, 2H), 1.42 (s, 3H), 1.38-1.36 (m, 6H) and 1.19 (s, 3H) ppm. (DMSO is dimethylsulfoxide).

### Step 3: (R)-2-(5-amino-1-((2,2-dimethyl-1,3-dioxolan-4-yl)methyl)-6-fluoro-1H-indol-2-yl)-2-methylpropan-1-ol

(R)-2-(1-((2,2-dimethyl-1,3-dioxolan-4-yl)methyl)-6-fluoro-5-nitro-1H-indol-2-yl)-2-methylpropan-1-ol (2.5 g, 6.82 mmol) was dissolved in ethanol (70 mL) and the reaction was flushed with N2. Then Pd—C (250 mg, 5% wt) was added. The reaction was flushed with nitrogen again and then stirred under H2 (atm). After 2.5 hours only partial conversion to the product was observed by LCMS. The reaction was filtered through Celite and concentrated. The residue was re-subjected to the conditions above. After 2 hours LCMS indicated complete conversion to product. The reaction mixture was filtered through Celite. The filtrate was concentrated to yield the product (1.82 g, 79%). ESI-MS m/z calc. 336.2, found 337.5 (M+1)±. Retention time 0.86 minutes. ¹H NMR (400 MHz, DMSO-d6) δ 7.17 (d, J=12.6 Hz, 1H), 6.76 (d, J=9.0 Hz, 1H), 6.03 (s, 1H), 4.79-4.76 (m, 1H), 4.46 (s, 2H), 4.37-4.31 (m, 3H), 4.06 (dd, J=6.1, 8.3 Hz, 1H), 3.70-3.67 (m, 1H), 3.55-3.52 (m, 2H), 1.41 (s, 3H), 1.32 (s, 6H) and 1.21 (s, 3H) ppm.

### Step 4: (R)-1-(2,2-difluorobenzo[d][1,3]dioxol-5-yl)-N-(1-((2,2-dimethyl-1,3-dioxolan-4-yl)methyl)-6-fluoro-2-(1-hydroxy-2-methylpropan-2-yl)-1H-indol-5-yl)cyclopropanecarboxamide

DMF (3 drops) was added to a stirring mixture of 1-(2,2-difluorobenzo[d][1,3]dioxol-5-yl)cyclopropanecarboxylic acid (1.87 g, 7.7 mmol) and thionyl chloride (1.30 mL, 17.9 mmol). After 1 hour a clear solution had formed. The solution was concentrated under vacuum and then toluene (3 mL) was added and the mixture was concentrated again. The toluene step was repeated once more and the residue was placed on high vacuum for 10 minutes. The acid chloride was then dissolved in dichloromethane (10 mL) and added to a mixture of (R)-2-(5-amino-1-((2,2-dimethyl-1,3-dioxolan-4-yl)methyl)-6-fluoro-1H-indol-2-yl)-2-methylpropan-1-ol (1.8 g, 5.4 mmol) and triethylamine (2.24 mL, 16.1 mmol) in dichloromethane (45 mL). The reaction was stirred at room temperature for 1 hour. The reaction was washed with 1N HCl solution, saturated NaHCO₃solution and brine, dried over MgSO₄ and concentrated to yield the product (3 g, 100%). ESI-MS m/z calc. 560.6, found 561.7 (M+1)±. Retention time 2.05 minutes. ¹H NMR (400 MHz, DMSO-d6) δ 8.31 (s, 1H), 7.53 (s, 1H), 7.42-7.40 (m, 2H), 7.34-7.30 (m, 3H), 6.24 (s, 1H), 4.51-4.48 (m, 1H), 4.39-4.34 (m, 2H), 4.08 (dd, J=6.0, 8.3 Hz, 1H), 3.69 (t, J=7.6 Hz, 1H), 3.58-3.51 (m, 2H), 1.48-1.45 (m, 2H), 1.39 (s, 3H), 1.34-1.33 (m, 6H), 1.18 (s, 3H) and 1.14-1.12 (m, 2H) ppm

### Step 5: (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide

(R)-1-(2,2-difluorobenzo[d][1,3]dioxol-5-yl)-N-(1-((2,2-dimethyl-1,3-dioxolan-4-yl)methyl)-6-fluoro-2-(1-hydroxy-2-methylpropan-2-yl)-1H-indol-5-yl)cyclopropanecarboxamide (3.0 g, 5.4 mmol) was dissolved in methanol (52 mL). Water (5.2 mL) was added followed by p-TsOH.H₂O (p-toluenesulfonic acid hydrate) (204 mg, 1.1 mmol). The reaction was heated at 80° C. for 45 minutes. The solution was concentrated and then partitioned between ethyl acetate and saturated NaHCO₃solution. The ethyl acetate layer was dried over MgSO₄ and concentrated. The residue was purified by column chromatography (50-100% ethyl acetate-hexanes) to yield the product. (1.3 g, 47%, ee>98% by SFC). ESI-MS m/z calc. 520.5, found 521.7 (M+1)±. Retention time 1.69 minutes. ¹H NMR (400 MHz, DMSO-d6) δ 8.31 (s, 1H), 7.53 (s, 1H), 7.42-7.38 (m, 2H), 7.33-7.30 (m, 2H), 6.22 (s, 1H), 5.01 (d, J=5.2 Hz, 1H), 4.90 (t, J=5.5 Hz, 1H), 4.75 (t, J=5.8 Hz, 1H), 4.40 (dd, J=2.6, 15.1 Hz, 1H), 4.10 (dd, J=8.7, 15.1 Hz, 1H), 3.90 (s, 1H), 3.65-3.54 (m, 2H), 3.48-3.33 (m, 2H), 1.48-1.45 (m, 2H), 1.35 (s, 3H), 1.32 (s, 3H) and 1.14-1.11 (m, 2H) ppm.

### Example 4: Synthesis of Compound IV: N-(2,4-di-tert-butyl-5-hydroxyphenyl)-4-oxo-1,4-dihydroquinoline-3-carboxamide

### Part A: Synthesis of 4-oxo-1,4-dihydroquinoline-3-carboxylic acid

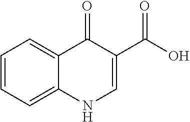 [SMILES: O=C(O)c1c[nH]c2ccccc2c1=O]

### Step 1: 2-Phenylaminomethylene-malonic acid diethyl ester

A mixture of aniline (25.6 g, 0.275 mol) and diethyl 2-(ethoxymethylene)malonate (62.4 g, 0.288 mol) was heated at 140-150° C. for 2 h. The mixture was cooled to room temperature and dried under reduced pressure to afford 2-phenylaminomethylene-malonic acid diethyl ester as a solid, which was used in the next step without further purification. ¹H NMR (DMSO-d₆) δ 11.00 (d, 1H), 8.54 (d, J=13.6 Hz, 1H), 7.36-7.39 (m, 2H), 7.13-7.17 (m, 3H), 4.17-4.33 (m, 4H), 1.18-1.40 (m, 6H).

### Step 2: 4-Hydroxyquinoline-3-carboxylic acid ethyl ester

A 1 L three-necked flask fitted with a mechanical stirrer was charged with 2-phenylaminomethylene-malonic acid diethyl ester (26.3 g, 0.100 mol), polyphosphoric acid (270 g) and phosphoryl chloride (750 g). The mixture was heated to 70° C. and stirred for 4 h. The mixture was cooled to room temperature and filtered. The residue was treated with aqueous Na₂CO₃ solution, filtered, washed with water and dried. 4-Hydroxyquinoline-3-carboxylic acid ethyl ester was obtained as a pale brown solid (15.2 g, 70%). The crude product was used in next step without further purification.

### Step 3: 4-Oxo-1,4-dihydroquinoline-3-carboxylic acid

4-Hydroxyquinoline-3-carboxylic acid ethyl ester (15 g, 69 mmol) was suspended in sodium hydroxide solution (2N, 150 mL) and stirred for 2 h at reflux. After cooling, the mixture was filtered, and the filtrate was acidified to pH 4 with 2N HCl. The resulting precipitate was collected via filtration, washed with water and dried under vacuum to give 4-oxo-1,4-dihydroquinoline-3-carboxylic acid as a pale white solid (10.5 g, 92%). ¹H NMR (DMSO-d₆) δ 15.34 (s, 1H), 13.42 (s, 1H), 8.89 (s, 1H), 8.28 (d, J=8.0 Hz, 1H), 7.88 (m, 1H), 7.81 (d, J=8.4 Hz, 1H), 7.60 (m, 1H).

### Part B: Synthesis of N-(2,4-di-tert-butyl-5-hydroxyphenyl)-4-oxo-1,4-dihydroquinoline-3-carboxamide

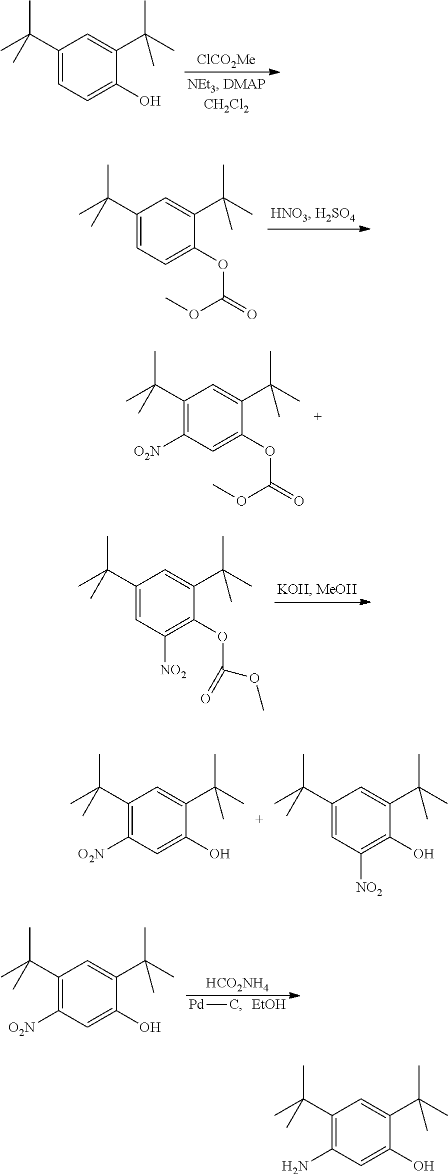

### Step 1: Carbonic acid 2,4-di-tert-butyl-phenyl ester methyl ester

Methyl chloroformate (58 mL, 750 mmol) was added dropwise to a solution of 2,4-di-tert-butyl-phenol (103.2 g, 500 mmol), Et₃N (139 mL, 1000 mmol) and DMAP (3.05 g, 25 mmol) in dichloromethane (400 mL) cooled in an ice-water bath to 0° C. The mixture was allowed to warm to room temperature while stirring overnight, then filtered through silica gel (approx. 1 L) using 10% ethyl acetate-hexanes (˜4 L) as the eluent. The combined filtrates were concentrated to yield carbonic acid 2,4-di-tert-butyl-phenyl ester methyl ester as a yellow oil (132 g, quant.). ¹H NMR (400 MHz, DMSO-d₆) δ 7.35 (d, J=2.4 Hz, 1H), 7.29 (dd, J=8.5, 2.4 Hz, 1H), 7.06 (d, J=8.4 Hz, 1H), 3.85 (s, 3H), 1.30 (s, 9H), 1.29 (s, 9H).

### Step 2: Carbonic acid 2,4-di-tert-butyl-5-nitro-phenyl ester methyl ester and Carbonic acid 2,4-di-tert-butyl-6-nitro-phenyl ester methyl ester

To a stirring mixture of carbonic acid 2,4-di-tert-butyl-phenyl ester methyl ester (4.76 g, 180 mmol) in conc. sulfuric acid (2 mL), cooled in an ice-water bath, was added a cooled mixture of sulfuric acid (2 mL) and nitric acid (2 mL). The addition was done slowly so that the reaction temperature did not exceed 50° C. The reaction was allowed to stir for 2 h while warming to room temperature. The reaction mixture was then added to ice-water and extracted into diethyl ether. The ether layer was dried (MgSO₄), concentrated and purified by column chromatography (0-10% ethyl acetate hexanes) to yield a mixture of carbonic acid 2,4-di-tert-butyl-5-nitro-phenyl ester methyl ester and carbonic acid 2,4-di-tert-butyl-6-nitro-phenyl ester methyl ester as a pale yellow solid (4.28 g), which was used directly in the next step.

### Step 3: 2,4-Di-tert-butyl-5-nitro-phenol and 2,4-Di-tert-butyl-6-nitro-phenol

The mixture of carbonic acid 2,4-di-tert-butyl-5-nitro-phenyl ester methyl ester and carbonic acid 2,4-di-tert-butyl-6-nitro-phenyl ester methyl ester (4.2 g, 14.0 mmol) was dissolved in MeOH (65 mL) before KOH (2.0 g, 36 mmol) was added. The mixture was stirred at room temperature for 2 h. The reaction mixture was then made acidic (pH 2-3) by adding conc. HCl and partitioned between water and diethyl ether. The ether layer was dried (MgSO₄), concentrated and purified by column chromatography (0-5% ethyl acetate-hexanes) to provide 2,4-di-tert-butyl-5-nitro-phenol (1.31 g, 29% over 2 steps) and 2,4-di-tert-butyl-6-nitro-phenol. 2,4-Di-tert-butyl-5-nitro-phenol: ¹H NMR (400 MHz, DMSO-d₆) δ 10.14 (s, 1H, OH), 7.34 (s, 1H), 6.83 (s, 1H), 1.36 (s, 9H), 1.30 (s, 9H). 2,4-Di-tert-butyl-6-nitro-phenol: ¹H NMR (400 MHz, CDCl₃) δ 11.48 (s, 1H), 7.98 (d, J=2.5 Hz, 1H), 7.66 (d, J=2.4 Hz, 1H), 1.47 (s, 9H), 1.34 (s, 9H).

### Step 4: 5-Amino-2,4-di-tert-butyl-phenol

To a refluxing solution of 2,4-di-tert-butyl-5-nitro-phenol (1.86 g, 7.40 mmol) and ammonium formate (1.86 g) in ethanol (75 mL) was added Pd-5% wt. on activated carbon (900 mg). The reaction mixture was stirred at reflux for 2 h, cooled to room temperature and filtered through Celite. The Celite was washed with methanol and the combined filtrates were concentrated to yield 5-amino-2,4-di-tert-butyl-phenol as a grey solid (1.66 g, quant.). ¹H NMR (400 MHz, DMSO-d₆) δ 8.64 (s, 1H, OH), 6.84 (s, 1H), 6.08 (s, 1H), 4.39 (s, 2H, NH₂), 1.27 (m, 18H); HPLC ret. time 2.72 min, 10-99% CH₃CN, 5 min run; ESI-MS 222.4 m/z [M+H]⁺.

### Step 5: N-(5-hydroxy-2,4-di-tert-butyl-phenyl)-4-oxo-1H-quinoline-3-carboxamide

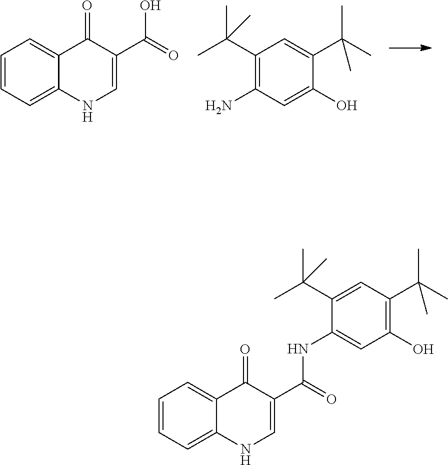

To a suspension of 4-oxo-1,4-dihydroquinolin-3-carboxylic acid (35.5 g, 188 mmol) and HBTU (85.7 g, 226 mmol) in DMF (280 mL) was added Et₃N (63.0 mL, 451 mmol) at ambient temperature. The mixture became homogeneous and was allowed to stir for 10 min before 5-amino-2,4-di-tert-butyl-phenol (50.0 g, 226 mmol) was added in small portions. The mixture was allowed to stir overnight at ambient temperature. The mixture became heterogeneous over the course of the reaction. After all of the acid was consumed (LC-MS analysis, MH+ 190, 1.71 min), the solvent was removed in vacuo. EtOH (ethyl alcohol) was added to the orange solid material to produce a slurry. The mixture was stirred on a rotovap (bath temperature 65° C.) for 15 min without placing the system under vacuum. The mixture was filtered and the captured solid was washed with hexanes to provide a white solid that was the EtOH crystalate. Et₂O (diethyl ether) was added to the solid obtained above until a slurry was formed. The mixture was stirred on a rotovapor (bath temperature 25° C.) for 15 min without placing the system under vacuum. The mixture was filtered and the solid captured. This procedure was performed a total of five times. The solid obtained after the fifth precipitation was placed under vacuum overnight to provide N-(5-hydroxy-2,4-di-tert-butyl-phenyl)-4-oxo-1H-quinoline-3-carboxamide (38 g, 52%). HPLC ret. time 3.45 min, 10-99% CH₃CN, 5 min run; ¹H NMR (400 MHz, DMSO-d₆) δ 12.88 (s, 1H), 11.83 (s, 1H), 9.20 (s, 1H), 8.87 (s, 1H), 8.33 (dd, J=8.2, 1.0 Hz, 1H), 7.83-7.79 (m, 1H), 7.76 (d, J=7.7 Hz, 1H), 7.54-7.50 (m, 1H), 7.17 (s, 1H), 7.10 (s, 1H), 1.38 (s, 9H), 1.37 (s, 9H); ESI-MS m/z calc'd 392.21; found 393.3 [M+H]⁺.

### Example 5: Synthesis of Compound V: 3-(6-(1-(2,2-difluorobenzo[d]-[1,3]dioxol-5-yl) cyclopropanecarboxamido)-3-methylpyridin-2-yl)benzoic acid

Compound V may be prepared by coupling an acid chloride moiety with an amine moiety according to Schemes IV-A through IV-D.

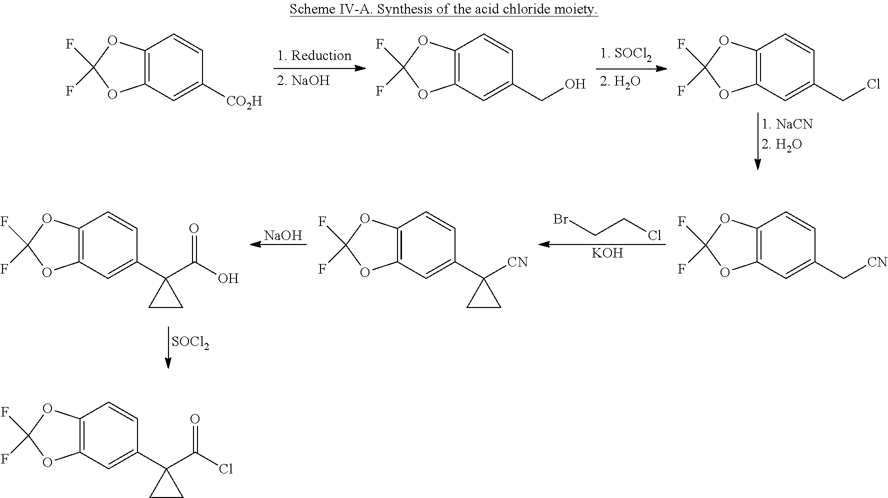

Scheme IV-A depicts the preparation of 1-(2,2-difluorobenzo[d][1,3]dioxol-5-yl)cyclopropanecarbonyl chloride, which is used in Scheme IV-C to make the amide linkage of Compound V.

The starting material, 2,2-difluorobenzo[d][1,3]dioxole-5-carboxylic acid, is commercially available from Saltigo (an affiliate of the Lanxess Corporation). Reduction of the carboxylic acid moiety in 2,2-difluorobenzo[d][1,3]dioxole-5-carboxylic acid to the primary alcohol, followed by conversion to the corresponding chloride using thionyl chloride (SOCl₂), provides 5-(chloromethyl)-2,2-difluorobenzo[d][1,3]dioxole, which is subsequently converted to 2-(2,2-difluorobenzo[d][1,3]dioxol-5-yl)acetonitrile using sodium cyanide. Treatment of 2-(2,2-difluorobenzo[d][1,3]dioxol-5-yl)acetonitrile with base and 1-bromo-2-chloroethane provides 1-(2,2-difluorobenzo[d][1,3]dioxol-5-yl)cyclopropanecarbonitrile. The nitrile moiety in 1-(2,2-difluorobenzo[d][1,3]dioxol-5-yl)cyclopropanecarbonitrile is converted to a carboxylic acid using base to give 1-(2,2-difluorobenzo[d][1,3]dioxol-5-yl)cyclopropanecarboxylic acid, which is converted to the desired acid chloride using thionyl chloride.

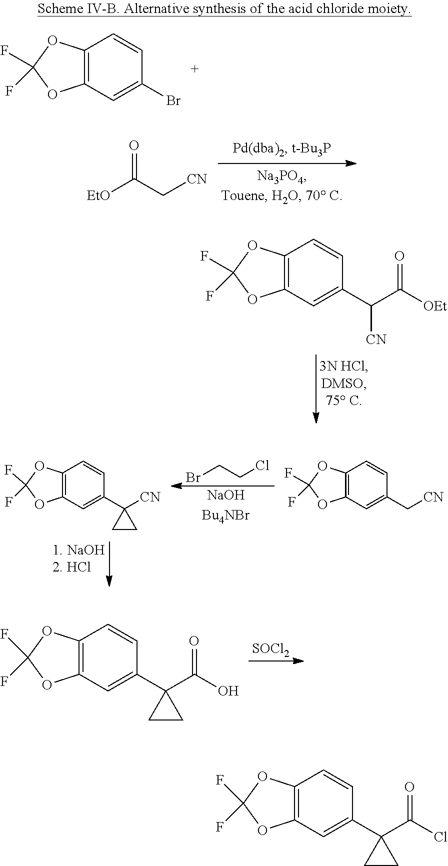

Scheme IV-B depicts an alternative synthesis of the requisite acid chloride. 5-bromomethyl-2,2-difluoro-1,3-benzodioxole is coupled with ethyl cyanoacetate in the presence of a palladium catalyst to form the corresponding alpha cyano ethyl ester. Saponification of the ester moiety to the carboxylic acid gives the cyanoethyl Compound V. Alkylation of the cyanoethyl compound with 1-bromo-2-chloro ethane in the presence of base gives the cyanocyclopropyl compound. Treatment of the cyanocyclopropyl compound with base gives the carboxylate salt, which is converted to the carboxylic acid by treatment with acid. Conversion of the carboxylic acid to the acid chloride is then accomplished using a chlorinating agent such as thionyl chloride or the like.

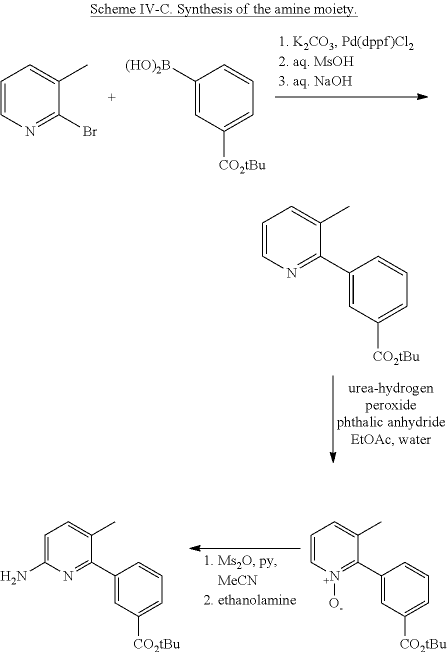

Scheme IV-C depicts the preparation of the requisite tert-butyl 3-(6-amino-3-methylpyridin-2-yl)benzoate, which is coupled with 1-(2,2-difluorobenzo[d][1,3]dioxol-5-yl)cyclopropanecarbonyl chloride in Scheme V-C to give Compound V. Palladium-catalyzed coupling of 2-bromo-3-methylpyridine with 3-(tert-butoxycarbonyl)phenyl-boronic acid gives tert-butyl 3-(3-methylpyridin-2-yl)benzoate, which is subsequently converted to the desired compound.

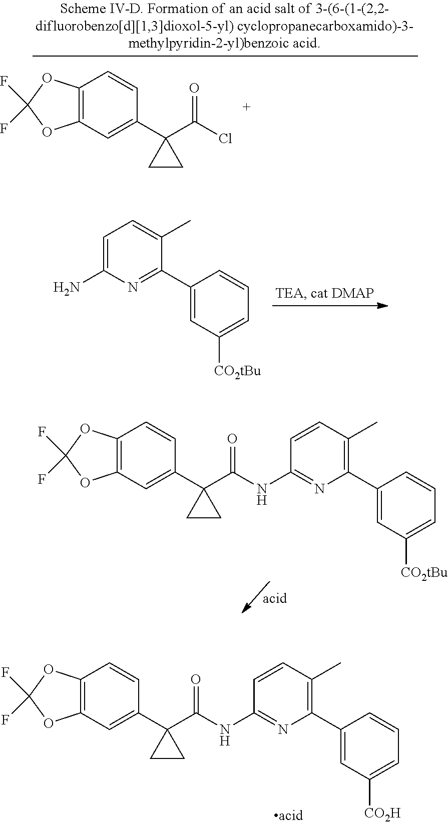 [SMILES: O=C(Cl)C1(c2ccc3c(c2)OC(F)(F)O3)CC1 ; Cc1ccc(N)nc1-c1cccc(C(=O)OC(C)(C)C)c1 ; Cc1ccc(NC(=O)C2(c3ccc4c(c3)OC(F)(F)O4)CC2)nc1-c1cccc(C(=O)OC(C)(C)C)c1 ; Cc1ccc(NC(=O)C2(c3ccc4c(c3)OC(F)(F)O4)CC2)nc1-c1cccc(C(=O)O)c1]

Scheme IV-D depicts the coupling of 1-(2,2-difluorobenzo[d][1,3]dioxol-5-yl)cyclopropanecarbonyl chloride with tert-butyl 3-(6-amino-3-methylpyridin-2-yl)benzoate using triethyl amine and 4-dimethylaminopyridine to initially provide the tert-butyl ester of Compound V.

### Example 6: Preparation of Solid Forms of Compound (I)

### Preparation of Crystalline Form a of Compound (I)

A mixture of Compound (I) and ethanol was heated. The resulting mixture was cooled to a temperature at which a seed of crystalline Form A of Compound (I) was added. Subsequently, water was added while maintaining the same temperature. The resulting mixture was then cooled and the resulting slurry was filtered and the collected solids are washed and dried to afford the title compound.

An X-ray powder diffractogram of crystalline Form A of Compound (I) is shown in FIG. 1.

XRPD, C¹³ solid state NMR, and F¹⁹ solid state NMR data for Form A of Compound (I) are in Tables 1, 2, and 3.

TABLE 1
XRPD data for crystalline
Form A of Compound (I).

| Position | Intensity |
|---|---|
| [°2 Theta] | (%) |
| 4.3 | 100.0 |
| 17.1 | 46.2 |
| 24.2 | 25.7 |
| 28.1 | 17.9 |
| 20.5 | 14.6 |
| 12.8 | 12.8 |

TABLE 2
C¹³ solid state NMR data for crystalline
Form A of Compound (I).

| Chemical Shift | Intensity |
|---|---|
| [ppm] | [relative] |
| 165.2 | 31.6 |
| 164.7 | 24.3 |
| 162.8 | 47.3 |
| 160.8 | 55.8 |
| 159.9 | 16.2 |
| 156.9 | 14.1 |
| 155.8 | 14.1 |
| 155.1 | 22.9 |
| 153.0 | 25.8 |
| 152.2 | 15.9 |
| 141.7 | 18.0 |
| 140.8 | 52.7 |
| 140.2 | 33.5 |
| 139.3 | 28.4 |
| 116.6 | 17.5 |
| 116.2 | 32.1 |
| 115.4 | 32.2 |
| 114.1 | 16.9 |
| 113.4 | 17.2 |
| 109.4 | 64.6 |
| 106.6 | 57.6 |
| 106.4 | 50.9 |
| 105.7 | 63.1 |
| 74.8 | 28.2 |
| 74.4 | 39.4 |
| 65.8 | 20.7 |
| 65.0 | 38.9 |
| 59.7 | 15.6 |
| 58.3 | 14.8 |
| 57.0 | 15.6 |
| 51.7 | 28.8 |
| 50.6 | 18.4 |
| 50.1 | 20.5 |
| 31.1 | 54.0 |
| 28.8 | 100.0 |
| 27.3 | 26.3 |
| 27.0 | 23.8 |
| 25.9 | 42.7 |
| 24.5 | 22.7 |
| 22.4 | 23.5 |
| 19.9 | 51.6 |
| 19.4 | 31.5 |
| 19.0 | 39.1 |
| 18.2 | 72.2 |
| 17.5 | 25.5 |
| 16.2 | 24.1 |

TABLE 3
F¹⁹ solid state NMR data for crystalline
Form A of Compound (I).

| Chemical Shift | Intensity |
|---|---|
| [ppm] | [relative] |
| −110.8 | 11.6 |
| −111.9 | 12.5 |

FIG. 2 shows a DSC trace for crystalline Form A of Compound (I). FIG. 3 shows a TGA plot for crystalline Form A of Compound (I).

FIG. 4 shows the crystal structure of crystalline Form A of Compound (I). Single crystal structure data for Form A of Compound (I) is in Table 4.

TABLE 4
Single crystal structure data for crystalline
Form A of Compound (I).

| Empirical formula | C28 H34 F N5 O4 S | |
|---|---|---|
| Molecular formula | C28 H34 F N5 O4 S | |
| Formula weight | 555.66 | |
| Temperature | 100.0K | |
| Wavelength | 0.71073 Å | |
| Crystal system | Triclinic | |
| Space group | P1 | |
| Unit cell dimensions | a = 8.6335(12) Å | α = 109.618(4)°. |
| | b = 15.4405(19) Å | β = 94.608(4)°. |
| | c = 21.977(3) Å | γ = 91.419(4)°. |
| Volume | 2746.4(6) Å³ | |

| | |
|---|---|
| Z/Z′ | 1/4 |
| Density (calculated) | 1.344 Mg/m³ |

### Preparation of Crystalline Form B of Compound (I)

To crude Compound (I), obtained as a foam after being concentrated from an isopropyl acetate solution, was added isopropyl acetate (3 volumes), resulting in a solution. N-heptane (5 volumes) was added to the solution and stirred, which eventually resulted in a slurry. The resulting mixture was filtered and rinsed with a mixture of isopropyl acetate and hexanes. The collected solids were dried to afford crystalline Form B of Compound (I).

An X-ray powder diffractogram of crystalline Form B of Compound (I) is shown in FIG. 5.

XRPD, C¹³ solid state NMR, and F¹⁹ solid state NMR data for Form B of Compound (I) are in Tables 5, 6, and 7.

TABLE 5
XRPD data for crystalline
Form B of Compound (I)

| Position | Intensity |
|---|---|
| [°2 Theta] | (%) |
| 16.9 | 100.0 |
| 19.8 | 93.9 |
| 12.4 | 63.9 |
| 26.1 | 57.3 |
| 23.1 | 53.3 |
| 17.5 | 52.3 |
| 23.9 | 31.0 |
| 28.3 | 30.0 |
| 14.5 | 26.3 |
| 11.7 | 26.0 |
| 24.4 | 25.4 |
| 12.8 | 25.2 |
| 15.8 | 21.4 |
| 20.4 | 19.9 |
| 16.4 | 19.4 |
| 14.1 | 18.4 |
| 21.9 | 17.8 |
| 22.4 | 17.4 |
| 11.2 | 16.8 |
| 32.5 | 14.9 |
| 15.1 | 14.7 |

TABLE 6
C¹³ solid state NMR data for crystalline
Form B of Compound (I)

| Chemical Shift | Intensity |
|---|---|
| [ppm] | [relative] |
| 164.6 | 15.5 |
| 163.7 | 16.7 |
| 162.9 | 27.4 |
| 160.9 | 50.9 |
| 158.3 | 12.9 |
| 155.7 | 20.7 |
| 154.6 | 23.9 |
| 153.7 | 22.1 |
| 152.8 | 35.1 |
| 141.2 | 27.2 |
| 140.4 | 34.5 |
| 139.2 | 25.8 |
| 137.7 | 25.6 |
| 117.6 | 46.2 |
| 116.9 | 30.5 |
| 109.8 | 33.2 |
| 109 | 26.1 |
| 106.4 | 50.7 |
| 105.9 | 100 |
| 74.1 | 60.8 |
| 65 | 39.7 |
| 58.6 | 22.4 |
| 58.2 | 25.1 |
| 50.7 | 36.8 |
| 31.2 | 44 |
| 30.5 | 41.8 |
| 29.2 | 73.8 |
| 27.6 | 28.8 |
| 26.2 | 25.2 |
| 25.7 | 82.3 |
| 22 | 15.9 |
| 19.2 | 63.3 |
| 18.5 | 55.7 |

TABLE 7
F¹⁹ solid state NMR data for crystalline
Form B of Compound (I)

| Chemical Shift | Intensity |
|---|---|
| [ppm] | [relative] |
| −111.7 | 12.5 |
| −112.1 | 11.4 |

FIG. 6 shows a DSC trace for crystalline Form B of Compound (I). FIG. 7 shows a TGA plot for crystalline Form B of Compound (I).

### Preparation of Crystalline Form H of Compound (I)

Amorphous Compound (I) (50 mg) was added into a vial, along with methanol (1 mL). The resulting mixture was vortexed until all solids were dissolved. Deionized water (1.47 mL) was added to the mixture and the resulting mixture was stirred overnight without exposure to light. The resulting solids were collected to afford crystalline Form H of Compound (I), a hydrate of Compound (I).

An X-ray powder diffractogram of crystalline Form H of Compound (I) is shown in FIG. 8.

XRPD, C¹³ solid state NMR, and F¹⁹ solid state NMR data for Form H of Compound (I) are in Tables 8, 9, and 10.

TABLE 8
XRPD data for crystalline
Form H of Compound (I)

| Position | Intensity |
|---|---|
| [°2 Theta] | (%) |
| 13.2 | 100.0 |
| 17.8 | 56.5 |
| 16.8 | 46.0 |
| 19.2 | 41.1 |
| 28.5 | 27.0 |
| 14.8 | 25.3 |
| 15.5 | 25.2 |
| 25.1 | 22.7 |
| 17.2 | 22.1 |
| 22.1 | 20.0 |
| 23.3 | 18.6 |
| 21.5 | 14.9 |
| 25.8 | 13.5 |
| 7.4 | 10.1 |
| 21.0 | 9.8 |
| 18.5 | 8.9 |
| 20.0 | 8.3 |
| 22.5 | 7.5 |
| 26.9 | 7.2 |
| 16.1 | 6.8 |
| 23.9 | 6.8 |
| 26.5 | 6.6 |
| 30.5 | 5.4 |
| 11.1 | 4.7 |

TABLE 9
C¹³ solid state NMR data for crystalline
Form H of of Compound (I)

| Chemical Shift | Intensity |
|---|---|
| [ppm] | [relative] |
| 166.5 | 22.6 |
| 164.8 | 21.9 |
| 163.6 | 24.5 |
| 161.1 | 100 |
| 155.1 | 25.3 |
| 154.4 | 26.9 |
| 152.3 | 39.9 |
| 141.5 | 60.6 |
| 139.9 | 29.7 |
| 138.7 | 32 |
| 137.9 | 44.9 |
| 117.2 | 45 |
| 114 | 37.3 |
| 109.6 | 45.1 |
| 107.1 | 71.2 |
| 105.5 | 53.6 |
| 104.9 | 77.9 |
| 73.2 | 43.6 |
| 72.7 | 45.7 |
| 66.8 | 31 |
| 65 | 30.4 |
| 60 | 24.6 |
| 56.8 | 24 |
| 53.2 | 35.2 |
| 49.4 | 30.2 |
| 31.3 | 43.5 |
| 30.4 | 49.1 |
| 29.2 | 77.6 |
| 26.6 | 48 |
| 24.9 | 55.7 |
| 24.4 | 48.8 |
| 19.9 | 44.8 |
| 19.3 | 52.3 |
| 18.9 | 41.8 |
| 16.9 | 66.4 |

TABLE 10
F¹⁹ solid state NMR data of crystalline Form H of of Compound (I)

| Chemical Shift | Intensity |
|---|---|
| [ppm] | [relative] |
| −114.3 | 12.5 |

FIG. 9 shows a DSC trace for crystalline Form H of Compound (I). FIG. 10 shows a TGA plot for crystalline Form H of Compound (I).

FIG. 11 shows the crystal structure of crystalline Form H of Compound (I).

Single crystal structure data for crystalline Form H of Compound (I) is in Table 11.

TABLE 11
Single crystal structure data for
crystalline Form H of Compound (I).

| | | |
|---|---|---|
| Empirical formula | C28 H36 F N5 O5 S | |
| Formula weight | 573.68 | |
| Temperature | 100.0 K | |
| Wavelength | 0.71073 Å | |
| Crystal system | Monoclinic | |
| Space group | C 1 2 1 | |
| Unit cell dimensions | a = 48.396(6) Å | α = 90°. |
| | b = 9.0743(10) Å | β = 94.034(3)°. |
| | c = 13.3215(14) Å | γ = 90°. |
| Volume | 5835.7(11) Å³ | |
| Z/Z′ | 4/2 | |
| Density (calculated) | 1.306 Mg/m³ | |

### Preparation of Crystalline Form S of Compound (I)

Amorphous Compound (I) (about 400 mg) was added into a vial, along with 1,4-dioxane (about 500 μL), and stirred at room temperature. The resulting mixture was subsequently cooled to 5° C. and stirred overnight. The following day, heptane (about 700-800 μL) was added and then the mixture was stirred overnight. The resulting mixture was transferred to a centrifuge tube and centrifuged at 14,000 R.P.M. for 5 minutes. The resulting solids were collected to afford crystalline Form S of Compound (I), a dioxane/heptane solvate of Compound (I).

An X-ray powder diffractogram of crystalline Form S of Compound (I) is shown in FIG. 12.

XRPD data for crystalline Form S of Compound (I) are in Table 12.

TABLE 12
XRPD data for crystalline Form S of Compound (I)

| Position | Intensity |
|---|---|
| [°2Theta] | (%) |
| 17.0 | 100.0 |
| 18.8 | 84.9 |
| 21.2 | 56.3 |
| 14.9 | 27.0 |
| 11.5 | 22.9 |
| 22.6 | 20.5 |
| 26.4 | 19.5 |
| 25.3 | 18.9 |
| 16.6 | 17.1 |
| 20.5 | 16.8 |
| 6.1 | 16.5 |
| 17.9 | 16.2 |
| 24.9 | 15.5 |
| 23.8 | 14.8 |
| 12.8 | 14.7 |
| 22.1 | 13.5 |
| 19.6 | 13.1 |
| 23.4 | 12.9 |
| 15.8 | 12.6 |
| 12.0 | 12.6 |
| 29.2 | 12.5 |
| 7.6 | 12.5 |
| 10.6 | 10.9 |

FIG. 13 shows a DSC trace for crystalline Form S of Compound (I). FIG. 14 shows a TGA plot for crystalline Form S of Compound (I).

### Preparation of Crystalline Form MS of Compound (I)

To a round bottom flask was added Compound (I) (10 g) and a pre-made mixture of 9/1 (w/w) dichloromethane/methanol. The resulting mixture was then concentrated in vacuo. Subsequently, the round bottom flask was placed in a vacuum oven at 23° C. for two days. The resulting solids were collected to afford crystalline Form MS of Compound (I), a methanol solvate of Compound (I).

An X-ray powder diffractogram of crystalline Form MS of Compound (I) is shown in FIG. 15.

XRPD data for crystalline Form MS of Compound (I) are in Table 13.

TABLE 13
XRPD data for crystalline Form MS of Compound (I)

| Position | Intensity |
|---|---|
| [°2Theta] | (%) |
| 13.9 | 100.0 |
| 18.8 | 87.2 |
| 25.1 | 78.5 |
| 23.1 | 78.1 |
| 21.0 | 63.3 |
| 17.0 | 59.2 |
| 21.8 | 58.3 |
| 25.7 | 48.5 |
| 27.7 | 41.0 |
| 28.7 | 39.3 |
| 16.5 | 38.1 |
| 15.6 | 35.8 |
| 10.7 | 35.2 |
| 26.5 | 28.8 |
| 32.6 | 28.7 |
| 31.3 | 19.9 |
| 30.7 | 18.6 |
| 11.3 | 14.1 |

FIG. 16 shows a DSC trace for crystalline Form MS of Compound (I). FIG. 17 shows a TGA plot for crystalline Form MS of Compound (I).

### Example 7: Preparation of Solid Forms of Compound (II)

### Preparation of Crystalline Form A2 of Compound (II)

Preparation 1: A mixture of iso-propanol solvate of Compound (II) and n-heptane (12.5 volumes) was stirred at 20-25° C. To this mixture was added ethanol (200 proof, 0.2 volumes), followed by a crystalline Form A2 of Compound (II) seed crystal (0.5 wt. %). The resulting mixture was stirred at 20-25° C. for more than 16 hours before being filtered. The collected solids were washed with n-heptane (2 volumes). The resulting wetcake was dried in a vacuum oven at 45° C. to afford crystalline Form A2 of Compound (II).

Preparation 2: Compound (II) was combined with ethanol (200 proof, about 9.1 volumes) and heated to 55-65° C. To the resulting mixture was added distilled water (1.4 volumes) over at least 30 minutes. The resulting mixture was then cooled to 43-48° C., after which a crystalline Form A2 of Compound (II) seed crystal (0.1 wt. %) was added, and stirring continued for 30 minutes. The resulting mixture was cooled to 20° C. over the course of 15 hours, after which distilled water (1.4 volumes) was added over the course of 2 hours, and then stirred for at least 1 hour. The resulting slurry was filtered and the collected solids were washed with a mixture of ethanol (1.5 volumes) and water (0.5 volumes). The collected solids were dried in a vacuum oven at 45° C. to afford crystalline Form A2 of Compound (II).

An X-ray powder diffractogram of crystalline Form A2 of Compound (II) is shown in FIG. 18.

XRPD, C¹³ solid state NMR, and F¹⁹ solid state NMR data for Form A2 of Compound (II) are in Tables 14, 15, and 16.

TABLE 14
XRPD data for crystalline Form A2 of Compound (II)

| Position | Intensity |
|---|---|
| [°2Theta] | (%) |
| 9.0 | 100.0 |
| 19.3 | 66.0 |
| 20.4 | 49.6 |
| 20.0 | 48.2 |
| 13.8 | 41.3 |
| 15.7 | 38.7 |
| 11.2 | 33.8 |
| 24.9 | 32.9 |
| 15.3 | 32.7 |
| 23.7 | 31.7 |
| 27.8 | 26.9 |
| 8.4 | 25.6 |
| 22.4 | 22.3 |
| 18.8 | 20.9 |
| 10.4 | 20.2 |
| 5.2 | 19.1 |
| 20.9 | 18.2 |
| 26.9 | 17.9 |
| 14.1 | 14.1 |
| 26.4 | 13.3 |
| 31.6 | 11.9 |
| 21.9 | 11.1 |

TABLE 15
C¹³ solid state NMR data for crystalline
Form A2 of Compound (II)

| Chemical Shift | Intensity |
|---|---|
| [ppm] | [relative] |
| 169.2 | 31.5 |
| 165.2 | 6.1 |
| 162.8 | 7.1 |
| 160.9 | 60.5 |
| 154.7 | 53.3 |
| 140.0 | 54.7 |
| 139.5 | 100.0 |
| 134.1 | 37.2 |
| 128.8 | 82.7 |
| 111.4 | 43.7 |
| 108.5 | 27.6 |
| 105.6 | 87.5 |
| 103.8 | 42.8 |
| 75.0 | 69.6 |
| 64.6 | 50.3 |
| 58.9 | 35.9 |
| 51.0 | 48.0 |
| 31.2 | 76.7 |
| 28.6 | 66.7 |
| 26.1 | 82.7 |
| 25.3 | 70.4 |
| 20.2 | 72.3 |
| 19.4 | 83.0 |
| 16.4 | 74.0 |
| 15.3 | 6.6 |

TABLE 16
F¹⁹ solid state NMR data of crystalline Form A2 of Compound (II)

| Chemical Shift | Intensity |
|---|---|
| [ppm] | [relative] |
| −117.5 | 12.5 |

FIG. 19 shows a DSC trace for crystalline Form A2 of Compound (II). FIG. 20 shows a TGA plot for crystalline Form A2 of Compound (II).

FIG. 21 shows the crystal structure of crystalline Form A2 of Compound (II).

Single crystal structure data for crystalline Form A2 of Compound (II) is in Table 17.

TABLE 17
Single crystal structure data for crystalline
Form A2 of Compound (II).

| | | |
|---|---|---|
| Empirical formula | C29 H34 F N3 O4 S | |
| Molecular formula | C29 H34 F N3 O4 S | |
| Formula weight | 539.65 | |
| Temperature | 100.0 K | |
| Wavelength | 0.71073 Å | |
| Crystal system | Hexagonal | |
| Space group | P6₁ | |
| Unit cell dimensions | a = 19.4681(7) Å | α = 90°. |
| | b = 19.4681(7) Å | β = 90°. |
| | c = 13.3151(5) Å | γ = 120°. |
| Volume | 4370.4(4) Å³ | |
| Z/Z′ | 6/1 | |
| Density (calculated) | 1.230 Mg/m³ | |

The infrared absorption spectrum of a sample of crystalline Form A2 of Compound (II) was measured on a Vertex70 FTIR/Raman spectrometer with RAMII Raman Module and Pik MiRacle ZnSe ATR attachment with a spectral range of 370-4000 cm⁻¹, a spectral resolution of 4 cm⁻¹, and 16 scans per spectrum. The sample exhibited absorption signals (wavenumber/frequency) at 1044, 1134, 1245, 1331, 1443, 1548, 1577, 1610, 1650, 2865, 2957, and 3068 cm⁻¹.

### Preparation of Crystalline Form IP of Compound (II)

A mixture of Compound (II) and iso-propanol (about 7 volumes) was heated to 60-70° C. and then cooled to 40-45° C., and seeded with a crystalline Form IP of an iso-propanol solvate of Compound (II) seed crystal (0.4 wt.-%). The resulting mixture was maintained at 45° C. for about 1 hour, followed by the addition of distilled water (2.3 volumes) over a four hour period. The resulting mixture was cooled to 20-25° C. while stirring for at least 10 hours. Subsequently, the mixture was filtered and the collected solids were dried in a vacuum oven at 45° C. to afford crystalline Form IP of Compound (II), an iso-propanol solvate of Compound (II).

An X-ray powder diffractogram of crystalline Form IP of Compound (II) is shown in FIG. 22.

XRPD, C¹³ solid state NMR, and F¹⁹ solid state NMR data for Form IP of Compound (II) are in Tables 18, 19, and 20.

TABLE 18
XRPD data for crystalline Form IP of Compound (II)

| Position | Intensity |
|---|---|
| [°2Theta] | (%) |
| 19.4 | 100.0 |
| 20.6 | 72.5 |
| 19.0 | 71.7 |
| 17.7 | 53.6 |
| 14.6 | 52.3 |
| 18.2 | 52.3 |
| 9.7 | 49.2 |
| 15.0 | 44.7 |
| 15.5 | 44.7 |
| 7.7 | 39.4 |
| 23.6 | 35.2 |
| 21.2 | 34.4 |
| 10.1 | 33.6 |
| 22.3 | 32.2 |
| 26.1 | 27.8 |
| 23.1 | 27.5 |
| 24.2 | 26.8 |
| 21.5 | 23.4 |
| 26.6 | 23.4 |
| 8.0 | 21.2 |
| 28.7 | 20.1 |
| 12.0 | 13.3 |
| 4.9 | 12.6 |
| 30.4 | 11.3 |
| 33.1 | 10.7 |

TABLE 19
C¹³ solid state NMR data for crystalline Form IP of Compound (II)

| Chemical Shift | Intensity |
|---|---|
| [ppm] | [relative] |
| 167.4 | 20.8 |
| 165.3 | 8.0 |
| 162.4 | 46.9 |
| 154.9 | 31.5 |
| 154.2 | 12.9 |
| 141.0 | 38.7 |
| 140.4 | 32.7 |
| 138.4 | 41.5 |
| 135.7 | 44.7 |
| 130.9 | 35.9 |
| 129.8 | 30.2 |
| 114.9 | 29.0 |
| 112.7 | 33.7 |
| 105.7 | 53.4 |
| 99.9 | 27.0 |
| 74.4 | 38.8 |
| 65.0 | 57.1 |
| 64.2 | 38.4 |
| 58.6 | 29.3 |
| 52.1 | 44.1 |
| 31.0 | 48.7 |
| 28.6 | 53.3 |
| 26.9 | 52.4 |
| 25.5 | 52.5 |
| 23.9 | 100.0 |
| 19.2 | 58.0 |
| 18.2 | 57.6 |
| 16.8 | 60.4 |

TABLE 20
F¹⁹ solid state NMR data for crystalline Form IP of Compound (II)

| Chemical Shift | Intensity |
|---|---|
| [ppm] | [relative] |
| −110.6 | 12.5 |

FIG. 23 shows a DSC trace for crystalline Form IP of Compound (II). FIG. 24 shows a TGA plot for crystalline Form IF of Compound (II).

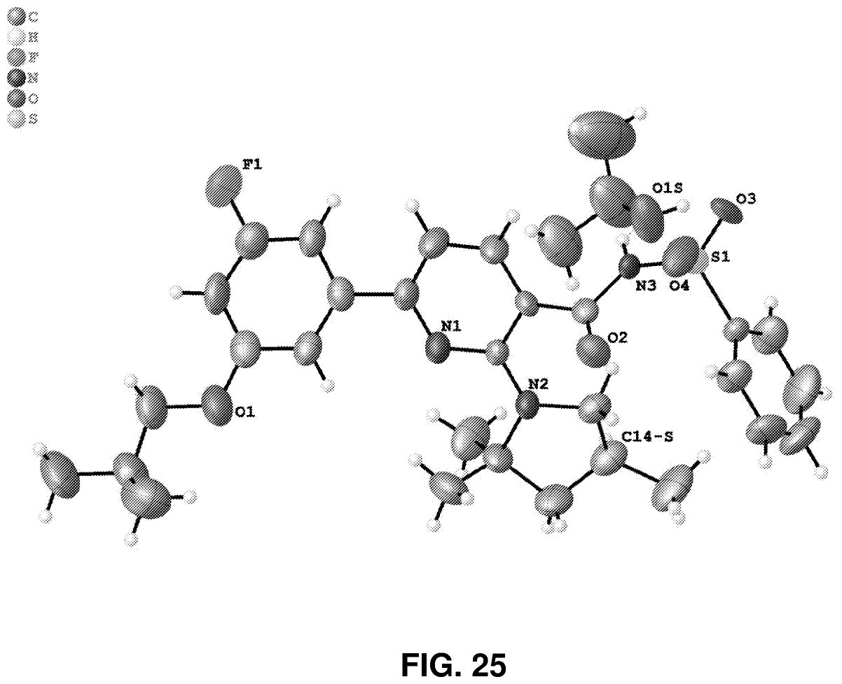

FIG. 25 shows a single crystal structure of crystalline Form IP of Compound (II).

Single crystal structure data for crystalline Form IP of Compound (II) is in Table 21.

TABLE 21
Single crystal structure data for crystalline
Form IP of Compound (II).

| | | |
|---|---|---|
| Empirical formula | C32 H42 F N3 O5 S | |
| Molecular formula | C29 H34 F N3 O4 S, C3 H8 O | |
| Formula weight | 599.74 | |
| Temperature | 296.15 K | |
| Wavelength | 0.71073 Å | |
| Crystal system | Monoclinic | |
| Space group | P 1 21 1 | |
| Unit cell dimensions | a = 11.7883(16) Å | α = 90°. |
| | b = 8.0019(13) Å | β = 104.170(7)°. |
| | c = 18.931(3) Å | γ = 90°. |
| Volume | 1731.4(5) Å³ | |
| Z/Z′ | 2/1 | |
| Density (calculated) | 1.150 Mg/m³ | |

### Preparation of Crystalline Form NPR of Compound (II)

Amorphous Compound (II) was slurried in n-propanol to afford crystalline Form NPR of Compound (II), an n-propanol solvate of Compound (II).

An X-ray powder diffractogram of crystalline Form NPR of Compound (II) is shown in FIG. 26.

XRPD data of crystalline Form NPR of Compound (II) are in Table 22.

TABLE 22
XRPD data for crystalline Form NPR of Compound (II)

| Position | Intensity |
|---|---|
| [°2Theta] | (%) |
| 7.7 | 100.0 |
| 8.0 | 51.7 |
| 19.1 | 50.5 |
| 15.0 | 48.5 |
| 4.8 | 40.2 |
| 20.6 | 37.3 |
| 15.5 | 31.5 |
| 17.7 | 26.3 |
| 12.1 | 23.8 |
| 14.7 | 23.3 |
| 10.1 | 21.2 |
| 18.4 | 20.8 |
| 19.6 | 20.6 |
| 18.3 | 17.9 |
| 9.6 | 16.1 |
| 23.6 | 15.5 |
| 19.4 | 14.6 |
| 24.3 | 14.4 |
| 13.5 | 13.8 |
| 21.6 | 13.5 |
| 21.2 | 11.1 |
| 23.1 | 11.1 |
| 13.7 | 11.0 |
| 26.6 | 10.4 |

### Preparation of Crystalline Form 2B of Compound (II)

A mixture of amorphous Compound (II) (approximately 100 mg) and 2-butanol (1 mL) was stirred for 2 weeks. The solids were collected to afford the title compound crystalline Form 2B of Compound (II), a 2-butanol solvate of Compound (II).

An X-ray powder diffractogram of crystalline Form 2B of Compound (II) is shown in FIG. 27.

XRPD data for crystalline Form 2B of Compound (II) are in Table 23.

TABLE 23
XRPD data for crystalline Form 2B of Compound (II)

| Position | Intensity |
|---|---|
| [°2Theta] | (%) |
| 7.8 | 100.0 |
| 4.7 | 69.1 |
| 15.0 | 47.3 |
| 20.8 | 35.3 |
| 19.2 | 34.4 |
| 17.9 | 31.4 |
| 17.7 | 25.4 |
| 15.6 | 24.2 |
| 15.2 | 23.8 |
| 12.0 | 20.4 |
| 14.5 | 19.7 |
| 18.8 | 18.8 |
| 10.2 | 17.2 |
| 13.6 | 17.2 |
| 9.4 | 17.1 |
| 19.3 | 16.3 |
| 21.0 | 15.2 |
| 23.6 | 12.3 |
| 24.2 | 11.4 |
| 26.5 | 10.6 |
| 14.1 | 10.6 |

FIG. 28 shows a DSC trace for crystalline Form 2B of Compound (II). FIG. 29 shows a TGA plot for crystalline Form 2B of Compound (II).

### Preparation of Crystalline Form MP of Compound (II)

A mixture of amorphous Compound (II) (approximately 100 mg) and 2-methyl-1-propanol (1 mL) was stirred for 2 weeks. The solids were collected to afford crystalline Form MP of Compound (II), a 2-methyl-1-propanol solvate of Compound (II).

An X-ray powder diffractogram of crystalline Form MP of Compound (II) is shown in FIG. 30.

XRPD data for crystalline Form MP of Compound (II) are in Table 24.

TABLE 24
XRPD data for crystalline Form MP of Compound (II)

| Position | Intensity |
|---|---|
| [°2Theta] | (%) |
| 7.8 | 100.0 |
| 19.2 | 47.4 |
| 4.7 | 45.6 |
| 15.0 | 43.7 |
| 20.7 | 38.1 |
| 8.1 | 31.3 |
| 17.6 | 26.1 |
| 15.4 | 25.9 |
| 12.0 | 24.5 |
| 15.7 | 23.2 |
| 18.0 | 23.0 |
| 10.1 | 23.0 |
| 19.0 | 21.4 |
| 9.5 | 19.0 |
| 24.1 | 18.4 |
| 13.7 | 18.2 |
| 14.6 | 17.1 |
| 19.6 | 15.8 |
| 14.4 | 15.0 |
| 21.1 | 14.4 |
| 21.5 | 13.8 |
| 18.2 | 12.7 |
| 23.6 | 12.2 |
| 23.2 | 12.0 |
| 24.7 | 11.8 |
| 26.4 | 11.3 |
| 13.5 | 10.1 |

FIG. 31 shows a DSC trace for crystalline Form MP of Compound (II). FIG. 32 shows a TGA plot for crystalline Form MP of Compound (II).

### Preparation of Crystalline Form NP of Compound (II)

A mixture of amorphous Compound (II) (approximately 100 mg) and pentanol (0.5 mL) was stirred for 2 weeks. The solids were collected to afford crystalline Form NP of Compound (II), an n-pentanol solvate of Compound (II).

An X-ray powder diffractogram of crystalline Form NP of Compound (II) is shown in FIG. 33.

XRPD data for crystalline Form NP of Compound (II) are in Table 25.

TABLE 25
XRPD data for crystalline Form NP of Compound (II)

| Position | Intensity |
|---|---|
| [°2Theta] | (%) |
| 7.8 | 100.0 |
| 15.2 | 82.1 |
| 13.5 | 70.8 |
| 21.0 | 69.0 |
| 19.0 | 48.9 |
| 14.4 | 47.4 |
| 20.5 | 46.3 |
| 13.6 | 43.5 |
| 19.3 | 43.5 |
| 24.2 | 43.5 |
| 7.7 | 35.3 |
| 18.6 | 34.4 |
| 22.3 | 33.0 |
| 4.6 | 32.6 |
| 17.9 | 32.4 |
| 15.8 | 30.8 |
| 18.5 | 27.1 |
| 14.9 | 26.5 |
| 14.9 | 25.2 |
| 21.2 | 22.1 |
| 17.5 | 21.8 |
| 26.4 | 21.0 |
| 24.5 | 19.1 |
| 23.7 | 14.4 |
| 21.6 | 13.9 |

FIG. 34 shows a DSC trace for crystalline Form NP of Compound (II). FIG. 35 shows a TGA plot for crystalline Form NP of Compound (II).

### Preparation of Crystalline Form EE of Compound (II)

A mixture of amorphous Compound (II) (approximately 100 mg) and 2-ethoxyethanol (0.94 mL) was stirred for 3 weeks. The solids were collected to afford crystalline Form EE of Compound (II), a 2-ethoxyethanol solvate of Compound (II).

An X-ray powder diffractogram of crystalline Form EE of Compound (II) is shown in FIG. 36.

XRPD data for crystalline Form EE of Compound (II) are in Table 26.

TABLE 26
XRPD data for crystalline Form EE of Compound (II)

| Position | Intensity |
|---|---|
| [°2Theta] | (%) |
| 7.9 | 100.0 |
| 15.3 | 44.6 |
| 13.6 | 44.0 |
| 13.5 | 38.5 |
| 4.6 | 32.9 |
| 19.4 | 31.5 |
| 18.7 | 31.0 |
| 19.1 | 27.7 |
| 21.2 | 27.4 |
| 7.7 | 25.7 |
| 18.0 | 25.0 |
| 15.9 | 23.6 |
| 14.5 | 20.2 |
| 24.1 | 19.2 |
| 20.6 | 18.2 |
| 22.2 | 13.8 |
| 26.4 | 13.1 |
| 14.9 | 12.6 |
| 24.4 | 11.2 |

FIG. 37 shows a DSC trace for crystalline Form EE of Compound (II). FIG. 38 shows a TGA plot for crystalline Form EE of Compound (II).

### Preparation of Crystalline Form E of Compound (II)

A mixture of amorphous Compound (II) (approximately 200 mg) and ethanol (200 proof; about 2-3 mL) was stirred for 24 hours. The solids were collected to afford crystalline Form E of Compound (II), an ethanol solvate of Compound (II).

An X-ray powder diffractogram of crystalline Form E of Compound (II) is shown in FIG. 39.

XRPD data for crystalline Form E of Compound (II) are in Table 27.

TABLE 27
XRPD data for crystalline Form E of Compound (II)

| Position | Intensity |
|---|---|
| [°2Theta] | (%) |
| 14.4 | 100.0 |
| 23.7 | 95.5 |
| 19.2 | 74.8 |
| 7.8 | 65.0 |
| 11.7 | 61.5 |
| 8.7 | 56.0 |
| 11.6 | 54.0 |
| 22.1 | 52.6 |
| 13.8 | 51.6 |
| 20.0 | 42.9 |
| 18.0 | 39.9 |
| 20.5 | 36.3 |
| 9.6 | 35.4 |
| 13.3 | 32.3 |
| 18.9 | 29.9 |
| 21.6 | 27.8 |
| 16.9 | 25.2 |
| 26.7 | 22.6 |
| 24.1 | 19.5 |
| 15.9 | 17.9 |
| 17.4 | 17.3 |
| 22.7 | 17.1 |
| 25.3 | 17.1 |
| 23.3 | 16.3 |
| 4.7 | 13.7 |
| 22.5 | 12.7 |
| 26.4 | 12.2 |
| 27.5 | 11.6 |
| 13.0 | 10.5 |
| 25.9 | 10.3 |

### Preparation of Crystalline Form T of Compound (II)

A saturated mixture of amorphous Compound (II) and tetrahydrofuran (2 mL) was stirred overnight. The solution turned into a suspension and was stirred for an additional 5 weeks. The solids were collected via filtration to afford crystalline Form T of Compound (II), a tetrahydrofuran solvate of Compound (II).

An X-ray powder diffractogram of crystalline Form T of Compound (II) is shown in FIG. 40.

XRPD data for crystalline Form T of Compound (II) are in Table 28.

TABLE 28
XRPD data for crystalline Form T of Compound (II)

| Position | Intensity |
|---|---|
| [°2Theta] | (%) |
| 19.0 | 100.0 |
| 18.1 | 96.1 |
| 15.0 | 95.5 |
| 20.9 | 92.2 |
| 7.9 | 79.5 |
| 15.7 | 61.4 |
| 20.2 | 57.5 |
| 23.8 | 50.6 |
| 18.5 | 43.1 |
| 26.3 | 43.0 |
| 14.6 | 39.9 |
| 22.0 | 36.7 |
| 22.9 | 36.1 |
| 19.2 | 27.5 |
| 10.1 | 26.3 |
| 10.6 | 25.5 |
| 23.6 | 24.0 |
| 25.9 | 20.7 |
| 11.8 | 16.9 |
| 5.1 | 16.7 |
| 24.9 | 15.3 |

### Preparation of Crystalline Form AC of Compound (II)

A mixture of crystalline Form A of Compound (II) (approximately 200 mg) and acetonitrile (0.5 mL) was cooled to 5° C. for four days, during which time solids formed. The solvent was removed by decanting it, and the collected solids were dried in vacuo at room temperature to afford crystalline Form AC of Compound (II), an acetonitrile solvate of Compound (II).

An X-ray powder diffractogram of crystalline Form AC of Compound (II) is shown in FIG. 41.

XRPD data for crystalline Form AC of Compound (II) are in Table 29.

TABLE 29
XRPD data for crystalline Form AC of Compound (II)

| Position | Intensity |
|---|---|
| [°2Theta] | (%) |
| 13.1 | 100.0 |
| 6.5 | 16.2 |
| 19.7 | 14.7 |
| 19.4 | 7.0 |
| 9.9 | 6.1 |
| 12.9 | 5.2 |
| 9.8 | 2.9 |
| 17.1 | 1.5 |
| 23.0 | 1.3 |
| 33.2 | 1.2 |
| 25.5 | 1.2 |
| 22.6 | 1.2 |
| 17.5 | 1.0 |

FIG. 42 shows a DSC trace for crystalline Form AC of Compound (II). FIG. 43 shows a TGA plot for crystalline Form AC of Compound (II).

### Example 8

### Preparation of Crystalline Forms C of Compound (I)

A flask of crude reaction mixture was charged with isopropylacetate (136 mL, 12 vol.). The slurry was heated to dissolution and then stirred at 70° C. for about 15 minutes. A seed of Form A of Compound (I) was added to the mixture and the resulting slurry was stirred for 2 hours. Heptane (136 mL, 12 vol.) was added over a 3 hour period. The resulting slurry was cooled to 20° C. over 5 hours. The resulting solids were filtered, the wet cake washed with 4 volumes of a 1:1 mixture of isopropylacetate-heptane. The wet cake was air dried on the filter for at least 30 minutes and then the collected solids were dried in vacuo at at 40° C. overnight to afford crystalline Forms C of Compound (I), a mixture of crystalline forms of Compound (I).

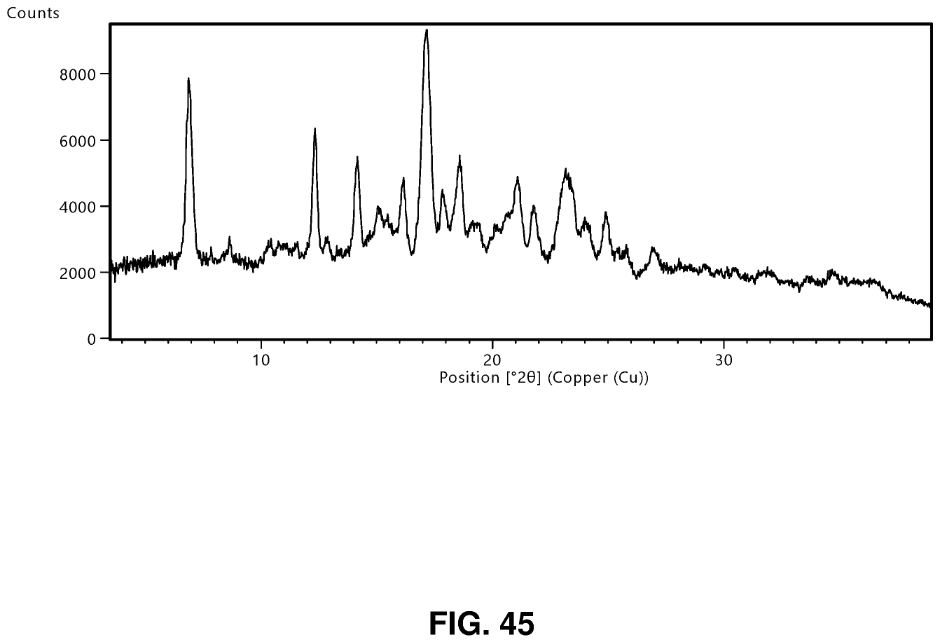

An X-ray powder diffractogram of crystalline Forms C of Compound (I) is shown in FIG. 45.

XRPD, C¹³ solid state NMR, and F¹⁹ solid state NMR data for Forms C of Compound (I) are in Tables 30, 31, and 32.

TABLE 30
XRPD data for crystalline Forms C of Compound (I)

| Position | Intensity |
|---|---|
| [°2Theta] | (%) |
| 17.2 | 100.0 |
| 6.9 | 85.4 |
| 12.4 | 63.8 |
| 14.2 | 53.6 |
| 18.6 | 47.6 |
| 23.1 | 46.5 |
| 16.2 | 46.5 |
| 21.1 | 44.7 |
| 17.8 | 40.0 |
| 15.1 | 35.0 |
| 21.8 | 34.4 |
| 24.9 | 33.8 |
| 24.0 | 29.1 |
| 19.4 | 28.9 |
| 20.1 | 27.8 |
| 8.7 | 24.3 |
| 12.9 | 22.6 |
| 10.8 | 21.8 |
| 10.4 | 21.5 |
| 11.6 | 20.9 |
| 7.9 | 20.1 |
| 26.9 | 18.9 |
| 25.8 | 18.9 |
| 25.4 | 18.6 |
| 30.5 | 11.5 |
| 34.7 | 10.4 |

TABLE 31
C¹³ solid state NMR data for crystalline Forms C of Compound (I)

| Chemical Shift | Intensity |
|---|---|
| [ppm] | [relative] |
| 166.7 | 8 |
| 165.3 | 34 |
| 164.2 | 14.2 |
| 162.8 | 39.3 |
| 161.3 | 55.6 |
| 160.5 | 71 |
| 156 | 37.5 |
| 155.2 | 27.1 |
| 154.3 | 23.9 |
| 153.1 | 40.8 |
| 152.8 | 42.9 |
| 152.2 | 33.8 |
| 142.6 | 38.9 |
| 142 | 38.7 |
| 140.8 | 47.7 |
| 139.2 | 50.6 |
| 138.7 | 50.2 |
| 117.1 | 34.2 |
| 116.4 | 71.9 |
| 113.5 | 33.5 |
| 109.7 | 79.6 |
| 108.6 | 34.7 |
| 107.3 | 30.9 |
| 105.7 | 100 |
| 104.6 | 25.9 |
| 75.6 | 53.5 |
| 74.4 | 9.7 |
| 73.2 | 40.6 |
| 66.2 | 47.9 |
| 64.4 | 53.3 |
| 60.8 | 37.9 |
| 60 | 40.3 |
| 51.2 | 54.1 |
| 50.8 | 41.4 |
| 30.5 | 73.7 |
| 30.3 | 76.5 |
| 29.2 | 65.7 |
| 28.9 | 73.4 |
| 27.5 | 61.4 |
| 26.5 | 58 |
| 26.1 | 75.1 |
| 25.8 | 81.9 |
| 19.6 | 70 |
| 19.3 | 63.8 |
| 17.7 | 99.1 |
| 17.1 | 67 |
| 15.8 | 61.1 |

TABLE 32
F¹⁹ solid state NMR data for crystalline Forms C of Compound (I)

| Chemical Shift | Intensity |
|---|---|
| [ppm] | [relative] |
| −109.3 | 3.1 |
| −110.8 | 4.3 |
| −111.7 | 3 |
| −113.3 | 12.5 |

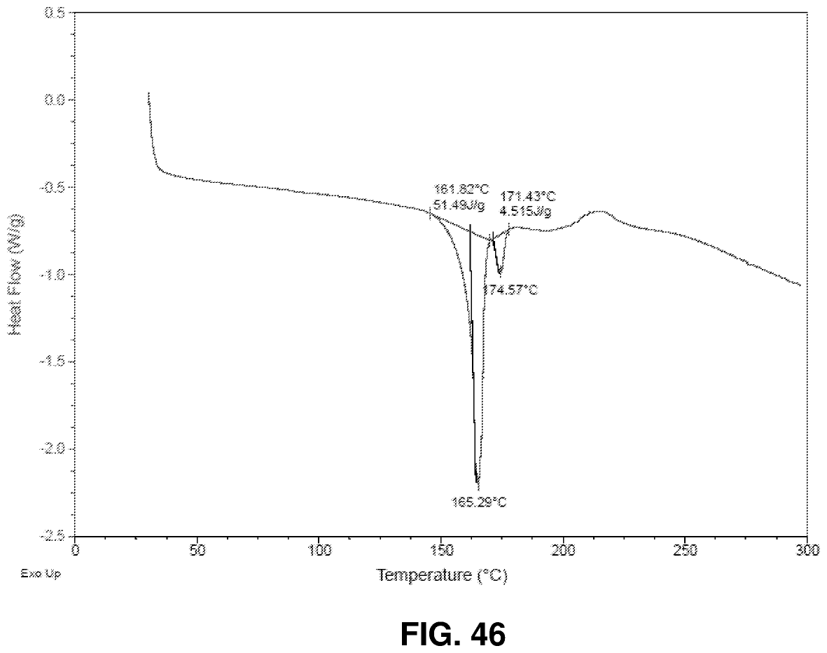

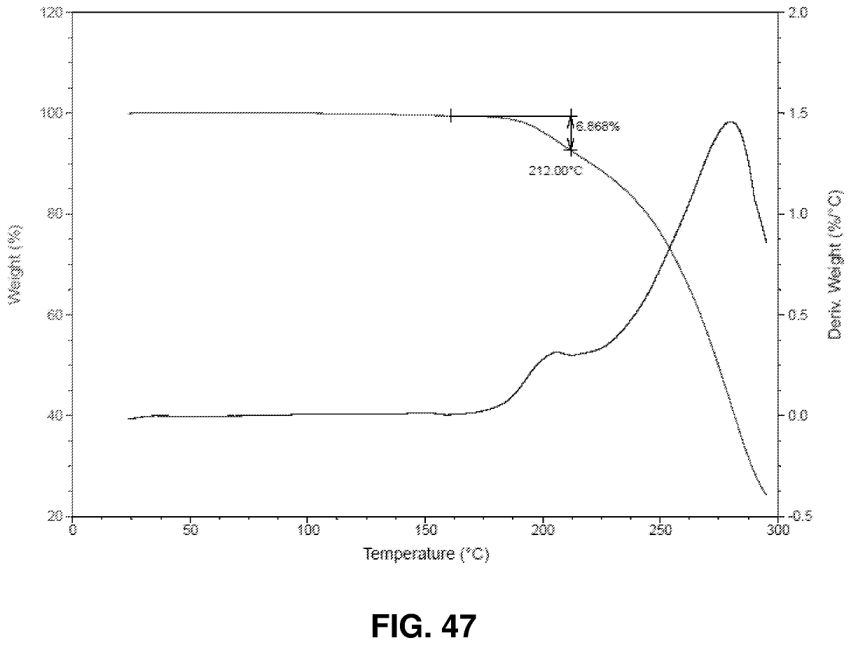

FIG. 46 shows a DSC trace for crystalline Forms C of Compound (I). FIG. 47 shows a TGA plot for crystalline Forms C of Compound (I).

### Preparation of Crystalline Forms FC of Compound (I)

Solid spray-dried dispersions of Compound (I) with HPMCAS-H (50:50, 80:20, 90:10) were stored at elevated temperature and humidity (for example 80° C./75% RH) under open dish conditions to afford Forced Crystallized Forms (FC) of Compound (I).

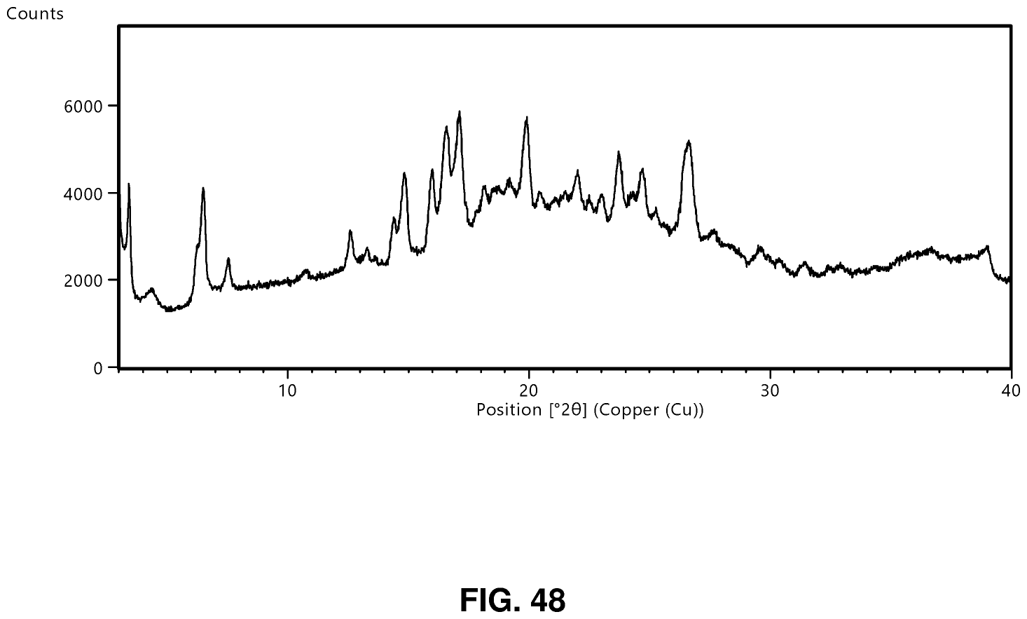

An X-ray powder diffractogram of crystalline Forms FC of Compound (I) is shown in FIG. 48.

XRPD, C¹³ solid state NMR, and F¹⁹ solid state NMR data for Forms FC of Compound (I) are in Tables 33, 34, and 35.

TABLE 33
XRPD data for crystalline Forms FC of Compound (I)

| Position | Intensity |
|---|---|
| [°2Theta] | (%) |
| 17.1 | 100.0 |
| 19.9 | 95.6 |
| 16.6 | 89.9 |
| 26.7 | 82.3 |
| 23.7 | 76.6 |
| 3.4 | 73.2 |
| 24.7 | 66.0 |
| 6.5 | 65.7 |
| 16.0 | 65.4 |
| 14.8 | 65.0 |
| 22.0 | 64.9 |
| 26.4 | 64.8 |
| 19.1 | 60.2 |
| 18.1 | 56.9 |
| 20.5 | 53.9 |
| 23.0 | 52.0 |
| 22.5 | 49.3 |
| 25.3 | 42.6 |
| 14.4 | 37.6 |
| 6.2 | 33.0 |
| 27.7 | 31.3 |
| 12.6 | 30.2 |
| 7.5 | 21.1 |
| 13.3 | 20.4 |
| 29.6 | 20.4 |
| 4.4 | 11.2 |

TABLE 34
C¹³ solid state NMR data for crystalline
Forms FC of Compound (I)

| Chemical Shift | Intensity |
|---|---|
| [ppm] | [relative] |
| 166.5 | 15.9 |
| 164.3 | 22.3 |
| 161.6 | 42.8 |
| 160.4 | 34.4 |
| 159.1 | 26.2 |
| 155.4 | 30.3 |
| 152.5 | 39.7 |
| 140.3 | 47.6 |
| 138.1 | 45.6 |
| 116.3 | 36.9 |
| 114.1 | 36.1 |
| 111.3 | 27.8 |
| 109.9 | 34.7 |
| 107.3 | 36.1 |
| 105.8 | 32.6 |
| 102.7 | 35.7 |
| 99.1 | 11.6 |
| 73.6 | 58.3 |
| 64.8 | 41.0 |
| 59.4 | 30.3 |
| 56.7 | 14.1 |
| 56.0 | 19.7 |
| 53.5 | 55.1 |
| 30.9 | 55.1 |
| 29.1 | 80.8 |
| 28.1 | 41.6 |
| 27.4 | 31.1 |
| 26.0 | 25.3 |
| 25.2 | 20.5 |
| 24.4 | 33.2 |
| 19.4 | 100.0 |

TABLE 35
F¹⁹ solid state NMR data for crystalline Form B of Compound (I)

| Chemical Shift | Intensity |
|---|---|
| [ppm] | [relative] |
| −110.3 | 9.7 |
| −111.7 | 12.5 |

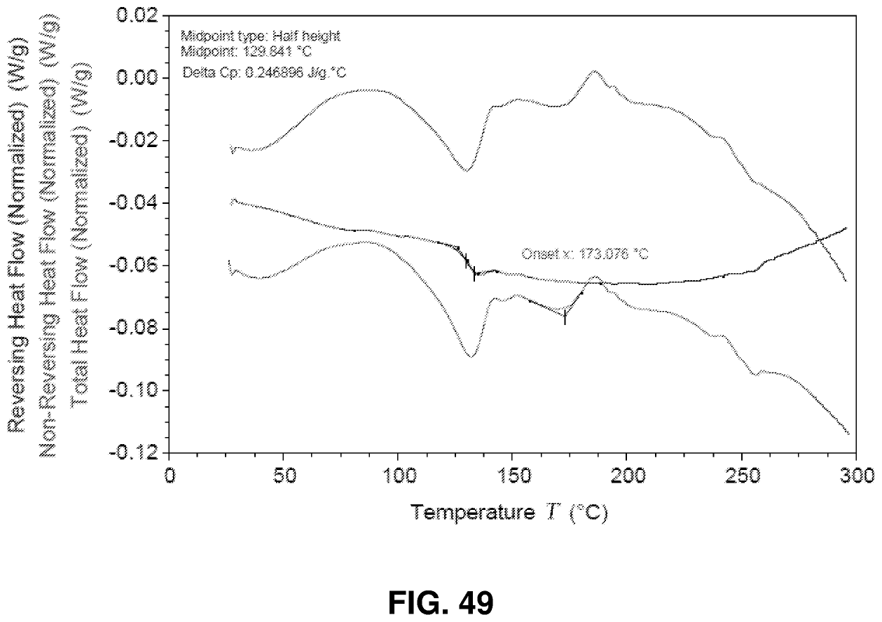

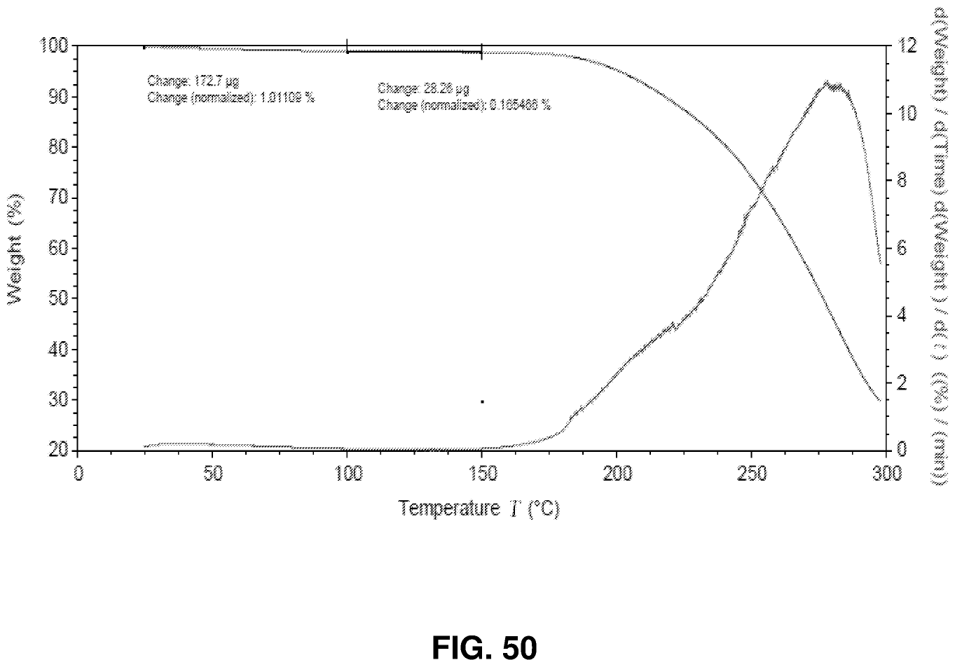

FIG. 49 shows a DSC trace for crystalline Forms FC of Compound (I). FIG. 50 shows a TGA plot for crystalline Forms FC of Compound (I).

### Amorphous Compound (I)

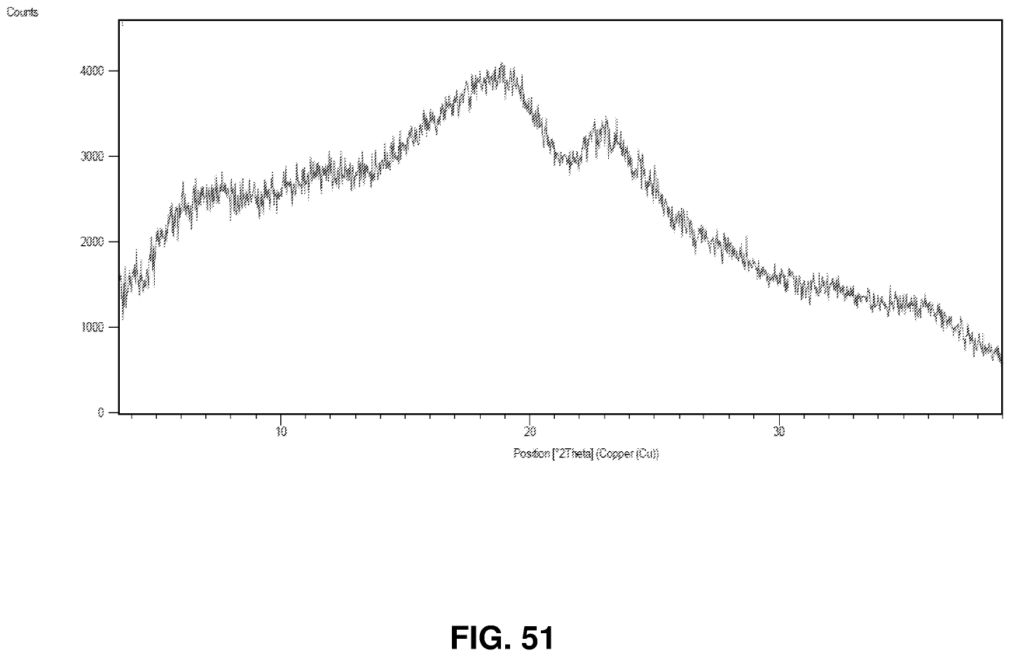

An X-ray powder diffractogram of amorphous Compound (I) is shown in FIG. 51.

C¹³ solid state NMR and F¹⁹ solid state NMR data for amorphous Compound (I) are in Tables 36 and 37.

TABLE 36
C¹³ solid state NMR data for amorphous Compound (I)

| Chemical Shift | Intensity |
|---|---|
| [ppm] | [relative] |
| 165.2 | 28.7 |
| 161.1 | 61 |
| 153.8 | 40.8 |
| 140 | 44.7 |
| 124 | 11 |
| 112.7 | 45.9 |
| 106 | 64.3 |
| 75.1 | 45.4 |
| 64.5 | 36.8 |
| 59 | 33.7 |
| 52.1 | 25.7 |
| 30.8 | 47.7 |
| 28.7 | 78.9 |
| 19.3 | 100 |
| 165.2 | 28.7 |
| 161.1 | 61 |
| 153.8 | 40.8 |
| 140 | 44.7 |
| 124 | 11 |
| 112.7 | 45.9 |
| 106 | 64.3 |
| 75.1 | 45.4 |
| 64.5 | 36.8 |
| 59 | 33.7 |
| 52.1 | 25.7 |
| 30.8 | 47.7 |

TABLE 37
F¹⁹ solid state NMR data for amorphous Compound (I)

| Chemical Shift | Intensity |
|---|---|
| [ppm] | [relative] |
| −111.5 | 12.5 |

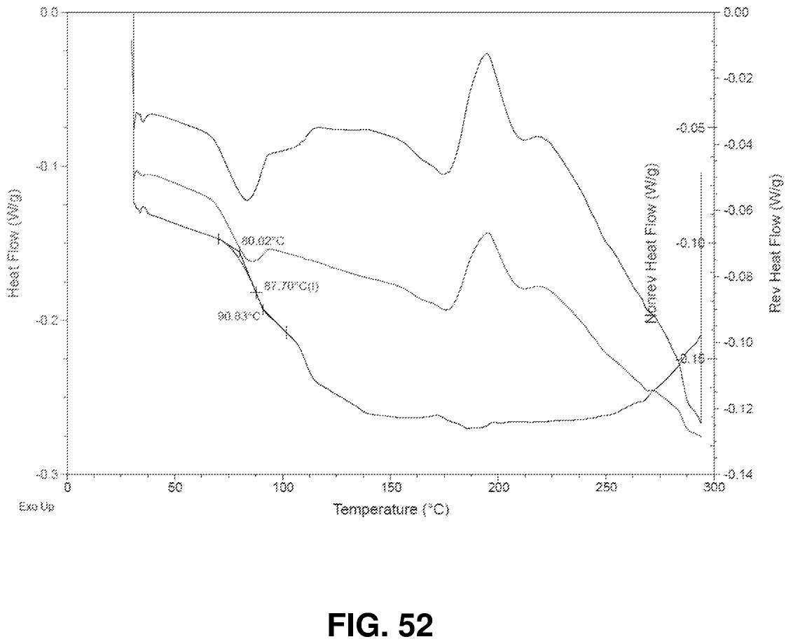

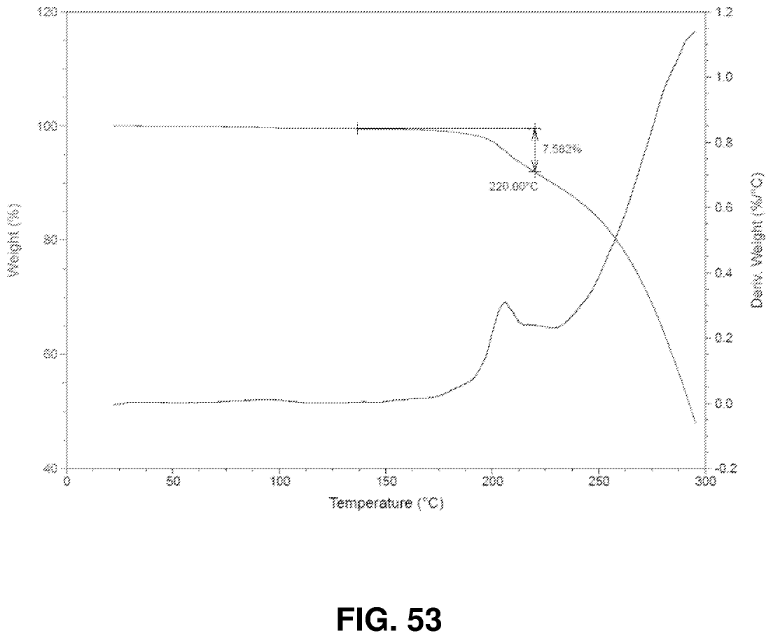

FIG. 52 shows a DSC trace for amorphous Compound (I). FIG. 53 shows a TGA plot for amorphous Compound (I).

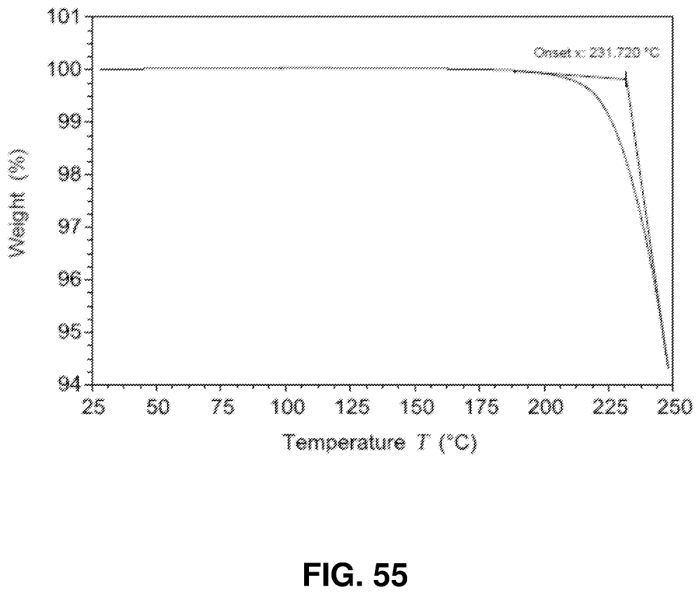

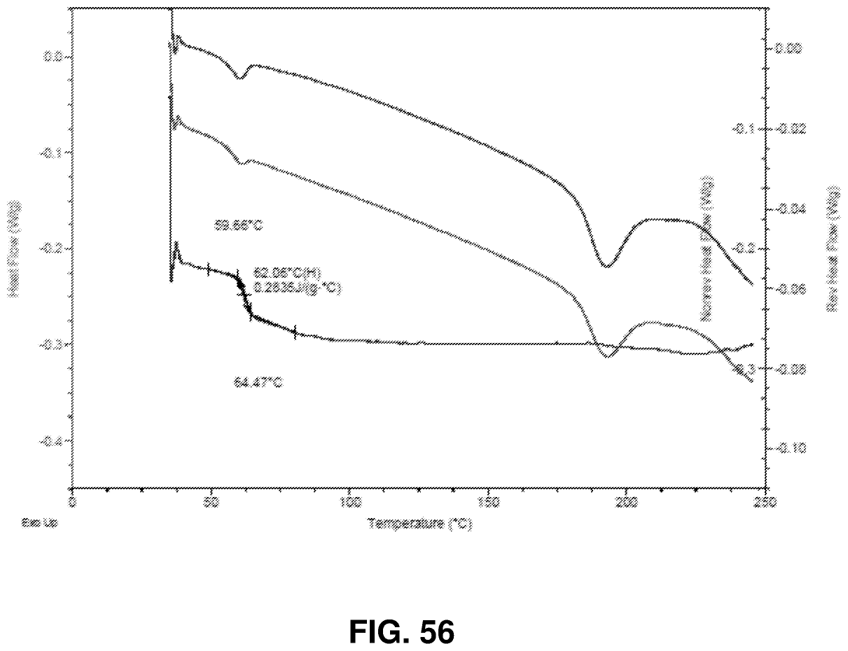

FIG. 55 shows a DSC trace for amorphous of Compound (II). FIG. 56 shows a TGA plot amorphous of Compound (II)

### Amorphous Compound (II)

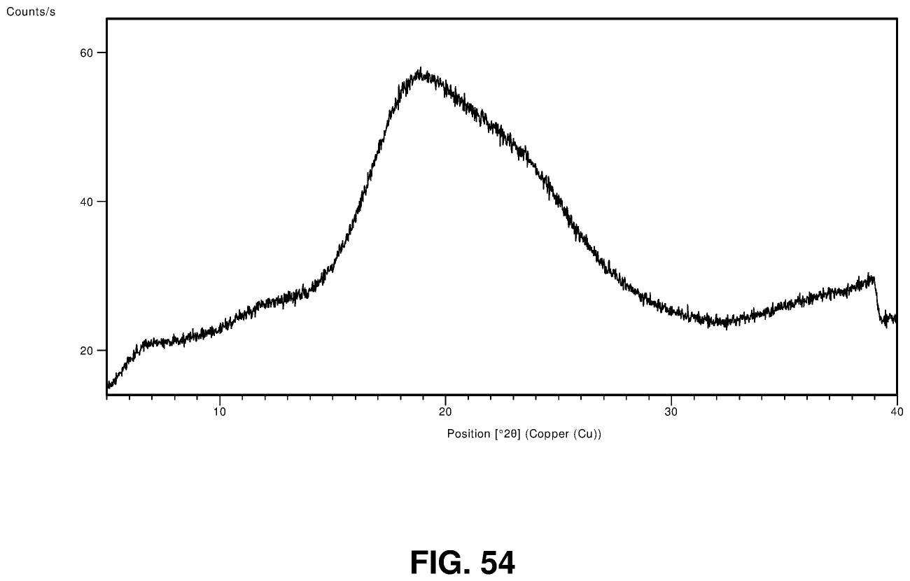

An X-ray powder diffractogram of amorphous Compound (II) is shown in FIG. 54.

C¹³ solid state NMR and F¹⁹ solid state NMR data for amorphous Compound (II) are in Tables 37 and 38.

TABLE 37
C¹³ solid state NMR data for amorphous Compound (II)

| Chemical Shift | Intensity |
|---|---|
| [ppm] | [relative] |
| 165.1 | 30.0 |
| 161.2 | 45.2 |
| 153.6 | 33.5 |
| 140.1 | 53.1 |
| 133.2 | 25.8 |
| 128.7 | 70.5 |
| 111.6 | 25.7 |
| 105.8 | 72.3 |
| 74.8 | 44.2 |
| 64.2 | 45.9 |
| 58.4 | 36.6 |
| 52.0 | 31.2 |
| 30.4 | 54.8 |
| 28.6 | 81.4 |
| 26.5 | 64.0 |
| 19.3 | 100.0 |

TABLE 38
F¹⁹ solid state NMR data for amorphous Compound (II)

| Chemical Shift | Intensity |
|---|---|
| [ppm] | [relative] |
| −111.6 | 12.5 |

FIG. 55 shows a DSC trace for amorphous Compound (II). FIG. 56 shows a TGA plot for amorphous Compound (II).

### OTHER EMBODIMENTS

The foregoing discussion discloses and describes merely exemplary embodiments of this disclosure. One skilled in the art will readily recognize from such discussion and from the accompanying drawings and claims, that various changes, modifications and variations can be made therein without departing from the spirit and scope of this disclosure as defined in the following claims.

1.-73. (canceled)

74. A crystalline form of Compound (I): 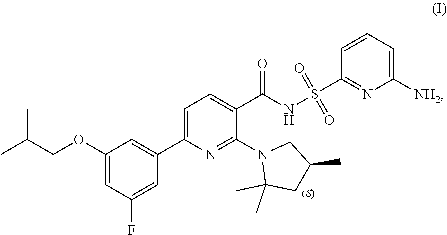
selected from crystalline Form A, crystalline Form B, crystalline Form H, and crystalline Form S.

75. The crystalline form of claim 74, wherein the crystalline form is substantially pure.

76. The crystalline form of claim 75, wherein at least 90% by weight of the crystalline form is crystalline Form A, crystalline Form B, crystalline Form H, or crystalline Form S.

77. The crystalline form of claim 74, as characterized by an X-ray powder diffractogram (XRPD).

78. A method for preparing the crystalline form of claim 74, wherein the method comprises:
(a) crystallizing Compound (I) from a mixture of ethanol, water, and Compound (I) to form crystalline Form A;
(b) crystallizing Compound (I) from a mixture of isopropylacetate and Compound (I) to form crystalline Form B;
(c) crystallizing Compound (I) from a mixture of methanol, water, and Compound (I) to form crystalline Form H; or
(d) crystallizing Compound (I) from a mixture of 1,4-dioxane, heptane, and Compound (I) to form crystalline Form S.

79. A crystalline form of Compound (II): 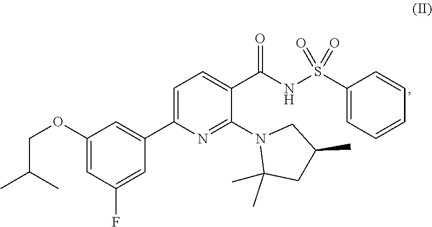 [SMILES: CC(C)COc1cc(F)cc(-c2ccc(C(=O)NS(=O)(=O)c3ccccc3)c(N3C[C@@H](C)CC3(C)C)n2)c1]
selected from crystalline Form A2, crystalline Form IP, crystalline Form NPR, crystalline Form 2B, crystalline Form MP, crystalline Form NP, crystalline Form EE, crystalline Form E, crystalline Form T, and crystalline Form AC.

80. The crystalline form of claim 79, wherein the crystalline form is substantially pure.

81. The crystalline form of claim 79, wherein at least 90% by weight of the crystalline form is crystalline Form A2, crystalline Form IP, crystalline Form NPR, crystalline Form 2B, crystalline Form MP, crystalline Form NP, crystalline Form EE, crystalline Form E, crystalline Form T, or crystalline Form AC.

82. The crystalline form of claim 79, as characterized by an X-ray powder diffractogram (XRPD).

83. A method of preparing the crystalline form of claim 79, comprising:
(a) crystallizing Compound (II) from a mixture of ethanol, water, and Compound (II) to form crystalline Form A2;
(b) crystallizing Compound (II) from a mixture of isopropanol and Compound (II) to form crystalline Form IP;
(c) crystallizing Compound (II) from a mixture of n-propanol and Compound (II) to form crystalline Form NPR;
(d) crystallizing Compound (II) from a mixture of 2-butanol and Compound (II) to form crystalline Form 2B;
(e) crystallizing Compound (II) from a mixture of 2-methyl-1-propanol and Compound (II) to form crystalline Form MP;
(f) crystallizing Compound (II) from a mixture of n-pentanol and Compound (II) to form crystalline Form NP;
(g) crystallizing Compound (II) from a mixture of 2-ethoxyethanol and Compound (II) to form crystalline Form EE;
(h) crystallizing Compound (II) from a mixture of ethanol and Compound (II) to form crystalline Form E;
(i) crystallizing Compound (II) from a mixture of tetrahydrofuran and Compound (II) to form crystalline Form T; or
(j) crystallizing Compound (II) from a mixture of acetonitrile and Compound (II) to form crystalline Form AC.

84. At least one solvate of Compound (II): 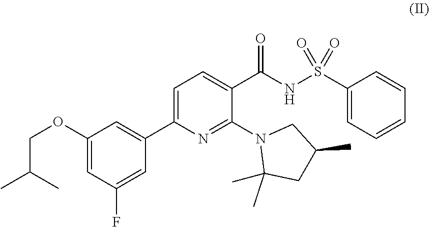 [SMILES: CC(C)COc1cc(F)cc(-c2ccc(C(=O)NS(=O)(=O)c3ccccc3)c(N3C[C@@H](C)CC3(C)C)n2)c1]
chosen from iso-propanol solvates, n-propanol solvates, butanol solvates, and 2-methyl-1-propanol solvates, pentanol solvates, tetrahydrofuran solvates, ethanol solvates, acetonitrile solvates, and 2-ethoxyethanol solvates of Compound (II).

85. A pharmaceutical formulation comprising at least one crystalline form of claim 74 and a pharmaceutically acceptable carrier.

86. A pharmaceutical formulation comprising at least one crystalline form of claim 79 and a pharmaceutically acceptable carrier.

87. A pharmaceutical formulation comprising at least one solvate of claim 84 and a pharmaceutically acceptable carrier.

88. A method of treating cystic fibrosis comprising administering to a patient in need thereof at least one crystalline form of claim 74.

89. A method of treating cystic fibrosis comprising administering to a patient in need thereof at least one crystalline form of claim 79.

90. A method of treating cystic fibrosis comprising administering to a patient in need thereof at least one solvate of claim 84.
